SEQUENCE LISTING (24 sequences)

SEQ ID NO: 1 - protein, 8 aa, Artificial
  Consensus Amino Acid Sequence
PGECCPLP

SEQ ID NO: 2 - protein, 1036 aa, Homo sapiens
MYLVAGDRGLAGCGHLLVSLLGLLLLLARSGTRALVCLPCDESKCEEPRNCPGSIVQGVCGCCYTCASQRNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPPVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTGKDCINGFKRDHNGCRTCQCINTEELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIEDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

SEQ ID NO: 3 - protein, 1037 aa, Mus musculus
MYLVAGGRGLAGCGHLSVSLLGLLLLLARSGTRALVCLPCDESKCEEPRSCPGSIVQGVCGCCYMCARQRNESCGGAYGLHGACDRGLRCVIRPPLNGDSITEYEVGVCEDEDWDDDQLIGFEPCNENLISGCNIINGKCECGTIRTCNNPFEFPRKDMCLSALKRIEEEKPDCSKARCEVRFSPRCPEDSILIEGYAPPGECCPLPSRCVCDPAGCLRKVCQPGYLNILVSKASGKPGECCDLYECKPVFSVDCSTVECPPVQQAVCPLDSYETQVRLTADGCCTLPARCECLSGLCGFPVCEVGSTPRIVSRGDGTPGKCCDVFECVNETKPACVFNSVEYYDGDMFRMDNCRFCRCQGGVSICFTAQCGELNCERYYVPEGECCPVCEDPIYPLNNPAGCYANGQIRAHGDRWREDDCTFCQCINGEPHCVATACGQSCMHPVKVPGECCPVCEEPTYITIDPPACGELSNCSLKEKDCVYGFKLDHNGCRTCQCKIREELCLGLKRACTLDCPFGFLTDVHNCELCQCRPRPKKCRPTMCDKFCPLGFLKNKHGCDICRCKKCPELPCSKICPLGFQQDSHGCLICKCREVPPSAGPPVLSGTCLSMDGHHHKNEESWHDGCRECYCHNGKEMCALITCPVPACGNPTIRSGQCCPSCTDDFVVQKPELSTPSICHAPGGEYFVEGETWNIDSCTQCTCHSGRVLCETEVCPPLLCQNPSRTQDSCCPQCTDDPPQPSTSHNESVPSYCRNDEGDIFLAAESWKPDACTSCVCVDSAISCYSESCPSVACERPVLRKGQCCPYCLEDTIPKKVVCHFSGKTYADEERWDIDSCTHCYCLQGQTLCSTVSCPPLPCAEPIKVEGSCCPMCPEMYVPEPTNVPIEKKNHRGEIDLEVPMWPTPSENDIIHLPRDMGHLQVDYRDNNRLHPGEDSSLDSIVSVVVPIIICLSIIIAFLLINQKKQWVPLLCWYRTPTKPSSLNNQLVSVDCKKGTRVQVDGPQRMLRIAEPDARFSGFYSMQKQNHLQADNFYQTV

SEQ ID NO: 4 - protein, 1048 aa, Gallus gallus
MYLAAVSAGRRRPGGDGGGGGGGWHLAAAGWLLLLALLLGQPGTRALVCLPCDESKCEEPKSCPGIIVLGICGCCFMCARQRNESCGGVYGLHGACDRGLRCVIRPPLNGDSITEYEVGVCEDENWDDDQLLGFEPCNENLITGCNIINGKCDCDTIRTCNNPFEFPSRDTCLSALKRIEEEKPDCSKARCEVQFSPRCPEDSILIEGYAPPGECCPLPSRCVCNPAGCLRKVCQPGYLNILVSKASGKPGECCDLYECKPVFSVDCSTVECPPVQQVVCPLDSYETQVRLTADGCCTLPTRCECLSGLCGFPMCEAGSVPQIVSRGDGTPGKCCDVFECVNEVKPTCIFNSMEYYDGDMFRMDACRFCRCQGGVSICFSAQCGELHCDRYYVPEGECCPVCEDPVYPVNNPAGCYANGQIQAHGDRWREDDCTFCQCINGNPHCVATACGQSCLNPVKVPGECCPVCEEPTYITIGPPTCELLVNCTLTEKDCIYSFKLDQNGCRICQCKTREELCTGLISGCSLDCSFGFQTDAHNCEICQCRPRPKKCKPIVCDKYCPFGYLKNKHGCEICRCKKCPEMPCGKICPMGFQQNSHGCVICKCREATASLMPPVKTGSCLSMDGRRHENEESWHDGCRECYCHNGREMCALITCPVPNCGNPTIHPGQCCPSCPDEIIVQKPELTSPSICHAPGGEYFVEGETWNIDSCTQCTCHSGRVLCETEVCPPLLCQNPTRTQDSCCPQCPDEPLQPSLSSNVSMPSYCKNDEGDIFLTAESWKPNVCTSCICMDGVIRCYSESCPPVSCERPVLRKGQCCPYCIEDTVPKKVVCHFNGKTYADEERWDIDSCTHCYCLQGQTLCSTVSCPPLPCAEPINVEGSCCPMCPEMYVPEPTNIPIEKTNHRGDVELEVPNWSTPSENDIIHIHRDMNHLQGEYRSGNGPHPSEDASVSSVALVTVPITIALLVIIVFLLINQKKQWIPVSCYKAPTKPSCLNNQLVYVDCKKGTMVQVDSSQRMLRIADPDSRYSGFYSMQKQNNLQADNFYQTV

SEQ ID NO: 5 - DNA, 5601 bp, Homo sapiens
ggcccggctgcgaggaggaggcggcggcggcgcaggaggatgtacttggtggcgggggacagggggttggccggctgcgggcacctcctggtctcgctgctggggctgctgctgctgctggcgcgctccggcacccgggcgctggtctgcctgccctgtgacgagtccaagtgcgaggagcccaggaactgcccggggagcatcgtgcagggcgtctgcggctgctgctacacgtgcgccagccagaggaacgagagctgcggcggcaccttcgggatttacggaacctgcgaccgggggctgcgttgtgtcatccgccccccgctcaatggcgactccctcaccgagtacgaagcgggcgtttgcgaagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccagttttcggcgtggactgcaggactgtggaatgccctcctgttcagcagaccgcgtgtcccccggacagctatgaaactcaagtcagactaactgcagatggttgctgtactttgccaacaagatgcgagtgtctctctggcttatgtggtttccccgtgtgtgaggtgggatccactccccgcatagtctctcgtggcgatgggacacctggaaagtgctgtgatgtctttgaatgtgttaatgatacaaagccagcctgcgtatttaacaatgtggaatattatgatggagacatgtttcgaatggacaactgtcggttctgtcgatgccaagggggcgttgccatctgcttcactgcccagtgtggtgagataaactgcgagaggtactacgtgcccgaaggagagtgctgcccagtgtgtgaagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaagaaccaaccatcatcacagttgatccacctgcatgtggggagttatcaaactgcactctgacagggaaggactgcattaatggtttcaaacgcgatcacaatggttgtcggacctgtcagtgcataaacaccgaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgaagaataagcacggctgtgacatctgtcgctgtaagaaatgtccagagctctcatgcagtaagatctgccccttgggtttccagcaggacagtcacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcatagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgttttcttagaaccaaagtttttaaagttgctaagatatatttgcctgtaagatagctgtagagatatttggggtggggacagtgagtttggatggggaaatgggtgggagggtggtgttgggaagaaaaattggtcagcttggctcggggagaaacctggtaacataaaagcagttcagtggcccagaggttatttttttcctattgctctgaagactgcactggttgctgcaaagctcaggcctgaatgagcaggaaacaaaaaaggccttgcgacccagctgccataaccaccttagaactaccagacgagcacatcagaaccctttgacagccatcccaggtctaaagccacaagtttcttttctatacagtcacaactgcagtaggcagtgaggaagccagagaaatgcgatagcggcatttctctaaagcgggttattaaggatatatacagttacactttttgctgcttttattttcttccaagccaatcaatcagccagttcctagcagagtcagcacatgaacaagatctaagtcatttcttgatgtgagcactggagcttttttttttttacaacgtgacaggaagaggagggagagggtgacgaacaccaggcatttccaggggctatatttcactgtttgttgttgctttgttctgttatattgttggttgttcatagtttttgttgaagctctagcttaagaagaaactttttttaaaaagactgtttggggattctttttccttattatatactgattctacaaaatagaaactacttcattttaattgtatattattcaagcacctttgttgaagctcaaaaaaaatgatgcctctttaaactttagcaattataggagtatttatgtaactatcttatgcttcaaaaaacaaaagtatttgtgtgcatgtgtatataatatatatatatacatatatatttatacacatacaatttatgttttcctgttgaatgtatttttatgagattttaaccagaacaaaggcagataaacaggcattccatagcagtgcttttgatcacttacaaattttttgaataacacaaaatctcattctacctgcagtttaattggaaagatgtgtgtgtgagagtatgtatgtgtgtgtgtgtgtgtgtgtgtgtgcgcgcgcacgcacgccttgagcagtcagcattgcacctgctatggagaagggtattcctttattaaaatcttcctcatttggatttgctttcagttggttttcaatttgctcactggccagagacattgatggcagttcttatctgcatcactaatcagctcctggattttttttttttttttttcaaacaatggtttgaaacaactactggaatattgtccacaataagctggaagtttgttgtagtatgcctcaaatataactgactgtatactatagtggtaacttttcaaacagcccttagcacttttatactaattaacccatttgtgcattgagttttcttttaaaaatgcttgttgtgaaagacacagatacccagtatgcttaacgtgaaaagaaaatgtgttctgttttgtaaaggaactttcaagtattgttgtaaatacttggacagaggttgctgaactttaaaaaaaattaatttattattataatgacctaatttattaatctgaagattaaccatttttttgtcttagaatatcaaaaagaaaaagaaaaaggtgttctagctgtttgcatcaaaggaaaaaaagatttattatcaaggggcaatatttttatcttttccaaaataaatttgttaatgatacattacaaaaatagattgacatcagcctgattagtataaattttgttggtaattaatccattcctggcataaaaagtctttatcaaaaaaaattgtagatgcttgctttttgttttttcaatcatggccatattatgaaaatactaacaggatataggacaaggtgtaaatttttttattattattttaaagatatgatttatcctgagtgctgtatctattactcttttactttggttcctgttgtgctcttgtaaaagaaaaatataatttcctgaagaataaaatagatatatggcacttggagtgcatcatagttctacagtttgtttttgttttcttcaaaaaagctgtaagagaattatctgcaacttgattcttggcaggaaataaacattttgagttgaaatcaaaaaaaaaaaaaaaaaa

SEQ ID NO: 6 - DNA, 4101 bp, Mus musculus
  misc_feature (3600)..(3610): n=any base
caggggagggcgcccgccccgctcccggcccgactgcgaggaggaggcggcggcgcaggaggatgtacttggtggcggggggcagggggctggccggctgcgggcacctctcggtctcgctgctggggctgctgctgctgctggcgcgctcaggcacccgggcgctggtctgcctgccctgtgacgagtccaagtgcgaggagcctcggagctgcccaggaagcatcgtgcagggcgtctgcggctgctgctacatgtgcgcccgccagaggaacgagagctgcggtggagcctatgggctccatggagcctgcgaccgggggctgcgctgtgtcatccgccccccgctcaatggcgactccatcaccgagtacgaagtgggcgtctgcgaagatgaggactgggatgatgaccagctaataggttttgaaccctgcaatgaaaacctcatctccggctgcaacataatcaatgggaaatgcgaatgtggtaccatccgaacctgcaacaatccctttgagtttccaaggaaggacatgtgcctttcagcattaaagaggatcgaagaagagaagccagattgcagcaaggcccgctgtgaagtgcggttctctccacgttgccctgaagattccattctgatcgagggctatgctccccccggggagtgctgtcctttacccagccgctgcgtgtgcgaccctgcgggctgtctgcgcaaagtctgccagccaggatacctgaacattctagtgtccaaagcctcagggaagccgggagagtgttgtgacctctatgagtgtaaaccagttttcagcgtggactgcagcaccgtggagtgcccccctgtccagcaggccgtgtgccccctggacagctacgaaacgcaagtgcggctcacagcggatggctgctgtaccctgccagcaagatgcgagtgtctctctggcttatgtggttttcccgtttgtgaggtgggatctactccccgaatagtctctcgtggagatgggacacctggaaagtgctgtgatgtctttgaatgtgttaatgaaacaaagccagcctgcgtgttcaacagcgtggagtattacgacggagacatgtttcgaatggacaactgtcggttctgccgatgccaggggggtgtctccatctgcttcacggcgcagtgtggggaactgaactgcgaaagatactatgtgcctgagggggagtgttgccctgtgtgtgaagatcccatctatcctcttaacaaccctgctggctgctatgccaatggccagatccgcgcccacggggaccggtggcgggaagatgactgtaccttctgccagtgtatcaacggagaacctcactgcgtggccacggcctgcgggcagagctgcatgcacccagtgaaagtgcccggggagtgttgccccgtgtgtgaagaaccaacctacatcacgattgatccacctgcatgcggggagttgtcaaactgttctctgaaggagaaggactgcgtttatggcttcaaactggatcacaatggctgtcgaacctgtcagtgcaaaatcagggaggaactctgcttaggcctcaaaagggcctgcaccctggactgtccctttggcttcctcaccgatgtgcacaactgtgaactctgtcagtgccgcccacggcccaagaagtgcagaccaacaatgtgcgacaagttttgtccgcttggattcctgaagaataagcatggctgtgacatctgtcggtgtaagaaatgtccagagctgccatgcagcaagatctgccccttgggcttccagcaggacagtcatggctgtcttatctgcaagtgccgagaggtccctccttcagccgggccacctgtcctgtcaggcacatgtctgtccatggatggccatcatcataagaacgaggagagctggcatgatggttgccgggaatgctactgtcacaacggaaaggaaatgtgcgctctcatcacctgtcccgtgcctgcctgcggcaaccccaccattcgttccggacagtgctgcccgtcttgcacagatgactttgtagtgcagaagccggagctcagcaccccttctatttgccacgcccccggaggagagtactttgtggaaggggaaacttggaacattgactcctgtacacagtgcacctgtcacagtggtcgagtgctgtgtgagacggaggtgtgcccaccattgctctgccagaacccctcccgcacccaggactcctgctgcccacagtgtacagatgaccctcctcagccttccacatcccataatgagagcgtgcctagctactgcaggaatgatgaaggagatatcttcctggcggctgagtcctggaagcccgacgcctgcaccagctgcgtgtgcgtggatagcgcaattagctgctactctgagtcttgcccttccgtggcctgtgaaagacctgttttgaggaaaggccagtgttgcccctactgcttagaagacacaattccaaagaaagtggtgtgtcacttcagtgggaagacctatgctgacgaggaacggtgggatattgacagctgcacrcactgctactgcctgcaaggccagaccctctgctcgaccgtcagctgcccaccgttaccctgtgccgagcccatcaaggtggaagggagttgctgcccaatgtgcccagaaatgtatgtgccagagccaaccaatgtacccattgagaagaaaaatcatcgtggcgagattgacctggaggtccccatgtggcccaccccaagtgaaaatgacatcatccatctccctagagatatgggtcacctccaggtagattacagagataataacaggctgcatccaggcgaagactcatcactggactccattgtctccgttgtggttcccataataatatgcctgtccatcatcatagctttcctgctcatcaaccagaagaagcagtgggtaccactgctgtgctggtaccggacaccaaccaagccttcttccttgaataatcagctggtatctgtggactgcaagaaaggcactcgagtccaggtggatggtccccagagaatgctaagaattgccgaaccagatgctcgattcagtggcttctacagcatgcagaaacagaaccatctacaggcagacaacttctaccaaacggtgtgaacacgggcagcagcgccgatgaggtcccgaaagacggagaaagacgaaaatctgctctttaaagtaaactaggatttgcgcacttgcttagtgtcttgtgtcagattgcgacttggtgtccagcgctaggggctggactgggatgggctctgtctacagcactgtgcagaacaagcattcccacttggcctcaagataactgaccacatgttttcatagaaccaaagttttaaaacttgctaaagtatatttgcttgtaagatagctgtagagacatttgggggaggggagaaagagtttggtgggggaaatgggttgggagggtagtgttgggaagaaaaaaaaatggtcagcttggctcggggagaaagccagtaaaaaaataaaagcaatttatggcccagaggaatttctttttcctgttactntcaagactgttggttgctgcaaaggtagggcctgaatgagcaggatcacaaaaggccttgcaaccgagctgccataccacaccagacaagcgcatcagaactctcgatagccatcccaggtctaaagccacaagtttctatagagtcacagccgcagtaggcagtgaggaagccagggagatggaaagcaacagttctcgaaagcgggttcttgaggatgtattcactttttttgctgctgctgttttcttccgtgccaaccagccagttcctaggagacacagcaggttgagtaggaacgaggtcacctcttgacctgaccactggagctttgctcgctacgtgacaggaagggcaacggcgaaggacaccaggcatttccaggggctacacttcattgttccttgttgttttcttctgtgctatcattggttgttcatagttttgttaaagctctagcttaagaagaaacttttgagaaaaaaaaaaaaaaaaaa

SEQ ID NO: 7 - DNA, 4010 bp, Gallus gallus
ccgcaggcaggaggcgagtcgggactcgggcagcgagcggggcagaggcgccgccgccgccgcacccaggggctcgcagccgctcgcgcgcgatcgccgctcctccggcgtggacggggccggcagccggcgggcgaggaacgcacggaccgacggacgccgcttcccgcaggggacaagcgggcggagccgccgcccggagcagaagtttggctggagcggccgccgccgagagaggaaactttcctcggggggccggaggcggggcggcgggaaggagccgccgctcggcggcttttcgccggcgcgctcgggggctccggcgcggctccccccggcgcgggccgggcggcggcggcggccgggatgtacctggcagcggtctcggcggggaggaggcggccgggcggagacggcggcggaggtggcggcggctggcacctggcggcggcggggtggctgctgctgctggcgctgctgctgggccagccggggacgcgggcgctcgtctgcctgccctgcgacgagtccaagtgcgaggagcccaagagctgccccggcatcatcgtgctgggcatctgcggctgctgcttcatgtgcgcccggcagcgcaacgagagctgcgggggcgtctacgggctgcacggcgcctgcgaccgggggctgcgctgtgtcatccgcccgccgctcaacggcgactccatcaccgagtacgaagtgggcgtctgtgaagatgaaaactgggatgatgatcagctccttggatttgagccatgcaacgaaaaccttataacaggttgtaacataatcaatgggaaatgcgattgtgacactattcggacctgtaataatccatttgaatttccaagtcgggatacttgcctgtcagccttgaaaaggattgaagaagagaaacctgattgctccaaggcccgctgtgaggtccagttttctcctcgctgtccagaagattccatcttgattgaaggttatgctcctcccggggaatgctgtcctctcccaagccgttgtgtgtgtaaccctgcaggctgcttgaggaaggtttgccagcctggctatttgaatatattggtttctaaggcttcaggaaagccaggggaatgctgtgatctctatgaatgtaaaccagtgttcagtgtggactgcagcacagtggaatgccctcctgttcagcaagtggtttgcccactggatagctatgaaacccaagtcaggctgacggctgatggctgctgtaccctgcccacaagatgcgagtgtctctctggtttatgtggtttccccatgtgcgaggcaggctccgttcctcaaatagtctcgcgtggagatggaacgcctggcaagtgctgtgatgtctttgagtgtgtcaatgaagtgaagccaacttgcatatttaacagcatggaatattatgatggagacatgtttcgaatggatgcttgtcgcttctgtcggtgtcaaggtggtgtctctatttgcttctctgcccagtgtggtgagctacactgtgacaggtattatgtgccagagggagagtgctgcccagtatgtgaagacccagtttaccctgtaaataatcctgctggctgttacgccaatggtcagatccaagctcatggagaccgctggagagaggatgactgtactttctgccagtgtatcaatggaaatcctcactgtgttgctacagcctgtgggcagagctgtttgaatcctgtcaaagtcccgggagagtgctgcccagtatgtgaagaaccaacttacataacaataggtccgcctacctgtgagcttttggtgaactgcactttgactgagaaagactgtatctatagtttcaaactggatcaaaatggttgccgcatttgtcagtgcaaaaccagagaagagctgtgtacgggcctcatatcaggctgctccctggactgttccttcgggttccagactgatgcccacaactgtgagatctgtcagtgccgtccacggcccaagaaatgcaaacctattgtatgtgacaagtactgtccctttggatacttgaagaacaagcatggctgtgaaatctgtcgatgtaagaaatgcccagaaatgccttgtggtaaaatctgcccaatgggcttccagcagaacagccacgggtgtgttatctgcaaatgcagagaggcaactgcttctcttatgcctccagttaaaacaggctcttgcctgtcaatggatgggcgccgacatgaaaatgaggagagttggcacgatggatgcagggaatgttactgtcacaatggacgggaaatgtgtgccttgatcacctgccctgttcctaattgtggcaaccccaccatacatccagggcagtgttgcccatcatgtccagatgaaataattgtgcagaagccagagctcactagtccatcaatctgtcatgctcctggtggagaatactttgtagaaggagaaacgtggaacattgactcttgcacacagtgtacatgccatagtggccgagtcctgtgtgagactgaagtctgtcctcctctgctttgtcaaaaccccactcgcacacaggactcctgctgtcctcagtgcccagatgagcctcttcagccttctttgtcaagcaacgtcagcatgcccagttactgcaaaaatgatgaaggagatatttttctgacagctgagtcctggaagcccaacgtctgcaccagctgcatctgcatggacggtgtaattagatgctactctgagtcttgcccaccagtatcctgtgagagacctgttttgagaaagggacagtgttgtccttactgtattgaagatacagtaccaaagaaagtggtctgccacttcaatggaaaaacatatgcagatgaggaacgctgggacattgacagctgcacgcactgctactgcctgcagggccagactctctgctccactgtcagctgcccacccctcccctgtgctgaacccatcaatgtggagggaagctgctgtcccatgtgcccagagatgtatgtacctgaacctactaacatccctattgagaagacaaatcatcgaggagatgttgaactagaagtgccaaactggtcaacaccaagtgagaatgacatcatccacattcacagagacatgaatcatctccagggagagtacagaagtggaaatggaccacaccccagtgaagatgcgtcagttagctctgttgccttggtgacagttccaatcacgatagctctgctagtaatcattgtctttctgctcatcaatcagaaaaagcagtggattccagtgtcctgctacaaagctccaacaaagccatcttgcctaaacaatcaactggtgtatgtggactgcaagaaaggtaccatggtccaggtggacagttctcagaggatgctaagaattgcagacccggattcaagatacagcggattttacagcatgcagaaacagaacaacctacaggcagataatttctatcagacagtttgaagaattgcacccttaccaaggatttaagaatgagagagagatagagagagagtgagagagagagagagagagagagagactaagtctgctattaaaataaagctagaattgtgcacttgcttagtggattgtattgggtttgtgactacatgtacagctctaagaccttactgggatgagctctgactcctgcaatgtgccagaaaaagcattcccacttttccttgagataactgaccaagtgttttcttagaaccaaagttttaaaacttgttaaggtgtatttgcttgtaacatagctatagagtttttttgggagggggaatcaggggctaggggtaggcaatgaggtaaaaggaagtttcagagaagaaaagctggtcatgcttggctggagaagaaaatgaacaaatactgccaagcagcaggacagtggcttttcttgttgtcctgaaattgcatctgttgcagcaaatcctaaccccaggtaaaaaaaa

SEQ ID NO: 8 - protein, 41 aa, Artificial
  C-terminal CR1M1 peptide
RVQVDSSQRMLRIAEPDARFSGFYSMQKQNHLQADNFYQTV

SEQ ID NO: 9 - protein, 22 aa, Artificial
  N-terminal CR1M1 peptide
KVCQPGYLNILVSKASGKPGEC

SEQ ID NO: 10 - DNA, 22 bp, Artificial
  Crim Primer 1
gctcagcaccccttctatttgc

SEQ ID NO: 11 - DNA, 21 bp, Artificial
  Crim Primer 2
gtgatgagtctcgcctggatg

SEQ ID NO: 12 - DNA, 18 bp, Artificial
  Crim Primer 3
ggaatcttcagggcaacg

SEQ ID NO: 13 - DNA, 22 bp, Artificial
  Crim Primer 4
cacagcgggccttgctgcaatc

SEQ ID NO: 14 - DNA, 22 bp, Artificial
  Crim Primer 5
gccggagatgaggttttcattg

SEQ ID NO: 15 - DNA, 21 bp, Artificial
  Crim Primer 6
ccgccagaggaacgagagctg

SEQ ID NO: 16 - DNA, 21 bp, Artificial
  Crim Primer 7
ctaccaaacagtgtgaagaaa

SEQ ID NO: 17 - DNA, 20 bp, Artificial
  Crim Primer 8
tggtcagttatcttgaggaa

SEQ ID NO: 18 - DNA, 20 bp, Artificial
  Crim Primer 9
ctcgctgtccagaagattcc

SEQ ID NO: 19 - DNA, 20 bp, Artificial
  Crim Primer 10
ggttgccgcatttgtcagtg

SEQ ID NO: 20 - DNA, 50000 bp, Homo sapiens
gaattcagcagtcgtcgccgccccccccccccatgactatctttttataccttgtctaaatgatttctacaaagtttgtatattttgagtaatgttttaggtctgatattattttgcattgatttgtaggaattataaatgtaaccatttatctgtgaacaaataattaaaaatacttaatttggggaggaggcgggaacctagatgtgctataatcagcagtgtgcactttgcttcacattatcttctgggaggaaattttgctcctagctcctgagtgtgcacactgatgaccactgcaggatttggcctggcttgagcaaagaaacagctcgacgccatgggggctatggttccaacctgctgctgcagttggtataaactaggcaaaaccagaagtcagaggattatccattcctcttgttctttttctagctctacctgttcttctgccattcttctgcctgaaaatattcatgagcatcgcctatgggtcttgtctctgtgctttacagtagtgacatcacttgtgtaagccagtttttctgttagtcgtcggtacctcctaggttcgctgtgggggttaagtgagataaaactgtgcttctgaaattttagtgtgcacacaaatcacctaggtatttaaaattgcaagtctgattaagttagtctggggtgatgcctgtgattctacatttctaacaaatttctggctgatgccagtgtcacaggtgccaggaccacacttttaagtagcacaaagctagtacctatgtatacagcatgtatcacagagtatgcatattaaaagacctgataaatgttacctgctattatagtcattatttttcagccaaaataaataagatatggtccttgttttcacacaggccatgatctgatggtaaactggaaaagcagacaattcattacaatcagttcagtaagccatgagataacggaaatgccaagcatgtcaggagcaagtaagagggagtctaacccaaatctgaccaccaggaaaagtttagtggaggatgcagcaacaagaaagacctgagggtctcctctcctccctccttttttcctgcacttcttgtgcaggatgttttaggaaaaagaaaatagggcttggggctagacaaacctgcattgaaatcttaaatctgttatttttttaatcgtgagatcagcttgtttagtttctctgagcttcagttccctcatctgtgaaactggaatattaatagctttctattaagtaaatattgtagtgaggattagagatttatatatatgattacatagatgtatatataagattgtgtgtgtgtgtgtatagtatgtatgtgtatatgctagtttctggtaccaaatagattttttaaaataatattaattttaatttccattctattttctatcctttcccattttcctctttgttagctttctctgtaagtttaaaaatttaccttgtgggcacacatttcctccaaacttaactaaatctcttcaaaggggtgggagtttcgagggggaattcaaatgtcagatgtttgtcaaagagaaaagaaccgtgaagtaccgagaacagggggctgggaatacagggtctatgcaccaaggaaacatgcgttggtttgatccagtgcaactacagttcccagcgcctttggcatttcagcagaatgtgaacacttgcttgattttgtggtttgagcccctacatccaaagagctatttcaaattaatatttatttaaaaatggccttttaatattaatggcatatggttttatgacagtgaattaaatttccttttattgttagaactttttgaggatttctggacatcactggagacgttggagaccccaaggatatagccaggccaggttgatatatctcactgaggttacagaaggaagcagcggcaagcctagccgattcagagaccactgcgggcttgggaatgagcaggagagggtgtgagggccagaggccatctgtgttgcttttctctctcccagtcattctagccatctttgatgtggactaatagtcgaggttggccatgggtttgcactgtctctagggagtctttgtgatatagtgcggggtaggaagaacacctagaatcaaaatatctaatgttactagctgtttagttcttagtgtgggtatggtcccattttaggagaacagaactatgaactatgtttgtgttttttttttttttttgagacagagtctcactctgttgccaggctgaagtgcagtggcatgatctcagctcactgcaacctctgactccctggttcaagcgattctcctgcctcagcctcctgagtagctgggattacaggcatgtgccaccacgcccaactactttgtgtatttttagtggagaccacgtttcaccatgttggccaggatggtctctcgacctcgtgatctgcccaccttggcctcccaaagtgctgggattacaggcgtgagccaccgcgcccagcccagaactacatttgcttaactgtgactctttcctttgtacctgaaaataagtatcatttttatccagtaaatattggctgataattcataaggcagatgctttaaattggctattgtagaacaaggagaaagaacttgatgaagagaaaaatccttggaatcagtggtttacaactttttttttccttcctgatgatcaaaccgtattatcgcatagtagaaattggattttcttccagaaactactttagtacctctcatttgtagcacttctatcaagagtgctggtattcggacatgtaacctacaaacaaaatcagaaagatgtcaaaataacaccctaattcagactatcctgagaacatttgtcttctgaatatttaagaaaaaaaaataagtgttaattatattgtagggtgttaccattttgtatttcaagttcctgagaagagaatttgaacagtttgctatttggaaattttagcaaccagctaccttgcctatggaaagattaaaaaaaaaaggagagggagtagatggtggtccataggctgtaactggaaactatgcctgtcttatttagcatttcaaaacaaaaaccataaacaaacatctcagattcaaaacagaaaggaatttctggcagggcagaatgaaacctgagactgcagagtcccagtctacaagaggtaacttatttagcttcttatccaaaggccacagcaaaacgtttcacagctgtgattaaacaaaaggcatcttttgttggagaattgtgatacttctttttaagactgcgaaagacctgccaaaataaggcaaagctaccttgaggatgtttgacctgaactagactttatgaagtagttggaccacttcaggccttatggtgtgaggagtgtgggatgcataggcttggacctttctgtgtgtgtgtttataagtctgtagagacagaatattccatcatgttatcaaatgcagatctcgaaaaccttttctcacttcccatatttgggagctttgctttttttgaaaagtatgggcttttacagtctgagtgaggaaaagggatggattttagacagttttgtctgtggaagtcaaagtagaaggatttttaaacagttggagacagaatgggtatactgtgtgcttttcagaaccactgagttcctatatttcaggcagattttaagaaaacttattaccactacctatcacatcaaaatgaaagaccaaataaatttctgtgttttaatcaagactgtgactgtatttgtgtatatcctagcttgtttcagaaagggtttgaggctgcccagaaaggtgaatattacagaattcttgtatgtgataacatgataaaggagaaaaaaacagccatggcttttgaatcccccttcatctcttaatagcccagtgacttaaataaaacccttcaacttcccttggcatgactttgccttaatttgtcaaaagggaattaaatagttacctacctcaagacaggggaactgaatcagatgatatcttaaggttctactggtcataagatctatgcttttatgacctagaagtgatagacaggagtcgaatattaccttgtgtttgcagagccagggaaggaaaaagtgatagtgtctacatgactgccaggtgctgtagacagaagggcttagaggaaaagatcaagagttcctccatggttatgtttatcaagcggcagtggagacagtaaagcgtagtattgggaagttagtgacagcttttaaatgcctgaagtcttttaaatcatattcaccgtggctgagtctggattcagggtagaactcaaacctctcttatgtgtttgctgccttccctttctccattttcttatgtacttcattaattattaagactatgaggtaggtaggtgacagagactgcaaagggagaagctagagcgaagacaggccaaaacttgcccaaattcacatgctcatttctgcctctgacactaggagagagttgagtaattgctcttcttcctaatactgcaggcagcagccaaattctccacattcttttacctttgctgctgttttcacttgccagggagtttccttaaggagcagactgaatttaaagaaacatagcagtaatagtgcatgaaagggggtgtggggtcgctggccaaacgtggcatgttggtgcctcacgttgggaggatgaaaggatgattcctgcagagggacatgctgcacactgcgcaagctccctcacctctgtgtacagctgagccactgaccaggaagctctttgcttgtatgtgagacagcaaatattcactaaatatgtttcaagtacaagccaactgcgaagcttggaaagttgtccagaaacttcacccttcctttgcatgttgtccagcggcaggacgttgagcaaggttaggtagtatgaacctgcggctctaaaacttaaagtaataggcattattcgaaagaaaatgatgcccagcacggcttccagtgtatattagcaatcagcttgtttgccttgtggttattacattttttgccaagagcagccttccagaatgaggtactagttaattcccattgggcatttattcattaataagtctgctgaatgtgaaatattccatttgggagtatgtcatatgagaaaactcagtggctaattattacatagctctcttccatgattctcactttcagcgcagttacctaagccaccatttcctaggtatgtaaacttagataatttttagttttctgtaatactttttgctttttgctccagaaaattttttttcactccaatctgtgaaattgttaaaagtttactaataactggataaacattttatgttgtactatggcttgctctttctttccattttaaaaagataatatcaaaagttaaatcctgttccagatcttaccgtctctcttccaggctagatttctgggatggggtggtgttaatttactaacctgagcttttgcccaatctagggtctcctttactcttttctggtgtataaataggtctttatttgcttttcttccaggttggttggtgcatattagcttaagaacttttaaacctctgcggcagtaatgagaatctgcatgccaagggcccagcatttctctacaaagcagcccatgagtgcatgtgtgtttgctgtagtgaagggtctatgttagaaggtgagttagagaccccacttgcccacatctgtgtgctgtggtttggccctcctcctctggtgaatccctaccacagcccagtgcctgctggatctcaagtgtcctgaaagccattggaaagatgctttccagcagtcttaacatttgcctccatgttctttccagcaaggatgagtgcacacagctttccagctactgggcaacccagccattgacagaaggtggtgcaagagtcaattcctctgtgaggatcccttccaataacagtgtctgcattaacgacaaaaagttctgtaaaaatgtttgcagggggagcttttgacctgcaaacatttctacagaactttttgccatataggaacatatgaaatgttatatgtaatataggtcagaaatgttgccatataggaacattcgagaagatgataaatgtgaggaaagtgataaaagtttatacaacaaggatctgtacaagattgtggtagatttgctggcctaaaaagatgtactcagggtagtgaactatttctgtagcatcagccactgggcccctgagtgggaatactctttgagagacaggaagctaattccttctttgaatgtgtagttaactttgtgccattggatgtagggtgtgcacgtgcatgtgtattttttcagttgctgagggcacaacacagacaaagaataccgtgcattgtattatggatgtgctggaaacaacgccccagcttgaagcaagttgactgtagtatctactctgtagatcagcaaactgaaaatgacttcttaataaaaggtcgcacattgttttacttactgcatttgaaaaaacagttctaattaagatgctttgggttttaaactgcctgttaattgggatatctataagtgggcaagtagagcttctgaatattgctgacttatttcttagtgattgggacaaaagctttttttttttttttttaaatagtatgccatcattaaacagtttttgaccttacgcagaaactttaaatgtgacagcagaaagaagcagcagaaatttacagtgagaaatggaagacctctgtaggtaaataagtaaaatgaaagaagccagaggtgttttttgttggcagtagatgaaattattttcaaacactgccctgggaaaaggtgagccctcagcaaccaagtttattgactgtctattcaggaaattgtattagttgctttggaaagagaaaggaaaaagaaaagactttctgccaactgagggctggaaaacttgagggagataaagtatttacatgctttaataacataaagcatgtccaaaagaaatacataattacagctatgtttggcactgaaaataatccaggttattatatttttaaaattgtgtgtgaatatgtaagtgaatgcccttgagtcagaaatgcttgagtgataatatttcttgagagaatggaaaagtagatggtattaggagctaaaaagagagagaagggataaaggatgaccactagttaagaaggaacagtgaactttttttctttagaggggacatatttcatatgcatagttattccccctctacttctaatcttagtataataagcatttaacttcccataggaccttagcagtctgcatgttattactaaaagcccacaggtaagggaaaattagacccctaaattaaagacctgaccatcttgctcctgaagttttaggttttccaactttatttccagtgtatcttggacaaagaccctttgagagctcagaaaattaggagatgtggtcaacacattcttcttaatatctagtgcaaccaaagacttggccagcaactaaggccagcaactggaaaatgggtgtggtgtcaggtttcagacgcccccatagttgtcttcacgtcctttaatatgtgttggcatccctcaagccctgccagtttatgctctttcctttgtgaagccttcctgccctgcccaggaaaaagtaatgccatcccccatcccccatcccccagtgaccataaatagcactttctccttcccctgttatagcacttttcatactgtgttagagttcacatttctgtctctctgcctcattgtgatctatttgagtgccaggattgtgtgttattctagcgcttggcacacaggaagaactcaatgagcatttgttgactaaatctagactcaaggtcatccaagatattatacaaaaactttttcatcgaaccatttggtctgcattgtggaaactgaaacgaatggcactccccgttaagagaatcccagtgagaagccatagcattgcaggaggaagctcaaactggttaactttgctcatgccgatcagtcttagaattttttgtaatgtttccatgatgaattagtttcagctgtatttttttggcaaatatttaaagataatttttatttgtgtggacttttcttcttgtcagtttgtcataaaggtgtcatctttcattcactaaccccttatcagcttataggaattttcctcgtgggtggttgcttgtaaccttctcttgactgccagggatctgatatactaaacggttgagatactgtggtgatcagctgcttgtaaacaataactgataaatcggttataaattatcaacacacttcagatgaccatatcatcatgacttggtgatagcaaaatagacttctgaattctcagctaatccaggattgtgtcttaacaaacatttatgttgattaatcaggagggtgtttagtttaagaaacatgtactgaatgttcaatatgtgagtgccaagtgcagtgtggtgcaacctgtttactgcttcaggtgatctccagatatcctaaggaatttttaactgtacagcttctttggggaaggaaatcctaagccaacaacctgagggtggaagtgtgtgactgggaataattaaaatgcctcacaatggttcgagcaggaattccataggaatgcactttaaggcatttaggaaagcaaaattccgtttggatttactccaaaccctatgacttcagagacaggaatttttgatgattagtggctggagccccaacttgttatttagtaattccacattcagtgtggaattgttttttctgtagcactagttataagttctttgtttgtttttctaaaggttttaaggaggtttacaaattaagatggaataagacattagaaacttaaggatacatagcaaattaaaaccttgatacatttggtgctaagcttttcagctgctaaagatagagagttttggccaagataaagtatatagagtcaaagacacatgcagaattctcctggatataagttcaggtacctgtgccattttatcactttagtcaatgtgcccattttcttagggcaggtttaattttctgctgcggcatggcccttcagctttcccaagtttacgatgctcatctcatacctgatggaaattgttgataatgttagtgtatggttggtggctttcccagtagcccacttcagatccttttattggtcatcaccagttacaaaagttttaggaaagggaaggagagaagctggaactgatggctaagtaaagttatagctgtagagctctggttgtccataatgaacagtggcaatgaacagtgaagatttaagcaaggttattttttgagggtccactgatgattttcctataactcgttaaaaatatttttaaaacttttatatggcttcatataaaaaaagcttaaaaatctgttcccagacttcctaggcaggtatgttttcttttcagtgttgccttctctaagactttatcagcagaaggagtatttgagctgatggcaaatgctgccaaaatgagaagtgtgtgtgtttctcaaaactcatgatcccacagttaaatcgagggtgtgggtaaaggacccagaactgttcttgctgatggaatagcacacttttcctcagacattagtagagtaaatgaatggaggcagaggattgggaaacactcttgaaggagggagtgcccatcagcctgtaagccaaagtaaacagttcttgtaactgaaaaatagacttcctggaaggagagaaccagagaagtgggtgtgaagtgcaaccagcaagtaccattttcagtgatacagacagctggcttaacctgagtcctgacaagaacctctcagagctatttggggaaatcagttgcttgaaccagcctttgactggcctatcgttcctacagctgtacccaaagatggcacttggggagaaaaggtcacatttgtttgtgatagagaaagcaaaccgatggggcattttgagaggtagaaatgaagtcatcatttgcggagccggtggaccagcagaatggccgtgggaaggtatccctgaacctgtatgtgtggaaatgcaggcattctctgtcagtcttttgtcacagtgagaaaatgtcattacagtggacagctatgcacacgttggagattatgtgatgtgtgcatgtgcatccatgtgtacaacatacacccatgcaaaaattgctcacagggtcatgtgcgtgtcctggtagtctgttatgtccattcttctgtaataggagtatgtttttcaaactgaagctgttaggttctgttaattccaacagaacaaaaaaaaatctgcctctggccatcattcgttaatttacatagattagatacatgtagtctgtctcctaagtggcttacataaagccgtgggcgtcttttcctgtgtctcctcttcggcagttcctaaggcacctaccaggtttatagttgctgatgtcctacccatactatgtgtcaagaaaggtagactcaaaaaggattgatgcagatattccttagaagttttcatgcccaaagattcttggaccttagttgcatgagggaagaggccttgcctcgtgaaaggagaccgaagccccttattaaaggggtcactcagaaaaaggtgttatctaaattccctctttgtagttttatttcttcaatgtattccccatcactagctgtgtgtgctggagccaaatgttgggcccactgccatcttttaaagaatgctactgtaaagggataactttactagacagtttgggaaatagaagaaacaattatctaaaatgctcttgttccaaacacaaaaactagcttcatgtttttgtgtataaccctgcctagtcttactctgtagacatccttccccccacacccatcgtgtatgttttttatccccatctaacattatatcaaatacattgtcctatgttgctaccttgtcttcattattatgatttgtcaagcaattgggttgtactgactgcagtgttactcctcatttcctttccccctttcttttccatactacttaaaaagcaagaaaggaaaaaaaattctatttaagagaaaatcctatgtagtcgtaatggaatttattttcctgagtgcctgggcgtgctccaaacagatagctgtgattgtgtcatctcttcttactaagcatctgaccactcactacctgtgcatctgatccctaggctcatggcatgagggctgtggctcttgatgtttttttttttgatgtgcttagtatcaccagaactgtattgactctaagagtatggaagctttgtaaagttaactaacgatttgtaggttttgtgcaaatagccagctctccaggtagagacattttaattctgttgctttttttttttgtagcatattattatattgcagatgtttacttagtatgaaaaatggatataattaagttctaaatgggtactagatattaacttcaaaatgaatttgcaacttgtatgctttgcatatgccgtatgctttgcatatgccatatgctgttgtttccaaggatgtaaagcagttttccacttccaccatcttataaattaaaaccagagggctagacgttatccagcaagggacctacaaaattcgaaaaaaaacttcggatcattatcaggattaagtttgttgaaattgtatcaggaaaatagacttttccttgtattattattgactcatataatgcaactcatactggtttcatattcagaaggtatatagcacctgttgtaatatttcaggttgatttctgcaatctttatttaaaaatacaaatccgcaaagcaaattgcataaacggtatacaaaatgaaatcaccaggcttcaaatatattttatgtaaatgttaagtgctctttagattagcaaaaggaagacaaacattttgattacctctttaatactaaatttttgtttctgaattataaatctttgaagatataattagtgtaagatatataccaatcaacatagtccagaaaaccctgaacctggctgcttattagttggaaagagtacctatttagactttctaagccacagtatgtggctaagtgattaatatgcactattacactttcacaaatactttttggaccaacatattgacttaagggagcatactttgtactaaaaaactaatttgaaggtgaaatcacagcaaatttaacagttcaaccttgtgtatttgtagagttttagttggatcataggataatgacattagctaaagtcagcaagcatgtaaaataaacattttgaagtttaatagtcatttatgttctacaataataaatatttggcacccaccataaccaccactaccaaaatatgtatgctgaaattgtgtctccaggatgaataaagaggtgtaaggcaagctgacacccattagaaaaaaaggtttagaaagggagagagggcctggacttccctaagcttaaccccaacatagggcctgtagctcctgttggaaccaatttataaaacttcaaggtgggaatcaatagattgtttccagtgttatatagttcctctttaatcagttgatcttcaaccaaagcttcaagtggtttttgtttttatttattgccaaaagcttcttaaccaatgtgattataaatgtttaaattcatttacagtgaaatggatttgtatcttccttgctctacagtagatagtgaagcctccgtgacctgtgtaagctgcaggttgaggagaatgtttaaacagtaggcgagcgttcatacagtgtccctggactgcatttccttgcttacagagccttggagctgggccaactcgcatgagcgggaggaggagaggctcagttttgagagccctgggtttatttctggctgtggggatacgagaatgtgggagaaggtgtaaacatagtttgagggaggtcgtagacttggaggttcttgccaggggtaaatgctggctggatttgtttatggatggatttgattatagctgcttcacgcttttacctctggagttggtgcttccaaggggtgatgctcattgtctggatctcctttagtgggcatctcagaattctttgctgcaccaaccacagcatttcttttccctttcttttggtatttgtattcaaatgcctgacttcctgacctgctgtgagggtttttaaaaataatgaacaccaagcttcttgggaaaagacaggtatgtaaatactgcaagcttatagcatttgggggatggagttgtgggattttggagtttgtgacccccatgtgtctgtgtatcactctctctaatgtagagagacagagcagcagttcctgagctctgtcttttgtctcttcatgggttggtcactgatttcccctttcagggtctgaatccctttcccttgcctccccccatgtgggttccggaaggcccacatccaacagctgcccttggtcaacccccagccctgcacacaggtgttttctgctgttcagagctcaggggcgctgtccagccagctgatgacagctcactggtggcgctggtctcactccagtgctggctctgccctcttggccctgttgcttcctccttggaggattagtcaccccagtcttggggcattttacccacctgtatgactagcacacattgcatccacttgccaaggagtgttagggatacagtctcaagaaaacctgcccagtagaaatgacttcctctggagaaatttagatgataaattccatagggagaaatcatgttttattaatgagaaacaaaatcgtgcactagtgggagttcaggctcagaagccagtgcatagtccagcctggcgacctggcatggtgtgttaccctggccaaagctccccaacctctgttcatgatttccctctgtaaaatgggaataacaatagtatccatttcttttgggtggttataaggaatacttaagttaactcattcatgtaaagcacttgaatagtggcttgtatacagtaagcacacagtaaatgtacattgtcattgtatctttgtacctcaacatctgaaactatggtttatacatagcacatactcattcatgctttgttgagtaaatgtaatattcagaaggtgggaaagtgatttccttctctcttatcataaaataagagttatatttggttgaaataaggcatcataggcctgaagacatttactccctggctgctggcaggggagtagaatgttccaaactctatgttgataactgggtgcctatgatttgttaatcatacctaacaaactcagtctgtcaggtgtttcagtaaaatttactgactcccaaggtaattttgaaaggacaaagccatcaggtgatttgttttcaataagcattattgggatatttcagactccaacatgtgcagtggagatattaacgagactttaccctttctaaacttaggggcttactctgcattgtaacgtatgtttgcttttaaggcgatagatgggtagatagatgctctgtaattgaatagcctactgggcttctgtgtagcacagcactgtgtaagtcgcaggtgttcagtaaatgtattttaaatcaatgagtaattgatgggatatttttcttcacattacatcacattgaagatgcttgtgaggaagttttgttcatgattgtcctcctaggcaggcgccacctaacatgtttctctctagctgcggggtattgaatttgaggctgctctcagcattactgctcatctcaaagaggaacaaatggtcccccaaatggtccagatgcaacagtagggaaaggagtgccaccattcattctgaggtgcctgggccatactctggatgaagctcacagctctaacgtaaggcggtaagtcattttttttttaaacttaaggaagttaatttgaaagtttatgctgatgccatttcatatgacagagactaaagggtatacgtccttgtgttctcttggtggggactgctgtcctaaaggtaaaagattttgatccagaggttggattgttcctcgtgtggcaacagtcctctgacctcagtcattttcaaagtctaatgtctttttggctaacaaaagacatcaagctagagattaaaatttgcagaagatgggagtttttttgttgttttcggttttttttttttttttttttgagacggagtctcactctgtcacccaggctggagtgcaggtgatcatatctacaaaggtgacaagaacatgaacctgcagtcctgttatcagcattttgtaagtgcttcttctcattctaaaaatatgtattgattttaaaaatgaattctgtaacaggatagcaacagtagtaaaaatcctttttatggagaaatttagagctttcagctaacatacacacatactcattcaaatgaaacagcttgtcttgttttaaagaaatatttctgttgtccattgtacaaattttaataacttgtctttcctttttttaggtctctgtgttccagatttttttttgtaactatgaaatccttgccttgtagtcaacttaaaatgaaataaatttatcagtgcaaatgagtacactgtggttcttgtgtggcaaaattatgcaggtggtttgaaggattcattgttttattaaaagactagaagaatgtacaaatgctttccttctttctcatgtttctctcacccaaactgaagtggtccatttaaaatctgatcttcataaaaggaagagttgtcacaggacatctgatttatgagccatctgatttgcttctgctgtagttccacactctgctggggtacaaaaatcaggcacttttatttcagccctgacaacagtgcggccttcccaaagtatgagaagagcagcgtgcagaggtaatgtgcagtcttccgaacagagcactgaactggtttttaatgaagggtaatatgtacttggggtccaaactctagttaattccggaaaacatatcctctcactagtgttgggtcttagtaaatgagtgttctgtgttgaatccttgcttgagaggaaggaggtcataattggtcagggattctatttctttctttctctcttttttttttttttttttttttaaagaaaagcaattgcttagaatgagatgaatctatatcagggtatgtttttgtaggatcatattaatcatcttacccacagactgactttgtggtcaaactttactcataaggatttattaaaagcaatggtttggttaaaaaaaaaaaaaaaaggtatgtttatgggaggaatgtagacatttagttgaagtacagttgatagactttgaccactcagggatattttctccatggactgtcttacagaggcagagcctaactgcatatgcttcttatgactctgatgcatgaatggcagccctaaatgggctcctcttcacttattccttctccccaatctccccagcactgtgttctgtttccttccctgcaagagtctgcgtttatcagtcaggtactcctgtcctcagagtgccaggccagctctgcatccttctccacaagaccaggctgaagtctgcctgttcacctgttgatgctacacagtttgagctcaagctccttattctttgccggcttcccttttttgagttccagtggagtggtactcttgtgtcagagggattcagcactgcccactatgtcatggtactctgcgctgctcctcttgggtctggatggggccacagttgtccagtcccatcatgctgcagccctaagctccagagggtgaccttgataggatgcacatcctggagcttaatttcataagccttgggggcctgcaagcaggaggagttggaagcccttgagaggccagatcctgtgggtttgagcgcagagtctacagagttaaaacctgcagaacaaagaaatcaaaagaaggacataaagaaagtttgggagacattgagtctgcaaagcaaagaaatccagtattgcaaggggaagattttgctttgggaggttgaagttttgtttgccctccgaatgaaagtggtatttctgttcgttagccaaaaaatcatcagactttgaaaatcagtgaggtcagaggactgttgccacaccaggaatcagaattgtggcattggaagtttcttcagaaaagcacctttaggcccgcccaactttacagattaaggaagtgagacccataaaagtttgtcatgttctcttattttaaaagatatctcgaccatgtctgcagttatggcctctttttcctgcctcacccaaaggggtcgaggagctttctcttgcccactctggagagaactcctggggcccccccgctgctctccctgccccatgactttgtcccagaatacagaacacagtgggctggatcagcctctgctgaacttttccctgccagcctccctgcccgccttcttgcccccgcccctaccccccacacaggaatctagagccctcccagtggtggtgtgaagcctctggaatgtggcactctgacccctggcagttgagtgtcctggatggacctggcctgggaaacaggaaggactcgggggagggggaaggagtgccccatggccttgaggtgggatggagggaaaggaaatgctgcccagcgcggccccacaggcagctcccgctagactcccacactgggggcggggctggcagggcagggagccggcaggtttcctcagaccaggcctgtgccccggggcctctggcagagccaggctcaggaaggtgaactgccagagcagttggggtgaggaggaaaggatgtgaggggaaggaggaggagcaggccagaatgcagaagcaattgtagtaggagaggacaaaggtgatggatgagaaaaggaatgtccagtgactgtgtggactagacacacttctcactttagaaaccaagtatattagaaagcggaaaagaccctcctaaagaaacttccagccaggtgcagtggctcgtgcctgtaatcccagctactcaggaggctgaggcaggagaattgcctgaaccggggaggcagaggttgcagtgagccgagattacgctactgtactccagcctgtatgatagagtgagacttcgtctcaaaaaaaaccttccatgccatttttattttagagtaaagcacaccccatttggataccccagtgctttttaacatgctcagccttggcaaagatagggtcactggggttagtgaaaggggtgggtgcacatgcaagtgtgaggccaactgttactcacctgtccctttctgttttctttcattggtggacccacctggaccacacttgggtctgttacctccctcaaagaggtttccagaaaaacctgaggacgtgcctcagggtgatgatatgcccagcagctctttgctcctctccgggattagggaaccttgtatcctcaacttcctcctaattaaagcctttggcagaatcaggatgcagaaccagggcctggaaccgcctcagtaaacccctgtggaaccactagcagatcatgctgtttccaaactgcgcgtgttggcaagccagcttttcactggggatgtttaaaattaaattccatgctcttgggaaaggaatagaaactataggcagagtctgtttctgaaacacctgttttaagtggaaagcgatcctaactgctgccatacctgaagggcttgtgtttcctgtgcagctggaatctctctgtgctgagtaaattaacttctgtgcccagggtagtcttgaatgataattagtgctccgtggccatgccacacatcctgtctgaagcaagggtgctaataatgtcctttcctctcttctcacaagacattatatgaagtcctaggaatagtggtcctggtgctaaatagaaatgtcgtggttatggcactaatttattgggcctgccagcccaggtgtgcagcatccctttcctccacccgattgataagacaccaggtgaggtccagtgtgatgctccaggcagtgctgcagctgagttggcctgtgtctcctggctcaaggggtgcatcttctggtctgccttgagaacttaccagtcttccataaattggaagttcaaagcaatggaatgaacaatggagagaacaagcacatacacataagttaaaagctgtgcatctctgtatattctaatcctttgaaacttggtgatcattattgttcattacataggatatggatttgatcagagcatcctttgggagttgtctgcatttggtatttccttttatatcctgaaagctgttagttcttgaaaggtgtattgaacctaataaaatagtaattagaggtatttccacaaattgtgagctttgtcatttggatagtgtttttgagacttcttttccactctaaataccaactttaaatagaataatatgacagaattatgatcttgaaaaatttatctacttaactgcatgtgggaatacatatttctgtagcatgtggaaaaaggactgagtgatgcatacctgttatatttacattttcttttggaagcaaattattttctacatgtgtatgtttatgcctacatgtgttctaatttgaaatgtaaaaatctataaaaaatttttccttgactgctatgttttaaaatatctgaaagttacctgcttcctagtccttggaaggcctgaattattataaagtttatttctttgcatctcaatcattcccaaaagattttaaatatgaatgcataagtagtgaataaacaaacctgaaagtttgatacctacagaacatttttaaagctgcttctttttttccatgcagtattgttagtagagtataatactagttatgtcgtagagcagtcacttactcatttatttacttcagcaagtggatattggggcctgctgtgtgttcagcacagagtgagtatagaaatgagggaagcagtttctgctctcaagaagctcagcaatgccttaggataagggaagtgctgggggcgggtgtgctggaaagagagagggatgggtggaagaaaattgaaaatcagacttctttgaagaaaagatacttaagctgggcacactatggtagtagcagctttctaggtagaccggaggaaaaaaaagtgttcctaagcagagaaatgggatccgtaaaggcccattggagtgaacatgcatagtatactcagggaatcagttgattgagtaaatatttatggaggccctactatgtgctgagccctgttccaggtgcagaagatacagcagtgaacgagacaaaaattcttaccaactggagctttctgatagacagaatcagccaaattagtaaggaaaacatacagcatacaagaggagagaagtgctacggagaaaaataaagctgagaaaggggacagagacaaccacagtggtatctggtttgcagttttaatggtctgccaaagtcttgccagaagttgaggttgattgaagacctgaaggtgtgctggagtgaaccatgcacacacctggagtaggatgttccagacagagggaacacaagtgccaaagccttgaggctgcagtagccggggaagggaagagcagctgtctgcacagtgggaggtaactggacggtcagctagggctaggcacaggcctcatttatcagtgtgaggggtcagacttgaccctgtggtcaggagagtgccatcatagatttatagatacggcttgtgctgtcatctctgggacccccaaagtgaggacccagtgagttggagctgagtgtctgttaggtatagaggtaagatactacacaaaggaaactcagttttgtgaaaaacgtgtaaatatgtttttcaaacatgttcttaagactttttaatctttctatgttcttgttgtctggtttatttcccatgaatgttagggatcagatcactgctgtgtaaataatagtccacccaaggtattcatatgcatttcttgacatggcctaaggcaagagtcctgctttccattgtcccctaatttatatttgcttctttctggagagttttaaaatgaatttcaagggcttgatgcctttaaaatcagtggatcaggccctgtgttgcgagatttcttaatgggccacagttgtctctgtgattctcatttccagtttatagcactactgtccacctaaacatacactaaccatgtggatcttaatgatgttttaagaaaaaaggttttgacatacatgccagacttccacttaagatgaaactgggcaactaggaaatggtatgccgtacatatgaagcactgtttattgaatattggtggcaaattttgttttgttgctaggaccagattcttggagtgtgtagtaggaataaataagaccaaacacaatcttgcttcactgaaaaaaaaaaaaaaatcaaaggtttgggtttttaggcaagatatttagggaagatatttctttgtagaccagtattagagttaatggcaaggatcagtagtataatcgttggaatttatgtcactctttcatttatcttccacttggtttattttgaagacttcagataaattattaaggtaaagtgcataggagtgctgaagttgatttgtaaaagcattccttgcaaaaatgattgctagctgatttcacaaagttgttatcctgaaaagattccttattgcaacttccagatcattacaccttttaaaaactattatttgcttttcaggagtattgctgtatatgaaggtcttagcaaagaacctctcgttttcctcgatgatagattagttgagatcatgtttatattggtgggttttcagttctgatggctctgcagctctgttccttggctgcaaggtgtcatagaggggcctctgcttgtcctggtggttaaaggaccatcagcaggagtctccaggccgtattactggagacggacctcaccagagtgagatgggagaattagtggtgttcctgtccttcttttccctgatgctcaggtcttccggattgacattaacttgaaattttgcccagcctttgactacctcactttcctgtttcttcagctcctcatgtcccctgctgggtgacagcctatatgggatttagacctaggttgctcagcttccctcctcagtaaaactgatgaatccatggagagtgcttattccagtgtctggcacatagaaagtgctcagtgagtgttagctgttatcagaagattagtctcagggggtgaaatgtgctttattatctgtgctacctcttcacctagtataatgaaaaatgtatatccttcacagtgcttattttcagagcaccatgtgtatttaattgttagaaggtctagtcacatttcttttcactctagtggttcacacttttggatgtgtgcaattcccatacctagtcttctggtttactcttggtgatttgactaacagattttaggttagagcatccattaatatttataaaatggtactgtgcattattcatgtcctcaagtcacattctggcaagaagcattaacgtgtgtcacattatctaattattgtcataatacacagaagtggcaggaagggagattaaaagtttattatttaagttattatttatttatctaacatggttgaggcatattatattcattcactcttttgacatgtcataaatattgatttgtggaagtgttgcttgatacagtattgaggttaaaataaaacttgcgctaatattaatatttcacaagtaggaagactgagatccaaagatgtttctctgttatttctaaataattgactttcttcctctaccaacaaacttactttccagaaaaagtttgctgctcaaagattcacattgctttgattaaattcatgtgctcgtcctctctatccctatttattccttctgcaggtatttattttgctaagctactatttgctaagccctatgatagttcagtgatgaagagtaaaaagtagatcgttagtttcaaagaacccacttcgggtaaaggagattagatgaacaatttcattgtgctgctgtaagtgaagtaattttaatagatatgcagtagaagtcctacactggaaatagtacttaattcagtccctatagagtggggaaacctaaaggaagtattaaccactgaggagaagtttccagtgcagatgaagagaagtagctttctaggcagagagaacagccatgaatgtcatgaaacaacatgggacattcaggcaattcatagtcactaagtcttactggaggaagaggaaggcatggcagagatgaggttgaagaagtttgctgggatcaagtctaaactggctttgtgttattccatgccgggtactcaaatgactgcatgttggacttcattttgtagaccattgctttgcaactccatatccttaagaagtttggttaggctgttctcttaacactccttgctgggtgttacttacattaaagcacctctgtaggaggataaaacactgtgaaccaggtatgtaagagacagctaatgacctggcttaatccaaggatgtctttttagagtatcctgatgaagagatcaaataggaaataattagacatttctccaaaatacttttttcctcaaccaagatttattaatttttaagtaagagtatgtttgataactgaaaattgatatcctttgaagctggataaacatgcagttacattttttactggacattcaagttcagagacagctgcgttgcttaatcatggcatgtgtctgaatagaaggctgatctagcacagcacaaacatggaacaggggagaagacctgagattttccttggaaactattggattttactctcaaagcagagggaatataagagagttaaggtctgtgccacaaagtgcagctaggaaaacatgtaaatgggcattaaatacctaccttggtgatttaaaaaatttcggaaatggagccattcagtgtctaactttgagtaacttcctggtaaaacctcaaaagagaaagtgattgaaaaaatagacatcagagtatctggatgagttaacatgctgtgtgagactactcattaaaagccatttataagatgtcttaataatctttctcaagaaccagttttttaattttgttaactgtttttgttgcttttactttctccatagatttattatttccatcctccttgattctttggacttactctgttgctcttttttcaagttggatgcttagttcattctttttttttttttcagcttgtcttatgccctgtgaaatttccctttaaacactgtcttaactgtgtccccatcaatgttgacatatggtacttccactgttcttcatttttaagtatttggtaagttccctataaacccctcacattaaaaaatctttttattatagaaacttccaaatatatacagaagtagagacagttgtgtaatgacagagtgcttcagcagttgtcagttcatagccaatcctgttttattaaacgagatttactgagacctcagctagtaaatcctctaagaatggagatactggaaaggtcctggaaagtgatatttattacattaaaagacctttggcttgccttactctacatagtgactttgttttcaaaacaaagtctaattggtctgataatggaacattattttaaatttaaattatgactgccataggcatatgattctctggcactgtccccaataaattgttagcattgtacaatttcaataaagatgctagaatcaaacacaaggataaagtctaaaggggcctcatggattatccattctatgtcctttttgtaattaaaaaattggagcttagagagctaaatgccttgtttggggtcctgccgtgaatgagtgacagagctaaggttgtggtctcctgggccaaggtcctctccattacaccatccccctctgggctgtttcttcgtatgcagtttaagatggcccctaggttaggcagaactgtgaagtaggagagttgtgtcccgtagccccaatttctcctgtccttatcatttcttgtttcatgaaacatgaaacatgaaactggtgcttctctgttagttgtattgctgaggtgtatgatgaaaagtacagcaatgggacatatacttaataacaaatattttgctgtaagttgatttattatattggatcagtactgcttgtttttcagtgatttcctcacttggacataatacaaatcagtgttcttgttgtttgttatacatttagacatttacatcagtttaaaagttagtcttcaacaaatatttgtgtccacactttagtattccattgtttgaaagattcaggctttgtccactctggtttatagttacatatgcttaaaattttttcactttgtggaaatgtctgtaaaatacatttttgtaacatgaatcatttctactgataaccataaatttgcccttcctgctttgcactgattgcggcacaaggaagagaaagagaaggaagcaccagctttgggaacataggccttaagcttgaagcagagagaatgaaagcagactgtattagattcctcctcgagcagaacggaatgcctgataacagccagccaggagctgggggagttaagagcagcgattttacattacactggagtttttaggctggcatgaaaagagatgccagtattagctatttgcatcttcctgtagccacagggatatgtgaacagctggagaattaatttgatatatattcccaggagcttctcttctgccaacagaattccattactgtgagcttctgcgtgtgtactgtgaatttgtttcttgctttttaatatctgctatagcatatttctggtaagtggattgccatataataggatgcagccatctcttggtttggagtaaactctattaacctaaacagatacaaggtggaaaaggtaagaatctgagcatacaaatgccttccaccttcaggctggcagaaatttcatgtgaacacacagtactttgtccagtacgtgcactcatacctgaaatgttttgtttcatgttagtggaatttcacacaccagcaagcagatggaaagatatgttgaagagcatagaggttgaatccaggaccactcaggctagtgttgccacagctcgttcctgtagctccttagcacagggggctatatctgctggagctgcttcttcacatatctggtgttcgttatgcctccacacatcttgtaagaggcctcatgcttcatatccttctcagtgcccactttcctgccctcaggatggaaacttctctccaactagcctttgcattgaaaatctttcatgtcaaatgtagaaggaaagaatttcaggcatcaccgagaatattgaaagatattcttagaggagaaagactaagtccaggacaagactgaatgtagctattggaagtacaggtgtttcctgccttggcaatataagttgttcactcttataaagcaaataaaaaaacctactcatattctgcaaaatcattcattacactaaaaataagagagctgttgggaaaaaatagggttggggaaggtcactcaagacctatgcatctgtgtgtatagaggataaatgcaaacaataagaactcaaataaaaatactggcccaggtgagctgtccctggcatttatttgcacctggatctctggtagcccatgctttgcagccctccacactccgaagctcattctgcctcctttcagggcacggggtgttgaagatacttacttttcatgtggaacggtgttaccagaattaaacataacatccagtagtttcttcatcattgtttttatgggggttttttgtttgtttgtttgttttgttttgtttttagtacaggggcaagtgtctccagccttctcatttctgcttgcagtttccccagatagtttaatacagaggacattttaaccacgagagcagtcatttcttgcactaaagaaaggaatttaagcaaaattttcagccttttcttcaggtcttccatatactggtaagacttaactgtgagaaaacttgctcctgatacttatttttatttatttatttttttgacagagtcttgctctgtcgcccaggctcaagtgcagtggtgcgattttggctcactgcaacctccacctcccaggttcaagcaattctcatgcctcagcctcccaattagctgggattacaggcatgcgccaccatacccggctaatttttgtgttttttgtagagacagggtttcgtcatgttgcccaggctggtcttgaactcctggggtgaagtcatctgcctttcttagtctcccaaagtgctgggattacaggtgtgagccattgcgcctggccaacttttgatttttttttttttttttttttttttttttgagttggagtcttgctctgtcacccaggctggagtgcagtgacatgatctgggctcactgcaagctccgcctctgggttcatgacattctcctgcctcagcctcccgagaagctgggactacaggcgcccgccaccacgccagctaatttttttgtatttttagtggagatggggtttcaccgtgttagccaggatggtctcgatctcctgacctcgtgatccgcccatctcggcttcccaaagtgctgagattacaagcgtgagccaccgcgcccggcccacttttgatttttttagaacattaatgtgctgggaaaaatcacatgagaaccagcagaaccgctgtattatactgacaccattttcctatttaccagttacatattggctaaatcacattaaagaaacattcatttttatggttccacggtaactgttagaagaggtacccagcattcttttgttattccttggcctaggtgtacctgcaggacatgctctttagaacagcagttctcaaagtgtgggctacagaccgcttagaagcctgaagccctttttgggtggtaaatcaggtcaaaactaattccataatcctattaacattttatttgcttttttttctttttccactttgcactaatggtgcaaaaaaaaaaaaaaatggtacataaaactcctgatactttagtatgaatagaggttgtggcaccaaattgtgctagtaagttactgtattcaccgtgtactttcagtaaagcaaaatggtaaggaattaaaataataaaataaaaattagtttccttgaatgcccttgatcgaagcaatacaaattattaagtttattacatttcagccctcaattatacatcttattaatgttctgtgggatgaaatgggaaatacacataaagtaattctgctgcatactgaagtacagagcttgtcatgagaaaaagtgttatatttcaagatgtcttctgaacatccaagtggaagtgttggtttagcagtttaatatacctggcacttcactgtagtactcaatattggacatatttgaacatatatgaatttgaatatatttatgaaattaggtttctctcttttatacttagagaaagtaacttatgtaatatcctaatttggtatcttgcagaaattataaacttaaaaatacatttgcagaggtacctcagttgtcttcacctttcaggctaggagactacatcttgtcacatttagggtaccaggaaaaaacctacttcatatagttaaaaaccagtacctctgtttttaactttttttttttcctgaccagtccaaggagaaaccaggagctatgttagagaatcagaaccttgattcttgtgctgtgtatgtggatttacttaggaaaagagagattttaaaacataatttgccaactgctgttagcagaactcttgggctcagatttgatttccataatccaacctcattacatttttgctttcagtccttgttgattttgttaaattattttaaaatatagtgttctgtaattgaataccagaaaagcatttgtctcagtagcatgttgtacttgatcctatgaagcggtggttgctagggaaaccatttcccaagaagaaaaacaaagtattgattaggagcgggcagagaattgaaagagtttactctcaaaaaaaagaaaataaaagtgtctacgatgttgttagctttagagtcagtgatgcttcctggctgaacatagtggcagcaatgtttctgaggggaatggtcacctttacctgagcatcttcaggtgagtctgctgctgctgccaggaaacttctgctgagaaagcagcagaaaacagcaaggggccagcggcctcatcaaaggccttttagctgtgattccagatttgagggaaatggtcttcctgtggggaggcagagaaaagcatctggcagtgaaaaccaattagctgtgagatgggctgcttggaaggactgttgatgccagccaggtgataagaagttgaagtcttagagctgaacggggggaagagagtaatgatagatgatgcctagtgagaaatgtgcccagtattccaagcatttctttgaaatgtgtgcctcatacagtgctgtaatgataagcttttaaaataggatttgtattgtttaaacatgttctttatattatcttgaaacatagttttccttgtgatgttttgtatatatgtgtgtgtatatatgtatacacacacacattttttaattagacactttttttgttaaagacacaactgcattgaccataccagtgacatatatgtgtgtctatatacacatatatgtataatacatatatatacatgtgtgtatatatgcatatgtgaggcttctttgtctaccggggaattatggcttacataaaaccactatggtaggggacaacaggtctgtgcagatgtttgcgggagcatagaggatggagcagcttattctcctggggcaggtagagaaggcttctcaaaagaggtaacatctgagctaggttttgaaggatgagcaggagatagcagaggaagaagggaaggaggtattccaggtggacgacactcccgagtaaagcatgggagcagttcctgcgtgtttctggtaagagcctcaagtgtgcctagagcattaggatgtgagagcgactggtgaaaaatgagtctgcagaagtagctacaagccatgccatgaagaagctgatattcagcagtaagaagctgggacattttcttattgccaagagagaattactgggcaggtttgttttttgaggaagatggatctggggacattgtggggaatgaggtggagagacaagagaatagtggcagggagccaggttagaagacattgtggtaattcaggagagtggtgtcttcagggagttccagttagaatggtgggaagggatgtttactgtggaaaaaattctgcaataggatcaactcaataactgcacatatttataagaacagtaataaagatttcctcttaaaactgtgaaggaaaggaggacaaatatacatggcttccagtagttcaaatgttacttgaatttcttacaagttttttttaattcctagggctaaactgcctttcagttataaatttgcatatgtagtcacaaaaattccttcatggcaaatctgactgaactgtagtaaaatgctagtactactggcatgttaacagttaaacagataagaaatcacttggatgcacattttgttataactgaagacaaatatcaccttcttttctatgttgaattttttcaatatggtgtttttcttctttttctgattttcctttcagagtactttactaatttcattgttgtcttgaagataggaatgtacctttatgtgtctatgttatatttgtgtgtgtgtgtatgttgcttctctctgtctatatatataaaacatatatatatatcttatatatgtatgttgtttctcagaaccaaatttaggctaaataggagaacagattttaaataaaacataaattgtgtgtcggtataaacttcaaatctgaaagataatcattttactaaaagctgaggctttgatcaaataagtgagcctgatctcctgaaatctaaaacaagagattttctattctcccttttcaaaggaccgccctgaagctctttatgtagtagatgacattcttgtaacgacctcttgatgagaaaattatttatttcagtcaggaagagcagacttttacttcacttacgtactgagcaatgatgccagcaataaaagcttaccctataaaaataagagccatttgtttgtactgtcttttttttttttttttttttttgaggcggagtttcactttgttgcccaggctggagtgcagtggcatgatcttggctgacgacaacctctgcttctctgtttcaaacaattatcccccttcagcctcctgagtaactgggaccacaggcatgcgccaccatgtccagctaatttttgtatttttaatggagatggggtttcaccatgttggccaggctgatggccaggtgatctgcctgtcttggcctcccaaaacgctgggattacaggcatgagccaccactcccagccaaaaacagatttttaagatggcatttatacctgccttagtttgagcaagaggtttaaggtttcttaaattcaggtactttaaaaaattctcttcagatatgcttatcagaaacatgtttactatgtaacagtcagttcccatttcaggtgggtaaagtatctgttaaagataatgttaagtgtgaaaaaaccccatataatggcccaaggattcacagagagaaatagtctaatttcctatgaggaatattatgaagaattgagcctgcatatgattctgtcatattcattgcattttttggagctgagtcccatgatagctctttttaaaatcgaccttatttattgtgacctgctccattccatgatcagaacctgatttggtctcagcctttgtattcagttacctcagcctgtcactaagctgcaccactttagtttttttgttgtttctggctttgggttggcagtgatggatactatcagtctccccaaacgcattagaggggatcccctccattgtaactcttttggtggtgtctgatttttgatgttgattattagctgccttaatgtagtaagttgaagagctgttttaaacctgaagttggtctctatcaatagagtaaaaaattagctttttaaagagagtctcaccctgtttcccagaccggaatgcagtggcgtgatcaacctcgtgggctcaagtgatcctcccacctcaaactcctgagtgctgggaccacaggcatgcacaaccattcccagctaattttttgttttgtttttgtagagactgggtctcactgtgttgcccaggctggtcatgaactcctgggctcaagtaatccccgtgccttggtctctgaaagtgttgggattacaggcatgagccactgtgcctggccaaaaaagagctctttaaaaaataattttgtagattgacaaatgtgactcttgtaattttattgaacatgaaaaaacccaggaatctttatttgatattaaacatttttaaaggcatctcagttgttgttgtaataacacattaagagaagtagtggttttttatttccaacctttgtgcatatagctatttaatgcctacatggatggctattatttcacttttttcagttattatgaagagattgggtttcattcatttgtaaagtttcagccagactgcctttcacaaattgatttgtcaaaattgaatgttaatcttgacatcccagtgcgtttttgcccgcgaacaggcctttgaatgaagctgcaaacacacattatctggttgttaattgttttacagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaaggtaagcattaatttttgttaaccatccagtcgtaagccttagcattatgtaaccctgagtagtaatagtatttgagcttgaaaaggcaaagacaagtttacagagagtggtagtttgtataatcccaactaagatgcataaagtttaccaggtgcttatgtggtgggtgtgtttccgagacgctatggaatggcggtgctgtggtggtgtgagcagggccctctgtgccagccctcctaggccacctctgcataatggatgatttcacaagcggtgtgcttgagatcagcatctgttgtcaccaggtccattctgtttgatttaccgtaggatggtgctccatgaggagtctgctaattagaaatgcggctcatctggcatgaaagagttatctctttagcagaaatgggagaattatgcagtgatgattgaaggcttaggttggttttgctggagggcaggaatcacatagggcgtggagggatcattaggctcttcacctctggagataagtcagaaattgcattcaagtaacaagaaaattatactgatatggctcataacttttgtacattcatacatacaaccaagaaaaaacttcaagaaattgaatatatgactcatcaccacagtaatcttgagtgaagtacccagagcttggtgccatggctcatagccaaaaggggtaaagcctgggtttgggtctcagctctgccgctaaccagcattaaactctcgtagccccagtttcttcttgagggaaaaaagtactgaaagaggcattcttaaatatacctcttatctctaaaatgatgtcgttgtaagatggagtgattggtaatataataccaaggattcttaaagtccaaaaatcaagcttataagggcagtaaagcaggactgaagttttggaaatgtacttagtttccaagttatattaaatggtaaggaagaagaaatttgcagtagcctacaaaataacatttctctttgtatttaccagtgctcctgaatcattagaaaagaatagagtagaaaacaatagaaataacatttgcgaattgcagaggatcctagagaacatttagtccagtccttgaacttgaacactgggaaactgagtttccacgcatttgatgagtatcagtgctagtactaatcacaccaagtattcttcctgtcacatcatgacttaaacaatgccatgggttacctaactcatttctgttgttcggttgccctttgctccccacacccattgcctgccacaaaagggtattttgccaacttcagtatgtaaacatttagggatatagatgacgtgatcacatgtctgtttacatagttaataatttacgcatacacatttaaacttactcatataaagtcatacacaatttagaatcattactttttgctattcttacatttcaggaagctgtcctaaatggtgagtgcgtcaccattgctgtcattgtctctactcaggcccccttccaggtcatttaatcccctgctcctgaacatgccctcttcactgccacctaagttgtttgagatgcagcagattttgaatctaaggatgatgctatcccaggaagttttgtcccaaaacccagtgtctcgtcacataaaattaggacaattgcatttgtaattagttaggcgtgtatttatgatctttgcaatgtggagattgacatgtggagattaactttattgatttttaaagtgattctaaaaaggctgtttgtgagatccagcatttccaattttgagatcattcccacaggaataattaccgtatctgattgaatttctttacttgttattttccaacttattatttcagatggcttctatgtaatccaaatgtggaattagttgaggtcatttaaggttaatgtatcttctacaaacagtgctttgttattcaaaataaagtaccaggcagtatgtgagatcttgtaagaagtcaggattaggggatttcccttttctgagaaggaattttgaaggaaaaagcatacttatattcaggtatatattgtatatgaaagaattagtgatgaaatcatacttaataaatagtcacgtcgttaatagtgacagagacttggaatgctataggacctcaatgccaagaaagtatgatttggaattgtgtgataaggtatcatggagggtagactgaacgatttagaatagtgatgatgatagatttcagaagtgaggccaacatggacgaaagaagaaaatgtggaagtgggaatctgcatgacttacatggaatacacttgggaaactggtctgaaagagcagagttttatgttggtggacaataggaaataaagattggagggaggataggaccagattatgaaatgtcttaaacattgtgcaacaagcttaagccgggtaatatgagcaatcagaaatcattataggtttatgaacagacagaatcagaaagaagatggtgaagtcaacattaggcagactttggttgtggtggaatgcaaactggattagcagagggagataatggcagtcaagaggctcgttggagagcttttcagctgtttagatgtgttaccacaagaaactggactagagtggcagcaggaaaacagcaaagaagagatggatatgcaccattttgatggaagaacctacggtatctgctagcaggaaagggaaggatgaaaattacacaaagatgattctagggtttctaacctgaaatagaatgatagtgcaagtaatagaagtaggaagacttttgaggagtctcgtgataaaatggtactgaaaataggggtaattattttagggcaatgtagaggtcaagaagagcttaagaaaatataggagatactacagcatgtttggatcatgaccggaatgatttagtaagaaggaaaagccaataatgtaagaaaggcgattgcaggagcaaagactttaaggaataaaaaggacaaaattgtttgtttctcagggaagtataaaagaaggtcatcatctgacattgagatggaagaaagggtgtggaaaatcaaagagaaagaggaaaatgtaaacagacatctttaaaggagaggacaaatgaattaactagggaaatgtagtagggtcacctactggtgctgacagcttacttgaggttagtggttatgcctttgaaaagaggctggccagcacagttgcgtgattttctctagttcttatgtaggtgtgcagtttaggtagagaggtagccggttgggtattttcttgaccattctggtagaaagagagagagggagtcatggggattgatgattatataagagaactttatggtgattgaccatttaatctacactgagaaggagacaaaatcagggagtcagtgatatggagagtagaggggaggttctagtgggattgaagaattaatggagtgagatagctaaggaagcaagtagaaatgaatacttgaagagaaagttattattggttgtaatgagggttagaaattactatggatgatgtagttgattgaagaggggtgaagaacaacatcactgggggtgggaggtcagataggtgagtcaggatggagtgcctctaccaatgatgaagatgccaaggaatagtagcagggaagagacatggagccagaggttctgcaggtaaatgccacacctccgtgtggctggtgataacctaattgcttatgcttcaagtggagaaagttttattttttaaaggggaaaaggaagaatgatctttaagtagcagtgagaacaaagaggatacctatgccccttccaggatttatggaatgtgggagataaagccattcccatttgagaagacttcaagggaaaaagagtaatcaggtactacctaggtttcaatttacacaagaaggtgaagggaacatccagatggaagaatgagggcagaaagaattttgttgataagctctagaaggcacagaatgggatttgggagggaggcttgtacagggcatgggtgattttgcaaagccaggaggggttaagagtgcagtgatgatggatgacctggcgaatttagacttgctctcgaatctcttacaggaaggtgaatagtctagttaaagcctccttttagggactcttctttgagtagtttatagtattaagggtgtgcagtggtggggatagggcaagcagaatggtattcttggcagagcataatgagaaaacatgggtctccatttctgtgtttttggtggtgatgctgaaaatgaattcgaaatgttgctataacattttttaaaacgtgtggaagtcattgcaggccatttcaatgttatagacttatggagaatactggaaatttctgggcattgaaaaagttcctgtgtattacttatgtttgtttaaaaggagttggtcctcaattttactcagtgttttcttttctaaaatacttgaacatatcaccataaaatcagtttgttaaaaatataaaggaaataggatactggataacagtcagccgtggcttagtgcagaatagtttgtcagagtgatctgtattttctaaggaagaagtctgataaagggaatgacctgcaagttatcccaccttgatcttaacaaagtttatgcttccgtgccttactacttattcatcagtgatttgggatatgttagactgagcaccacaaaagttaaattagtgcaccatgggcacatcagtttgatgcagtagtaaaagcagtgccagcctctatagcgccagcctctgcagctgccacaggtgtgttctaggggtctctctgggctcagtggtcataagtaatgtcaataccaaaggtttccatgatgcagtgaataagttgaacttttaagtttgcctataattccaagttgggtacatggcttttaaaaaaattattgtaattcagtggtcataattcaagaattaatcagaaatgtgaaaccatgccacatttggtgaagaatataaaacaccctggaaatcattcatctgtttatttcatccagcaagtatttatgaaatgccagatacatacgtgacactgttgtagttgttgaggatacagctctcatggagcttaactccaaagggggacttggactgaagtcctgccaccggaactctagcatggagacactttcagctgtagcaagaaaccttctgccatcccctaagtacagggaagacagaggctcccagctctgtgcccacccacatttttatacagggctgaaggtccatggggagcctgggtgtcatgcaccaagggtgtctcctaggtttctcaaacttgagagagcctgatggtcacacaggcagtaaatacataaataaacaagaaaatagcatatggtatgataaaagctttggaaaacagtaaccagtatgctgtgatagagaattacttggaaaattgctgtattcagggacctcagggacatattctatgaaggaagggcagtccaaactgaaatctcaaagcccaaaggagtttgccttatgatgacctgaggagagagaattccatatggacggaacagcaactgcaaaagctctaaggagagaacaagcttggtgtgttgaaggactagcaagaagaaacagcgtgaccagaatgggggggagggaggggtaagggccagaccacctgagcccttgtagaagatgatgagatgtatggattttgtgatgagtacacaggaaagccattaaaggattgtgagcaaaggtttgacatgatttgtcttttaaaagatgaatatggtggaaaaagagactggttggggtagggagatgtaagagtatgaacagagctggctggtaggggttcttttagtagttcatgtaagagataatggggactcgtacctgagagctggagatgaagagaattaggtgcattcgagataccattggaaatagaattagcaggagttgttaatatggatgttgagggaaagagaaatcaaagagtactcttcgatgtggagtttaagcaaattgtagtgccctttactagagggaggaccagatttaggaaggacccaggagttttgggcaaattaaatttgacatccacatagaactatttgtcacaagcaatttttaaatgtaattcagtaactcattaagtagaattcatacatacatgctctatggtgtataattacaaagagatatccctgagaaaagtcaaatctctccagaaaaagttataacctcatcaggagtttgtatagaaaaataagaataacctactttgcagtgatttatcaccaggcagaaactggctgaagaggtgtctgtgttatccataaaatgtgttttcatcattgctgcatcacacatcacatgggttagattggtgtagaatatgtggaacctagtgctgcttgcacagtgaacattttgcaaaaggaggaagacacttagggctttagataattctcctctgttttagctgaaaacgtttggagaacgttgaatggtttatgatttttggctgttttgaattatttcctctatatagagaaaaaatagacaatcaaaaattggcgtgtgatactatacatcattgttttatattgagtggacaaataagaaccgtgttgcattcataagatggataaatcaaggaaggttttgtggaggaactaggaattgggtccaactctgagaaatttgttgattggctcagcagaagggaaggagagtttaagagcagccttggatagaaaagagaggacggacagagaggtacagataggaagatactaaggaggccaaatcagtgggcttcagtggctgattgactttcggggatgaagaaagggaagacttccagattgtgggaggactattatcaagataaatgctggagaggaggtggtcaggatagattttgggaggatgataggatatattgtggtgtcatttattagaatgtgaaatacaaggagagcagctttgtgggaggttggatgaggtaggtactctgtttggttatgtttgaggtatctatggagaatcaaggggaggatgagagttacaggtctggaactcagagagagatctgagctggagataagaatttatgagtcaccctctgagggagactgtggtgtctaatgactttttgagtacataaatgatgtgaggagtatgagcatttttaggaagattaatgtgacagtaatgtgcataatggaggaaaaaagtgactggcaatgttaacacaccaagagtgataccaagtgtatcttatacaaaaggaccccgtgcagggaagctggcacaggaagtgactcctctgttagcatggtagaagaaaatccaagggttgctactgttagccaagtgaaagtctaaaacacaattgccagtgcccatgatattgagatgtataaaatggaatacattattttaattaattgttaattgattggttgactaatttatactcccttgttcaaaaaagcctttaaaagtatctgactatagtagagctggtcagacccacacctggaattagggctagttcagagtagaagcactgaaatgattaagtaattggatagagaggtcaagaaataggattatagtcatagactctttcagtccagaaggtactcagcattctctacaagtaccttccgaggcgaccgctttcagtttagagctgctttaattgttgattcattcatgcatttattcatgtattcattcaaaaagaatgtattgagcacatgccatgtgccactacacctcagattcactagggcaaatttagtttgcttttacctgaggactattttctaaatgacctaaattgcaacacattttcaatatactatgggagcttgctttgtttttttgtttttatattttgattttacattgtatggctatgttctctgaaatgaaactaatttttacacaaagacttctgttttatagccttttttcttttcatttttaaaagcgcagataacagttgcctcagatcattaacttaattatcctgtactctaaatttactatttcagtgctacctaaggaaatggtaatatcatgagttttttattccatttttagttttttacatttcacttatttcacttcctttttttctttacacaagtaaagcgtacgtgctaaattttattctcgattgttacctcgtccttggtctagaggctgtagtacaaccctgtgctgtgtgtggcctaataaaatgttgtaaatcttatgtcatctgtaatgtttgtcagtaaacaaatctttagtatttagaaagaaatattatgccattttttatttatataactttttttctcaaaagcattttgggtattttttatgtgcgtgggagaaaatgctcacagaaaacacccaagtaaatgtgatctggtggagctgggagtattagcattcatgccgcctttgaaatagtgcaacagaatttcctcttaatgttcctgccactagaaccgcttcatggagacacttttcagctgtagcaagaaacctgctgccattgcataagtacagggaagacagaggctcccagctctgcccctacccgctgtcccatacggagctgaaggtccatggggagcctgggtatcatgcactgagggtgtctcctaagtttctcaaacttgagagagcttgatggtcacacagagagctggcttaactaggactagactagggctagggaaatctgcatttccagcaagccccaggagaagctgctgctgcccattttcaccacagtttgagtagaaaagttatagagtgtttatgtgaatcgacttcgctgcacagagaaatggatttgttgtgctggtgtcagagaacatcatatcttcaagtagtcactggacctaattttgatttccactacttctttcattgtttttttaaaaaagcatatcttatacattgctttgcttcaaatgttaatacatggtaatgatttcattatgcaattaaaaaatttttttctcaagtaaatgttaaattaaaaatgaaatatttcatattatacaggacctcatggtaagggacaaaacaaaccctgggactcggatgctggaaaaatcatttgcttttaatagagttgatccttttcataggtttacccatttggtctttgctaaactttacaaatatgtctaaaactttatgtctatggattttttttggcaattatattgatctcctgtatggcattaaatgccattatttcaaaatatttcctaatatgttattgactgtattagaaataatcattaattcacaaaatcagatatactgaaaattaaattatatgttctccaaaaatataattgctgtttactatgagagtattatatagcagagttaaactaataagacagtcgttacaaattatcaccactgtaattgttccattggagggaggatggaaatgggtgggttttgtacattttataaggtttatgaagtttaagggaaaggattttggggaaagctcttggcctttccaaactgctgttaatatactcattgatagtggaatatatgacctttgatggcacttgggaagctaaaggaaaaatcagctgggaccttgatgataacgaggtccctgacccacccggaaggtgaggttaaatgtgttttcctttggtcaagggaagggaattccagatcctttgctcatgtcaggaaaaacctttgtgggggccaggacttagtgttttaaacttgataagtcttactagtttaactctgctaatatttgacccctcccccccccccaaaaaaaaacaaaaaaagataacctctcaaactgattctcttactcgtcctgaaaccaagcattctgtattgcaagggccatgccctctttgcttcaggactttcatgtgtacagattcctaaattttaagttacacagctgcttcatcaaacttactcatcatagattctagttttcttaggctagttacatctgaaagtggtattatgcttgtgaatgtgttgctttagctcatataatttgtttaaaaagtttattttagaggaatccagttaacactgcttatcaagtttagaaacactaagtcctggccccctcgaaggtttttcctgacatgagcagaggatctggaattcccttcccttgaccaaaggaaaacacgtttaacctcaccttctgcgtgggtcagggatctcattatcatcaaggtcccagctgatttttcctttagcttcccaagtgccatcaaaggtacttgtttataaggcagtaagaccattatgcaactttaaattttatcccactctgtttgtaaagacaatcacaaattgacaatattctttgtcttttcttttttggagcatatatacagaagcacacacacatatacatattcgtgtgtatgtatatgtatatctgtaaacacagatatgtaaagtgactttaaaatacaagtacatgtgtttcattttaaattctagcgatgtcattgggaaagaaagtgtagacaaagttacatatttttgtaagtagtgtacttttatttacgttgacagttaatatttaaaaattttttgttacagatacaaattttggggagaattgtccttagaaacatgttttaaatcgttgttctgatgtcttgataactggtaccaggaaaataaaagggttaccaaaaggcagaaaccagtttcaaattatctgggaaataaaaacaggaaactcagggaacttggcctgagaccagttggagaggagcagttgttggttgaataattatacctgcattttaactgttggaagtcatacagcgaccttctggttgttgtgatttcttgtgcccgtcaaaaaaaaaaaaaaaaaaggaagtcacgattaaatatcttatgtccagttaccaacaggagtgtgagtgtaaaggagcctctttattcctacacattaggttacaaaaaggattggtataaaaaacaaattttccagttgacatttagagtgatactttctatacagtcatctgtaaacagcataataatgttctataccattcattcatggattttttttcaccttcttctttgctttaactcttaactctgagctttgaagccatctcttctgtccaaagatgtgcctagttcaagcacttttctgtcctctttccatacctaatcccaggttggttaaaggagcaccagaagttgtaggtgagcaggttttgctacatctgggctttatttcaggattaaatagaatgtacttactatgtgtttttaaaagagacttattgttaatattgcaaggacaaaaacaacctgacaggttgagccagataaatggtgttcttgagggccgtgcctaaaacttcaccaacccaggatgcctaaaaaatgttggtctaatgactgaccttagcagggtgacatgttcactgctggggtagccctgagcactcctgcgcgtgtttgtggtttctgacttggctgtaggtgacctctcagtttttcccagggctcggcgtgtgctctgtaacacagagctgctgagttgctgctttgcccagctgtgtccggctaagccctggcccccacaaaggtttttcctgacatgagaaaggatctggaatcccagcccgggaggccttctgagctgctggcaaaggatgctgtgagtggagaccactccccatggcagagctcccttccctctaaggggagctggagctgtcccttctgtgtccacaggggttctccgcctcctgcagagcggagctgcagccccaggcttgtctccatcaaccagagggtttgcataagcaggagctgtcaggcaattaaaacctcgttagttagtgtaattagtattccaagaccatgatctgggattttggacaactcaaccaagctggacagtttatatcctgcttatgagaagaaaaaaaaaaagtctgtgcaagggaagttcaagaaagagtctgggagcataaccactttagcataaactcacagacagtcaaaaaagtgatcatttcaaatgttcttataattttatcactataggtaaggaatctctattagacactgtgttttttctactctcctgtcttgccagtattatatatttactagtatcttgattaagagatcctcagaggggaaaaaaaaaaagctcttttaaataaagagagttttaattaaaaaagaaaataatttgctaatgggaggaagcagggctgaatgcataccatcatacgtaaaaatataccactaagcaaagccctactttcctccagttcttgttgaaggttaatagagctaaccttacatgaaaattgtaaagattgccaaatttgtctttttcccactatgccctccctattcttttagtttttttccacctcagaagcccagcatcatctttctctcgtttccaccagtaactataatctaggaaaaattttaaatttaaatttaccctttttgattggttgtaccctagagtgttacctctgccagcatccccacattttaatcttaattgtgaaattcatattgaattcaatccagaaagaaggagcaaggatttctgcagtcaatgataccccttcctgactgagccctgctcacttggggaggattgtcctggggctggcccattgcataggggcatttgagatgaatatggaaaggacctcgccctaagaagtctagtgggagagatcatagacacaaggagttataatgagggtagagcagccatacccagtctgaatagtgttatgcccggtagacattctagaagacattgataaacaagaatcaaagctaactttaatcagtttaaaagctactttgtgatttatagacacatatgtttacttctagaattggcacttctttgttctttgtgaaatgtgtaacttaagaaaggtatttaggagcactaaaattaaatatttgagtttttttaaagaagcattctaataataaaatttgactcattccctagttcatattgaccttcactaagcatactgttctggggcatttgaataatttgtttcaaaagctatgagaccattcttttaatggtgcatacgctctaagataagaataatggattcccattggttgatgatacatgcaaaggtagggatatactcttacagtgtgccttcctgcttttgattcgtggtttctcttgctaagtgttttctgcatcaggaggaggtgacagcagtaagtggtgcctgtaaagttggaaaagggaaggaattaacatgaattaatgtctgtgtgagtttttaaggaactggaatgtccctctaaatcgaggatgtgttttttttgtttctggttgacaacttcctagttttcctacacttcattattcttttctctgtcactgggatttttttttcaatttcccgaaaagcaaatgaagtgctttatccttatcagtagcctttcttttcctgttccaataacaaaagaacaatacaggaatttaataatgttaaaaaattgctgtgtctttttcttaatatatccatttatttgtctgcagataaaatccaagttttacagacatagaggaaagctgtttatataagtaaaatgagctactattgtatcttgtccttgtcagggttttctttttttgtcttttcacccagtttagaaagtggattttttttttctctttaaaatgtgtgcccatgttctgttattaagaggaaaagtcctgaaaatacaaagataggtgaaaacactggttatcctctaacagaaggacactttaaagattcctaaaacacattctgatcttgtttatgtatctatattctattaaatattgttatttactcttcctgctgctttggaaaaaaaaaaagtccaatctgcctcacagaccttttataaatatcagagttgtggttttgcaacagtgtacatgaaaagacacttgtttcagtgtttgagttcatggcagggtgcattaacgtggcgtctgcaagtatccttgaagccacattgaggagggaacttgggacttgttccatggtggtcctgctctgacctcagaggaccttgcttgctgacttttttaaggcatgaaacaaattctcatattctcagtcgtgtctgacgcagatctgggtactagtcagggttctaaactgcctaaataaatatcttgcatataacatctaaagtgacatttatctctgtggtactaaaaagaaaatactattccacttacatgttcttggtgccctttggcattttgattgctaattacaataggaccttcagattgccttctgattgagacctaacttatttctgtttcatctggaatttttcaaatacaattattatgtggtgttaacacttcattgcagcatggaactcccaaaaatgttgtgtatatattctttcatgcctctcagcctggcttcatgtctgtctaggtacaggtcttattataatgttgcccttgggggtccttttgatatatcaagaactaaacctctttagtcaggagagtgtattctgtatggatttataataaatagtctgtgttcatatgcataggaaataatgcattttctgaaaaaaattatgcatgtcacttgtattttttgtactttatgtgccggaagaacattgtatacaaatatgtcccatgtgcatgtatgaatttatatagctacatgtatataagcacatatacatccatgatgatatcataggtaatatgtggaaataagtgcaaccatgtgattatttttatcttattctttgtgtgtgtgtgtgtgtgtgtgtacagttacctgtgtgctattaaatgtgacagtgccgataatagtgttacattctctggttaagtgtttcctgagaattttggtcttccttaatggtgcacattagttatagactcttaacccgtgagatacattccagtggttcgtttctaagaatcgatgtaatattatgcactttgtaaatagcttatagaggataagctatgactcctgaaataaatattattcaaactgcttccattttcatttcccttatcactataacaaccacacccaactttgcaattaaattgtgttgtctatctgcttcccgcacactagtgaagctgaccctattgaattcaaaggaaatatgagagataaagtcgaaaactgtcttccttttgttgacatttctaaagcaacaagtcagaaagcttgactccctttaggtagaccagagctgctacgggtcatgattagatggtatgtccactagtaactgtgatgacgcccaacagaattatttgaaacattctcagtaaataaccaagaactccagtaagcaaaaggggagtagctgttaacttgcagtttcagttttctacaaatgggggcgcagggggtggggggcgcacggaataccgaggacaaaggtaaagatcgacatagaatggaagtcaccagatttctgcgtttaacccattcttgcccagcccaagtgaaattgtgttttggagaagcctccatatttggccatcgagagattggcagtgggaattcttggctgtattttactgctctctttattgaaatatttgccacatctgacagttctgtcactgttcagttcagagggatttttaaatcatatactgactttgatgcttcatggatttgtattatttgtgtattcctcagtcatactaacctttgtatcaagatttttgccatatgactcatatcaaagtttttaagacatatttaaaacaaatgaattttaaatagtggcccatttgctatatgttaaatttgatatacaaaattttatacatcttaaatttatatatctttgatatcttaaatttggtatacaaatggtggcccatttgatatatgtttgcttaacataatgtaacatgattgattttgttatatttcccatgagctgaggtactgttgttttctattaccagagaagacatttcagatcttttaaatcttatgtgaagtacttcctaagaagagaagtaaaatgagtatatctcacattagttactattatctgcagcatcaaaaataaatgaatatccccaggaaaaaggttataaaattacaacacctagacacattgaggtaggtagtaaatgtgaaatgggcttacgaatgactttattcacttactggcatttgggagggactcaggctgtcaggaaaatgaaccaatctgtattaatataaagcgctagaaatataaataaatcatgtttgaaagcgataccaggattttccttgaatgctgctgaataaattctgcttgttaaatgtcagtgaaatgtatgtcacctgcatgcataaagtgagagggccacaaaaaacatccacttaccactttgaaatatgtaagccctttaatgttcctttgaccttcagttaaaggacatagtctcatagcagaggaaatgcctcaaattctagcatctgggtttgtttctttatacttttttggcatagttacaacagtagggcttttctgtcctttaatttttttcacagtacagtcaacacaggcttttcacacaagcgggaaaggacggttgtgaggccaaaagcaatatgtttcttct

SEQ ID NO: 21 - DNA, 50000 bp, Homo sapiens
ctgtgctccacggcagagcctaattgaatggcttgttgtttcagagagagcccagaaccaacaagcagacaaacagaatcccacacaaatacaaattgcgtataaaattagatacctacacacgtgcgtatcaatggatatgtactaaaaatgcagtcagcgatctcttattttgagctcattggcttagtcaccgtgttccaattctttcatctttgcagtttttggattttcatctgaattttcagctggctaaagatgtttcttctacagctgaaaagagaacctagtttcattacatacacttgtaaagtgtatgtatttggatgggggtgctctatttcagccctattcatatgtcaaccatattgtcatttatcattagcaaaaggtcacttttaagctttgcagaatgaaacggtgagatccgattttccattaattaaaaaatatacatcataacagtatgctggagacatcttcatatatttgtgttttccctgctttacatccaataggtttctgtgtatgagcttctcctgagactgaataatagcagcacaccctaatgcaccttttttaatgggaattcagttgggtaccatatgcctttctcactgctgaaggccttcatgctttggcatctctgctctcatcttaccggtgattgtgggccatttgtctggttctgacatggaaatattaggaagcagtgctgttatatacaaatccttatagccttaaaattaaatgcagctctcaaaagcagcctaaaagttaaatgacacacaaaagcttgttttgtctgaagtttatatgtttattctgtagagttacattggatagggtagttatcagcccttagcaaatgcctacttagttttccttaatctgttattcattcattcattcaacaaatatttattgagcgcctgctctgttcagacactgtgctagattcagggcaaacaagacagataaggttctagtccccccccgagctttcagtcttaagtggcaaaaataaaattatgtgattgctgttgacgtacagggtactgagactgagataggatagcttgagaaagtctctcctctgaagagctgatgttttaagttggactgaaaggtaagaaggagctggctgtgcaaagagctcgtggaagaacattctagacagagtgaacttgcccatgttcagtgaccccagtatgggagaaagagttttgtgtcccgagggacttaaagttgtgatgtggctgtagtgtattgagtgagtgttggggagagtggcagaagaggaagctggagaggcagggaagggctcagttacacagacaacatggtaaggagtttggattttattctaagtataataggaatccatgaaagagtttgaagcttcagatggagatgagaggatgccctttttaagatcaactgtggctgccatgtggaaaatggattgtagcgggaagctagagtagaatcagaaacattatttaggccactgaagaagagcccagccaggagactgctatgtggctggaccagggtgctagcagaggagttggatagattctctgttccatttggaaatacagttaaaagggctcaattaggttggatttgggggtgaggttgggggtagacttcagagtgaccccagggttctggcttgagcagttggtaccatttcctaagatgggtaagactgggcaaaaactaaggggagtcaaaattacctgcttgagaaactgagagtatactggaggcagaagtgattctgagtccttttcttcgtgtggtgatgctatttggaaatcaagtcattagagtgtttcactacctggaacacatcagtaaaattgtgaaaattccaccttggtagctcttggttacagctcttcagctttgtcttgtgctgttttatctaatcctctgtctcttctgtaggctgtttctctgacctttggagaaggcttttgtttcttccccagggatgtcaacacaacccatatcccaatttgtttggcttccctttaaaattgttcatagattctttctgtaagtttttcactacatgcatctactatttcaggcatgattatcgtcacactagaatcccggtggaaccaaggctagctgggggaaatgaaagattgtgagtgtcatatctcatgcacactccagagcatgtggagaagcccataccatttaaatcattcttcctccatcccctaactgaggacttatgtggtaggaccttattatacttagtctatgtctgtgtgcttatcataccacattttattttcctgtgtgcctgccaatatcctctgaaagcagggacggtgcctggcatctaggggatagttaatgagtgtctgatgatttaaaaaaataatggattggggctgggcacagtggctcacgcctgtaatcccagcactttggggggccaaggcacgcggatcacttaaggtcaggagttcaagaccagccagggcaacatggtgaaaccctgtctctactaaaaatataaaaattagcttggcatggtagggtgtgcctgtaatcccagctactcaggaggctgaggcaggagaatcacttgaacctgggaggtggaggttacagtgagcagagatcgtgccactgcactccagtctgggtgatagggtgggattttgtctcaaaataataataataataataataatggattggaactatggaactaaattataaaagataactttcatgatatgctaaaaaatgggactttattgccattgaggaacctaaattataaaagataactttcatgatatgctaaaagaatgggaactttattgccattgcaagcttgtgaggagagaggtagcatgtgaagttaagactagaatgaccggttccttgattgaaaacaatcaataaaggtgctgtgaacagattatcagaaagtatctgtaagtataaaacacattcagaatttagaaaacaagatgtaactcctactacaacactgcgtttgtcttctgaaaggaagagacatcacaggtttagtattttttgacctgtcatttgattttagtttacgcctaatttcatttaagcttatccttttggccatagtcccatttcttttcagtgtttttgctgccagtttttcagatacagaaggaaagtcatatttgctgtgtgcagttggaagaaaccctggcacagttcctccagaaaccaaagacagaaattattagccatatccagaccagtagctgactggctccaattcttcctcccatgagctttcctgattaatgggagaatctgttctgtagtcgaacagcctctttcctgagcccagtctgtcctggcttcagagcatgaccagctgtgcagctgaagccattgccagggctctgtttctgattctgctcctcagcatgttggaggtgttggacggggaagcagaggggtgggaagagcatccaggtcagtttagatgtttcagcaggtaacagacgtggcaccgtccacccagaatcccgccactcccattggactctgtcctcttcagaaggctgtcattctgcatggtgcagatgttgcagtccttcccttattcttttgtctttttgtccagaccacttagctcctgtgtctgtggtgattaaatgctttgtaactttttctgacatccttttcttcacccctcttctctcttcccctactgtctcgcccaccatacctcttctgttttctgtttgcctttctttcctctcagctattgcattttcccctttacactgaaatagctttgtttattggcctgtttccagcaatattaacatatttcaattttttactcacctgtttaccaatttaacaaagctaaggtggttcatggcagtatttggggcctgaaatatgaaatactgtcatagcagattgtatacgttcaatttctgttacctttctcttacctttcttccttattctagactcaatctatttcatttcaggggtctgttatctgagtgtttggagtttgggatccttgtaggatcttcaaagctaaagacctatctccactgcatctctgagatcttaaaaacaataatatgaagatccagaataaattggtgtcagttaaactctaaccaaagaaggtttaaatgcagtgagcatttgagtgataaataaggtacggcccgaactcaagaggcttccagttttcttggggagacaggcatgaacaccactcactagaatagaaagtagaatgaaataagtactttcataatttacaggtacgagaaaaatgcacaggattactgagaaaggagttatattctgactaggaaaatcagaaattgtggcattttctgtgaacattgaaggatgaacaggatttcagtaaatggagaagaaaggcaggagggcttcctagaccaaggaggtgatggtggtggtgggtggaggagaattgtgcatgccgatcttctgtgcagcactgggtggcaggcacattgcacctaaatgagcactgactatgctttcaagaagttgggactaagggaacaggtgcaggttagaattgtagcctgaaggtaaagtaagagctcaaggaggagtgtttacttcctgaattttagaaatgtagcatgtttataaatagagccagcagagagggagaaattcaaggcacagtgcaggagtgcaaggccccaggtaacacgacagactcttctttgagtcctgcctctctcacttttcatgtttgagctctttttattgtttgctttttgagatggtctcgctctgacacccaaggagaagtgcagtggtgcaattatggttcactgcagcctccacctccccaggctcagcttcccaagtagctgggaatatatgtgcacaccaccctgcctggcgaatttttgtattttttgtaaagatggcatttcactgtgttgcctgggctggtcttgaactcctgggctcaggcagtccacccacctcggcctcccaaagtgctggcattacaggggtgagccaccgtgcccagccacttgagctctttattctgccatgtccgggttccccttttctatgcaatgcagaggtggtataaagccactaaaccaaggaatcagcttcagaaacaaggtaaagctgccttcactcaatgggaaatcacatctatagatcaccgctctgttggcagggtcagatgactgaggtgtcttgtgtcttgatcaaaagttttgtttccactgggtctctgtcctgaggtctccatggatctgtgaccttggacaagtcatttaacccctaggcaccttccttgccctctctgttaaaatgagatgatggtcccagcgctgtcttaggcctctgtgtcaccaccactggagttgtttaaaatccaggctgtataggttctgtctcctcttctcctcccactgccttccccacttctccttcctctctcctgctgtagtcattagcctgatgtatcctgacagtatccctgtaataaatataagtggaatttgctaattaaatgctagcatttttaaagaaagtttccagcaattggtagagtagctgagtggtttataggcttcctattcaagagggaggtgtcctaaggtttaaaattaaggttctagtcataaaggaggcttttattattaggacaacatggtaatttggttgagagaggacttactctctgcaacattaaaacatacatatacttttgacagtttcagtagtagctttgtccttttgatttacttctttggacagttgtgattgacatcagattgaacacatggttcagtaaaaactacagctccttgaagtacttttgttaatattctaatctcctatttgcatcattaacaagagcacattttattgccgcttgaaatagactcattgacatcccagaataggtagttttgatattagctacttgttaatgctgcccaggccattcacattccatgttcttgataatgtaccacgttcttagtacacagcccttacttaccacattgaagagactgaatgcactgattttaagaaggaggtcttgtgcctcctagggggactagctcttttagtattatttctcttatttctgaagcttatgttagaatctgagcataaagcttcatttcattttccctggagccaattatgatctcaaaatgagaagtgcatgagtaaataaatttaaagggggcttttgggaaatgttctgacataggcgatctgcgcagcacatctgaaatacctgggatgtggttatatacagtggtcatcatattcagtggtgacattcagaaatgatggcagtttataaataatcctaatttgtagcaaatatgtggctttaaggccctgtgatgtgattgttttctttgttctgttgatcttgtgactttaaagtggcattcagttctctgttcattttaaatgtttgtcgcttctgacttctgtttcctcttattgttttctgtttttgtgtttgagcccttaactcaggtgtatttatttccccctcccaacttcctgccctctttcttcttctgatatcttctctttgataaccatgtcttccatgtttagtgttctcttgccctccagcaacctatgttagtttggttcatttgtttaaaaacaaatctcctatttttgagaatgagaatgtggtgttattaactaacatttagagcatgcttatatatcagatgtccccaggcattttctatggattatgttatttaatatacccaagaatcccatgaagtaagttaaaggggtgaagtaacttgcccaagatttcacagcagggagaggtagagccgaggttggaacccagatggtttcattctggatctcctattcataaccaatcctgttactttattgtctcaactgttcagtccgtcaagatttaatgatcaccagcgttgtcccaggcacagtgcagacacaaggacactagatccctttctcttgccatatgtacggggtcttaagtgccatttgagaggttcttccatagcacctgtgcatgccagtgcctaactcttttcaaaagttcaggaggtcttcatccctgtggagctgaggtttgcaggataattaagagtttgccagctggggaagcagagaaggcatagggacatggggtaagaaaggagagcacagtcgacactgcaacagctctcatgtgactggagcataggtgctgccatagcagaacagatgggcagttggggaatacgtcgtggggttggttgagtccaggtggccaagggctttgagtgtcatggcacagaatttggacttcatttttcagccatttaggaagctagtacaaccttcttgagtctctcctgcctgttttgcctcattccacagaggccactggagatctggaaaccccgtggtgtttaacattggcagctgagacctctacgtgatcagcttagaggaaattttattgcagtagcattgtaaaaactagtgttttcatacagtcttttgatccttggtcccgcataactactgttaaatccaaaaccataagcagaaatttaacatgagtgcttatattcttaacttgtaagatgaatacagagatactttccaaagaagtttttaatgtaaatttaaatgtatgacttttatgcaggtatggtatgtgctttttcctgattaaatttcctttttgggggagaaattgagaatttaaatgatgtcatgaaacaaaaggctttggatactgttgaaaagctgcaatcagtactgtttaaaatgcttactcactattacaattaatctttataaagcaaacatctttcttgatgggacttttttcaaaaaccatttttattagtctgttttctggtggtagtaagatgactagatatgagcccagtttaaactcaaattcttacttttccactttcagtaagaaaaataattagcattaacatttcagtggaattgcaaatatattgccctagcttactcagggaaagggaattaccagctggcgaaaaagcagtagaatgaagtgtagtcaggcgttatctcacattgagattcttaggtggggactctttgaaacaaatctcaaaaaaattttaaaatccagaagatggcctcttttcacagccattaccttggaggcccattttcttgtcctaacgtagctgcccttgcatgaaggaggaatgttttttgtgtctcttgctctttccctcctttctttccttctttcctttttttatttttaattgccttcagtgtttgcagcagttttttcaatgcctttggtaatggcaaactttgattttttgaagttggatttaattttgggaaacagccaaaaggcatatggagtcatacctgattaataaaataggtaagcactttggagaattctatttttgctaaaacataagcttaaactattaggcactgaggcagattttattgtatatctcatgaactgcctttaaagaaaatgtcaaaaggaacattacagaaaaatgttgagaagtggcagaattgggatactagatagcttccttaggtgactttgaagaacaattttattgatgtgaatttataagtcctattattttgtttcttgggggatttgtctctattttgcggttttatcttatatattcaatatagttaatgttctttaattaaaaaaaatctttaggatactttagtagcactcagtctttaatttgaatgtctgttagtgtagtactatatatgtggggatatacctgtaccagttaatagagtgtgtagttaataaaaggtggcagggtggtaaacctttctccccctgcataattgaatagttttaagtgccatttgaaatcctgaatatccaagattggattccaataactgtgtagcctgagaagctttgctgtcagcagctgctggtgtttgtaagagttatttcactacttatcgctcttcagattcaccatgaatttaggtagtaatttaccattgcagaaggggcaagctctgcttctgatagtgttatgatactatgtttttgcttccttaatgatacgattcttgaggtagaaaaattaaaaatatgtggtataaatcagcttcatgaattaaattccccagatttaatgatgacagttttattcctgggatgcttacgctataactccctaaatgcttcatttaatacttcggaaatggttgttctcataggagctgttattgcaaagtcagtaatggctctggggctgaaatttgctttcttaattctagtccattgtcttttgttgtcatgtttcctcagtttccttagatatgtcacaccaaggactggttacagaaatactgcagtgcattatccaatctaagcctgctttactttctttcaaatgagcttttaagcatataagtagcgtatataatggaaacagaatttagggtgcttaggtaagaaaggaattacttccatactagagtattttacagctttgtttgatctcttaattgtctagatcagagattcttgggtactttcaaattcattttaatgtaccatagccctttaaaaatgacaaataaatttaggcagtttgttttcagaactgatttgaagaatttcagcatactatttaaggtaagtagtcctatactggggatcctaagctagagtcagcttggactcgttgaaccacgtgaccacccagggtttcctatagagcacgtttttagctatttaaaataattaccatgctggacctggagacctgtgaatagtccataaagttaattacaccttgagacacaccctctgctcagctactcaagtaccgtatccactttacaatgttcctttgttaacaagatctatgtggttgaagtatgtattatagtaatgtgcaattcattgtgtacagacatttccaaagtaagatgatggtaataattacacagtaaaagagttcaggccttttgcgattgttgtgtagctaggatcggaaggctttgtaaacttgatatgtttttgcatactttgggttggtgctacctgattatggtactaaagtatttgagtcctctgcccttgtatcatttgtttgttagactttacattgttgactgggttccatgatttataaaaagatcccagaatcttttgtaactatttaaaggtcagcagctaatgataatcagaactaataatagatgcttacttatgcaccaggtattagtctaagccattgatgtgttagttaatacctctcaacaagtaagtgaggtagagacttttattattttatagatgaggaagctgaagctaggaaaggtgactgaggcccacagacttgtagtaagtggtagaacaaactaggcagacaggtacgtaggtaggtggatagaaagaaatagagaaaaaaagagatttaagaatagtagaaggtgtcagtaaaggatataatagctgatttcctgcctgctgtggtctaggcattatgttgggctacaaggaaacaaaaatgtatgaaactcaggcccagtcctcatggaaatcacatttggtcatggattaccctgatataagtgtaaataattaatgatgcaatgtgaacattttataaagtaggttgtgcataaagtctacgaaaatagactgagagaaggaatccaacttgtaggggaggcagagatgaagagatattcacagaggagaaggcttttgcttaaagatgaccacagtgccctttgagagtgggaaccatgtattgttcaaccgtgtctctccagatctctagcaaagtgcccaggatgtcatggttgctcagtaaatgttgggtggtaggtggtagcagtctggccagatgggtgaatgttggggaagagcattgtaggagatggacatatctgatgaaaagcacagaggcagataaggccatggtatgctcatccacagtagggctgtgtccttgggaagtaggacatatgggagcatggccactgtgctacttcacacatcactctccttttacttttagtctctactcccaacatcctcactttacctgctttaaatcgtatttgtatttcaaaaccctgataaaaatttctcacttcctccgtgaagttttttcagcagttgccatgcaccctagtctctcctttctctagtcacaatcctttttgttcctgccactcatgagcctcaatggatatctcacttacactgatttgatctttacaaattgtatgcatgtattaaattatcatatgtacccacccccaaaatataacagattgcctgcagtcacctctttgattattgttttcttttgttaaattatttcctctattctctcttcttggagtgcagagaaccttaggattcatatgctacccttcctattggtacaagaggacacggaagcccattgagatgaaaataattgtccaaatcacgtaagcagttaaagtcaggcccgggactgagtccatggctgagaccaatccatgctagctaatgttcctcttgttcttccgagctcagtagtgagtttgtggaggtgagggactgggtatcatactcactgtgggtcaagtagctgctcaggaacatttgcaaatgaaagaatgaataccacttggctttctgagtaatataaggataatgtggttcccagccttaataaacagctctagcagactaaacatattgatagtaagccctcaactgttagatatactcattcagaaataatatcagaagatcaactgctgagcagaattttcatgttaaaactatttcactaggagtactggtacaggtcggtgttagactcattactgaaaactcacttgaagaactctattagctttacatatttctagcagattaaacttagattacttacttttaaattattattggaagttagtctaagttattttccaatatagaatcattgtcttaatttaataattcttgcttcacaataacataatttggaacggaaattagattccagagtgcccttgtaattacaaattgaaaaatccttttctgactcaaaagggggcttactggtgacaaattccattatcagcaacattctcatcagaggctcagatgaggtggtgccttctgatgcattcttaagtgggtaatgggtagaagagttaggtgtgaggttattgctaaatacgaataaccatgaattatacaagatttattactcagaaaccaacagagtagagccttgcaattcattgctttaacattcatgaattcagttatttatgaaacagtgacccagaggatccatgtcatgtagtggtttgtcatttgaatcaggtgaagcccctgtgcaggggaaaatcacttagctggtgacaagccaaccacccaattgtttttagcccattatcacatatgtaggaagtcatgaactagtcaaaactgttttctttgtgaaagaaggcctggtgatgggagtgcagaggaataacatgttcctagaaagtgatgattctttaccaaaaaacagaaatctgggaccaattaaaaagccggcatgctgtatttggcaatagattgaatctctgaaatgcatagtccagaaccaagaagagcaatttctggaaatacctcagtgagtgtactggccagtgtaaaatttcactttctgggacaattatgatccatccgttttttataggaggcccagtaatgatatcttgactgaattgcctttttgagttacaatcattaatcttgaaggtcatttgagatgtctcaagtctcttatttaagtctttgagatcaagtaaaatccctagcatgtatcaatagccacctgtatcaaacattgtgtatgttttttaaataaagaatatctggctataggattcccttagcctctttggaagaaattatcttgaatagggaagatagtgaagaactggtatgtccaagtagaactaggaattagagcaatagtctggatcctggagctcagaaaatgggcagtaagtcagggacaaaaggtggcttcttaatctgttgttaaaaattacttagagtagaaattcacattgagccagtccatcaagctcccagtatcactaaagaaaacctgtggtactaccactcccccttgtttatatctgacctcccacaaggctccttttttgtatgtgtagtgctttccctattccagacacatttttatgttttttaattataacatggacaaaggatagcattgacataaaccccaaattttaataaatgactttcctgaaaatgagagtagcagtgactttaaaaatggtaagaaaaggagccgtgcgcagtggctcacgcctgtaatcccagcactttgggaggctgaggcgggtggatcacgaggtcaggaattcaagaccagcctgactaacatggtgaaaccccatctctgctaaaaatacaaaaattagccgggtgtagtggtgtgcgcctataatcccaactactcaggaggctaaggaaggagaattgcttgaacctgggaggcagatgttgcagtgagccgagatcgcaccactgcactccagcctgggtgacagagtgaaactctgtctcaaaaaaaaaaaagaaagaaaagaaaaagaagaggagaaaatggggaagaaatagagaaggctggcttcatcctcctcatttgctaggtaggagatctgcagtgccttcacaagactagggaggcagatgcatccactcaatatagggatagagtctttcagccatcagcaggagatgttcaggtaattaacttcccctcataaaaccaaggatcagaacaagagcagagaagcactgtttgaactgtgaggaagccatggcagcatgagagggtcttgtagacagaaagctttcagaggtgggagccgtaacaagccaaggcctgaagcagacacacaacttccacaggaagcattctaccatctttcatgttgagacaccctgtctagtcttaccagtggctctggtggaagagcttctactttcctctggctggacttcatcttctgaatgggttaaggagactgccagaggtctaaacttctggggattcagttgttacagagaagtccctatggtatggaggccctaggttgttcctcatgggcgctctctctgtctagggctccagccccaagacccttggtgtacagacagattgcccttgtattccatcttgcaggcctgccctggctcccattcctagcccatttgttcactgtgtctcagggggcgaggctccgcctgtgtggctgggtagcctatcagcacctggctggtggacagcactatagctgtcctatgactaaaggttgctcttctgacctttcttagccactccactaataaagggttatttttgtgttggtttggtttgtatgtttagctgatttcatgataaaggaactttgtaatcaaacactttttcagctagattctaagcaattttaaatattatgtaagagatcaaataactatcagtgtaagatgcagactacagactgaaaaatgagaaggttaggatgatgactgtgtgttttgatggaagcagtgcaggacttcccaggagactgggcttctggccctgccttgacattcataggtaaatgacccttgggcaagtcatttggcttttctaagtcactgttttctttactcatatctacgaatttctgattaagacaacttttcttctcctttgacaagtaagtcttgtagacagaatcattcccacatttgtatgtaatattgattcccctattctcttgtagttaaagcttcaaatctctaattcatcttttttgtaggaaggattagattgatttattgaaaattctgagctatagaatactgtagaagaagcctgggctttctgcttaaagttcatatatacagaaaatctaggcttgtggatgacaaagcgttttctgtacctcattgcttttattaaaatacccaacttattatagtgtctcttatttagcagttggttaaatattcctggagttaatgagtaagaatcttctatcttgtaacaaatagatgtagagctttggtaagaagtacacatattacttgtttgagtgtttctcaagtgattcagaatgctttacttacattcgctcttaaggcaaaatacttccattattcttacagttcattgtcttaggaaacttgttgcagaagtttctaattgtccccatatacatgtgttcattctcctcttcctgcgtagatataaaatgtttaaagatacagtcacccagaataaagacaccgttgagggaggtgtggccacgtgactgagtcctggtccatgaagtgagcataaatgacagtgctccttttcccataatgctgtttagagttgcgccttccccttctacttttcccctctcccacagttggaataccatatggacaagggctgcaccgtagggattgaatgggcaaaaaagctgaaaggaacttaggtccctagcacctgtgtggagcccaatccccataacatttatagaattaaatttcatgacactcttattgcacataacacaaattcccaactactggcatgtggctttgtgagagctgtttcaccataatctgttgagacttatttgtggaatcatgatgtcccgtgacatttgaaaatggaagagaatcctggaatatgggagcaacactgacgcagaacacagtgttcactttggaagcagtcatgatctcttcttgctgtggaagcctcaacagtttgcctgttccctccattgtactcctctctgaattctctctcaggcctagatgctgggctctgtgtgggttgctgtatggtatagaagaggatgagattggcggggacggctcttgttcaggtccatcaagactgtcgctgtcctctataccttgagccagctctactgtgcatttatagaaaagagagaccagcacacagaaagagagaaagcagctgtttttagggggaggagagaagatggtggagcttacaagagactgatgtttgcaagtggatatggtataaagtagtagttctcaaagcacggtgcctagacagcagcatcaggagcctgtgaatttgttagaagcacacatcctgaggcttcacccaagaccaggggataagatttggcaatctgtgcttaaaaaagcgtttgccttgctcacttctgagaaccagtggtctaaagagtgttgggaagcctgacagtggtagttacaagggtgtaccctgatgggagtcctgggatcaaaaagacttgaactagaagtaagggaccaggtgagggagcattcccttcagagagagttgttaaaataagccggggttctcatgatgctgctgaaaggagtgtaagtactgtacctcctgtgtttgggaccaagggtgtatctagccttaggcttccattccagcacatcccgtcagccagatgtgcaaaagttcctccgtatacacactgtactgcttaaattgctggtattttgaaatagaaaaaggtgcttataaacccatactgactgtgatgaacagggagtactttttccacctacctctgcttgggaggctgaaccccatcttgccctggaagatggctttgagccagaagccaagccctttaatggctatgaaggggggaaaaagtatgatttccatgtaagggctaccaagaatataagtacctatttaataaattatttgttgaaatgccttttctattttggtagttgaagtttgaaaaatgtttggagtactataattatcatttctgggaaaaaaatacacattgcagtatttttgtttttgctacaggtcaagttgtagttcaggatggaaagtaaacacatccctttgctcagctgcagctcacatatttgtagtctctgggtgccctcttggcagtcgtggtggatacccaaaggacacaaacagggcatagaaacgcggggctccatcaggggcttgccccagtgcaagtctgattaggaagtttgctggagccgaaggaatggcccagaagggtggagatgccttcacagaaggagtcctaggaccagaatgaggctgttctcagaggcatgcccacctctgggaggggtctgcagaagcactttctcaccagggagagcctttatggtatgggtctaaacactgggagatctttgtttgtttttggaggcactccttaccattctatgccatcttgtcaatttgacaactttgtaaacagggcccatccacacatcttgaatgtagaaatagctgagaacagtctccaggagaaattacctatataaggctgcttccttccttctctccagagtggagacgatttataaagagaaagtattggaagctgacagagaaacagcacttgagagtaaaacttttggcatgtaagaggactgtgtgtgtgtgtacccatgcctatgagtgtgtgtctgtgtttcagaatcaactttacctttctgagacatagcaagcatggagtgattacatctgtgccttgggacatcgcgttaggtgttaggtttaaaacaaactgaggagaaaacataagaacctgtgagtactctgtgtgtgtgtatatgtatatacacatatatgtatgaaatgtttttatacttacacactttatattttacataatatgaaaaagtatatattcttttactctccaaggcagtcttgtagaataaatgtctttatgccctgtacactctattcagcaccctctttttgttcttgctatatcattttaattacataagtaaagtcactttcagaaatctcagatcaacacacgatattctttgaagtctttctaaaaacatctgaaatccatatgccacctctgggactcgggaattttttgacaatattacgataatacccttggtttattgttccttgtctttaaaatcacgtaagatcacactttctactacactactaaaaactctatagagtgttgacaaatactggaggtttaaaggaacatgactggctgctcctttaattcagcaagcgtttgtcggtcttccttgaatgagcaggaactgagcatccttggaacctgactgcataggaagggctctcagaacatctggttggaatatgttcataggagacacccacaagggccagcccctcattttacagatgaacaccagacccttcactctgacatatttaaaatctgacattttattatgtacttttttcaaattgctgtcattgttgcacaaaggaagaaccaagttaccgtgtcccttcctcagtggaggaaaagttgatagaggttttatttgttgttctaaaatggatgccaccttccggtaaacattacttggggagttcttaatttctaatgtttatgcctctgtgacattttcaagctgttataaagagtcttcttaaaatgttgctgagctggctatacaaatttagttcatttaatttacctcattagtcatttgtcccattctctaataattttgattccttttttgtgggtcagtgtttatggcaaaatgtgttcggtgggggaggtattcagagaagactgtagtttattaggtaaaattaaacgttttgaaaccagtctttagttctttgtacatttatctaaaccatccttaggccttattttattgccagatttttaaaaaaataaattcattgttacattttgaaatttgttatggtgtgtgaatatataaatataattatttgaatggcaattttttctgggagtaggttggatatatttttctggtgtgaattaacagatgaaataaaatgtctctgaactcagtaaatatttgaaaactgttttaaaattcagtaggatgcaaacaggccacatttggcatccggctctcccactccccagtccttccaccccactacctcccataaaaggcaaatcctggaagccactctgaacttttttccttcctcccaggaagctctgacttttccaggaagattccagggaacactagttggatccgatttcctaagaattaaagcagctttaaatctcctgggagtgtagaggatgaaggggttgagggtccctagaacaataggtcagcattgactgcagattggagtcagctggggaactgtaaaaaaaaatatataccgataccagggtaccacttccagtaattctgtttaattggaattggtctggggtgcactctgagcttttggatttttaatcttctcctagggtttttaatgggcagccgaggttgacagtcattattctggcactaaattttcaaactatagtatgcatgagaatcacctctagggtttgttcaaacccagattaccgagccccactcccagcatttctgattctgtaggtctggtgggccttagaatttgcacttctagcgagctccttaggagatgccgatgctgctggtgcaaggatcattgtttgagaaccatcactttagagcagttcatctcaaactttactgtgcatcttgctaaaatgcagattctgattcagtaggactggagtgaggtctaagtttctgcatttccagcaagctcccaagctcccaggtgttgctgaggctactggtctaggggccacactttaagtaacaaatctctaatctaaagcatcattatgaatgcaattccataacatcgtttgggaagaaaagatgtgtggttcaggagattcctagaggcacggtggggccgatatatgccttagacctggtggcaggcagagatctggagggctcagtgcttctttggcacttggccagctgtggcttgccagggtgttggaacatgcccttggcgaagctgtactgtcaatagggagcttcattgtaaagattctctgtttgcttggagtaaggcagtggttctcaggcccaattgcacaatagaatcaccctgggagctatttaaacaagtactaatgcccaggctcctcccccagaatttctgatttaattggtttgaggtgggatccagacagtggtatttttaaaatctttgtgagtgattttaatgcacagccagggttgagaaccactgaattaagaaatcacgttacagtcttacagatcacttttttccctttatatttggctaagttatactgcacagtgatgcaaactagctttattaggtagtcaaaaacaatttaatatggcatttttgacaaaccaccaagaaaagactctgcaaagattccgtcgctgttaaatcctaaattcggagtcttaagatagtgctgggaaagtgcagtgggtgtttctgaacgagcttgaaatatgaaatatttctgggactgtatcaacaagtctaattttttccccttggggttatgattgtgcggaacaagtcttcacagcaaagcttgccaaaaaaggttatgataaaatcatggaatgtgtatgaaaagaattcacagtgtaactttaaaggagtcagtctctaaatttaaaaacttagggatgacatacgtattacatttgtaataaaagttttaagaacaaagtaatgctgaatataagaaagttcctccttttatcacatgatatctctaaggccaaggaacaagtgaaatagaacagccagcaagaaagtaatgaggatacgctaaagacactgaacctctttatctttgcaaattaattttgtcctctgtatttaagcaaaaatgtagctattgactaaatatacagataccagcaacgccgagtaggaaagttgaagctcttcttgcttttggcattatggacgactagtgggaaaatcatgggctttggcagttctgccacttgctttgtgaccttggccaagtttcttatcctctctgggtctctctcttcatctgtcaagtagacatgaagatataatctcttcttcacacaggagtacaaaggcgtgtgagaatgagtagaggcgtgccccacacgtaggagatgctcaattagtagagatttctttcatttctccagcatgaccatgagaatttaatgcttaataagaagcattatccatcaaaacatttttcttgttgagtctttattggtattttctgcctattaattgtatcagtttagatttcttattaataatcatgaagtctgtttttccacttgaagagtaggccctttcttcttgctttagagccatcatttttgccattccctttgtctggaacactcttgctttgtgttttgtgtgctttctcccttctttcatgcaagcctgtgttgcgatgccatgtcagagataccttccttcactcgcctggtgacacatggcacccgtatcactgaatatgctgcaaagcctttatcataacctggcatggtgctgtgtagctttttattgtctgtcttctccagaattgtaattctcaaggttggtctccctgtgctactgagtgaccaagggcccccgtgatactctaggctgataccacggctactctagggctttagattataaaagacatcagagggaggtgtcggaaaaaacagtgaccccagactcttctcttgactttgtgttctacctactaaatttccgctatcaaatataatccaaatttggggtcctttgaattccccgaagtagcttgaaccgcccaaggattgtctcaggatggctgtgtatctcactgtgtgtgtgtgtgtgtgtgagtgagagagagacagagagagagaatgaagaacgagaagagatttcaaaatagaagttttcagtgtcagatgtatgggactctacaaactttaaatagaaataatggtaatctgccaatgtaatttttatcttctttttttttcctcttactaccagtagtttttcatttgtctttgtgttttatttaaatttgcttttaagcagaagctcttgcatgcatttgcttctcgtagatcatgattgtattaggttggtgcaaaagtaatcgcagtttttgtcattactttcaatggcaaaaacagcgattgcttttgcaccaacctatacttccttgacatatggacctgaagagggcctaaaaatggccaagatccagtcatacacattaaagtgaccaggattgatagaaactttccagggttatggtaaaatcattttatattgggtgagcctgtcaatattttaatactaacttcttaaagattattaagatgatgaaaccaggttatttttccatagttacttctgttgcaagtgatattgtcactttggtctttctatatctgggcaggctttttttttttttaatgtctgttgcactgttatcagtgtaatcagacatagaagttattttaagtattatatatagatctgttagacaccatagggaaagagttaggagagacttcaaatagataaaattaaaaattcagaagtaatgtcagaaaaactttctaattgatggagtggctgtagttttatccttattttctccatggaaaaaacatagaatagagacagagaagaataaataaaatctgatgggagaatttatagaaaaggctgaaaaaaaaggtttcagaaaaacaattcctgatgttatggtaattgtactacatttaagtgacaagctgatgactagttcggggcagaggaagatgcagggtgagtagggattctagataatttattgaaaggtgaatagagggttgaaagaagagaaaaaggaatgttaattacaaaataagaaagatggagacttttgaaatctaaattatttgaaagcataacttcacaatttgaaggcttgaatcttatgcctgctagtcatgaagttaaaaacatagctttgaaaaattaaagtaagtgctggaaatttgaagagaagttactggctcgtagtagattttacagatccctcttggaaatcaggtaaaaaatgggaggtataggtaatcagaatgtcataattaagcttgatttaatagataacgcaagattatatgtaataaacagaggatatatgttcttttcaaatacccatctattttaaaattggttatgagaccacacatacatacacacacgtgctaaaaaccaaaaacatgcaaaactcaagaagttaacaaaagtagagaattgtacagcctacattccattctctggcaaccaggcagtaagactataaattaaaaaagaaagctccccaaaatgtagctatgcagaaatttaatatcactcttctaaaaagtctttaaaaaggaaacatataaattacttagcaacaagtcacactgaaagcaatacattatcaaaaatcagaatataaccaaagcaatatttgattgaaatattcaaagcataattaaaattgtttgtataataactctttaagaaatgtgtttgttataagacaaccaaatagaaatgaaccaagcattaatcttaaaacactagaggaggctgggcacggtggcgcacgcctgtcatcccagcactttgggaggccgaggcgggcagatcacgaggtcaggagatcgagaccatcctggctaacacggtgaaaccgtatctgtactaaaaatacaaaaaattagccgggcgaggtggtgggcgcctgtagtcccagctactccggaggctgaggcaggagaatggcgtgaaccggggaggcagagcttgcagtgaaccaagattgcaccactgtactccagcctgggtgacagagcgagactccatctcaaaaaaaaaaagagttagtgtaactttgaaactaaaattggataaggccaacataaaaagagagagaactaaagatgaacattactcaagtttataaatttaaaaatcttaaaatgttaggaagtcaaattcaacaggatctttaaaaacagtttattcttagaacacaaggatgatttaaaatccaaccgattatattaataaagagaaagccagtgtgattgatcatcttgaaaagttgaaaagcatttaataagatttaacctgcatttttaacattcttacatcccttctagccgtcaaactctgatcgacttacacttaagactgtgtctttggtagcctaccctattccacttttattacaaaggcagctcttaagacataaatccatctcttcaggctcgaactgcaaaacacacgtcgagaggtgacaatgttgggactgtcaaaaagaacttaaagagtgggataaccagccgggcgcggtggctcactcctgtaatcccagcactttgggaggctgaggcgggtggatcacaacatcagaagatcgagaccagcccggccaacatggtgaaaccccatctctactaaaaatacaaaaattagctggatgtggtggcacgcacccgtagtcccagctactcaggaggctgggacagaaaaattgcttgaacccaggaggtggaggttgcagtgagctgagattgtgccactgtactccagcctggcaacagagcaagactccatctcaaaaaaaaaaaacaaaaaaaaagagtgggataaccagtaagatctaatatgttgggttttaatggagatgtttaattgaagcatatgtttcaaaattctgattattacatactaaatattttgtaaaataaaaatgtaatagaatatgataagaatatattaatgacagcaaattaacaagagtaggaattaagtgctatttgcaatcaaagtcacaaagcttttcttaattataaaacaatgtgtttgccagataagcagtctaccaaatgcagtttacaaactccaacttaatctgcatacattcaagattcgccttctcctcctcacctctttgatttattctcctccacactgatggggctctgagagtggaattccttattagttaatcatgttctcttctcacggcagttccttggggggttatactatgaaagtgggctagatggattctctgagactcctgtttgacccaagcacatgtggcgtataggctgtagctcccatgagacatgcagtgagataagactggtaagagggagcagggatgtagctaacccggtagctttaacaaaaaccacatttctgaaagggaaggagctgtgcccagagaaatcagctgatgactcaggatcacagtagtgattcctgagacaggagtcatattattacagaggaacatagctggaactggatggaccaagtttcacatcaccacatcaccatttactagctgtgtgaccttgggcaaataaatctgaatctcagtttcatcatctgtaaaatggagcaaataataactaaccttctggggttattgtgacatgagcacatggcatgtgtgttaatgaagtctgttcttaggacagaggctgttttattttccattctgctttaagctgcttattggatattggcatgtgggtgatagccacagggagggtagaggcctgcaaatgaatgtttcccctgagtgaggcacaggtgcatgaagtggtattctatccaacaggaaaattaagtgtagtcataaggtaggtggcagaaggaaggcagcattaagactcaacccatctcatctcgttctccaacacacccggtacccagctgtggtaaacagtgctagtatttgtcttcctccatattctttcttgccaagtgcctttccaacctcctcccacgtctccttcctccccagatattgtgtgccttcctgttgttgcccaggttcccaaagtcctatctttctaagaagggacctgtttttctgaattattctcttcctctttctatataatcaaatctctaatttgtcccatagtgaacggtacagtcttgaaaatggcataaggggttctaaatcgccaggtacccctgctgctcatccaacctcatctcctgtttgtcctctcactgtgcctcagccacactgacgttcttactcttcctgggacacacaagcctgcgtcagcagttccttctgctgggatgctcttctccaaagtatctccgtggctcgtcccctcactccttcaagtctttgttcagccgtaaacttctcaatcatgcccatccattctcaccacattgtaactgctacccacttcttctaagcattccaattccctcttactctgttcttgttttcccctcatagcacatatcaccctttactactatataatttgcttctttataatgtttgttacttatgttctgtcatcctccaccaccatatcaactaacaaggtcaggaacctgtgtctttcctctttactgacatatcccaagctgctgaagcaacaggtgctcagtcaatgtttcagtgaatttactagaactataagtatgaagagtctcctatattatgtcttaggtagacaagaaatagataaggaagaggaaagatacagcaatcaaggtagaaaatgactagaggaacaggcatggttttaaaaagagatccactgctatgatatcaagacctgagtggactggaatgaggtgacctggattcttgctttaagtttaccaatgaccttagccaggtcacttaagctctggggcctcagacgttctttatctacaaaatgagaaaactaaactaggtgaccttttgggtcttatcctactacccaattctgtgtttgggttttactttaggaagaccaattaacacttacggaaacattgcctggtactaactggcatcaatttcacctaatgtctcctgaactccccatctcccaccaaaggtcctgggctgggatgatgtcttttagtctatttccatcttttcctcaagggccttatgttcgtataggtctcttaaaactcaacccaagaagctaaactgtggactggggtttcagaagccagggctttgctcctccatcgtaaaatctgagtaaacccacattgttttatttttcgcatctctaaaattgagatacagcaatgggtcggcagataatattagacagaggggaataaggtgcatttgatggagctctgctagaaatagaagcacatcaggtgccatttcaggaaattacacgggaatttggctcattatccatcttgctaagctgccacatcgtaaagtcacagactattaatagaataccattgataactcagagtgtgcaggtagagttcctccatgttgattgtcgtacacattgtctccagtttctgaagtagccatgatttgtttgtgttttaaggtacactgttttcaaagaacagcctttaaagctcttttgaattcaaagatctttcagttactcaaggaatgggtccagttttcagattctccacgccatgggtgcatcttcccttcagctttcttcattttggtcattgctccagatgttcattgtttaacacaagtgggtagggagacggagaagaagggaatttcaggctcatattttggtgtcatgggcagatcttgggtgaggttttcattgttagttaaactaagtttgaaggtttacactgaatttctttccctaccgtcctctttggctacttaaatagatcatcaggactgtttgttcttctgttgaaagtgccccacactgccctgggcataagctaaattctttctctcccttttagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccaggtatgcacgagctctgtctcagcagccttgttcctttgcatcagagggtagcagatccctcctcagccacccctggcctctcctttcacacgaagatgggccttctcaccggtgtcctgtgaatctgccactttgggctaatggcagcttgacttctggccttctagtcagagaaccagagtgatcatccactggtacataagcgccttcggagagcgagcgtgtcttttcctcttttcttctttttttccttcaaagtatgcttgactggggaggtagggttaaaatttatggtactatggcaatctgcttcatttccccagagaaggtcagagaccttatttgcagctatatctgccactttgaattattacatttaaaaacaatcatcatacgtaaaacagaaagttatttttagatgaatacatgcatcaaaacgccagaagtttcataatgcaacactcagccccaatctttctctttgccaattcccccgcatctgccctctttactttatagcatgtgccaagtcatctttgactgtttactcaccctctttgccaacatttgatgtactgttgtatcccagctgtttcttgctttatattatttctaacatccttgctcttgtggcagcatttccgatgtggtctgtgaacacttctgagttatagcgatttcctctttgcaagtttctaatcatcggccagttgtttttttgtttgggtttttctttttctttctttcttttttttttttaactgctagataaacgctccaagcaaagtgatcctttgtccgtctcttgaactattctatcaatcagaaatgcctctggctaactgtcactatccatgtaaccactgctggtttaattctttctctgtcttcattgtagctcttaaatatctcatccccacctctttccttacacagaaaatttgaggaatattgcagttctatagcattaactggagtaacatgtcgacactaggactgggtttgtggaagcaaggagactttggacatttgtcaagagctagtatttcatttagggactccaaaagcaagtaaatataacaataaacggtgcctctctgtttgcccctttcagttttcggcgtggactgcaggactgtggaatgccctcctgttcagcagaccgcgtgtcccccggacagctatgaaactcaagtcagactaactgcagatggttgctgtactttgccaacaaggttagtttgccattagtttgtcaagttttctcctcatttgttagcatcatctaaggtacattttgagcatgcagtttgttttcccatgagaaacctagtcctttcctgtttctctggaagaaatcactgaactgtttgcattacttggtatttatatgtatctttcctcagatcaaaaccaggtgtcatcaggttaagtcgttaaaccttatagattctttctgtaagggtgcctgtacacttttgagggctgggacctatataagcaaatatgcccctctgtatatagatagggaggcaggcagtgaggtgtctatcaagagtagacacatcatttgatcctctgcagtgaatcttgtaggtgcttaaacaacatgttcaatgtgatgaaacattgggacccttgatgtttggaatcacacatctacccagacccatttcttgctgaggtcaagaggagtcttaattcttacaatgctttctggaataacaaacacatctctctttggcttgactgcggggaagagccactgtcattttaaccctagtagcagggcatgcacttagccccctagaactagtttttctctagaacatgaggatctccgggtcctgtcctttttagtctacaaagcaccaccatagagctgtccctccttcgtacttggctttggggaagactgttcttggaaatgtagggtttacagtctgttttcccccgactactcacccctcttctctgccatgacctcccttccatcttttgcaggtcaacaagccatctttatgactaagtcatttcatgtttttcagctttcgttctgcgtttaataggatgagttaaaactgtgccttatttaactcaatttccaacctcttcttcctcagtcccagtgctaggcatttagcttctgggtttgttgcatgagtacctgtgattatgcaaatggttggataagataggagtagatgtggtccataggtaaggtaagtatcagttatctgtccgtaattgaagagacatccagaatttgttctgataattactttttccttctttcttctcataaaataagttaaatccttggcatctcctagcctttgttctgtcaaacctagtattctctaagacatatcctttatactgtattataaaacagatgcaaagatttttgtgaagataacatacattggtgcagccaccttcctatgacattagaatatgataatttatgtgcagatgcttaggaaggggtgaaatatcttgcaaacataaggtattactaatcagtaaaggagataaatggagcatcgctaacagtaacagatgtcccttcagggtcttcctgaaaaggaaggtggtacattatattggagaacctaattaatcaaaactaagccggttgttaacgaggtagtcctttagtgaggaaaatggacaattctcagttcactattataaatgccacatagagccatctgcacataggaggcacggtcagctattttttcagtcactctagggggttgaaatgggtagaagctacaggaaggtatatttagtctcaatttaagaagaaactttcccacaactagtgatgttctgtaatgaattggctgcttcctgaggcagacagagctccttggagctggatagatttctttccgtctggatggccacctgcccggaatgcttacaagaattgtattaggttggaaaggaggttgaactagctgccttccattttatcctcaagcttctgtgagtctagaaaataaaacttgaagatgattattactgtgttgacataattttgacttcagcattcaaaaatgaagaatggccagagttaaatttgcctctaatgaagacattgaaaacatagagaaggaagtcattcaacatcccactgtaaattatttgcagagaagggataggttctcacagctctttagtttcctgtatgtcagatcatcagtgacccatactcatcccctgctcatttgttcgctttgaacagggtctcctcctccagctctctcacgagtcctgtgcctttgcagttgccactacctcttcccggactcccttctgcatacatataggcagcttctacctctccttcaagactgcatttcctgctctttgaagtgttgtcactcttcggcccctgaagatgacttaagtgtccctctctgctcccttgtcatccatcccctgtgtatctttttatcatagcacttttcataacacattgaacatactgatgtttttttgtctgtctctcctgtcctgtgtgattgtcaaggaaagggagtattttgtagcctcttgtacctaaatataatgtgtcagcccacatctgttggtgaatgaatggatgaacaaacaggccctctcttagccaagatgatttatatgttgaagaaaagaaaaagaatgaatatttttagtttctagttaccttcctgccattgggttgtaatactactccctgtctcattttcaaatccttgcacttttcccccagtccccactttatggtgcggtactcctcaatcctgtagaaaccctccattccaagcacagcagtcatctctcacagcatgggatgcctttcactctccattccatctcttttcatattgctcatccagcaggactctaattaatttctcctctgtccacgtggtcttcatgcttcaaccaacaggtcccactgcctagaaacttcccacataatcctttcctatgctgtcagttaattatcgactttcatacattgttactgaattgtcgaatgcgtgaagtcttgtcttatcagctatacgatgttcctaaaggtcaggaattgtgtcatcttttcttttcctctgtatgcattagtgctgaatgagtcggtaaatgcagaatatactcaagctgtggcttagacccattcagcctagtttagcatattcagtggattcctgcttactaaggagggtgatgggatgataggttgtacattgattagagaataacaacccaaataatcatgtgaaaatggaaaaaaaaaagcctaacatgtaataataatggtagtattttaagagaaaaactacgtaactttattcttgtgtgtgagccatgtcaaatgtccactctttaagagcatatttaggcataaagaagaagattatagaacatataagatcctattatgattatttgacttttaccagcatacttataagagcattgttaagaaattagagccatttgagagcaaagctgattatgtggagaaatcaggggagaaagctatttacattttaggacataataaggtaaaatatctgaaactttagctgtaaagaattacattcagtgaaagtaacaactccctttttataagaatttattgcactttcatgggctagaaagtgggtttgtggacttttctcccctatcttgaagaatacattcctccctccttcattcccatccaaatggaattgcccttccctatgagactatttcacccattcgggcccttaactgccccatttctgtgtgagcttgatttgttagttttctctggaattgtatgttttcagttgattcattcatctattcatcaaacatgaattgagttcttaagagtgacagacactattctccggatgttcttactgtgcatgggtttcactttccacttctatccctcacaaatactggttttcccttctacctgtggcctttgtgagtataggctcttccttatttccttgctgcctttctgtcttgtttaatggcctttcattttcacttcttttgtgtcttttctcttgaacttcatcctgtttaaacatcattcttgtgtgtgatgtaattaatttttaaccattatttgattcttccttccctcaacttggcatagatttccaggcatcccgccgtttctttttatccttttttcttctgttggcatgaagacatgatatgagttatatggcagtgcattctacctttcaggagagggtggagagggataagaattagtggctctgggacttatactgggcctaaacactacacagtgacaaaaagggaaaatgggactgtctgtgaggtttaggacaccttgtggtccttgaaagtccctttgtgagtgtctgcatgtgtgtgcatgtacagatgctgctgtgcataagagaatttacaacagaaacttaaaagacagatacattaatgcaaattatttctttgcagggaagaaaaaaaccaagatagtctttaagtgacatatagataacaatagcagtcacaccttaaatgtgtaaagtgccttgctgtttataaagcattttccctattcatatcttaccatcactttaattctatgaggaatttataggtatgagaatggagattgagataataagagacttgcagaaaatcacaaattttccttggtacaagagacagagcaggttcaaggcttcaacccaggttttttaattttttttttattgaaaaccctgtgctctttccactggagcttttttgataacaatgttttgttgtttttaagtccaagaaatatatcggggaatatgaatttatatctcaccaacacatttctatctgcataccacatacgtgtttcagttattcactgctgtgtaacaaccgccccaaaagaagtagctgaaaatgacaaagatttattctttgtcctgattctgtgtgttgactgagcagttcttctgtagccagctggcaggttgactgagactggatgtccctcccatgtttggaaactcagctgggatggctggaaagacccctctccctacatgatgtcagaagcaggagaggaagttgcaaagcctttggagaccaagagttggaagttacagaacacttctgccatgttctgttggtcaaaggaagtcccaggtcaagccctcattcaaaggtttggaaagtaggttccacttcttgatgagataagccacaaagaatttgtggccatttttaatccaccctaatctacatttaaccaagatagatgccctttagttttcattttctgattgcaggaatttggtggtgtgtttcagaaacgtgattcttttccaaaaactcatgcccttctggccctggaactctgggtcttcaggtgaaattcgggcagcattatagttaagccccctgtgtgcccagcagctctttttctgcttctttcttcacttgccatttggcagtccacccaggagcagacacgtgatttaagagcctctgagccaccgcttcagctttaatatcatttagccaaccattatttttaaccttccgcttcttgaagagtgctgtgaccacctaggttaaactaggttttctcccatcatacaaaagggaatgctactttgggataccagaaaatctgcctgataaaaccgggtccatttttaaaacaaaacaaagcttgcaaagatgttgaatagagattctcctctgtaatggttgagcttacctgagagaaatggattcaccagaggcagatgcatttgaaggatggatccagcctgggctatacatctgagagtccatgttctcctggtgtccaccagcccagacctcagcccaactgggctatttctcaagaagatagagagttctaagaccacagtgttttctctgcacttcctactccttgctctctccagtgtttggacctaattttacagactcttcactttgcctccgtaatttttcaccagtgtttcagatacaggcttacacggtagacattaactagttaaaaatctagacagtaatacctccaccttactcactttcactgtaagtcacgagttttttaaataccctacgccttttgataagtaactttgtacctttttattattgacaagtcacctattttaaatttatttttatgaaatttgtcccaaactttttgttctaaaagttctgttatcaatttgttttataaatatgccgttcttaaaagttattgagaaaagtccaatccagctattttaaggtagaaataaattaagtcattgtttataatgtccaaaaacttaaaatagagtttcttctgggttgtgtgcatttcagaaagccagattcccaagctccctcctggctcctgcctctctctgaccaagggcctgatactcttccagaacctatatctggaacatgtgtatctcttgtgttctgagacactagaccaaaaatcctattttatttttaaatcaatcttcagaagtatttttttgacttgtttttttttttttccttagcttcaaggtaattaaaaatgttagattatctgtcagctaatatgttgactaatatgtgacttctgttttcaccaaaaaatgtaatagaatatgttggacacatgaaattctaagtacaatttcactagaaatattgacaagccagaatcacagagctcaacacaggaggcaggctgtatcactctcctgtttcatagagggagcattgtggagctggaagggtcctagccggtgcacatcagcggaacaggtctgtctggcacttaccactgagctgcactgtcccttgtggccctgggctcactcaccaccaggccctctccagtcctgcctctcaggagccctaacattctgtgtcatgcactttgttattgtctgtaagcagacacatacctgtgagtaaaccattccgtagctaaagaatactcctctgcacatcacctggtgctgttcagaaagggcacgaatcatgtgtccagagctagaatccaggcccacttcggcatgagtttgggtgaggctctcacattgtctggctcggttttccagtcgaagaaaggaggggaggaaggatttgattgtctcaaaagtaacttctacctctaaattctttgactttccaaaagcctttagaagggaaggaacccaagccgctagcgacaggagactcctgggaaacaaaggtaaagaaaaattttaggaaatttcagagctcagaaaataatgggagagatatatggctcgcctagggataagaaaaggagaaagtccaggggaagttataaaagaattatgggttttttctttaaactttcttaaaaaaacaaaacaaaaaaaaaccttagaaacagcaaaatgaaataaactactagagtaatgatttaagccagggtaatcagaactggttggattgtgaaagctgagtaacaattcagtaaaatagattacgtatttggtcagtggttagttgactattgttagttaaccttttttttttctttacatctataaatagatagtccctacgtagcagaagcagagaatatctggccaaaggctgactccgcaagataaggatttactctgttaaactgctttctaatagaatgtcgatatctctgcctgtcagcctgccttggtaaatgtgctggagagggccatgtgtgtatggttcctggtgcccacacaggctcacctgtgtgaaactgcatcagcccatacatggctacttgctggctcctggacccatatttgcacttgtttctccccttctgttgccacatcactttccctgaggtcggattgtgtgtgtgtcttctctgtcttcagctgctccgtattcatattgttcttttctgtgttttacatttaatcgtgcatacacacacagaataatattcatggacaagttttaggttctcttttcctaactttatgattggaccatttctttctgtctcttaaaatgaaaaaagatctactcaaagtgacttttttccttgtttgaacctcagctttatttctctcccataaaacattgaccttagaaggcaatttcccactcatatcctgtcctatcatcacagctccaaactctgtaactccaggaaaggaaataagataaaaggaaggatggtagtcccattgtgtctgaaaaatatgtcaaatatacagcccatgaccagagcgaaggtcatcaatgtcgtttgctttcactataacatctaaatatttgcaccaagctgcttcaatttgaaattgtgctctcaagaaaattcattttgttggtgctgtcttgcattatgataatagaatcttagcagattgctagcacactgtttcttgtgaatagatagagatgtttagaagaagttttctggggttccagaattcacagcctgaaattgactcatactggatgaggccagccgaatggaatctttggcaggttatcccttcccttcagaagcacttgagttaaatccaattaatctaaccacatagctacagtgagtgaaaacaaggtacagattttaaaatgctccaatcggttaaaacagttaaataattacatactcatcgaggacctcatggaaggcagaacactgggcttggtgtggggtatagtggtgaataaaatgtggcacatgctttaaagagtcttaacatcttacagatatgaccaatgccagtttcaaaaaggctcccaagttttggaaggtaataagaaggaaacacacaacatgcacacacacacacagtcatctgccctcaaggaaagaatcaattaatagacagtctttctaccaaacaatgaaaaccaaatttaagaaaaaccatacagtaatatgccagctttaaatccaaactgagaaatcaaggacctcaaaaagcacatatacatatggaacaacagatgacaattttttcttatccctcccttccctcctgaaaactcaataggacaggagggattttcttattcctctgttctggaacatctgtctgcttctgagaaatggaacgagcagcaggatcaagaatcctttagtgctctgtgtgctcaaatgcttatccttccgtggtagagctttccaaagtttgtcaactcttatctccagagctacagcccataactctgcggtttgaagcctttaggagggtgttgggaggtgggaggatcacgcaagagtcagaggctgtcaacacagtgcgcatcaacccaacctgacaaatatgtcaaagccagaaacttcctgagggtggcttggaactgtcagggtcatgttcagagctcagaaagcacttctttttttcttcttggagacagttttaagtttttttaggccatcaaataatatttcctgtagatccatgtgctttttaccattgtgctaggcaattaagttgcttatatcatgcctactcaagctaaccagttttaactatccctgcatatgcattttcaaagggactttatctaattaagagtgttgtaattcattatcatgtccatctgttctaagatagtgtctcagagaagatggcacttaacagtttgggttttttttttttaagattgatctgttagtagacaaatttccataattcagttcatttttgttgttgtcgttccctttaatttgaataatttctccagctgctttttgggagtaaataaaattaagttatataataagtaaaatatgcattagcacagcacagatttaaaacattcctgactcctgacatactattatggtagcattctctctgtttttaaacacatgcagaaaaaaatttggaagtgtatttgaagctcatctttctcaactaagaaaagaacatctattctgttgccctctgtcacagtaatttttggcccaaaagagcgagtcaactcatgactaaaaaagccagagtctatattaaactctgatgtttttaaaacaaaataattgtaggaattccatgttattattggacctatccccagattgaagaaaatatgctttcctagaagtaaagccattgcccattggaaatgaactgtctccctagtagagtgtttgatttgtcaataattctcttcagatgcgtgcgtgctgtgtttcctgtactagggccttaggctttttttatctcaccttaaatttgacctagtgtctagccttattttgtatttgtcttagtcccgaaaacaagcagggaaggctctcagtcagtggcttccagtttaatccgagtggtcatggatatcaccagacactgtatatgtggccctccgagtcggactgagagaaggtaacttaactgaattccgtaagcctttcagtaaagttgaaagcggaatccacagcgtgataatattgaaactcagaggtctctccataccctcttttgctatgtcatcatcatggcacatgcaaacagaacagctctgcaaaaatgctttgtgaaattacttgcatagctaataagcaagtctaaaatgtaaaagccctacctgtaatgaaaataccaaaagcaacccaaatcacggaatatattttactcttcatgtacacctttacattggatatggcttattgggttccccatcattgtagttgcaacagtttgcagataattcagggttatatttggtagacatttaattgctaattagaacccaggaggtagagacagctggtttctcttggccttggtcagtctgttgtgaatgtattatacagcaagtggtttgtaaattattctttcctgactttgcttttgattgaaaagaaaggaagttatatgtcatggctcttatgataatatgatcagggactttgatttccatagctagatcatgaagagttttaatgataattcctaagacattagattgtggattctttgagggcaaggacccaccctatatatttcattctcatcagtacctagcacgggtaagtaatgaatgcttttaactaaattagatagacaattagtctgcagcaaaagtcctagcatcaaaaccccatagtgttcttggattgttcagggagcagatattttgagcatttgtcatgtgccaggcattgggctacccagcaggaataaaatatagtcgctgatgtctaagagcatatattataaagggaaggcaaatatgtataagtcattgtagtttaaagtgttgtgtgctttggtagtagaagtgtgcaaaaaacacaaagttagcaaagcaaacacatgctaattcctatttggagaacggtcagatttgacttttgaactgagtctttaagaatgagtcagagttcacaagatggagaaagagagaagggcattctaggctgttgttgatttttcgacttgtaatgtgtcttgcagcttaaaacattaacactaatcccacctacctttatgattcaagaaaaagggaattcatgacaatgttgggttttcttgagataaccactccattgcaagactgcattacttgtttaaggtgttttgttactttcctgtttcttacaagtgaagacgcttcttagtatggtaggcaaggccccatattactcagccctgacctccctctctggccatgactcctgccagtacccccataataaccccagtcctcctctcagtacatagtgtacttctggtgggctaacctattagtttaaacttaacacactgtattctttcttgcctttgtaaatttagtgtgttctttcctctatctggaatgccttccctctgtgcccactcccccacatttgatggtcatttcacaaactgccactcacacccctcaccaccaccacacacaaacacacacacacacatacagcatcagctgtttatctttgtgctgctagttagagaaggcatcatggaagaagtgatcgtcaggaatgaaccgaggtctctgaggcatgatggggggcttcagggtagaagatgcagagtagctctgacttttctctagctgctcttaacagcttgagtatcagcacagaggaagtcaaagggttgaatcacagtttgcagttagtagttcttggggctgaattttaccccaagacatgttttctttaatgtgcccctttttttctatttttaaattagtcactgatatttaaaaactggcatacttcacgtaaaaatctggcttctcttgagtacatggaaggtatgccctcattgagtttaccctttggcatggaatgagtagtgatgccctgttagaggtcacagaccttcttgaggtgtctagagtccccctctactcttctaggattcctgcctgattcaggtttgccatctctgggattaagtaagttgggtttgggagacagatacagaaggaggataaagaggcaggaaaggtaaaggcgttaggaagatggtggttcaagtgaaacaccgtgggttttgggctccgttgttaaagaggtacagtcaggaggaggttcaggccgcagagaaaaggaaacgatctggggagtagatgtctcagtgactttgaagtgtcagtgtggtagagcatgggccagggagaactggaaggatggaccattgtgatcagcagataggatgtgtgacttcagagagttcagagagctgtggctgcaggactgagtcaccgaagtagagggggcaacaagggtcactggattccaaaagatcaagtgaggccagagtattgaaaatactttgtccacatagactctcaagcctctcagattataatcatataaaacagctaacaggtgttgtgtgcttctgtgttccaggcactatcctgggtgcttttcatcagttcgtttaatccacatagcagtcatatgatatgtgggtactttaatcatccccactgtacataggctacaattgaggcttagagagattacttaacttactcaaggtcacatagttcctaagcggccagaacttgaacctaagactgtgttgttgccaaagtgcattccccaaacctctgccctctaaagcgttggataggagacagagtcaccggaagggaaggtttgcatgagagaggaaagcaaccatgtgcaagtagccagtggtcaacttggagaatgctgacctcccttccatcccaggtgaataagtgagtaggatgtggggattctcagtctcactggagatggctataaggaaatagaatctttaggggagagccaggtaaatgttagaaacaaattcgagtggtgcattgtgtgatatgttcggaggatgggggagtttgtctgcagaggaaagtttggaggaagcaggggagtcagttggaagggatgagtcccagagcagtgctctctggggatgtgcattccttggaggatgagagacaatacggtctttcactttgcactgatgacaaggatgagaggctgggaattgactgcccaaaggaatccaagtggatgcatgcttaaagtcatctgtttcagcactagctcaggccccagatggttctggcggcctgaaggagtgttagcagccaggcaatggaggctttgctcagattttcaagtagaaggttagtccagacatcgttggtgtttgttctttgtgtgctctggggagaggatttaatagtcaacctcccattgtgttctcatttgtggttatatataaaagcttccctaaactaaattccctaaacttagggccagaaccctgtgtatccccagccgcaatccagcttagtacctgctgcatgtacacactcggtaaacagctacaaaatcattacgtgttcataaacaagctgattcattgttccaaagcatctattttctgaggcagttggaaattccactctgaagtgggaataacctatgttgctaaactaaaacttattctccacttcccagcccagttctcctttccacagcgtgatgtgccagtttcccttcctccttttccaaactagtccttcatgcagctctcactcccatcctgtcttactctctaaagccatcctaccagccgtgaaagcagatgccattgtcttcccctagacctacggaagtgaaggcagggagccttccgttcctgggacttggccagctctcgatgtgtgatatcggattgttgagctggactgagtggcccaggatttctgccagatcccagctgtaacatttcaacaggggcacgggccctgcgattactttcagctgtgtttctgctgtgtctgtctccacccccccaccacccaccgctggggttgagtcccttcaaaactaaccctgattttttttttttttagccactcacgtcaaggtagtaatttgtgaaagatgccgttgttttctccccaccacacctactttctgtggggcagagtcctagaagataattgttaaattcttataggaaccacgtagacttacaacttctgttctcctgcagcttataccactttcctttcaacttagcaccttaagataagcttccggagccacatgccagcctcgaggaggaagctaggaagagaagagcttaatggctgctttattattattaccagtagtaggaaggcaagggaatattttattttatttttatttattttataaattttttatttccataggtttttgggaaacaggtagtatttggttacataagttctttagcggtgatttgtgagattttggggcacccatcacctgaacagtatacactgaacccaaacctaattgtctttaacttataccttccctacatgtgagcatgtcctttgattcctccccccagaaaaaaacaaagcgtaaaacaacaacaacgaaaggcagagtgattttttctggtggtcttcaaagggaaagtgttgaagaaggtacagtttctgctcactgtacacaggggagggatggctgcggggggagagtgtgatccccagggcagggctgcacttgctgtggctacagcacatctgtcagtgttagggggtgtaaatgccaggaaatcacgcaggaataagcttgagacaggggagaaagggtgaaggagggttagttattcatacacagatagtagtaaatctcttatggtgaggcacgaggaaaactgtgagcctaaggaactttcatagggaggagtgctgttttccataagaagggttggaccctcagataggaaacctgtttctttttggatattttatttgcagcctttaaaaaaaaaaaaacttacacattcctggaaagagtaagaaatttaatcagtagaaagatagtacatcgaatttaattaaagttgcagctactttttatagaatagcactgtgttttatatgtcattataatttccattcccgacaagctatttcattaacacactgtgtagaatcctagtaacacaagggctttgccctttttgctcctcatttatccttgtttcctgactaaagggcgacctcccacccacctgtaagtagtttttaatttataggcggtcatgtctccacttttgaaggcctcaagcactcacttccggtgtgtagtagatccagtattgaaagaaatcttttccagaagacctcactgacttgttttcctctaggatacgggcagaggaagatcagagtctgctggggaagatgaggaatatgatggcttgctgcttccgagctgcctcctctgcagaaggagcctactatggggaatgttaggataacatcagcacccacaccaggaggatacctctggggtgacggggcgaggcagaggttgaggggcattcctgttctttgctgaaagggctgtgggaggggcaaacaggactcctagccctgtggcctcctccaggaatctcctgtaggttatcttcctttaaggtttttctggtcattgtagtaaaacaaactgttgacccttgcgcttagttatgtagggtctgaaacatgcctagtggtagggagacaacagcatctagcatgttgtttcttttccggccttgcttagagttctttagtagggaaaaaaaagttatataattattatgcccccacgaaaaacaaaacgtcaccagtgcaaaggccctatcttgagctgggcctgaaggttgaggtgcttggagaggaggaggatcaggggagtaagggacacaaggtgactgatgttcctgaaggacagggtgcaggtgagtgagaagaggatgagctgaggtcagtttgcagcagtgacgggaatgggaatggcagtgatagaatcaaaggattttatatttagagaagagtttattatgatccacctgtctcactttatggatgagaaaacagtctgcaagagtttaagtagcttgctagcagtcacacatttttcttctttgggtgacagtattggataaattggtaatatttctgttcttttcttccctcgtggtatctctcacttttaagcaatggtattcaggcaggaaggagtctttgtaagtgatctaaaacaagtactagtaatcattgttctcattataatgttaaaactagttgtacttgttttttgcttttttgtttttttttagtggaaagtacaaattaaactgtgaccacccaggctttgcgcatctaggcacaaagttcttggaaagtggattgcaaattcccatcagcagactctcctggagagcctattatatgggcagattctggagccgctccccaaacctgcagaaaaacaatgtttgagtgggtaccctggcatctgcattttaaacacattccatagtctttccttccacatactaattttgaaaagcactgtacctagaagcttgcagtctaaaatgtataaaaataaaaaatataaaacaatacatggtctaacccctgacttagtatccatgaaaaagtttatttcagattattggttgggtacctttatcatttttttactattatttataagacaagtaagacatgccccctgcactagaggaattcacaggagaaacagtctgattttgatttagtatgatgggccgatgcacttcaagagttactaccgttacgtactgttgcaagtcagaggagggaaaattaatcctgtttgttttttgaccagagaaggtcttacagggtagatggtcttaaggttgtggggaattcaccaggggcaagggagaggcagggatttgaggctgggtggacatgagctgtggctggagtgataaggggtggtggcaggtgattgagtgacatcagatcatcattgttgctggagtgtagggtgtgtgcaggaaccagtagaatatggaaactaataacaggtagaggcagacttaggagcaccttgcaagctatgcccaggagctcggactctgaccttctggcctgcctgggattgtctgtgaagtccaggaagcaaaggacatgaagggaaagcttaaatgtgtactcaccgcccttcagtcagcagaacctcctaagggggtgagtgccctctaggggtttccatgatttctgagttccattgtactcaggcactcaggtgcccactgagcttgaatccattacctacttgagaaaaaaaaaagacagctttaccaagtcccacctggctgcagctgtggttacattatgcaaaaatttattaatgtggtacctcgattgatcattatattaactctaaggattacattagaggctttatttttctggttacttgcaaatacaggttgcatcttctttgaagttaaaaagcccttagcccacatattgtatgattccatttatatgaactatccagaattggcaagtccatagagacagaacataaattagtggttgctaagggccggagatcaggggaaagggagtgactgctagtgagtacaggatttctttttgagatgatgaaaatgttctgaaattaggtactagtgatggttatacaaccttgtgagtataaaaccactgaattgtgtactttaaaagggtgaatacttatcgtaagtgaattatatctcaataaagctgttttgtttcattttttaaaaccttagcacttctgctcaaggtcatgcaaatagctctgcctggggtggcctttatgaattcacagtagagaaatctcagtgctctcctcactttcttttgtgtttttgttttgttgtgttactgttattcctgtcttgttcctctgcagttcacttccacttaagaaacatttatatgactagacatgataggtactagaagagcttatgttaaggtgagtcctcaagttaccacaaaatagagaaagagggtgctgtaagaaaagtgtggtgtttgggggttcagagaagaagctgttatccgaggttggcctagaaagacaactgggcataagggctggatgaggaatgtgcagaggccattagggcaggcagtgcagagacagcaaagtgcggacttatttggagaaaagagcatgaccgtagcaatggaaaatgaagggaaatactgggaaacagaattgagaaagggttatatggacttcagagtgttgaatgctaggctgagaaagtgatacataaaatgaggagtaatggaagataaattttgactgcagaagtttgggaactgagccttaggatgggtgtagcatggtttgggagagacagagacgtgggcagggggagattggactagaactggaaattggggagagggcttcagaaatgaggggtaagtgttgaaatcagctcactgcagcttttctcatgaaatgctgtttgctctgccccttttgtgttctcctttcctttgcttgctccttatccagagatggggataaagggagggaaagattagggaggacaaaaagaattaaaatgaagagaaagaatgtggtgaggacggtacaggttgcatgcgtcagttcttctgtagttcagcatttcctgttcatgactcagtagaggaaaatctagtctcttctagtggaaaacacagagaacaggaaaccctatttaaaatcaggtcacttgactaagctgtgtaactcctgttcccatcggtgaaagggaagaatacctcctgcagtgtgtgttaaccagagaatctagaataatcttactgaagaattttttaaaactacatgggactcatatcatttgtagatataacagacttcctgggcaaatatgggtcatgatggggtccccttttgttacctgacagagtgaatgtaaacatacctgttaaaagataactcatgtgagttgtattatgttgaaaataaggaattgctatttgaccatttcgaactgcaaaactggcagtttcagtaaggttttaacctaatagatagctacccaatagatatctgacctaagcccagatttttaccttttgtggttctttcatcagctctttcttgattaagatgctaatttcacgttcaggggaagctgtgaaaagtcaaaagatggcaacaggtagatcagaaggctttgcagggaaggaaacaaggagacaggagctggggtcacaccagctagcaaaaccaggatctttattatgcagtcagtttagttgtaaagattcagcataaaacattttcctttcctaggtgggttaataggtggttgggtgcaacttcagctcttctgtagcctgctggagacaacttgtaagggactttaaaaaaaaaataggaatatttaaatgttaggaagacgtaatgcatataatgtttactctttgggtgacaggtacactaaaagctcacacttcacctctatgcaatatatgtctataagaaacttgcacttgtactccctaaatgtattttttaaattgtaaattttttaaacaaatgttgggaaaataggtcaaacataaaaggggaaaaaagacttcatggcagacttagtcatggtgttcagtgtggtgtgactcctaacagcatagcagagaacccagcacgtaagtgcacgtgttcagtaaaggttgttcaagtgttgaaggacaacatctgaagaggtattttttggttgactgtacactctcaggtgtttagagtagaatgattccctggagcacgtgtgtaggcaagaatgtggaatcatagaagatttgtaagagaaggttaatcagctattcttgcgggatgctttcattgtaatcacatctacacattggggccagggtgtggcctgtcagtattctagccccatttatctataaactccagagagttttaagttgtacagagtgccaagtacaataaatgtaaacaatataatgttactgttatatcacagcagtatgattatgtgcttaccactgcaatggctagggatataagcgtgacaaagtatgggatgaccttatcaggcactaaaagcccagctcacatttactcagcgctagaaaatgattgtcctgcaggaatatagcgtatgaattgagagctccaagctctactcacacaaaacaggcaaacctttacttgaaaccactggtagaaatacaattgcagcaaaacctgaatgacctaacaccctcttctcccaggaatccaatagaactgccaccccacatggaagaatcctcaaggagcctaccttggggtgatagtggggtgtttaaagccacctttccagtggctttaatagtccttgatgcaaatcataacctaaagtaatttcttttctagttccatggttaactataaatgtaattaaatctctgggcctcattattgaagtacttgtggactataagcatattttaatttattaaagatccaaagaacaataagtgaacaaaccaattacttaaataagaaggggggacaaaagcacaattaatgaatgtattgatagttttccctgctgggaacattacctttcctgatcttgacctctaccgtatgtaaatacccacatacttcctgccaggatgtctttttttaggatgtcttccttttactcctgtcaacct

SEQ ID NO: 22 - DNA, 50000 bp, Homo sapiens
gggaactcccgccgtcatcttctccaccctctgacccatacatctctccccaggggagtcccagggcagaaatctagcggctctttcctcatcttacattcacatgctgacacccagctgccctaagtttcctgtgtcctgcctctctttcattgtagcagatgcaacattttgtctcctcttatttcacctgcccgtatttgccaggcttctgcgtttagcaatttttgcagcagcatcgaagttaggggtgagagccagcacagccagcttgcagctcatggaacacccccattcatgcgtgcagagagatcgatgctcaggtgggagcttaagatccaaaaaattaagactttcagcttccaagatagctgtcataaagttcacttactacagcttaggccctctctttataaagagctcctcagtcttcagtccgtttcttttattgtgcacagtcataaattaaatattgctcaagatgagagtggacttaagacaatcacactgacaagaaaatactaaaaagctaataacttaatcattgaaccaggagtcagaaaatctctgaaaaaccctaaacatcagttgcagcaaaggagcaacatgaggccatttagcacgggaacactcactctgtattagcctgtgcaagcatcgattcacagaagagcataacagatacatatgaagatgtccctgagtcagccagaaaatgtttgttacgctcaataccagtctggttaagtaagaaatgaggtaatgcattttagaaatatttctgtcaaaatggttaaagttaaaaattggggtgtacataaaggagacaagccttagttatttgaaagcaaattatggtagaacccctcctgccttcccatttcccagtgacaccagataaaggaggcagtgtcgtatagaccaggtactttgccacagccactgccactttgtttgtggtctcccgacttctatgttgttgtttattcatggggttttccttcccttttctttggtttcagatgcgagtgtctctctggcttatgtggtttccccgtgtgtgaggtgggatccactccccgcatagtctctcgtggcgatgggacacctggaaagtgctgtgatgtctttgaatgtgttaatggtacgtggggtttctcttgttctcagagagtgtacatttgtgcagaggaggaggagggagggttcaacttagctgcttctgccttttacaaagcagggattaccttcaggggcttgctgaagggcactcaaaaaggtttgcttaagatccacagtgcagtaaagaaagttaggttaggaactatcattccacccatcagaaggaggattctggaaatgtaatagcagattgcatgtagctgtcatactcagctgtgttacagaatttccaaaacttagggatttcaggaaaaggatcctggagcctgtgagagttttcctgctgtcactcagtaactggtcccatggctgtcgggaggtgacctggctaaaactgcattgggcctgccacacttgggaacaagaaaaacatggctcatcttgtttagcattttcctacttagaacatctttgcacctgatcttagccaaaaggctgagaggcggtggggaagacatctttaatcagtggttggtcactgatagcggcctgctgtaggcttgacccacgtggccctgaaaaaggattcttgggtcacgagattccttctgacagaatctgcctctccctttcactttatatgggaggatttggtgtttggtggcttgatgttgattgagtttaggtgctagaaggaaggggaacctgcaacatgcttctctaccagaatattcattaacaaaaaataaaataaacaagttacatgtcaataaccattcagagcattatttagtcaatacttgtatacacaggaagatggagatggatttggacaaatacaacaaaattacacatagaaatattcatatcaaatacttcctgtaggaattcagatgtcataaggtctcctgccctcccaaggcaagaaaggttatgttgcagttgagtcctatttggttgcatcctgtgatttttaggcccgtggccaagttgttttttattgtttatttttgctttgaagaaacacttgcagtatggctttatggttaagaaagaacacagactagaattaataacctcagtttgaaaattgggtcctccactcactagctgagttaactttgaggaagcttcttggtttccctgggcctcagtttctacatctctaaaatggactgaataacagtaaattaaacaaggtattataggtgaactaccttgaattttgcctcagatgtaaataatctttagtattcttttttttttttttgagacagagtctcgccctgtcgcccaggctggagtgcagtggcgcaatctcggctcactgcaagctctgcctcccaggttcacgccattctcctacctcagcctcccaagtagctgggactacaggcgcccgccaccacacgcggctaatttttttgtatttttagtagagacagggtttcactgtgttaaccaggatggtcttgatctcctgccctcgtgatccacccgcctcggcctcccaaggtactgggattacacgtgtgagccactgcttccggccaataatcttctgtgtgcacactttctattcacatccagaggcacagttctggacattggggatgggggatagagatggtaaggttgaacttcctgacttacagaccttataaatacaagcatacaggaaaggggaacagaaaaagttctgtgcacttaaatcacctctcggtgcttctccccactctggtcattatttagccagtaatcacatccattaaacacaagcaaatggccaagcagaccttttttctggaggtgctacagtttgatttatgataggtgagattctgtgaatacttgtaccactggagacagaaatagtcactgcttccctgggttcccagcatctcagatatgcttcatccagtgctcatcacttggaaaagacttaacgctcatgcctgtctctcactccaaaatgtgaactgctggaggacaggagtctgtcacattcatccttgtgtctgtagttgttagcctattatctgcctcacagaggacacccagattttgttgcatgagccagtgaatgatctaattcgtatcatcttagttccctctagaggtgtctgttaaatgtcagcccataaatgggttactttgtaggaatcctccatctatttactgccctacactttaatcttaagtcatttgtcccctgaaaatatcatctctagtagaaagatttgctggtgtttcctatgggtgaggaaccggagtgcccttcttctctccttgcttcttggttctagatacccatgggtcattcctttcttatcagaacatagactttatgtcttgctccagtctgatgtgtctcaaactactaatagccgtacctccaccttagcaaccttgggatcagtctgcagggcctgtcctcatttattgccacttattatagagccctgacttctggaataggatttgccatctgtcctgagaagatgttgagtggacaaaacacagtcactacatctctggtcgttgcttctttcttgccaccataaacttgttgagtgatctttgctttcttcctaatcctgtggaactgtgtctattgatatatcagtcattcacctgtagaggagactaggaactaatactgtgaagattcataatcctacagaatattgttaagaacattgtctcaaagcctgcatttttcttcttttgatatatacattcatactcatacacacacatatatattgatagatgaatgtgtctgtgtatgtatatataaatatacttaccacctagtgtcctggaaaaatctgttgttgtaaggtcaccattagaagatactacatatcaaagattcattatcatcacttagaaaagcagaggtctattttatatgtataaaaccactcgcttatacacagaggatctgaggaaactttacagtgagaacagattctataagatagtgataaaatatttatgagaaaggagactcacagccatagaattgtaagtaaaagttgaaagtgaaaaatcaaacagaaagcacccacgggtatacagtccatcagtgcctatgcagttgtgggagttaaatgttaaattaggcccccagttttctggccatcaaagtgaaaaaggagatagggtcaaatacgctgttcttattttcaaagaggaaaaaacctgctggatgggacaactgtctctctaatagagatttttcatatggaaaggaatggatttcatcccactgaatgatgaagttgatcatgtgcttaattgcgttaacaccacaaatgcagaaactggtttcctgggattgttctcaataaaaagcaaaggcaaaatattaaaatacaactcagttccaaaaggtcagattgacacagctagaccgtaggtgcttggaacattgtttctcaattttgcaaactgcccaaccatggtgaggtagatccaaagaccccagttcagaagactgtgggtagaatgttaacatgactgaccccacatgcagaaatggattatgcactgccgcactcacttcctatgctgccatccatgtgatccatatgcttatgtttttttctttttaaaatcaactttaaagtgaaaacatacaattttaaaatacacagaactcgaaaactcgtgagttttgaaaacactagcctgatgagtagatgcagagagagcatcctccataaatattactttgtttttgtagtttgattttattttgttgtggcggagggatgggagagttgcagaccagttcatgacagtttgtaactgcattctctgacaagcgccgaaaggagaacttatgaggctttggctgaacaagatccttgttatttagaaaccaaaggaacaggtggtatgattatgtttcttttatgtgaacttagaagtcatcaaaatcatcacctagaaatatgacctccaaagcagtgttgtaacagccctccaccttcgtcaagaacccactgtatgtctgctcctgcccacaaacaaatgtcccatcagccgtcagctggagctggacaatggcctctactgttagatgggtggatgctctccacttaacacagtaccccctgctgtcttacacatggcccacctctcgttcctggaggcatttgggtttgaaatcctaggtgtagagtgtgcaagtgattatttggtttcttagatacgagcatttgtccaacgtatatacacaaacatctagataaaatggaatatcttggttggaaagtcgtagccaaaagctgactctgtctgtatgacctacttataaaacgttccccaagtgagtaaatgaattagtctatagtattgttattggatagaagccagttgaaactaccagaaatatttttagccttacggtatttaatactgaaccatttgttctgacagcatagttagcagggccagatatatattgaggctagcccccattcacagctctaaacaagtcatcagcacaaaagaggaagagtaccctcttcccagtggtgccaagagtggtgggtggatggggtggggatgggagcattacttgtgtatggcacaactagccaaaagcaacttcagggggttatgaagtaagttcagagaaaggggatctgaatacaacttagcgtgtgtttttagcctgttttgtgccatttgaaatacttctgttttggagcactgtccctgtagcactctgcttctcacagcagtattaaaatgcagcactaacagtgaggagcctggatgaaggtcatgttgcccacccagtaattagttcctgttttattcaggcatatacgaaaggctaataaaaactggaagtaatcttatgttggccctgacagaattttctctaagacagtaacatgtgaaatcaccatttaaaggggagccgtacccagcatcagcttgctcctcatgtaaaattttggcctagaaaattcagacctgcaggctgttaattttcaaaaaaaaacactagtacagaaaacattaagaacccactgtctatatagcaattatgctcataatgtttctaaggacgtaatgtcttctactgatagaataatggagatattctggcactttgagagcaatactccactcagtatgtgtttattgagcacccctcgtgttcctggggccttgttaagctaattttgttaaagagtgatacacattgttgtaatcctttctaagggttgttctcttccccttttcctgaaataggatatgcttggtgtcctccagcacacacacacacttcccctcttctccctgtggaataagccaattgaaactagcaaacatatttttagtcttatagtatttaataccgagccattaattatgagagtatagttagcaagaccagatatttattgaggctagtccacattcacaactttaagaacatgtcatcagtataattaataaagggaaaacttaaggatttaaaaacacacagaactcaagaactcctgagttttgaaaacactagcctgaatgaacagatggagagaacatcctccatgaatagtccttctcttttcactttggttttaatttgttttggtggagggatgggacaagctgcatgtcgaagctgcagagagttatctagaagatttagctatgaaaagtgatgaaggtggctacactaaacaacagatattcagtgtagaggaagcagccttctatggaaagaagatgccacctgggactttcataacaagagagaagcagtcaatgcctggcttcaaagaataggctgattcttattaggggctaatgcagctgataactttaagctaaaccccacggctgttaagaattatgctaaatctactctgcctgtgctttataaatggaacaacaaagcctggatgacagcatggtttattgattattttaagcccaccattgagacctactactcagaaaaagagatttcttccaaaatattactgctcattgacaatgcacctggtctcccaagagctctgatggaggtgtacaagattagtgttgttttcgtgcctaacacaacatccattctgcagcccatggaccaaggagtcatttcaactttcaagtcttgttaattaagaaaaacatttcataagattagctgctgtagatagtcattcctctgatggatcagaactaaatcaattgaaaaccttctgcaaaggattcactcaccattctagatgccattaagaacacaggtgattcaggggaaggtgtcagaatattaacatggagtttggaagaagttgattccaaccctcatgaatgactttgaggagctgaagacctcagtggaagaagtaactgcagatgtagtagaaggagcaagagaactagaattaaatgtggaacctgcagaatgtgactgaattgctgcaatctcatgataaaactggaaaggatgaggagtttcttcttatcgatgagcaaagaaagtagtttcttgagatagaatctactcctggtgaggatgctgtgacaacatcttcaccagataaaacgacaaccagggatttagactattacataaatttagttgataaagcaacagcagggttttagaggattgactccagttttgaaacaagttctactgtaagtaaaatgctatcaaacagcatcacatgctacaaagaaatctttcatgaaaggaagagttagtcaatgtggcaaacttcattattgtgttatttaaagaaattgccacagccaccccaaccttcagcaaccaccatccttatcagtcagcagccatcagcatcaaggcaataccctccaccagcagaaagattactatttactgaaggctcagatgatcgttagcagtttttagcaataaagtattttaaattaaggaatgtatattgttttttagacataatgctattgtatacttaatagactatagcaggggtgtccaatcttttggcttccctgggccacattggaagaagaagattgtcttgggccacatgtaaaatacactaacactaatgatagctgattagcttaaacacacacacacacacacacacacacacacacaccatcttacaatgttttaagaaagtttgcattcaaagccatcctaggcctcatgtggcccgcaggctgcaggttagacaagtatggactacagtacagtgtaaacataacttttaaatgcactgggaagccaaaaagtttgtgtgacttgcttgagctggtctggaactgaacccacaatatctttgaggtgtatctatcaccatccaccaatcagtgtagtttggtaggcagtagcatgtactaatagtctatattacaaaattctcttttaggagttctgaagtatattcaagaaggaagggaagatcccagaacttagaaacttaaagtttaattttgcagacgcaaaacacatacaaaaaatcactaaaaaggcacttctgtgcttcatattgagaagtacaaattacacagtaaattatttcttcataacacttttccacatccacttaaatcatccttttgtggagattgtggcaatctccacactgttctgagattcacatctacccctgataggtgaagaagtaattggtcattgctcctcaaaaaccacaggtagcctttttatcatatacttttcttaagagcctctataaatctctttgaacatcaaacagatttcattgtgttgtgtgacttttttcacccttaaagattgattgtgtttccaggctgtgtttttaggattctccatctttctcacttccactcccttgatccttgtgcttttgctagtgaaacctaagtgccacagttctctcgtcagtcccatcccctcctgcttaagcttctgcacaccctaccataaaaacaagtccacttgaagcagctttattcataaccagaacctgccctccacacttcctcagatcatgcaccagggttgtggcttcacccagatggtagctgtggttgatcaactcaaccaaataattagtattgattggtttattttttttagtttccatggatgttcattttgttagagtacagtattttgcctcgctctacagcctgcaaatttaatgtataaaagtactccgttttctatactttttggaacttatagtgtagctgcacaattcctttcaatcccaactcagattagtttgttagctaaaagtatatagaagaattctgagccatggtaagatgattcttttttaacattaattttctgaaaaaatgttgtatccatgtagcactaaggaaggagtttaaacattcacatgtagtacgtgtgcacacataaaatcactgcctcattcatgtgactttaagcaaatcacgtcatctctttaagtttcatcttcattatcgaaaaaactggtactaataatactcgttctgtcttcttagcggggttgttataaaataaatataaagtaattgtccttgagtacaatgcaaatgcaaatatcctccaaatgcggtgaaagtattttagacatgcaaggggacatattaaatgatttggggatggacagaagagcccttgaaagatggggatatcatcatccagctttcaccggcagattgagcagggctcatcacctcaggagaactttcttcagcttgggagtactttttttaaaaatagctttaggagacaccatgcaatttaccaacttaagttgtgcaattctgttgttttcagtgtatgctcagagttctgcaatcatcaacacaatcagtttcagaacattttcatcaagccatcagaaaactcctgaactgattgcagtcactgcccattttccccctaagctctcagatctaggcaaccactaatctactttctatctctatggacgtgcctatgatgggtgtttcatataagtagagttatataatattaataccttgtcttttgtgactggcttctttcacttagcataatattttcaagtgtcattcatgttatattgcatatcagaacttcatttctttttatggccacgtaatattttattgtatagatatactactttttgtttatatatcagttaatggatatttaggttcgttctttcttggctcttgggctgctaggaacatttgtttatagtgcttttttgtgggtatatattttcatttctcttgggtatatacctaggagtagagttgcttgatgatatgataactcaaattaaggaactcacaaactgttttccaaagcagctgtgcttttacatcctcactaaaaatctatgagaattccagtttctccacatcctcatcagtgctcgttactattttttaattatatccatcatagtgggtgtgaaatggtgttcattgcagcttagatttgaattccccgagtgactaatgattcgagcatcttttcatgtgtttattgtccatttctgtcttatttgaagaagtgtctgttcaaaggtttgcccgttttgattagattatttttatcgtttaattatgggagttctcggtaccagtttttttaaatcagatatatgattgcaaatactttctcccaatctgtagggttgtctttttacctttttgatggtgtccttttaagtcccaaaattgatgaagtccagtttatctgttttgttcctttgttgcttatgctttgaatgtcacatctaagaaaccactccctaatcaaaggccacaaagattaactcttatgcttccttctaagagcactgtaggtgtagctgttgtatttagatcataactctatgattcattttgagttaatttttgtatatggtgtgaggcatagggttttcttctttattcttttgcatgtagataatgcaaaagactttcaacctcaggcctggcatggtggctcacacctgtcatcctagcactttgggaggccaaggtggttggatcacttgaggtcagaggagttcaagaccagcctggccaacatggtctttactaaaaatacaaaaattagctgggcatggtgtcatgcacctgtagtcccagatacttgggaggctgaggcaggagaatcacttgaacccaggaggtaaaggttgcagtgagctcagatgacgccactccactccagcctgggtgacagagtgagactctatatcaaaaaaaaaaaaaaaaaaagactttgaaccccactgagtcttggccatcttgtcaaaatacctgtaaatgtgatagtttattcctgaactctcaattccattgaactatatatctatccctgtggcagtaccacatagtcttgattactatagcattgtattatgttttgaaattaggaagtaggagttctctaactttgttctcccttttaagattgttttggcaattctgggtcccttgcattttcatatgaattttatgatcagcttatcaatttctacaaaagagccagctaggagtttgatagagattgcattgaacatgtagatcaatttggggaatatttccatttaaacaatgttgtctttcaatccatgaacacgggatgtcccgtttacttaggcctttttagttttttgaaaatgttttgttattttcaaaatacaagtttgtacttatgttattacatttattcctaagtattgtatactttttgatgctattgtgattggaattgttttcttcattttctgattgttcattgctagggtatacaaacacaatttggggagttgcaattgtactctgcaaccttgttgaacttgtttatacattctgatagttttttagtggatcccttaaaactgtttacatacaagatcatgtcatctgcaattagagatagttttacttcttcctttccaatctggatgcttttaatttatttatttttcctaattgctctggctagaattttcattactatgtcgaattgaagtggtgagaacacacctcattgtcttgttcctgatcttagggggaaagcacttggacttttgccattaagtatgaggttagctgtaggacttttgaggatgccatttatcaagttgaggaagttccgttctattcctagttggttgagcatttttatcatgaaagaatattagattctgtcagatgccaaaaactgtgtctatcgaatgatcatgttgctttttttccttattgtgtgacatatcgtattaacattaatattttggatgttaaaccaaccttgcattcttggaataagtctcacttggtcatggtgtataattttgtttatatgttgctggatgtggtctgctagtaattttttttaagatttttgtgtctacatttataatatactgatctgtagttgtgttttcttgtcttggtgggagttattttaaatcaactaaaattaaagaatgaatgtagggagagtgtttcaaggcagagaaaaatttaaaaatcattgatatcgtgatgagacttctaaatatgggactatgagtcctcagcagaacataaccctgaagcacaatggaaggagcacaaggtttcagttgaagagctactgttttggcttctgtcgtttgctgtgtgactttgagtaaactacctaggatttcctcacttttaaactcaggataataatacctatgtcagaccttcattatagggattaagtgaggtgatacatatagagagtattttgttaaaggccaaacactattaaaattatacgtgtttataccatgtacagagaaaaagaatttaagagagagcactgggggaacagaacatcaccacttcaaaaaagaccataataaaatttttgaagcagaacaaaacctaatgtggaggcaggatcattcatttgaggtagacaatgatttaaccaaacaaatagaatcactttaaagcacataccagatatgtgagtgtcatggtcataaagtttagctttgtctgggatggaaggaagtcagtgtagattttcctagggggaatttctgttccttggtgtaaactggataacagtgtgttttattttattttgttttgcttcactttattgttttctgttacatgaacttacattgagttgagtaaatttccatcaacttataacctcctattagaaattttctagaggctgtttgaaaaacatcaaaggacacaactaaaaaatgactacaaatatgccttgtttgttttaactcttttcagatacaaagccagcctgcgtatttaacaatgtggaatattatgatggagacatgtttcgaatggacaactgtcggttctgtcgatgccaagggggcgttgccatctgcttcacygcccagtgtggtgagataaactgcgagaggtactacgtgcccgaaggagagtgctgcccagtgtgtgaaggtaagaaaaggtgctaattacagattaacaggagcatacatggtatagagttctaaaatcaaaatcgtcctaattcagtctcatccaaaagtaaaggaatattgaagttccttttttagccttttttaagacaccatggtctgtcttttagcatgttaaagccaggtagagtgtgtggaaacacttccctaagggaagctatgcctgggtgtagacaatgggtcacctctttccacaggcaacgtgagatgtcccagagttgttttccgttcctcccctgcagttctagcaattcctcagcaagtcctagggcaaagctttatcttcaccctggcattagagctcaccacctcctcacctcagattgctcagttaggataatgtgggcctcaccctgacagtctcagcctgccggcccagagaggaactaactctgccactgtcgtaatacccaggaccctccagggagatcagggacctgctactgcttctctgaaaatatgtggtggttcacatggggtcgtggtatcaggtcaccacctggtgacaccaactcaaggcccagccaggtgtcaggtgtgactctcaggaatctgctttgtctggattccccttttctctccaagaagtaatatgttcatgttccttcaacaggctgcatcttcccatcactgatgttcataaacaataagcatttcaaaacagttgctctgaatatcactccctgcaacccacacattttacagattaggatgttgaagttcagaagagaaaagtcacttgcctgtaaagtcactttacactgcagccagtaacaataaccccgtgcctgactgtgtctatagtgttcttctgacaacacaacactgtttccccagaaaggaaggtcttgtgagccttcctgtcctaaccaggccccagatcttcaatctgaagtcgcatgtgttaatcttatgcctgatctctgcacacaaagtgggagggcacagtgtcatgccactttccatacttcctcattttcataaaattttctccttttgatatttgagcaatatttacaacataatattgaagatctgaaaatgtttaaagtataaaaattgcattcctaacatagttcataacagtgagttcttttccctactagaggttattttacccaacataggagaaaagtagtttattttttaggtgatgttgacaattaaaaagaattacttcaaatttttaacctttctaattgttttgttccctacctgcccccagtctgcaatagttctgaaactgtgcagcagttgatgctgtaaaaatgtagatacgaacctctgtgtagtcatcatactgagttgtctgggcatggatccagttatgtctcacttcctggaattcagatccaggctcaacgtagaaaaatatgtattctaatactttttacacaggaatttaaaaaatcttctcttgctcaggtccattggccgcctccctctctggcagactgccttcctaacgctgtgatggcattattttaaatgctgaccttctaactgactttattataatgtcccactatgtccttagaataatgatgaccaaactctatgaactgtctggattcctctctcagcacagcagtagttgtttgttgaagccagttttttatgtatttctgagaaagaaaattgagtaatttatctggttgctctcaatagagggataagagagggtttcatatgcagtatttatatacttgtcagtaacctcagaaacttttttttttttacagtagctttggctacttctgtctcccacctggggctgcagcttagcttcccaacttatgggagaaaaaatggcctaagttcacagcgctcctccaaggcacagtgacccatggtatgtctagactagccttgtcatgaacaagtccacagcaagaccactgttgcccacatggtcgtgcaggtacatgagatagagttttagaagtaacaatatcactctgacttagggcattgttgcttccgctttacagttagggaagttgaggtttgataaacatttatgtctacaactgcgaagacattgtcagagcctaaaatggagtcatgttttctaattaccaacccacagctcttgccacaaagctttccaactttacagagtgataacacctgcacattggtttttagactacagatcctcttctctgggactgctgtgtcaggatgagtagacataaattcagtgtgacagcttcttttgcatgcacccccagagactcctcatgcaaccctgcgcttctgttgatcacagtgggaatagaactgctgatgactagaacccagggaaaaaatgtctctgggtactgacagagataagatttagaagatgctcagtctaactctgaactcatgttctcatttagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaaggtaaatcttgcagatgctaatgagtccctggtgaatgtcttctgttggagaagcttctaccagcgtacgttcttgtttgtttatttccttgggcataatgtctgcttcacgctgttaccagttgccaacaaatttatcaaacagctcaaactgatgatttgaggaaaggttgcacatgtgaaatgagaatagtgtggtcattattttgccattgcaaatatgcgcgtctgaatactttacagtataagaatatgtacattttaattaagttcactttgaacctaattaacacccaaaggtcagcctaacaaaatgaaattgaaaatgagaccgatgagtcaatcttagcacctttgacttgtgatgcttccatatgcccaagcgtcctggcaactagaagaaaagactggaaccatgaaagccaatttccactgcctagtgccgtcaccgagtatctgttctacttctcatataccaatggctgtggctttgcaattcctctagtgctgttatttatgtcgtggttacctgcatttgctatctctcttcttgttcagtggagggaagcctgaggcatcctggactagtttttctgtcccgactcctattccagctcatttcctccatcctgggaagattctctgcatacttaactcatgtcagtctttaatcttgtgtccatgtgtgttagtgcagacaagggcttgaatatttgtacagacatttctaatagagggtatccagcattaagaagtacatttgcatagtaaaatgaactaatgtggatcaaaaattaatccatgttgaagatggatctttctagaatttctaaaaacagatattgaaaaatggacttatctttccagaaagtcagttaagtagactaagtcttttaagtgcgaagaaatcttgtgcttccaccgaaatgttaataaactcagactccagagtttgcctgactagcttcatcagcctctgaggggctagacttattaaagaccttaaaaatcatataaaagtttaagcttgaggcctctctttctgttgacatcaccgattcccttgtcattactgtactcagagtgcagctgccacactccagaactttacagcttccagatgaccagttttgtggaagtcacccacccctgcatgtatatatactcctcccattccctaaaggcagcatttcagtgctgaaaaaaaaagagcatgtagaaatacatatgatggttgtatagtcggccttacaggaaagggtgccttactgaagcagatgaagataagaatgtcagtccctaagaattagggacaatctgttgccaacttgaaatatttaattacgaatatttgggacacacggtaaggaatatgaaaagcataaattcataatccagtaaacattagccttttaacctcatgggtcttatgtcataaattcaaaataaaactttaaaaacttgttgaataaaagagcctttcctctggaagtaaatgaaaggaaggctgatccttttgcacccaccttttgcatttatcagcatatccatcatgaagagtggagaaggaaggcagttggtgaacaggtagatatatatataactcatttattaccattttcttagcacacattgatgtttgggtaaggagtgaaacttagtgtggtcacttagaatttggtctctgcaacatctgcatagagttgtaatctaatcatctactgtttgagaaaagagttacagttttggtgagccattaatttgaaaaaattaagaacctcctggaattcagatacagcattggacacaaagataagtaagagataacctttaactttgaagagcctatttaggctgagttttgaattaaaccagaagaaagggaatgtccccgttgaggtatggtggggtaagtgcatcacatttgtacccaatttcatttaaccttcagagcaacactgctgaagcctttattatcccattttggagatgaagacattcaggtttaaggcgatttagatcatttcccccaagggtatagttgccattgtgtggctgagtgaagatcaacggaagtttgtcagattcggctccatttgtgcttgcttcccctgccccacacagcatcccagcctaactcatgccctgcttctagtgtcctcacccatctctactcacctctggcttttggctctgttgaggagtgagggcagtcagcggaggcaagggaggaaatcattttgcagtttgaaaactactccaggtattttgaatgtgatcgtccacctactgtctcctcgcagtttgaagacctcaggggaaagatgcctttgagatgcatccctgagttcacttctagagcctcaggtctgggcagccccctccacctcaacagagctcagcatccttgtcatttcagcggtaaagttctgctggccttttttcctctctgtctctttcatgctctggctctgatctctcatcaaacgtttcagaagattccattttctggttctcagaaataatttatcttgcttttcttgtttttgtttttaacttagttgagtagtgagtttcatatgaatgtatttactctccaataatgctttcattttaagatgtattaataataagaacacagattattttagtataggattaaaaaacagtgcacagcttaagataatgatactaaagggttatttatggctactaataataaacttacaaaataacacaaggtgaatggatcttactgcttagtttatagggtccaagaaagtctctgggatcatttgaagacattttttctcagcagtcattcaatatcatacttaaatataatttcaaatgatttcagagtagcaaaaggattgtatgaaataagaaaaagtgcaaggtgttaatgtgcaataaataagcaatcacccagaaatagttagaggaaaaccagaaacactggtatttacttagtttatacacacaataaattcctctgtgcacagttgcgtaaccagtgttggcccttaccctattagctacagtctctaggtacatcgtcagagtatgaaattagatgaaaggaccccacacaaggattgtggttttagattctaagtaaactaacagcatcactaaatatggcgtgttaggacctccagggaggaagaaccttattgtggctgttgttactggagacacataccttccaaatgacaagataatgtgtcttttggttactcctggaggactagggacatgaacctgggggcccttaaagactaggctgttacttgacagctaacttgattcacaagtacctaagtgctttttaaattggcacttaagtactctcatgatgaattcttagcaagttattgtcaacacccaattcaggtatacagataagcctttccagtctgttaactgattatattctcacattttgctttgttgacttttctctacatctcttgtttatcgatctcattatttttattttctgacccctgtgaagagctagggtggaaacgtgtggaatagcatataaagttttcagttgaatggcatacatagacgacacagccttaatctccttaaaaactaaaaacaaaaaagagattggacggcagaattaggtcttcatctgcagggccactgttatttgtaacccttaatgacagtggctaacatttcatacaattgttatgtgtcaggtttaagtgctttaagaggagcaactaatttgagatcctcacagcatcccttcggtatgggtgctgtcatcccagtttaccatcagagaacacaaagttcggaggccatgtaatttgcccagggtcatacagctcataagtgacagcacagtgtgtccccaggaccagaccctggaccctgaatggacagtgcctctgcgctgcagtgcattataccctgaaagaggctccatccccctgtaaagaaagaatttggcatattcttttgaatccctaaaacaacatgtgatgtgtaagtagatgctcaaaatttttaaatgagtaaataataagtgaggacagtgaggaggaaagaaagactggagcaggaagtcccaaccagacccttcatacagatcttctttttagttgagtgagggagcagaccctgatacagggttaccttaaccagagcgtcaaaagctgatggccattgggacccctgatctactagttgaaggaagaataatgtcagcatgcttatcactgaaaacacattttgagaacataagtaagagtgttatggaagtgtgtgacatgaggtgggcctacatgtctacaggccagttggaccagtttcctgcattgacggttcacccttctgatgggcactcagctctgggctccatccctgtgtcagcctcatgggccttccaagactgatgtgtattctgtggtttattctgtcccagttcagcccctgaagtccagaatcacagagtttaactggggcttcccagaccctctaaggctaatgcttgctgccacgttaatgtactgtttttaagacgccttgtgcatggtgtaggtgccactgcatttcagttacttacacgggcaaggattttttttacattgtcattagatattgagaattatttgctgattttttttttcaataatagcaagtataaatgctgccaaagctgattcttaatgtcagagggatcctgaggagaggttgccatttccattagaaatctattttgtgattgaaggtaagataaggtatcggggagggaagttttggctacatgacatctataacatccccgtgattactcaataagaaaaattcaagagatgtggacaggtgtgtggccagcttagctgtgacacctgatgcaagagattagagcttcctggggactttgaagtcttgatgtcagacctgatctgccttctcatcttgggctgcggctggaccctgcccaggtggccactgtgatctgagtgttcttcaggcctccttaggtgcaccatctcctgcacggaggagctgatgattttgatgaccttagggattttgaagccaacaccagagtatcacttttcaagaactaggaacaacttaggtaatattttaaaaggtagcattcagaattatactcaggttgccagtaaaaaaattccgtgctagcggttgggcaaatgggtagtttcccctctactatttctgtctgtctgaaaacaaagcaaaatccatgttctatctctaacaggaagtcaaagagttatttgtagattgttaaagctggatgagaggtatatcagagtccatagtattcagtattttttaagttttcttcttaataaaaagttaaaattgctatttgcagacatattatcagatacccagctgagtttcaggcctaataattctgctaacctggacttaactcttgaagtagaggatagaggaataagagattcactgtgcatggattccttctaattctgggtttaggatggaatgtggttgaaccaaaaaatccactatctttcttcctgccacctggctttcttggatctcttacttagggaatcctggtcctacttgggacagtgttgaagactggatgtcaggcacgtcttgacctatgtggatttagtaagccacttcagtctcagctcacccaatactgtaagttgtgctcctctaaaaggctggagttctgtgagctgtcaaatatattgtcgtcttcttcatcaatcccaaatttatggaagcactatattctctccctgaaggctgatgatatagcttgaatcaagctgcccttctaatgagatttccactgggctgccagaaaagtctgtaccaggcacatatctgggtgggaattctttgaaacaagaaaaaatgtttccagccctgcccataatgcttcctcactgaacagtatctcaaatgattaagctactgcttttgtggcacggagtcaaacatagtttggctgaccccaagaggcagtgaagtcaaagatgccatagggtcacatgttattaacattctggtacataacagggatcaaaactcagtttatgggtggtattgcttcagtgccatatacgatgctattattagtaagccaagaataataaaatattcgtaatagcctaaagactgttaggcatgtgttttatcctttaactctaaacgtcctggaaactagttccataaatggaaattaaaatataacacagtctattttttgcttgtccaaataaggtcatatgttgggtaacaaatgcgtagcaaatgactgtttcttaacctaggtctttaacataaacatcagaaatttttcaatgttgaatattgtcatattttgatttttctctgatcatttaaaatgtctttacatatttgcaataaaactagaatataggtaaagtaaatgcaaaagacactcacatccagccttaatatatcactttcaagattttgtgatttttcacagctatatagaaatatgtacattatatgagtggctttctttacatgttagatccaccacttacattaatagttaaacaagtgtagaaataaatgttgtagaagtagtttaatagagaagaaatgtttattcttacttatacctaggagttctgctgctggttctccaagtccagcatgtctttgcttgtgtcggtaatgccttgctctttgtacttgattttgagtaagactgtcatactgaaccaagttttacgagtaaaaatgaagattggtcttatactcttaatttgctagatgaccaaactgatcaatgccttaaatgttcagaattgttgagagctaattctgaaacacatgattagaaattcctctgcccctgagtttacagagctcagctgttaagtatttccctgttgaagtggcgtgcaagggggtgggttgctctttatttcctctgctgtctacttcccatacctaaaagggtattattgctgggagtctcataattttcctactaagacaataattgcatctaatagatcagacttgggcctgctaattttcatctaatcaacagaggcaggaaaacatagtctggaatgctaactatgtgaatgaaaaatatttagtcattcttgaaatgataggaatgtttttccaacaagcttaaataaacacaatctcttttgagactgtgctaaactttttttggatgagacaactcttaggcaaccaaacagaagttaatgaaggttattaagagacatgcccttcagaaaaggcctgcagactttaaaaagaaaagattccatcttctttatgctgcaaccattgataaatagcagaattggtttactttttagcttttgtctacccagtagaatttaacattttaattagtaatcatactgcccaatgttcaacagataccattttacctaacatggctatatgagaccatcttattttaaaatcctcatcttcatctcctactgtttgcctcagttccaaggaattcattggactcaacactcctgtatttatagcacctgattacacttttcctatgaagcatgtttgtttattcccaccaacacaaggtcccgccagataataaaattatttttaaacatactaaaatgtacatattcaagtgaagtgtttttcctttcacaagttatatgggaaaaatgtatacttactccagggatacagtcattcagaatatttttggtgcctctcttttgaaactgactttagagatcatagcacatccttttgaatatccttagctttgtaaacccttgactttgaaggtttattagactctaggaaatagccaaagtcatttatgttgaagtctggtttaaaaaaaaaaaaaaggtggccgatctagctgagtaaaactgtttagaggaagcaaaaagtgacagtaaaataaaggcactttgtcttctatgacatagtaatttgctctgaaagcaactatgaagaggtttcaaacagaattttaacatgaacagcattttggcataagaatatagcctctaaaagtgagtacttttatcaataacacttatttgtaagtataactcctggtgtgtttataaaaaaaatcaattcacctactacatagtatttttatgacttctagggattagaggcatgtatttaaatggtcatatactgtattacaacgtttttctgaaattgcaggtgttctattccaacatttttttcaaatacattagactaaaataatttgcctacagaaacaggtcaaaataggttgcgattctaatataatttatttttagtattttagttatccaattccacttctagttataccatactgtttcctggtatagtcttaagtatatcctataaaaacagaatgatttcacattgcttaatatatgagggttagcactgaagatagtgaaaaatccagtgagtcactgttaaacccccattagaacttttctgtgttgtatctatctgattattgaagatacagtgtcacacaagcatataaaccatctgttgcaatactattttaaaatcgtaggacaggttcaaagaggggaaatgtccattaaagaacgcctcttgcgcatgtgtgggtctccccatcacaggtggctgatgaggtggcaggagcagcatcatacccacctatcagcctcttaatttgagagccccaaatactcccctgcatgagctcccagaatgcagattgggaaatctgttgccaggggactcagcaaagccagatcttggtccaggggtcataccatcagtttaatctaaatttgatgtgctttgagcaatacgggacatgtaacatgtttctgaaatttgccagaaagcatgcattttgttttataaatattttgagtttaagttgatcagcagtttgaccaaatttttatttaccatttatatctacagaatacctgtttacacctcagtggagtttaactttcgcatctgagagattagctggatttatctggtttgtttgaaacagcctagttcaggtatatggaccatttgagatcacagaatttaatgctagctgccagtaaaaagatgcagtcccaggctccagctcttactttgcagcagcctagtgaaacacaactaagaatcctcaagatcctaaggccaagtacttgatccttgtggaccctgcaaatcatgtaactttcacattggctgccggagcaaaccgagagttgatttgcaagccaccgctcgaccagcaccagaactcaaagcatgcgttctgtgtgcttacctcatccctgactgcagcttattcttttctagctaaaatttacctggatccccatttccttactttaaaaaagtattcattcatgatataatttgagactgccttggatctattacataatctaatgaggtatatttatgtgcttctttatgtataatcagtgcctaccatttgcccgtgcctgtgcctttgtgccttgcatggtggttgaacccccttagtttgcctatcagtaggaagacaggagatcatgttgtttttcaccttaatggacagctattgccctgcatgtcagctcacttttaaaacaattcaaggagtctctatttgttagggaagctgtaatatgtttggagataataagctatccacttgggatggcctcgggccttgcatacggagagggtcggcaagctcagatcttttcaaagccataaatctctcatcctttctgggaatcaagtttttaaaagattccatggagattttatgttagacgaattccacagggggtgtgtcccatagtcctgtgacagagatactaagaacatgactcattgcatgaggagcccctacaattcttgttatgaaagtactggttgaattttctcttactgttgacatatgtttgagatccctgacttcctaacactgagatctgctcgttaaagacagtttttagaaataggtcttctgtgtctttcaacttgagagtggcaagaaacactggcaaagataaaaagtagtataaatgattctgtgatgagtttgcccttgtccctaaagatctagcaaggcccccatcatgagagaggaagaacaggggccaggcacacagagtcattgtttcgacccacattggattagctgattactaagagatttctgttctcttggagacatcatagacatagtcaaaatgtagaacaatgatatcagactgctactcagcaaatgtgaaaggcgtcactggctggtcactaacgtgctccaaagcttaccagaccatatgaaaagatagcacagacccacccaccgtctcagatgcttttcacgttatcagtggagacaaaaaatgatgcttaagttacaacaggcagggttcagaatgctgaaccaattgccatactatttatcagctgtgcatttgaggtctgtgaaagggctaagaattcattaattagccattacctgagaccagttatagctctgagcagacaagatcagcatgacatcaataattacaacaagaagtaatgttgctgtcacccttacagtatagtcttttcttgacaagcttgtttgagcattattggaatattgtgaggtagaaataatgagtattcccattttctggaagcccagagaggtccatcataacatgttagcctaatgagagagcagggactcaagctatgttttctgactccaaatccatgaccttggcattgcttcccagtagacccccattgtgagaagacgggatgagaaaaataagatgatgaatgtcaaagtacttgatcattgtggcttagattcaaaggccagtccactcctggctccatccccattgctgagacccggttactgttattctgaactctgtggcccagcctttgatcccgggttcttctgatgagcctctgactggaattttgaccttgctgtttgacttctgggagagctggatccgttttctgcttctacttgtttattcctgcatcctcccacctagggacattactcaacccaagagccccgacccactctcccatcctgtcaggcttgacctctgcccattcttgtgcatatccatccaaattctaggccccaggccctaaccattggcacttaagtccccactgagtaactgagctgctcagcctccatcagtctgccaggcaccaggataccatcatcagcacgtgtgcattctgctatgcactcttccttttctttctctgattcttgtcagtctctaattaagccggttgtcaatttgccaagcagtcagcaaaagtgcccataacttcacgcagtagaagatgagctcctgagttgaatgactgcctagctgttttcaggattattttaataacactttttatttggcattatcaaggagtagggtagttccagaagtgaattctccagataattaaaatttgggatgtcagatcttacattaagaattttataggaggccttctataatgctttgtgtacttttctgccttcttaagccaaagatacagatcatatgatcgtattttaacccatctcaggaaaccgagtgtcatcactgagtaatctcattttggacggcttggatttggggatttgcactccattttcaccattattaagtgtgtcaggtttacaacagccttaagatctggctttctgcttcagtggagctcatttattgtgtacctcctatgtgcaaaagactgccacagtggtgtggaccctaaggaacttactgtctaatcttctaaaagttagaaaattagagatacagcctcttaggacaattttgaatgggagctcttctttgccaaataatgttacactgactttaggtgcttagatgtctttcgttttcccgttttgttcattcattgagcagttctatagaccaaactgtactaggtgctgaggttataaacaatgaatgaagtattgtccctaacgtgcagacccttcctccctggttttcaaccatttgccagaagggtaggttccagttaccaaaagtctgaccgcctgacccatcctccaagttgaaaatagctacttaaatgtgcacacacacaaacccgcatacctattaatgtaacacattatttccacttaaagctaccaaaatataattcctatgaacattagcaataaaaagaataagaataaaatgtaagacaagtataaaagatctagattcttagaatccagtgataattttgtgagctatctcaagctggattcctggaaaaagataccccactctataaaaaaaaattttaatttgtcttttgtggtcttttaataatctcaataatgctaccatcttttcctattaaatatgtttttggtaaaggaagaaaaatattaatatgtctaacagccaagattccttgggaaaagcatacaaaattaataaataggtctaaaataaatacatggttgcttccttttccgatcacaaaataatacaaatcttgggaacatttgcacactgttcattttaaaataaaataaactgctctgagataagtacttcagaaattacaaggatgtaggtgttgttatgagaagtgtacaaagggaaaaaggataaccaaatgtgtagcggctaaaagaatttgaaaactccaaccgtaagagacttttttccatgacttaatgaggctttcttaagtacacattcagatgctacggaaattcccacttccagattagggaatttttgtatcttcaattttttttttttcttattgtctgctttagtagagttttccctctagcttcatttgggaaaatttcatcttcactgtactttccttcccaagatgcaattatgttgacaatttagtgatttatttgaaaggtgttctgaccagttatgggtcacatttgaaaagcatctgtgttaacagcttgacatatgaaaatacagcaaaacagacaagctttgatgctgtcttggccagccttatctccagttttatggccctggctaggagacctgatatatttagccattgaactgccatgtcaagtcagtgacattttggaattttcctactcattggatattcacgttgcttaaccacgttcagccccctccaaccccccaatccctgcccaaaatagaaaaataaaactaccttaagaaagagcgcctgaagtaagtctgaaaagaaagcataattgttcatttttgtctctcataatcaaataagcaggcttgggaatttacaaagaagagctatgtaaaatctgcaaagcattattaaagcctgaaagaagaggtaataagagaaacgagagacagataggaattgttagcttcatttatcttaatacttccacctgagtattttttcacttgtaaagaaatgtaagaatgtgtgtgtctttaaaaagtactttttctgtaatcccagcactttaggaggctgaggtaggaagattgcttgaggtcaggagttcgagaccagactgggcaacatagtgagacctcatctcgaaagaagaagaagaaaaataaataaaaaggaaaaaaaaaagcattgttttctagaaatttgcctacaaagaggtttttccagaaagacttttcttagtgtgtgggtgtgtgtgcatatgaatgcataggaacagagggtgctggcagacagctgctttccacagccaggcctgcagtttaccagaggacagcacagtgaggcagcagtcctagagaccatcagaatctggaaacagagttaattaccctcctgacaagacagtactacacacagacccaacagggatggctgtgtatgccttccacagttgtacatttacagagtcacccacccatactcaagaactgctaattggttgtcagctttcccagggctgctaatcagaaagagtgcttcacacatttctcaaaggagataattaatattatttctgagggagaaccagctcatagtaatctatctaagaaccttgtatggattaattcaaaaccagcagttcagtggagtaatacagattagacagcaatttaaaagcatccattaatattgtcatttaggctacatatacctgaaatataaaaattattgtattgtttcagaagtgaatatatataattatacatttaaaaataactgaggatcaaagtatgttcatttttattgttctcccagagttgttgaactatataatgtgagggtaagactgatctacaaactttataccagcatcttaaaaggctgaatttgagtttcagcttcttacttccttggccagtttttggagaatttctcataaaataaactaacaaaaggcaagaaagtagagagcttagatttgtttgcttaacacatttataacacctgaataaccgaatatgtatttatggtttagtaccgtggccttgaagttagattcatgattttcattttaactttatatttttaatcaaattaattgtgatcccagtagcttaatttcgccattgagtagataaaattgctcctaggaaatatgtgagcatcaatataaaataccgatggtgtttttcaataacagtgtctgaagctctctaatttgtaaaaaatactaacattttcctggaaataattttactgacaagacatctgttaggctttttaggttatgaaagaagtaaccctaaatttttaataaatgtaaaataaagaatcaacatgtgtttgtgtatgttattgcacgttagaagatattttctttcaaaattttgttttctaagacttgggataagaattggtggctgagcatctgcactcctgacacaatcccaagggttctttaaaaatagctcttctggacagtgtaaggtgcttggagggtctctaaaaatcgttcaagaaaacaacaggtagaggagaggaaagtttcccactcagatgctgagtagaaaacagcaaaccaatgtagacataacaaacacccatttttgttctttattgcttctaggctcagaatattttagaattcttctccacaaggaagctgttggagggttccctttagaaatagttgtagccaaagacagtgaattcctgctgtttcaagccttcttcctttggaggacattaaggtatttctttgattcctcagctgattcaatacaagatgagatagaatttcaaggtgatcctataaacagtacttcactgaatttcccctctccaaaaccccacctctgtgcaaggatttcctcattctcctaaatgaggaaatctaagaacatttctctcctttgaaagaggcctactaacacatttctaccaagctagtttaacgggggacagttttctttgctccaagcagcaagtatcatgtacagggatgcatcagtgccgccagaaatcttagcaggacctctgttcaattgaactttgtttacgaaacacgccaaagataggggatcaccatcatcctagaaaggcttgtattctccgacacacagtttcagtgccataccccctgtaacataggagcacagaaatgtaggatttaaatgagaatcttctggtcttcagaccaaaatccgacaggcaatttccgtattcaaagcactgttgcatagactagagaaaagtctgggataataacaattcctgatgagcaaatttcagaatgctctaacttttagacaagcagaatgtatatttcacatactttgctcacactccctcaagcctctttatttatttatttatttttttttttttttttttttggatgagaaacagtcatactgagatactatgattctagattctttaaaatcacctcttctcccaccaggaactctcttggataaaagctatgttaagaatctgaccagctcttggtcattattctttaagaactataaatataatattgatttgaaatgctttaattgagaacagtgtcaaatatgtctcactttggagctaattatttagcataagaaattgaatctcagttgtctcatggcacttgtttccaaaaaattgccctgacattagtaataggatcagttctgagattatataataatatgttggactggaagcaaactctcaaatatgtttaacaataatgtgagatttttttctagataattcataaataggtagatagtctatatacttaccggtcatatttttgtcgtaatgttgttctcatggaaatctaactactctggctatggtctcacatgataggaaaccaagcactaaaatgttgggagaatgacgaaaggcagtacactataatggatgacttcaattgaaatgccaggagacaactagttgcctgcaaatgtgtacaactcaacagttactccaattacatagggtattagcagtaaggacccaagacacatgtgttaccacccggaaaaaaactttaaaatttgttccagtttgtgtctgagtaggaaaatgagatgtatttgttaaatgtaataaaatgagatgccaagtgttaaaatgtcttcttaagaaaacctagatttctagctatatttaacatatcacaatgactattgttgtgttcaccctaatctattgaggggaaattgaatagttctaatgtaaattgaaagttttcatgaataaaagattttgttgtgtttttctgtgtttaatgttaaatattgagggtataaaaatttgaaatggctgtacttccctgtttctttcatctaatataggtcataagtgaatgggaaaatactaatttcagaggcacaaaatgattttttgcttcttgcatgctacagcgttgaggcttggaaggtatcccatgggacttgttttacctgaaatatttaacacacacattgtaagaccagcacatactgaacggccctttatgcaattttatttgttcaagtgaacaaaacctgttatgctgaatattttaggaaaacatattgaacatatttcctctcctggagaaatgtgtgttagatgtacatctgtgactgtgtacctaactgttgaagccatttggatagattggcttctgtgactgatacggggctgcccaagtcagcattgtggttctgatcctcaaataacccagtccatttgttcctttcactttctagccacagatcacaaccttaatcgcatgttaatctaatatattttttaaaaaacagcacatacatcactattgagagcaaatatcatttagaggtagaagatcatctttgtaaaagttggacgatggattctctaagcaaatccctaaaattcactgctgctaaaattccatactaccctatgatatgatgattatacatttttcaaggtcagtttaatcttcagtatatacttttccatagtatgttttctgaaggcaaaattttctcataaatttcacatattaaatgtcagtaaagaatagaagattttggtatgtgtagatatgcttaagacatttctttacccataagctgtccaggaaagcagtgtgcctagagttcagaattcttccctcaactttggcctaatcgctctagaacatagaagtcctttctcactgggataacctgtggtgggtttacttaaacctgatacttgccttctacgctctctcatatttgtggttagagtaattttgacaaaacattggcagtcttgggactgaagactggaatagaatcattatttggggtagctaagaaaagtagtgataaaagaaagactaataggtttgagaactaaagatgtttgcagtaaattttaagagaatggaggggagcagcaatacgctaactcaggatgtcttaaaacaactaaactttagttattttttgatgtttacagttatctgtcaggaagaataccttggttattgaaggaaattagaaactacatcagtgaaaagtttatataaaaattttagtaagatgctgttctttgtaataagaatagagaatgtgctatactcacttgatacatatttttactgggagaatgctgatctaagcatctctttagtaagtcatcgttgcctgctacctgtgaagagttgaatcactgtccccttttagtagatggataaaaatttagaactcttgatctcagcagtggcagctaaaacacttattttgatctctcttccctggtaacaggagatgaaacttgagaaaacagtgtggactaatggatagagtgttggacaggaggttgggaacctctgttctgttctattcccagcctgctactgggttactatgtaaacttagataagccagttaccctctctgcacctcatattttccatctgcgaaaatgtaggtagagacattctatactcagctctttcacaaagatattgtgagcaacagcttactttgaagacgttagtatatttttgttgaaaagatgttgcataagtatgtcagtgcctctgttccctttggcaacaccgttcccacaacctggcattatataaactgtcccttattgatgcattgagaaacaggtaccattacagcagattttgtgttgcacagaagctgagagtgtgactggagtcagactgcctgggtttgaatcttgggtccaccacaaactaatgctgtaaccatgggcaggtcagtgtgctagatttcctcatctgtaaattgaggataataatagcacccacctcattggaatatggtaaagctggaaggcatttatgaacagcactttaaaccttatataaatacataatatgctttatgtaagtatatgtagttaccgttattacccaaagcaattcttcattaatatggtaaagcgtcacctctggtcttgctttgtttagtgcccttattcccagcatggctgctcactctttgtcctgaatgtgtgtatctccctctgctcccacaagtctctgggccctgaggataagtgtgtagttatctgtcgtatcatctaccataagtgagggagtgccacataaaagataaaagaaatccaggtgttttaagctagtctcaaaaactgatttattaatttggaaaaaaaatagtaaccgatttcagaagtattacagaatgggactgagataattgatagttgacagctgtatcctgattattataaaaaataaaccggaaaacatcaggaagaaatagaagagcagttaacaatttaaacagagttcatgttaacctctctcgtctgcttccagtagtggtgaccatcagagaagaattgtgtgaaggtaccaaaaactgcatgaattgctcaaacgcaaagaagcagagtgttcacattttcaaaaatctgaacacaaatatatctgtttcaggggttaaagatattactggagcataaaagtttacgatttgatgacaattactgatttgatttttgtaacattatggtgtggaatttcaaaaattgaatagcatctaaaaggtaatcatatgtttcattggttatttttttctctatttattattagaaccaaccatcatcacagttgatccacctgcatgtggggagttatcaaactgcactctgacagggaaggactgcattaatggtttcaaacgcgatcacaatggttgtcggacctgtcagtgcataaacagtgagtagacagaagactgtatgttttttctgaggcctaatatgatattgtcaaagcatattatgtaattgaatatctttttcacttctgttcagacattcccttgacaacctgaaaaaaaggggaaaaaagttatttttaacacaaacaaatacatagtcatttgggagaaaggagctgctgctttattgtgtttttttctctgtttccttgatggccagttgcatcaggagtgcaacctggagtggctttggagtgcatccttccagccacccagaacgctctccctttagtacacttgccaggtcatccagtgacaaggaggtcacccacgcatacagtgtcacattccgcatatgacagccataatccttagactgtgcttcactttgttgaccccagctttgcctccatgcagattccaatcatgtatccaggccctgccctctagagtatagaagagtatattgtatttcttccagaaaaaaataaaataatacacacacaccccacccccatatatgttaaaaccatttcgaattcccctaattcttctgccggaataaacatcctcattctccttgaccattcttcaggtgacagttcctagaactttgttgatctttaccatcatttgacacacaatatgtcattgtctttacataaaatatggcccacaatcaagatactccagaatgggcaggcatggtggctcacgcttataatttcagcactctgggaggctaaggcaggcagatcactggagctcaggagttcaagaccagcctggacgacatggcgagatcccagcctctaccaaaaatacaaaaaaaaatagctaagcatggtggtatgcgccagtggtcccagctactcgggaggctgaggtgggaggatcgcttgagcctggtggggcaaaggtcacagtgagctgagagcgcattactgcactccagcctggatgacagcaaaagacactgtctcagaaaaaggtactccacaatttggtctgctgaatacagtgctactgtgtggcctaagtttgcattagctccttgaattgcacttcgttggctgatattgaggttataagtagctaaaaacactaggaacttttttacatcacctgctgttaggtggttctcatctgttctgtaatagaacgtttgattttttttttaaacgatataaatacccggccaaatataacaagaaattatccgaatcattttaaaccattttctaactagttgggatatttttgaatctcaataatgtgatcttatactttatgcctcccagcttatattaaataagcacaacttatatatccttatctaaatcactgaattaaaataaactgcacagaaccaaggacaaagacctgcagaatgatgctgaacatagctgcttacagtagtcctaagcatttataccttgagtagagttatttagctgacaacatatatctccacaagaaatcttgaaagacacttcgcccgtatcttgtctgaagtcaaaatatattgcaaaatacatcagagcattcctttgctggaaaccctatttttaaaatgagctgaagttataggggcctggcttatttttccagaatctgtactggctcctggtagtcagtgctttattcccctaaatgtcacaaatcatatatttaacaattatttctgaagaattttcagttgtgaatgtcaatttatgtagtttttagaacactttgtcccttttgaattgacatttggcctattttcttaaatctctttccattctcttcgatctctctcacacacagtactggttctgtaataagcgggctttctgtgggcctgtggttgtaattaccatggctcagagacttggacttaacgtagacagatgctcactttctgatttatcagttatctcaggctttaacataccacttaaccttaattatttgtcactattttaaaaatatttttgaagaagatacaaaataggagttaagccattctgcttcctcagttacatgtgaccatcacactatctacagtggcgttagtgctaaaactgagtttataaaacgcctgttggccaggcgtggtggctcacacctataatcccagcactttgggaggctaaggtggatggattgcttgacctcaggagttcgaaaccagcctgggcaacatggcaaaaccccatctctacaaaaaaaaatacaaaagctagccaggcatgatagcaagggcctgtagttccagctactcaagagtctaaggtggggcgatctcttgagctcgggaagtcgaggccgcagctagctgtgattgtgccactgcactccagcctgggtgacagagcaagaccctttttcaaaaaactaacaaacaaaaaaaaaactcttgttgagcttgtagttactaccatctttaaatttcctataagcctctacccatttgggggtattctgacttcctacacttgattctgttcggggaagggtcagtccctgttgccttttatacctccctttagaaagccaagttcactagagttgccttggcagcccaagttttctcaaatggccaaccttgttttttgaacaagataattagtaattgtcatgaagtgtttatattttaatccttctaatgcctcttacattaagaaagctacatctttcttaacatgggatcatatatattaatacattttttctgagtttctaaaaagtctaaaaatacagatttcattgtgtccagtcacctcacctacacttgcgcctgggttttgctattgtgaaatcttggccacccggtcatgcttatctaagacttcggtcacttcacacggaatctttcatcattaagttcaaagaagcagtttccctcaactgcctttgaaggaagagtcaaaatatgtcccttggcaaatagtcactggcaaatagtcatttggcaagttgagactataaggaggatgcatatttaccctatctttaggttagtgttttgtttggcattttaaaaggatcatcagcatgtgctacctgagtgcacctctgtttcattcacacttaggaagaaataggtacaggaggagaccattgcattttgtatcaataaatttattatttgcaaattgtacccatttatacagatagctggccttccttccttccttttttcacctaatataaacagagcacctgccacattctaggtagtattctaggtattaaggccacaaagatgactaaaatgcaagaccttccctaaggaaaaaagtcttctgtagcataaacactcatgaaatatcactactttaagccaaaagctataacgtacaaatgtaaattattttaagaggaagctttggtccttcagcagaacccaggcttatgttcacaccagagtagagaaagaactgcccgtctcccagggctctacttggtgctcagcagatactggctgagtgaataaaaaggaaaccttcaggatcagagatgcctttggatatcattcttgcctgagatctccctttgaggtcttgctattctaataatatatccatcttattttggattcttcttaaagttagtcttgtcatctcgctcactgccccactgtatacctaaagacttacttcaacactgattggaatttttcaggttgtcctatacagaagtctagacatacagcattctgctgtcacctcccccattcgtattagcaatccaggttatttggcaactttggaagccacacctttgggatgtcagtcaaatcaaaccaaatttccaggtcctgggttttgtcagattcttgcgatcgtatgcaactctcctctcaggctctgcacagcagcttttgtgttgcacagaagctgagagtgtgactggagtcagactgcctgggtttgaatcttgggtccaccacaaattatgctgacttccacatgggtaataaaggcaagttgctttacttctttgggactctgtgtccacatatgtaaagggcaaataatagtacctaactaatgggttattttgaggactaaataaatgaatgaatgcaaagctaactctggctggcatatggccgttgcttactctgcactgtaactattttatagcatcttcttctgagtcggcccaagctagccaacatggaaagcccccagcgtccccgcccccctttgttcccaccccatcagcctgcctccagttcttcctgccctctaaggacactcacaccaacagctttccttttactttttcatttcattgtaaccctcaaatattatctttctcctccttggttttagagaagggagtttcttatagccaactatggaataataggttgttttttttctattgcagctgctttatataggtatttattttgtcccaaattcacccttacattatttttctgcatcccatcaaaacctttttcacatacagtagtcctaagcatttatacctgttttctcttaaggattaggcctagtttccattagcttctagattttttttttatacagggtcttgctttgttacccagattggaattcagtggtgcaaacacagctcactgcagcctcaacctcccaggctcaagcgatcctcccacctgagcctcccaagtaactgggactacaggcactgccaccacacccagctcattttttttgtatttttttgtagagacaaggttttgccacattgtccaggcaggtcacgaactcctggcgtcaagcaatctgcccgactcgatctcctaaagtgctgagattacaggtgtgaggcaccgtacctggcccttcaagatttcttaaaacaaggccagcaccccacaagtatttcttctgtcttcgttgatttgttgttcttcattcactcattattttattcatctagcatttgttgagtgcctactatatcctaaagaagattaaaaatagtccttacccttgactcacattggtaatttgttggtaatattagtcatatgttaatatgttgtaaagagacaacataatctagcatattttaggcagtcacattaggattgaaatcaaaaccctatagaaacatgtgcttcaaaagtatgtggcaagtatatgatacagaattctttcatttgaacataacgtaatttgccgttttaatcggtaacaaataacattcttgtaacactaagaaaagtagtactccaggacatacagaaagtttacattatccctgactcagttcctcaaaaacttgtttagaataaaaaatgtttttaaaagtatgatagcaacaaagtttaaaactaaaatgattttaaaatgatataaagcaactagtaacctaacactttgtagatacaagcttttaacaacattgctctttaaccagtgtcttttgctaatagacgtaatatacctgcttactacataaactctgtatgaaaggcatctgcatgaattaagtagcatcagtcttttggcatcttagtgtacacatctttaaatcaagtggatttgatcagattatttgtaagttctcttccaattttaacattctttaattgtagacattaatagtgctttttctagaaactgatgcagacttgtagaattctgcatcagtcagcagagaggaaagagccttcatatttttcatgaaatatgtatctgaatcaaacatcacaagaaggttgcagcatcaaatatagggaggcaggctccttgttttatttcactgtaattctaaactaacacctatcagggtcaaaattgggaccaccatttgtaatgttgcatttctttacttagtagtcttgggaacttcatggccatcctgaactgataataggtttgtaggaattgccagcatctaagagtttaaaatacactaagtcctcacttaacatcctctgtagattcttggaaactatgactttacgtgaaatgacatgtaacaaaaccagttttaccagaggctaattgatataaacaagagttaagttcctaccatatatttctggtcacgaaaacatcaccaaacttctaaataaagacctaaaacatttctaatagtaacaattaaagtaagcatgagttatatatacatttaagaaaggttaacaaaaaacaagtacaatgatgattccccatttttaatgaatcagtgagtgatagtggttgtagtggtgctgtgttgaatgacatgtaacaaaaccagttttaccagaggctaattgatataaacaagagttaagttcctaccatatatttctggtcacgaaaacatcaccaaacttctaaataaagacctaaaacatttctaatagtaacaattaaagtaagcatgagttatatatacatttaagaaaggttaacaaaaaacaagtacaatgatgattccccatttttaatgaatcagtgagtgatagtggttgtagtggtgctgtgttgaatcaacgaataaatgtttgcaaagcaaacattgtaaggagcacctccttctgccatgcagttcaaacacataccatacccagtgtggcaggctcactgaacgctgttgtcctgcagtgtttattgttgtacgtttgtgtgattatcacctgctttaacagatttgtattttataagaatttgtatttattcactcatttattttccaacttgcttattccagtccagggttgagggtggccacagcccatcccagcagctctgcaagctgcaaggtaggaatcgaccttgaacaggacaccatcccattgcagggtgcacacacacacacacacacacacactctctctctctctctctctctctcacacacacacacacacacacacacacacacacactcagattaggaccatgtagataccccagttcacctaacgtgcacatctttgggatgtgagagtacccagagaaaacccacacagcatgagagaacatgcagtctccacacagagagtggccccaaccgagaactggtttttattcccctcctaaaagttatgacacaatgatgttgaacaaaaccattgctgtttgaggacctgctgtactacagcaaagaagtatgcagtactcctctaggggacaccatccacagtgtgttctccatggaggttacgttgtatttatatataatgaatggcgcccccgacagctgtgtcacgttttgagcataggagcaaacattcctatttttccagaatgaacagagttttactacatagttgctatcgaccggccatgctgtacaggccctgtactttgccattgttcatcactttctcttcaatcgtaacttctccccatgtattagaaagcaattttagatcacggtaaagtcatgacgctgaaaaattaggaacataaagccacctttttctgcgtggttgcctcaagtgatgtactaaagctttacaaacaaacatataaacccctacccttctatgaatactgtttttctactctttatttatgtttttatttatttattttttttttagacacaggttctcaccctgtcacccaggctggtgtgcagtggtgcagtcacagctctcactgcagcctcgacctcctgggttcaagtaatcctcttgcctcagcctcccaagtagctgggattacattcactcaccaccacacccagctaatgttttattttctggcagaggcaaggtctcactatgttgcacaggctggtctcaaactcttggcctcaagcaattctcccacctcagcttcccaaagtgctgggattacaggttccagccagccgcagtcagtctcctctttatttaaatgtttgctatgatgatgcatttctcttaagaaaacttcataattgacatatgtggatttaggactaccttccagtttttaatgttgtctatttctgaactagaagatggtattttgagatgttttggcaagtgctagtatttcaggaaaatttcaatgttatcagagtaagttattttcatgctgtcatttctctgataatatagcagtcttgatgacagtggaggagtaggtaacaagaacattgattgatgcatttagttagcaaatgttgttgaatacccactctgtaccaaataccatggttgtccctatacagtgtgtgcctaaagatactagggcctgggcctagagctccctctattgggaagctgaatgtagatgggccagagcataacatgcaggagaatggtcccaattcagggcagcactagagcatcccactcctaggaggcatagccagtctccttctaatgaatgtcctgtatttttatttaaatgtaccagcctttgtttgaaattccattccattgcaaagcagaggtagattacagactagtttttatcacattgtttaaacttgaatggagacacaacagttttctttaaaggtgaagcacattcaagatcaaggtctgtggaaaaactccttctttattgcaggaccagaaaaataaaggcaccaaggtgctcaggctccaaaattgcagggaatttaatcccaagggatgttgactgactctgggaggttttcagggtgtttttaattcatctgtttcccaacataattcatttgtagccatgacctaagcattcttgtgacagaaaaatgggttcacaattgacatctgtgttagagaaacatgactaataaagtcttcttttttcaccattcccttaataatggggaatgaaaaattatgacctttcaagtcatataattacaaatttctaaatgaaccatttagaatttctcttttaaggatgcaaattagtaatcagttgctttacagtgtaatacaggcacatgaggttatatttatatgtgtaataaaaggtttatgataatgacagagtagaaaattacaccataaatcacattttccttcttaaaagatagcaaattaaatatgcaaatattgccaactgagattcacaaaagatgctatcttgaaatatttatgtaaaacacggacccatttcacctattcattgtagttaagttttaattttagattataatataagtgtgttttgaggggacagatgcgttctgtttttcccagtgacttctgtgttattacaatattcatgcttaaaggacggccagataaaataaaaatttgttcatcattccaataattagactctgcttcccatttcaccagtccatgtagaacaatattcaaacattgctgtactttggccatcaccatattaacttcccttctttaaatttgattgtctttcaaattctatatttaattcacacaggtctcaacttgcttgctttatataattaagcatgctttttttctttttaaccttaaacatttctatttcccttttctctcgtaagccctatataaccttttaaaatcattttagaagacttctattaggctaataacttcagtaatgttgctagtcaccattgctactttcctttttgctcttctaaaatagtattgtaactgactagcagaggacagttaaacatcactccagattctgctaacgggatactttgaacacttcaaatagtggttacttaaaccttaatatgagtattttttcaacacacacattttttagcctgtgaactaaaaagcctattatgtaagtatatccaaatacaggggtatttgtcagcacatagagaaaaaacatatctaaatgtaacactgaatattttaattactatcattttgtaaaagatgtacttttttgaaagtaatagtaagaaatcccttatgctgcatacaatatgcagtattatatgatagcatctaacacccataaaagtagttgtcatgtggatatttaaaaacatgcgttatgggatgggcgcagtgactcacacctgtaaccccagcacttgggaagctgacactggcggatcacttgaggtcaggagttcaagaagaacctggcaacatttagtagagaaggcgaaaccgcatctctactaaaaatacaaaaattagctgggcgtggtggtgcatgcctgtaatcacagatgcttgggaggctgaggtgggagaatcacttgaacccaggagacagaagttgcagtgagccgaaatcacgccactgtactccagtctaaatgacagagcgagagattctgtctccaacaaaagaaaaaaggacatgcattattatggacatgcaatagaaataaaacatataatgaaaattggactgtaaatttctaattcccacataatgtaaatggtcatcagtggtttgaagtgatttataaagttcatggaaaaatgatctcagtcattaattgtaatagttatcagcctgctgttcactcagagcctaccagatttgagtacactttagaaatctatgcatttcaaagttggactttattttgtttattattaccaaaacatggagtcatgtgataaccatgaaatagcacagtctatgctttttaatgataaccagtgccatggtagagctgagaaattgagatctgtggaagagtggaggattcagggaccgtatttcagcatcgaagtgttctgctctgttcatctttggaagaaacatgccgtctctgtgtcttcacagccgaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgtacgtatttgttaattcagaaaagctattatgaagtgcaacttggtgaaatgtttctacacattttgttttatatgttgtttgtgaattaatctatggtatattgagcccagaatgtctatacttgttttgttaggtgattcagaaagcagggagaaggttgtatctccaaaaaaaaagaatcaagttttacttttactttttatggacatgggccagacacaccctttgtgcctgttatatgagactttcccatagcagagtcctttagacactgctggcaagattaggagatggaattgtgaggtcccagtgttggaagtgctacaactcttcacacctgtcccaaggtctatttgtgatcatttatttaacacatactttaagatcaattgaagaccgaccccccactcacttgaaatacagtgcatccttagctgccatgcctaggtgtctcaccttggaactagtcagcgtttcgtatttttttctttatctagcgttagatttttatgaattttcttggagctaagggccattacttttattttttaatatactttattgagtcaaaccccatgagaatctagattattccagggggggaaatagacacttcatatttgtttctttcgtgtctgtataagcacctatatttgcctttatatcaggttgatgcctttagaaaaatcaccttaatttatatttttccctttgctgtctttctttcatttcaatctgtctttccaatttgatatgtttatgtagtgctagattccttttcttttttttttttttttttttcaggcagggtagtgctctgccacccaggccgtagtgcagtggcacaagcatggctcactgcagcctcaacctcccaggctgtcagtcctcccacctcagcctcagcctcagcctcagcctcccgagtagctgggactagaggcatgcgccaccatgcccaactaatttttgtattttttggagagatggggtttcaccatgtttcccaggctgatctggaactcctggctcaagcgatcagcctaccttggccttccaaagtgctgggattacaggcatgagccactgtgcctggcctagtgctagtaattataagggacttagacctatcatctctttcccatcttctgtcagtcatctttttcattcttcatttaaaaattctaggtttgggaggggtcgtctttataattttcattggataattaaattggaaacattgttccgtaaaattccccaaataaaataatgaatcatcatcattcttaatcctaaaataagaatatgatgtcaataagagtattatgtcttagcctaactgctcatataaaatgaagtagttgtatcacaatagtaggtagcttcaaccacttgctacagtatcatgacagttcagaatcaaggctgtgttcaaatccaaccgtcatgttggagaaaaatctcttaggagattgtagatttcactttgtattcctgtcagcacattcagggaggacccagttggacaccttggacaactgaatattatactggctagaacacatgtgttaaggacacacaccaaaagagaattcttggccagacaccacgtgggagtgtcaggatgaaggcagcagttggtgccccttttatgtacctgctgactaaagaattactttctgcatgcagcagatacttccagaagggagggagggagggagggacggatggatcgtaggtgggtgggtgggtggatggatggagaaacaaatgagtacacagataaaccaacaaaatctccttcactttcctattcatggagttgcacattcatctttttattgtggtgtaccactctccctaattaaatcaacagtcatgtcattgagattctgagagccacatcctagaagcacgtaagtgcttgggcaactctctgccttggtcatgagtaagtggtggtaatggccacagggagctaattacccatcactccaggaatctttgagagcaaaagtcaagtcattctgtttaatagcaatatcactttattgacccttggtctgagtgcttgggcatcatttgagactttgtgattttctgacctctgacaaaattttaattacccaggaagaataagcacggctgtgacatctgtcgctgtaagaaatgtccagagctctcatgcagtaagatctgccccttgggtttccagcaggacagtcacggctgtcttatctgcaagtgcagaggtaagtgtgtacacatggcccttccccctcaaagcagccaggggcaccgaaacaaggaacataaggacaccctgtaatgaaatggtttcggtggttgctgcctattctaacacaaatgtcagtctctgtgggaccgtcccacaggactcccttttcctttcatttttatgaagtaaaagctcagaatagaatgtaaagtaagtatatgatggacaagcacattgactaaattaatcacttcctagctctttttacttctatcaggaactttagcctaaagcacaattatctgaacctctatagagcttctctctgttcaggagtaagattaataaaactgcaacagtaaatagccccatttcaaagttctagagggtgggtatagctcagaagttagctgctgtatttccagataacagttttccatgagaatttagcctttggctgaataagcacatgccagcccacagctgtgaataagcacagagcctggatatccctgctgcctgaggcttctccagggagcttcctgaagccactgagcatgcaccctgaccgccgtttctgcctttaccccagctccagccctgctgcctatccctttttatgttgtacttgtcagcatgatgcacagattaaactataaaagattcctaaactttgttttacgatttttcaaaactggacagttaccaccttaatggtgtttctaaggggttggagtaaacaaaaatctagtcactgctttaattgttctgctgaacaaaactctgttatttgcaaggggcccaggatgtccccattggatgaagcttctagagcgatttttctgaatacattcaacatatttgtaaaatttgttataaaatcagtgcttgtttattgtataaaatatgagaaagtacagtggaaggaataaaaaatcagcgatcatcccacgaattggagagaaactatttgccggtttttttctctcttttcttttttcaagattgagaacatactgacttaaattcttttcatgaacatgtcccatgtcacaaactgtcctgtgttagacgtaattgttggtggctggggtccagtccatgtacagactctgtagcctgtttctcct

SEQ ID NO: 23 - DNA, 50000 bp, Homo sapiens
gtgcagtagccacttctttaccaggctgccttttgtctgtccagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcaggtaaaagctggctgccatctgtgtgctccataagcaaatgacttctgccttgcagggccctagacacatttgtaactctggggaggttggaggggacaacccctggaacacttttcagcactgtgtggtttttgtcaaccaccaccacagaatcatccaggcactatcagtgaagaaccaaagaccagagaaaattctatcattgtagaaatggctctcctcctttttgaatgattgactcagttatgtaaggtctttggactacatttttgtaaaggtttgtgggctgatgttcttacattccagctctccattttattgatagcatattaagtctgaatgtcctcattaatatgaggggattggtactagttttatcccataaaaccttatcacatttaaagtaatgtttgagagggagaaactttttgaagtctgtggctattgctcttttttaaattaatagacatgtggcactcttgccaaatggagagatttagagtgaccacacgtccaaaacatttaaaatcataatcatggtaatcaggataagttactagaaaaccagaacagatttgtaattcaagggagtctgaattatgattgtcaataacagtaattataaccataaacttcatctagtacgttttatccaaagagctcaacacagtcttatatctattatttccctctctgtgcaggccactttgagttagggaaaaacacattgaggcagtttctcatttggaaaacagacacagaaagattgactgtcttaccaagagtaaaatagtatgtccaccaaaaggcctgggagaaatgaagtctctcaaatcttgtatcagatttctttcaaagttaagtctactgtcagattttggaggatgattgtttttcctctggtcagtccaggtcctgtatctccctgtgcgtgagtccacctgtgcaaaaggtgtagccgggagcctgaggcctggtccttgtgcaacaagactagcctgggagggaaactgaccttgttcgccatggttggggagcaggggagagggacacttgaagtccctttcagctctgagattctgcacagctcaatagctacagtatcagagtgacaggcagctcccataccttgtagaaaacgagattacaatgcagaagagatgactctttttattttgttttgtttccctgtagttcagttagaggtgctccatgtcagaactctttccttttttccgatattttgctagttaagtgtgaaaaactgctagagtcccctgcataagaaggtaaatgaggactttaatatgatagaactctgtgttgactctttggggagggaaaaatcatgtgtttgcatgaagacccctgctttaattataggtgtgaatgcatagaggagggatttgtacctgggtctttgctaaacatgtcctcaatgaagaattctaacagctagattggtagaaatgcaaagacattaagaatggataattgacctgagaactgaaaatcagtagaaccagaaagatctttctctattacagagtcatggaaagttcttacgtaagcacacgtgaagttgttgttgataccttactgtgggtgtgtcaacatctggatatacgtgtatgaaaacacatttctgtagatggatgtgtagaggtaaatgttttctgcaaatctttagcatgttgccacataagcctccttctgattagagactccagagctgttgcattcacggactcctactttaaagaatatttttctcccctcttcattccaagatcagctgtttttcttcttgggatacatgagtatctcccgaactcttctaatgaatatttactaaatgatagtgtctctcagattttctgtcttccatacactctacatttgagtttctcttttgtctccatgtctctggcccctcttgtgttttgccttgaggcatatcgtacggcaaaaaggtgagccagaaagcatacctgcaaacacgcacgtcagatttcttctgaccacttccgtacatgtcagcatttaacaaggcagtaattgtcagtcctctctaagccctttttgaaagcaataagggaggcttttgctatattcatttatcgatactctttgggactttttgtttctctgtaaaccacagatgcacatttaccatcttgttcagcacagttttcattgtaattctcttcctctctcttgttcttatactggaaagttggttacagttgcttgtgaatgtaagcattgattgatgagtatcagttttctgtgatactgtccctgggtctaagttccagccttttgtaattcccttgagataaagctgtattagaagttgttaccctcttaactggaaatcagcccacatcacttaatttcattagtattgttgactctcatgctaaccaccactttgaaagtcatagccccaattcactcctaagaagatttcagctctgagcatctctgcatatatgggcccattctggggcgtgacttccactgagctccaagcatgagctagccgtgatgtctccagactctttctcggcgggattcaacactgtcaacacattcctgaagtgcttgttgcatttggattacgtgactatggttaatgtcttcatacttctcatttccccagcaaaacttcagggcaactcccagcccctcttcagacattttcaacattaaattccctctctatcagtgtcatctctccaagagaccaaaactacctctcctctggggaaagaaatgatagttttcaccagcctgccagcatttacctctcttcatgatgatattgccaagcacatgtttagtctcaggtgagggttttcagactgttgattctacaggagcattcctgctcagtttgctggcagctggcttagattggggtgtggcctgctcgacagcaggacatatttccaagcataaaagtactcaggatcccagaaataataaagcagatagggcatcgatcatttggaagttttatttaatctggagacatatccatgatccaaacaaccatagacttgggttgcagtttccctcgtctctccctgagctctcagtttcctcatgcataaaataaggtgactgtaaggaataaatgaaataacacatgtgaagtgcttagcatggaaataactgaaccgtaccaagtattctgtaaatgagagatctgaatccgagacagttcctacacattcacagaaatgccagaagaggcccctgttctcatctctagagtgggagtcccctccctgtaatttaatgatcttattgaattctcttattactgttgacagacttaaaccacttgaacctcacgtcctaatattagctcttttgccattgtcttctgtttccatgcatggaatattttcttcactattctcttaataagaatagagcgagatgctcttcacagttcatctcttctatcccttaaagcaaacagcaccaggctttcaagagttggaaagattgcagatgaataatgtgttctgattttgtgtcatttcccagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacaggtaagcgacaccatgccttgtgggtgcttggtggggaaggatgcagctctgggtgttgtcattctgtgggacccacagggcaggatcagccccatatgggagtgtgccaaacccagtatcccatcaggaacagctggcagcctgcttcctgcatgtattctctttatcaaccacctatttatgcagtcagcatgcctcacacctgagatgactgtgaaacaaaaaaggagactgttgctcttgccaatttttttccttaaccctgcatctcaaactcgaggcgtatacctaagtagagaaagtcccaccgcaccaaattaaccaaacgattctgttactgtgaaaagtcatctttatgtaacaaatgagaagaatagttatttataaataatgtaaacaaaattctttgtaaataaaattctttgaaatataagggtggcgaagagactttgaaatatgcataatgccaccccctccaagttgggatgattagtaagttaatgccaaaaagcattaagtgactagcactgcacatgtataaggagtgctattattttctactttttatttctcagataatctgaccttagcaggtaaatgtatggccatgaaacttgttagaaggaaatttgtcaagatgttaatcataagttactggttttatataactttagtagtacttttaattaatactagatgtgactttttcaaaagatatttaaatataaaattatcaagttgacaggactagttttgttttgttgttgttgcttcttattcccatatttcaacttaaatcaataaccatttactttctaaacatattcttagagcatttctgctcagcaaaacataaacccttgtattttattacttcttttttttaacattggattatctagtcagctggccagaatctgttgttccgtcattatgtgcagcatcatcagtgtctaaattttttataagtttatgttgatggcatttttggaattgatcagatgaacttatagggatagccacattactggctgttgtcatcatgcacggcagcccaagtattaccattttagtacagaactgttaatctgagaagcttaattagcactacagcttttctagtataagaaatagttcatatgtgttacatttttaggatatgcatgacctatgatttcctaatttgttgtcatcacaattcactcagattcaaaccctactgatgttttttgttaatatatagcagtgactttacttacatcaccttggtagaaactacttagagtgatcttttccttggtgataatgaatttatgtacttttagctatttagtttacaaatatttgtagaatgcctgaattgaactatggggattccaaggttaatgaaaacagacatgattcctgtcctggagcaatagccgaaaagccaaacaatccaatattccacgttaagtcaataatgataaagtaagaggtgtgccttgaaagggaggattcaagaacataggcaatccgggaaactaaagcagcaaatacccctggtgctatttgtgttaggatctgagtgatgaacttagggattaactaagggagtggcactgggggaggaaatgcctccaggaccactagctgaaattcattgatgggacaccttactgaactcagaggttccacccataaagagaaggagagctatgtggaggtgggaacctctgagtatgctaacatgggtggcatgtgcagaggcccatgggttctagccaagcaaggcagtggtagtcctgcattcctttccatctttccaaagaacaggcaacccctcatgtgcctggctttgacttctcattcattaacttaggcctggatttttttctttacctctaactaaaggatgtttctggactttttcattcatcgttgtattgagatacaaaaaaataaaaagccttaaaactcaaattatcagtttcttcccatcacagtaattctcaacagtggcattgataagtggcatattacacatgtagaaaaatatcatctctaatcttaggacacttgcattctaaaatggcccaaaagtcatagctccacagtgggataacagcttccaaagggtcgaatccctcatgatgcttccatctgtagggtgccttgtcatttcctctcttcagctttccctcttctctggcctggtttaaagcaaggtctggtgaggaaatgaggaaagattgcacatcactgcaagtgaagctacttcaccagcatacaaaagggagagggtaaaatagcacttgtttctttccataagtttaaaattttctttcctttatttttagagttggaaaaaagcatggcagagaagagcgagggaggtagaaaatagaaaaaaggaaacactgaaaacagtgaaaacactggtaaagatcaaataaaggagtaaacgtttggaaacagaagtgtaaagaaaagggtctagagcaggaaaaatgagaaaagaggcccaaagaagggagatgctgtgggtaagattccaaatgacattgtcggtcagggttgggccaggagcctcctgcctggtggaaggaaacctgcagactctacctcggctccagccctcacaccgtcactgccagcgtatcctgatgtcctctgggttatttccaggcctcagtgagagccctgcagattccaggctgttcgtttcactgtcattccatagcttcagcacggctgtcccttcttcttcttttcagtttttagacagaggttgggcaatgcttgcatgagctaagttaacgtaagtcacaaaaacctcatggagatttgcagggcctagtgtgaacaagagggtgttgtccttatttctagccaaagcaaaagttagcactggtcagtctcttagagaagaaattatccctcgacgtagataaccacgaaaaggtcattgagaaagtcatcccatcttactatctctaacctatgtgggtgggatctgtggaatcatatctgtaataggacctagagtaccatggagccgtggaaccaatggctgttcttcaacttatgcctgctgcctttgggactagtcataggagatacagttactcagtttaaaggagagggtatgggccaatcccattatgctggctttcctgtccctcagagaaggtccacaatgaagtaactgccttggtcctcaaaaaggcccttcttgtcacatcctgcttactggctgtggggtatattcatgaggtggatagaaggaaaaagtggcatgtagagcttctggatcatatggcaactacatgtctcagttgtttctggttcttgactaaaaataacagagctcatgaagttggttctgggaagaacatacgacaactcttttgattgtgtatgcattgagaaaatgtggccctcttagagctttgctaggtagggctgtcgtggctgtgtctagactcttgagtggtgaagttgattagaagtgctctcttatcagttggactatgacatttctaaactggaaacgcacccacttacattttcctttctgatatctcaatcatattgctgcttgaagttacaattttcattgattttagtggaaaccgatgcccaaagtgctatctggattttctggtgacattttctgttgcaagttgtaactagatctggggacttccaggtctcagtctttagatagcaaagacacagatgatttgcacatgatttctctgtggttgcatccttatgagtgatttagatttcttatctttcagtcattaatgtgaattcctgaacattggaatgttgcatttaggtggtttttggtagtctggtacaatgagaatttgtctgaatattgattggtcaaaattgcatgtggccacaagtaagagaaaatccagctacaatttgtaagtaaatagaggtctattttacatatatgacaagacatttggaggcagatgtttatcagcattgattcagtggctcagtggggtcaaggcctttctcttctgataggggtgtcacagccgtgatcaacgcactcctatcaaggcaggaataatgtggaaggatgatggcagaacatatctacccttctttgaagaaagtgaaagctttcccagaaatcccttcgttagccattaggtcccattagctacggctagatgatggggccaccctagtttcaagggagcctaggaaagcagggaacagaattgtcccatttacttacatgcattgtaatccaagtcctgggacttccccaaagaaaaccaggatttcattaaggaagaaagagcggatgttatataaacaactaacagaataactgtaacatctcttttttgatctttgttattttgtttaaaaatttaagacccttcccgaatcatactttctgatttgttactgcgtcatgtatttaaggaggcaccgaaagccatcatgactgggtgtgctttgaaacacactatgttgcatagttgctggccaacagcatcttctttgctgacctatatgttcatttttccagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcataggtaagaccaaggaaaaaaaaatccctcatctctaccagttgtcactaaatctgtattgacagccatttgctagatcaattaatatttttgaaactggttttttccctgcttgacatgtcacacagacccagtgtagcaacactgagcagaagaaagatatggaagatgtactcccaagaggaggtgctgtaagaataaataaatgaggccaggagtggtgtcacgcctgcaatcccagcactttgggaggccgaggcagacggatcacttgaggtcaggagttcaagaccagcctggccaacatgatgaaaccccgtctctaccaaaaatacagaaattagctgtgtgtggtggcacacacctgtaatcccagctactccggaggctgaggcaggagaatcacttgaacacgggaggcggaggttacagtgagccaagattgtgccactgcactccagcctgggtgacagagcaagactccatctcaaaaaaaaaaaaaagaagaagaagaagaaggagaaagaaatgttctctgagaatgtcatactgttacagcactgtcgtgttctatcggggtccttggccagttcagggttttcaatttcagaactctgtttctccagcctattttttacagtggttttttttttcttttattagacagattctcactctgtcacccaggctggagtgcagtggtgcaatctcagcttgctgcaacctccgcctcctgggttcaagtaattctcctgcctcagcctccctagtagctggaattacaggtgcctgccacctcgcctagctaatttttgtattttagtagagatggggacagtggtgtttttttccagatagaaacaccaagtctcatattagagcccacttagtgtataaagcttgctgctgccttgagatcaactaaatatgaaatattaatttaatctcccagttgttcctgtcttctggcacttaatggattctgtttcaaccacgtggttgtccctgtcagtggcctaggcacacagagtgaggcctgagtaactctagagccctcaccttcttgtgacccgtggtggtgatgcatctttgtgagcagaggatctatgtatattagcacctaacatctggagcaaaactagtcctatggacataagccagatgctgttccgaggtccaaaaggtgagacagctggcagttctatcaaggacagcccgtgttccctgggctttcaccctcttttaacgcttttgtgtccagtaaagctcaaagtcaaatgaaagacaatgacacgtcttaatcagatagtgcacctataatcagagtagcagatgaaaaagtttacagtctgtactaattgcacatttattgttggggattttccatgttttctaaacacaagtcattgcatatatctggagtgatagatggggaataaaggagatctggctacacagttttgtggcatttctgatagataagctgaggaatggtggccataactcaagctcatgaggacatctccctgtccttgtcttgtgacttctggccactagacagggccactctgagtcttttactctgcgtgttggtatatgagatggaaatgcctgttcttaggaattttacacagattttcttactgtaatggtcaaagagtaataaaagttctgttttaaaagctagatggccaaagagtaataaaagttctgttttaaaagctagattctctcttttaatagggttaccatccatataatttattcttcaaaccacgatgcttttgagagtgaaaagggggctgttaataattctgatgggacaatagtcatacatagatcaggagtgtcccaagcaaactggatatgtggtagccctgcattttagcaccggcttctctgtactctacccctaagctttctagctcctaagtggagcgaaaaatgtatcaaggcatatatccaatttagtgctacttaccttttcaaagagctgaccaaaattcacgcaacaaaatgacactgcctttggtgagatctaaatgaaagctataactaagctattctttcaggctgggtaaggggaaagtatagccaatatccccatagttatatatcaaaacttttttttttaactttttaagttcagaggtacaagtgcagtaaagctttaatactaaaggaaatagttcacaagtcaaccagggatacttatgtcatacttgggaaaccagggcccaggaggtctgagaattgcatgttaccttcagaggaaaaatctaacagcagatctctcgtgtgttgaaaattctcagattgttttgtgtaaatttattgttgttttgctacttaattgttaaatttgtaattagtaaattattcgactagtaaatttgttttcatgtgatagtagataatcatctctcacaaaaccaatttaacaaattcaaaattatgaagagaaacctgcaaatcgtagtattctgtagtctttctttttattttttatttttttgccttcatactttctattccaattcctgcatctagattatacattcttcattggtgtagaaaaaaaggaccagtttggctcaaatttgtgattttgattttaagttggactgcagcacttttgctaaattgtggttcatatgagctcgtaatggtatagtggacattttgattctgatctgtctccctccacaacttccctccccctcccaaactgatgcacgtataattttatcatataaacccatgacttagcttcaggggaccatgttgggaagacttaacatttgatgaactgtaaccgtgtaccttattcactcgttcatcttaataatttatatgtgttgtattattgaaatagtcattaagccctcttctgtctgcatcatatttactttgcaagtagcgttgttagataggccttcatcctttcttatctagccctgctatatctaaacatacgaagtcaagttcatgactatgatatagggatttcctgtctgactctccggtttgcgtttgggcttgttctcttatcaactaagaagtcctgacttccagtcagcttggtccctatataagggtgccaagcctggccagcacaagcacacccaaaggcagttgccagctgtgcttcattggcagagcctttatcaccatccagaggtcactgagcaaaggaaaaacatggaaacacaccatcaggcggcatgtcttaaagcacccaattgtttttccagtgggcaccaaagaccagtgctcgcattgtttcctggtttaggctttctcagttttaactcagattatcatctattctcaggatcttgtgtatctcactgttagaatgtacagactatctcaccccagcccacagtgctgacctcaccttggctgtagggctcttccatggatgaatgttctccctaattagtcaaatacagatctctaaaaacctcaaactcacaattaagggacctcttttattagtggtcaaaaaaaaagtatgcatgtgaagtatccttttttcctactgtaagaaaaatgaaaagtggtgattctaccaaaaatagttttctgattacaagtatttgtgttccttgtgtcaaattgtcaagtcactaatgagcaatgataatgcttctcttttgatgttcgagggcctcatattatgtgtgctaatttaataggatatgtaatatagacataattaataggattgatgattaattttgaaaagtgaacagctttgtctagtttcttataatatccatcattagggaattcattgttctactagaaagaaactaagtagtaattacccattcactttaatggaacatcttctgattccctaatttttttttgtactctggttttctaagtaaatacattcaaacttagattttattttcataagctgactataatgaagggttaaaggatgaagaacaagaatcaaaagttgacaagtatgacgcaggtggagataaaaggtctggtgggaattctgatgggtgaaaagagatttcatatgtggttccttgtaaacagcaaatgcctctgttatgacctacttagccatgcaagagtgtactcgtttctaaagcatcccatggccatttcactgatgacatgctacatgcctttctcaaaccttgccagagcaaaaattatttcagtgctttcagtgaagtctttatttcatgacttcaaggaggatagcagagaagtaaattagcagccctcataccacaccactcatatgcctgtggggatactcatatccccatatgagccctgaccacatcaaccttgctctaatgttcgtgttggtgatgcttctgcccatactgagtgccagactctggcagcttctggaggggggaccgtgagtctttcagtggatcctccctgtgtccaggggtcagtcattcctggtcagatattaaaaagaaaaagtactactttgatcgtaacgtctatcttaggttaatttccaggattttcaagcccacttcattgctgcatcatacctgttgactttggaagtcaagatgctcaggtatgtcccccagttaagccactctaggtgatcaggactgacgttgaataaatagctcatgtgttggctaagtcttaagtgttagtatgtagaacattgttttgaaaattatgacttggcaagtatttgttagtaactacgtgttattttagaagatatttcaaggtcagatttttaaagtcatttgttggctagaaattcttgagtcagtgtatctttctcacttcagaaattgtggatgattgtttgtgtttattatatataatataaatatttacttatacattatataaaataaatacaagtatatatgtggctcataacttccatgtgaactaagttaaaatgttctgattacatgccttctaatcagcgagtatcattgaagtgtttttaagtgtcatatatctttaaggatctgtgaaaatgagttctattataggtagatggtacactaaaaatttcttacaggttgtattaatacctctgtttgattagaagttagctatttttatatttatatatttactaatattcgaagtgggactttgccatggagctggcttagcttgtgttgtgactgtatttcttctcaactggctgtgatactgtcactgtatagtgatcttctccatagtgatcttcaactcgcaaatgattaaataatacttaaccttaatagggaaatgtctataccttatatcaagtcaatacattattccttttgcttagatttctgtttatttaaacataactctactaaactggtatcttagagatatatagcatctctttggaacatctgttggtcattgtgtatttaattcagcccaagaatcaattatagtaaatatttaattcaggcatctttaagctatacaacccatgaaacttaatgttacgcatttcgaaaccataatgatatgatggtgcccaccttcattggaacatttttcaccgctgcttctccagtgtattttcttaaaattaaaagcttctgcacattggaaagagaacttcccttggagccagagcattataatcacagctctgtcacattctagctttatgtctcggtgatatgatgtaaccctagttcagtttcctcatctataagatggcaataataatattgtcctgtgcttcacaaaagtgccaagaatattaaatgacataatgtacatatgcaaaaatactttataaacagtaaacacaatttaagtgtatattgattcagtagaattgctgctctcgtaacacagattgcagctgatctgagtaacattatcaaatagattctagtgtctcactgttaaatgtagcgtctggctttctacaagagaaatgcctggatataacatctcagtgatgagcttttcgaggccgagaatatcttagataatgctttggcagtgtacctcatcctgcctgctcccatcttaaaaagcccatgtcactttggtcactccatcgtaacccaggtagaaacagtgtgctttctgtagctcagctgtgttgtgacagggccttgcccacaaaccacactgttctttcatacctctagacattatcagaagtgagaaaatagggctcatttattttggtttatgttgagaggctagtcccatttgaagagtctgaacttactgtgcatgatccactgtgattcctgggaacagagtacgtggcatttatcatcagggcgcaccctcatagggagatgtatacctcctgcacattcttcagtgctgtcagttactgtggacctacacggaggcccaggttgtcacagttagtgctaaaaagattgaaacaaaagatgagaggtgtgatttgaaagaaggggaaacaggatgattttctgtgtattgtttagagagtataaaaatgaaaacattctggttgtcgtgcaccatgctaactttggaactggtctaaagttatcacaacaaaaataagcagtgtttgatttcagtttgtttgggtcttaccctatgccctaggaagcaactgatcttgctgcttccataagcagctttctcttctcacttcagtatcctttccaggcccccggctgtgtaaacagaaattcaagttgttctggttaacttgggaaaaacccctaagctgcatagtctttaggtgtgctccgctccagccccacgttcttaatttaaagagctctgctgcacggaagttaggctgctgcactgctatttccagagagatccaacagtatctgcctttctcttattttggtccaggaggccaaacctgattttgtaccctctagtggtcaaggatatgggggaagaccccatcacaagagggcccacctgataagaaccctctggagactgctggcagagtcttcttaacaggacttggactgtgaccagataaaacatcttctataggtgcagaaactggccaatctggaaggtccaatggcttctacgttaaggttttcttaacaagatggtataccccgacttttacatggattgcttcttttaagtcctaaaagaagttgtatttcttcctccttatttgggccaccttgatctttagttgtttctgcctgccttttaaaatgacatttttatatttccaaaatgatttatgtgaataatgtccatgtacctgtagatgcatcttggattgtaggccctactatgacatgagaccacatttaaagttgaaaataaaatgtttccttcaacaactatccatgaagtgcttaatcaacccataacaaatgcttaacacaaggagtaagctctattactgagaaattgtgtcctctgcatgcagtggttctcagccaaaagtgactgtgtttactagtttggtgagatttctttttgtagaactaggacttggaagcctgagcaatcacctatttctttctagtatgttgaagaaatctgataggctcttttaacctttataaattacttgcacttcctaccctaagaaataaatgaaataaaataaaacaaaaatggaattctaagaaaagccttaaactgtggccaaatctgctttcacagcaattaagattgaggcagtatggagaggtaggagtcagccgacatgacagcatgtacctcatttttttaaaagaaagatggattaagatcagatttttttttattggaagctaaaagttataaagtaaaatacccaaagatttaattaaatcagtaagcatttgttgcatttttaaccagaattctttaatcatttcctgacagaatgactgtgtattagatctctgcctcattgaaattgtctaggccccctgcatctgttttgagcaagacttcagttgagttccatcttttcattcttttacagtctgtaaaggcctggtgagtcagcgcattgaaaatcagtcatcatcttctgtctatgagttcttagtggcccgtacattattttctcagatccctgcatcattacaatttccatgtgtttaagtctcttctttatgttttttaaaaaaacaagtgaagccagtttctcaaacaggaggtgctcacattctatttattctccaagtgtggagtttgtgcccagcatgtgaccgacaatacagaatgaaaagcacatgacaagccagggatttcagtgtcttgttagaaagtgaaatgaaagtggtattttattcctttgtggcttagagagtgctgtaatgcattgttacaggtataaactaacatttttagaagtacttcccctattcttgcccaggtcagctatgtcattggtaatacttggctgccttttctaccgctcccctttctaaatctcatgttacccatagtatacatgggccctttgtatgcctgcctaaatgacttcactgaagttttctgtaaatccctcaaacctcaaacttaagaacccaaactgtaatatcatgttattagcaggaccagctgtctgcgcgtcttttccagacactgggctcatccatttggatatggactcccacaaagctgacgagaccctctgggtgcaggagcgtctcctgaggaccaaggccttgaatgccttgttcacgtctgcatccccaacacttaggatgacactcggggggccttgatctcaatgtgtgggaagaacaaatgcccccgttatagtactgtgttggcttgttttgcagtgagacttattctgagattaagagctgcatttagcttcttggccaccaggatcccagagtaaattacttttcaggactctaaatagtacgcaggagatttgaaagaactgatgcaatttctaaagcagcctgacttcctcccaggaggttactcccacaccaagcctctgacttcatgaccaccagatttagaaattgaagtatctatgtaagaagttgcctcctaggcagaaatcaagaaatccaactataacataggttagagtccattttggtttttatatccttccacagaggaaagaggaggaagaatctggagatgcgtttttggtttttggttttttgtttttttttttttccagaggctcatgtatatcctacatcatggtcagtttcagagcagggctgtgccaccatctcagtgactcctggaatactaaattggatctttgtagaggaagaaaataacacagttctagattttccctagctgttaattagttttatggcataattaaaatagctcaggagtaaaaacaaagtccagccttaacagcctgttaagtcttcttttcttatcttgaaaagaggtaagataatgaagtttaaacagttgaagaagttaaccggaaaggaattaacatttcaaggccttgccgcttcttcctcctcttgtgatatgaaccagaattgagggaaaataggcaggagggaacccacactgaattttccagactctactgctgaaagacattgtatatttttattgtaatcatatgtgatgcaagataatattgctcatatctgaatcccaaaagaaaagaagatgtttgtctgagcatcccatgaggtaagcagccccatggaaggaccagctgcatccagcaaagggctccaggtccctgacgtagttgacggtgatggcagaagtaaatctttgtattcttgcagagactttgtttctgaaagaggccaaaagtcatttcaaactgaatctgacggataagataggggccatagctgagaaaaataagtagcgaagccgatctaaaccaaggtgtaactgtgaaatagtaaccatcttctttgcacgtttcaaaattggccctggacgccatcgcaagaggaaaattccaaagctgtgcagcagcggcagtaccattggaagagtttacagcctaccaggactacttagaaggggatcatgtttatttggatatataacccctgattggtttgttttaaaaataaacgttattatgttagtgtcatacctcatggtttccttggaacattctcctctaatcctctttgagagagtgggagggagagattgtgaggggagagagaagtgttaaacagagatgttcctaaggaggaccaggaatcctaaattacctaaataacccacttggttatctccactccttgatagcactgtgtgaggtatgatgcagtcaggtaaaggacaatttatctttgaagacaaagataaattcgagtccccattttcaagagtcagtgagaagtaacagcttgtttgtgtggcactgattgatccttgtccgggcaagtggtccctccacaggttatccggcttggcacacaacagacagaggtgctggcggactgtggaaccagacccgctgtggttcccctcctcaccctgccacttcctagctgtgcatcttggacaactgattgaatcttgtgcctcatttttctgtagaattgaaacaataccctgacccattgggcaatggagatcaaatggcattgatgcaggtaacatgcttaacacagggcctggtatgtagtgggcattcagtaagtggaagttacgattattattaggtatcaaagaacaaaacttaattgtcagaataagcaaaccagttggaataataaacgtagcctttagagttaaatggcacaaccctgatagccacacataaactgttttggttatttttcgttggatattcccagtctttatttctacatccctcttcaggctgagtgcttaaaatacatttatatgtagtctcaattagattatgctgtttcccccaaaagactacagtgactattattttaatttatagaacctcgaattgcatggaatagctaccacatctccctttaaacctggtagcttccaaaacttcctgatggcgttttcgcaaatgggtccctatgtgtcattgaaggagacagtgtatctaggaaaagaaatgaggatactgagcaaggtgttggtggctcatgcctataattccagcattttgggagactaaggcaggtagctagcttgaggccaggagttcaagaccagcctggccaacacaatgagaccccatctccacaaaaaaaaaaaaaaaaaaccctaaaaattatccaggcataatggtgcatactagtagtcctaactactcaggaggttaaagtaggaggatcacttgagcccagaagttcaaagagctatgattgcaccactacactccagcctgggcaacagagtgagaccctgtctcttgagaaaaagaaaaaagttattggttttcccattgcctccaaggcattcagagtggttgttctttctcataatatcattttataaatattcttttagacagtgatctcatacagttactgaaagagtatttacataaaccctgtatgctgtagctcagcatttgatctttgatatttcttctgtggttttgatacatcagttgaaattattctacataggtcagtatcattcagaaaacaccacacatcatagccaggtgctttaggtccacgctcagtctaagttcttcccatcattattctcgtgtgtggagagaggaagggaaggaaagaaggaatgaaggagagagggaaggaaagagggtgggagggaaagggaaggaaggagagagggggaaggaaggaaggaaggaaaggagggagggagggtgaaaaggagagaaggaaggacagagggagaaggaaggaaggtgagggagtgggaaggagagaggggaaaggaaggaaggagggagggaaggaggggaaaggagggaagaatgagggagagggcaggaagggggaagaaggaaggaaggaaggagggagtgggaaggaaggagggagaaaaggatgggaaagataaggaaggaaggagggtggggaaggagggagggggaaggaggaaagaaaggatggaggggacaggaaggagggatgagaaaggaaggagggagggggaaggaaagaaggagaaaaggagggagggaggaggagggaaggaaagagggagggaaggggaaggaagttcagagtactacttaaattaggcatcattggatgattctgttcagccaatttgacacgtgcattttggataccttagtgaagtcgcctcttttaaggattccatattaatcaagaacttctatcatgcttttcggtatggaagggagggagggggaaggaaggaagggaaaaatacagacaggaaggaaggggacaggaagggagggagagagggaggggagggacaggaaggaaaatataggcaggaaggaagggaaggacagggagggagagagggatagggaagggagggagaggaaggaagggagagagggagggacaggaaggaagggaggaaggaagggagagacaggaagagggagggagggacaggaaggaaggaaggaaaatacagacaggaagggaaaggaagggaaggaaggaaggaaatacaggaaggaaaaggaagacaggaagaaggaaaaggaagacaggaagaaggaaaaggaagatggaaggaaggaaggaaagaaaaaaaaaagaatatagacagtatagaccactgcttggccttgagtcagaagagttatggacaagatggaatgaggagttttacctcctgtctggtttctttgtacggtaacactgttactcaaattttattcagtgatattgcattgtatgggccaaggttcatgtaaaatataatcagtgatactgctctatcaaacattgcttcttttcataaagaattctcagcaaaatagactctaaatttgatggccaaaactaatgtcctcaatgcagtgtccaggctcagtctaagttcttcccatcattattctcatgtgtatggagagaggaaggaaagaaggaatgaaggagagagggaaggagggagggagggagggagggaaggggaaggaaggagagagggggaaggaaggaggacaggagggagggagggagggtaaaaaggagggaaggaaggatggagggagaaggaaggaaggtgagggagggggaaggagagaggggaaaggaagttcagagtactacttaaattaggcatcattggatgattctgttcagccaatttgacacgtgcattttggataccttagtgaagtcgcctcttttaaggattccatattaatcaagaacttctatcatgagtttcttttgaaagcctccaaatgagtttttagcctaataactagtgtgccagaaaatttctgtggagcatctacaaacatagggaagtgcattttttaatagatactgaaaaattgatttctgattacaactagaactttccatgattacttaatgccccttgttgaaccttagacagcagtaatgagaaaaatgatagaagttgttgacaacataagagatgatgcatttaacactcgatctcagtttcttccttggacttgtttctaacaccctcccaatggattgagatttgtatctgtggagtaaaaattttacaatattaggccattattagtgccatttataaaggtgcatatgcttgagtattgtctgccctccttatccataaccacagattgaaaatatttttaaaaacatacaaatttcaaaatatagtataacaactatttacatagcatttacatcgtgttaggtattgcaagtaatctagagatggcctaaagtatacagggggatgtgagtaggtatatacaaatactacgctgttttatattagggacttggcacatgagcgtctttggattttggtatccatggggtcctagaaccagtctcccaagggacaattgtaattggtaagaagaaaatgtagagaccacagatcggtgagcatttctgtctgtgaccttcctctggaaaagctcagtctgaagcacaaactgggtcttcatagagaggcacttccagctgcctcagagcactcgctgcaaagcttcgggctgctcagagaaaatggctcctgatgccattatgggcttctcgtcgtgccctgagatgtgctgggttctgtttcttgtgtctattcaatgaattctgacccttcaagagcttcccagatgctttccaggaagaaccctgggccctggggacgactgccttcctaaggaactttccttctagatgcctttgtagtttttcatgtttaagtgacaagtagtgagtggcgctgcttgtagaatctgaacacaaaatgtccccggattagtaggtgtcatccagttatcagtctcgtgctcacacccccgccaccgggtttcgatttgcccatctctgctttgctgctggatctgctggtccttggggcctcactctgaaccacattcttgtgtgctagagggaaatttctgtgtgtgaaatgtggccactagggagtcagtaagttcaggtacaccaggaaaaggcgttgagtcccagagaaaggaaccttcaaggagaagcttggaagagggaggaaatgagtgtgacaagggaccatgggaagaaggagcagaatgtttaaactgaaatccctgtatatactgtacagacaaatagctctttttttctcaaaaaccagttttctgggggaaaaaaatggattgccatgaaatgaagctgtttttaaagtagaaatgtccttgtaggtgagagaaatctacgcagcatccctggaaaatgaaggaagtggttatctcggtgctgactcccactcaagggttgcagaggaaacctgctagttccttgtgatgttgccattatcactccttaccattcccctgtattgtttacaaaacaggaacttcttgaatccatctctgtaaataaactcagctggactttttatcaatctctttatctctttctagatgacagtctaaggacacttatctaacaggtaccattccctgccggttgtggtagtgaccaccctgtgctagcctcagtggccacaagatgagagccctgagggtcctcctaaggaaatgaaagactagggaaacaaaagtttcaccaagttccagaagtcctaaagcatactgtgattatacaaagctatcccttgatctctcacgtctaataagatcgtgtgcgttgtcacatcagcgactccatcatgtgctgttcttttcactagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccaggtatctaagccaccatccttccatttgtcaagctgtaatgttgatttaagatgaaatcgaagcagagctcgacctgccccttctttcattcgttcacacggcccaagtagcgtgtcaggcccagttagcgcctccacccaaagaccctatgggtaacttggaaaactatgaccatcccgatgaggggcagattttggttccatgccagaattcctcgggttcttttcttctcatttgcattctctaaactttagtataaccaacctgttaatgacacctgccctattagactagcacagttttagtgcagtgttggactagcctagagaagatggtccaagatgggacccagaagggaagatgcttaaaagccattacactgtgaaaatatattagaaccaaagtggaagacctgcatttaaagaatatagttctggttcattctttttttacttcatcagttccagccagtacccataccatccacgcagccttctcccaagccctcagctgaactgcccatctctttccacacttaacgcaaccactgagtgtaccccagctggctgttgtgaatccccatgcctacatctttccccacgccccactctttgtgtctgctgcattatccctctcatagtttcaaacctaagtccttctcagctgcccagagcctcacctagcccttctcctagtttccatgcctgcatctcctgccaaacaattcctctttcccctgcgtctaatcccagaaatgtaagacctgagccctctccctggggactgacccctagacacacagatttcccggtggtatagaccacacagacccaagtgaaggctcactctaagatctcattgaggtcctcagcgagtcaggagcttttctgtcttacctcgattctcatgttttctcgcagcattatattagagaaccttgttgtattcaatacagggagctgggaaccatgccaattcacaagtgcatctgtactatgcagttgtaagatagggcagttgttaaacccaaagagtttaagtgagctctgcacatgttggccttttgtttaggcgttgtgcagactagccccaattgagcatttctaaaaatgtccatttatctcctaaaaaggataggatgttctgcaaggtaagggggtggaaccatttttcagagccttcagttgggttctctttatagttttccatatacacacttgctgtgtatatggaaaaagcaagtcttaagtgctttaaatgagaataagaaaaaatatatggttttccactggaattattttatctagtgtatattcacctatgtttcacaaatttcataccagtttccccagtaatatgcttttaatgatataaatgatgaaacttgaccttatttatttcacgaagagtcatttaatgccttttatgtgccaagcactgttctaggcactggggacacagcagtgaaccaaatagcagtgcacacggagcttacgttgtagtagggagaagccaggcagctaacaggtaaacagtaactaggtagccaccaagatggggatccggctctggagaaaggcatagcaagacagagaggctggggagaagtggggcaggcaggttgttttgtattatgtagaatggtcagggagcaccttaccaacaagatgagatttaatgaagagacccagaattgcagttctcatggggaacaggcagaacctctgggtatagccagcgccaaggccctggagtgggaacgtgctgtgcgtgttcaaggtagagcagtgttcaaggtgcatgttcaggaggagctgtgtggaggtaactcagtgagcatggaggaggtaagaggtggggcagagaggcagccacggccagacacaaagggtctcgcagatgagtgcaagattgtattgagtgagatgaaaccttggaaggtttggagcagggagcgacaggatctgacttacctttgaatgggatttctctggtgctgggttgaacatagactgtggggcatggaggtcaatagtgaaagcagagagaccaacttggagacttttttaagaatccgggcgagagatgacagtgacttggacagggtggtagccgtggaggtggggagaagtgggcaggttctgactatgccatatgagtcgcatcagtaggatttcaatgtatgcggggagtgagtggaagaggagccaaggatgactacgacattttggtttggggcatttttagcttgaaatagtcattcagcatcaaagtggagctaatgaatagacaaatatctaatctgggcttcaggaaagaaactgaaagtagatctacaaatttgaaagtttagccataggtggtttctagtctcaaggtttttaagccttgagactagatgagagcaccgaagggtaagaataaatagaaaagaaaagagagcgcaggaccgagccctgagaccttctgacttttggagactgagcaggtgcagagggcctagcaaaagaaatagagaaggagcaccaggaataaaggaagaagccaagagggtcagtgacatggaaaccaggtggagaaagggtttctggtaggaaagagtaaactgctcaatcaagcgccactgttagccacgtaagatgcgatgtgggaacagacttgtgcacttagcactgctgtaatgaacttcacagaagtacagtggtggaggcaaagtctaatcagagtgatttcaagaaagaatgggagaggtgaattcaattcacctctcccattgcgagttatggttatgcaactctcatgcagaccataagtgccaagttcatgaataagacagactgtgaagttgagggtccttggagggtttcacagagaaaggacatgagctagattttaaaggacagatgtgctgtcttcattggagtgaagaggataacccaaccaagacctgagatgggaaaagtcaaggccggtgcctctaaggtcaaggccagtgcctctggagcagtgagttttagtagcagtaggtaaggacagaaacagtagctttggcaaggatttttcaggccttaattatcaaatgaagagtgtagattttgttctttatagggaatggggaggcttatttttgagcagctgaatgacatgttcacaaagatgttttggggtaacctttgtgtaacagggtggtgtgcaggaaggactaaagcagagagagaccagaggccaggacagctgtaacaggcactgcaaatgtcctcggtgtgataacaaggacccaaattcaggtagtggtggtagaaatagaggtcaattcattctccttggtttgtgagtttgataagagagagtacagatattgctaacatccttgttcatcattagagatttctccagtgacttctcagttaagatttactattgggttgaaatttaatttctgttaactctcctttctgcatctgctgtattcctgatgaaacatttttctatctttaagcaaatactgcaatcagttgagcttttgccaccttctgtttgatccagtatgggctgccaggaaggctgtattcctgtcccattcttgcatcggttcattgtgtgatcttagtcaagactcaccaccaaagtttcacgcttccatttctcatctggatagcacatgccacctcccagaaatgctgtgaggattataccatctcagaaatgatggtgtgggagcacttcccggtctgtccagcaaacaagaaccttgcttcttcctttagacatagcaactaacattctgaatatcctctttctttacaaaacagtttcacctttaccatctttggatatgagtacagaacctatgagctgttaggtgattagcacctgtctctttacagaagaaactgaggctcaggaaaggtaagagggttatccgaagtcatggagctggttagcagaggatgagacccctgctcctaaattgacctcaggttccatctcccaaaaaaatgctgacctcagcttgtaagcaccaagcacctcgccccgtattccagaagaggacattttaaggaaggcaactcttagagatgaccagatgcctctgaaagtaaaatatatccccagtaaattatcttgagttcctccaaagcgcaggtcaagtatgagcacaggagcgattgtgaccgtgtgcccgtgatcttcgtggtagggctggatgtagctttgtgtaaatctccctggcactgtccatctgtaatgccaacagctgtaaagggctcttctagtaaataccagaagggttatattttagagtcttgggttgaaggcggtctttggccatagaaggcggcctgggtctaggcgtggaaacaaatagcattcccacttctttttgtttcaacaagcagttttttaactgaaagaattgttctttttggcaaaactgctcagaggcagcttcccttttatgttcaaattaaagggaaatgactctcttcccttggacttgaggactgtgaactggagctgtgcgtgttctgtgataaagaaggaggtgtgggtggattttaatcagagctgaactgagtggtggagtgagctaatacacaggacaggttatgagctgctggaagtaagaagtggctgtcaggcatcactcatcaggagagacccatgggggccaggattggtctcactcaaaactgtatgcccaaaaccagtgcctgccatgtaggaagtgctcaggaaatatttgttgaacaaatggtaatacaattaatcactgaagaagacatcctccttcaccatagacagatccttctaactttccctgctgtttccaagtagagcccctgtgggaagaggactcactgtcatgcctcagcttggtggagtttcaccggaaatctacccatatgcagggtcaaggcaaaagaattccaaagttacgtctctccctctcactcaggaaaaaacctgaggtggaactgaatcaatcccagctctggggcctctgcagaaacttttactacttagccattgacatttacaggtttgttggaattggtgtcgcatttgccctttacactgaggcctcagaaaccagtggcagttcggcccctattgtcataggaaggctcagtgtgaagccctggactaggcaggggaggggcagaagggtgtgtgtggcactgaaaggtgtcaagtaaatggcgacatggcttttctgcccatattcacccaggaaattcataccacacatgagagtttttcagtgggtgcattaaataccatccccctgcacacacctcccggaatcacctagtccagacctaccgaatcaggacctgtgagagtcagcacattttctaagctctttgggtaattctgatatgatttttaaaaatttgagagtcactggtttgtgctcaggcccctgcacctgcaaatggtaatgttttcagtaacttctatttttaattctcgagtttgcagggatgcactgaacctgtagaaactataattgttcccagttctttttatgtttaaaggttttataatataactaagaaagcagaagactattcttggaggcaacaagataacttgggctttggaatcacagaactgcatttgaatcccagctctaccacttactagatgcatgattttgagcgatttaacttaacctctctgggtttctttccctggctgggtacccagttaactcatgggggaagcttagcaagtactctcttctctatcactttcctcttctctcaaagtcatcttaaggtttacttctcaaaagcctgttaactttgagacagaaaacaatcactctgagcagaggggattattaatcctattttaatttatatacttatactgtatattttatgttggtctaaattaggctatggtgagtatttaggatcctacgtgttgagtaattgctttataaaaaaatcagtgaataatatggcaggatctgcccgaaaggaaatacgtgcatgacacaaacttttattgtcacattttgaaaacaggttctgaagaattaattgctttaaaatgtggggctagttgtcgttagctggatgaagtataaatacaaagaaatatttgtaggagctgagaaaagttctgattttttttttttttaaaaagacctgatatttgagataaataatattcttctgtttgttttatgttgtagtataatacctatctgatcaaactggataatgtaaatatatttactgaagatcagcttctaatctaaatggttccagtggtaacataatggacatctgagacttatactcatgaggtctacctacagtccattcccatttaggggaagagaaaggagaggaaatggccaagaagtccataagaagtggacagtgctttaactttttcaagttttgctaacatattttatgtaagttattcaatacccccatattaaagagcagcagctattaaacaccaacatttattagctgctggaaagtaataaataacttcagctagtccccttcagtttccataaaattataaaaccaacaccatttgacgtgaaaaagcagtttttgaccctggtggagtagagaattctttcatcttttaaatgttataagacatgtttctcctcactgcaaagctcgtgattctctagcatgagtgatgaatgatgtaaatggcagaaactgcatctttcatgtcccaggaaatgtggtcttgaattgagtgcaccatttggattgagaatacaaaagccaacaatacagcagcttcatcatctcttaggaaaaatgttcccttcttttagaaatgtatgtcccagaaccaaccaatatacccattgagaagacaaaccatcgaggagaggttgacctggaggttcccctgtggcccacgcctagtgaaaatgatatcgtccatctccctagaggtaagcattgaaggcagctgagatctgctagttttctatgtggtctacttaggtgttttctaatttttaaaatttcctatgagtaacttttaaaatgacttgctgaagtaaattcaaataactattcccgggcagacctgcttggctgaaacatatttacaccacacattgataaaataagtcagcaggtaactggattaaatgcctccttcctggccctctgtgctcagagactttgtgatgacggaaataaatggaaataaatatgttcctagtcataagagcctggtatctggaacttctaagccatctctcaaacgtatgtatatataaactttgtaaggagagtaatcatgaattagtcactgtctcttgtagattccgcactgagcatttgtacattaggttaccctctacttctgggcagttttcacttcatgtgagctaaacttatcacctatgttgatttttctagtcaccaggtttactccatgcctttttatcttgctctatcctttcagtctgtgcgtaataaagatgttattttgtctcccaatagctgtcactatttccttttctataaaagtagacctcttcttgatcagtagtataaacttgctcttagccgtgactcagtactgcctcctgtgtcctccaaccatattttccaaatcattgctatataacttatatttcttttggaagaagcaatattcattgcttgagagagattaattgtctgttactctagatattctaaacgttatgcttggatcaaaccagatgaccagaaagtcaaattcctggacctagtgtttttggctctctgtataattgttctgagtgtgctttagcattatgcttacattcattcagtcagtaaatattagtgcctgctgtatgccagaccctgttctagaggctagacttcaacatactgtaagtacgactattcttatgactgaagacctgtgcattgcatagtatctatgttataaagcccctaaagagaaattttctcatgcatcaatagcaagaaaatgtatgcttctcctaggcaactgatttgttttaacctaaaagacattgtgaatcgtattaaagactgatagttgtttccgtttttggctttggctgctacaaagctcaaagctaactttgatggcttggttttagcacagaaacagaactggaagactgaattttgtaaacagacctcacttgcagcttcatttttctttcaccaagaccttccttaatttatatcttattcttcttgtatgacacatattcctaaaactgctttctatttgatttggaatatgggaaagtaaaattcattcttttattcattgtatatttaaaatctgaattcatcaataaaatgttactctttgacatttttcttgatgctggaaaatatgtttctataaagttttattgccatctgcactggctaaaatgattaggaatgttttctacctcccattcctccctgctcccaccacccctccagaacacacacagaagcatatcatttaaaatctgagttttaaaccacagtgttacataccacaaagctctgtcacttcatgagctgaatttccagaacaatttaggagtaactccttttgatactgtgtgtcatgtgtctttaactaaattgatttttaatgcatttagaaaacctgactacaatgatttatttgaaacaaaacagaatataaaaataaggaaactttagggagtgccccctattggtatgtgtttaaccttatttttgtctgcaacattcattgtaagagagatattttaaaagaacataaaggtcatcattcataagtgcaaaactaaatgtagtcacctatgcgatataattcattaaggcagaaaaatgatctaataggcctaagcaagattttcaacaagtttatgaaaagtaagacaatattgcttagagttcagtgaaccagagtatcaaccaaaagaccaacttcctgattgattagttttaatctaattagtcttagctattaacactaattaattaatagtaaaagaacatactttgttctagtctcctgtttgccaataaatcagctaatgtttctccttcgaaatctgagaagaccatgtatgttaacagtaatcaaaatattttcatggtctaattcactttttcaaaatctacctaagcaccttcttatttccttgattagctagagccggcatcatcttacccaattattaagattctcactctgatttttttttttttttttttttttttttaacattcatccctaagcgctctgctctctactaagagctctttggatcaacattagattttaatcacatttttagcctcaaaaaactgggaagcaataaaaccaagttgctgatctctatttgcaatgtcattaaaataatccttaacaattctggtttaggtaataaatgcttataattttttttctcctagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaaggtactgtcttgcaaaagttagtctcttgaatgatgaatctaggaaaacttacactcatattgaaattgcttgaaaccttgtgtcttaaccagtgaagtttttactttgctcttcatagcctcaaatccataccaggctatagtatgaatcaaatacatagtacttgcccagggctggactgatcacaaggtgaattatccattatccattggttaatgaagcctaaccttacatggatccagttttctcgatgtactctgaagttttcaaatgaacaaatactcctggatttattcctaaatcattgtttatattagacattaaaacgtgatcacctggcatttcttagtcatcagtcaacttagaattgggagagaaaaaaagtcccaaagggatggagataatactcaggagagggctggaagatcatgagggtatacaaaaggcaaaaggaacaggtcagtgcaccaaaggcaagcaccaagtggaaaggtcgcaaaagatgaagaggtggtggaaagtaatagtagacaaggtagggagaggcagatttcacactaagggagaaaagatgtatgtttgttttcagtacaaacataaagggcctctaaatctgcctattttgaaagacaagaaaaaactgtattaattaagggaaataaatcataatcccttccaactctaattctaccttttgccttgagttggtttattttaatcataaaccttaagaattggaaacagcctgaacaattatgtaatgtaatttaccatctggttaacccttccatgtaaaaattctgtcctgcggttatcctgcctccgatttttctttgaacaaatgtacattgggcacagcctctgtgctgggtcaggatacagttgccagtgtccaaagctgggaaacagacccgctcataaagtgtatggagtgactagcaacacacaggctggcccagcctatgatagtgttgtacagtctgagtggttagaagtcatccctttgtttttaaaatctgcctaccttccactcattcttcagcccccttgaaacacaaggtctgcacttcaaggatttgaaatcatttcaccagtcttttttttttttttttcagttccttcatctccctatttgacagtttcttgacatttcaccttaaaatacagtttgtcagtgtccctcataaaagtatagttggtactagaacaagatgtgataatctgcatacaggctatcaatcaatgaattgtgaaactgttatctcacctgagttaggtactaaatttctgttcatttgagataatgtaaagcaactaattttttgtggttttattcctcctctcattaaatagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgttttcttagaaccaaagtttttaaagttgctaagatatatttgcctgtaagatagctgtagagatatttggggtggggacagtgagtttggatggggaaatgggtgggagggtggtgttgggaagaaaaattggtcagcttggctcggggagaaacctggtaacataaaagcagttcagtggcccagaggttatttttttcctattgctctgaagactgcactggttgctgcaaagctcaggcctgaatgagcaggaaacaaaaaaggccttgcgacccagctgccataaccaccttagaactaccagacgagcacatcagaaccctttgacagccatcccaggtctaaagccacaagtttcttttctatacagtcacaactgcagtaggcagtgaggaagccagagaaatgcgatagcggcatttctctaaagcgggttattaaggatatatacagttacactttttgctgcttttattttcttccaagccaatcaatcagccagttcctagcagagtcagcacatgaacaagatctaagtcatttcttgatgtgagcactggagcttttttttttttacaacgtgacaggaagaggagggagagggtgacgaacaccaggcatttccaggggctatatttcactgtttgttgttgctttgttctgttatattgttggttgttcatagtttttgttgaagctctagcttaagaagaaactttttttaaaaagactgtttggggattctttttccttattatatactgattctacaaaatagaaactacttcattttaattgtatattattcaagcacctttgttgaagctcaaaaaaaatgatgcctctttaaactttagcaattataggagtatttatgtaactatcttatgcttcaaaaaacaaaagtatttgtgtgcatgtgtatataatatatatatatacatatatatttatacacatacaatttatgttttcctgttgaatgtatttttatgagattttaaccagaacaaaggcagataaacaggcattccatagcagtgcttttgatcacttacaaattttttgaataacacaaaatctcattctacctgcagtttaattggaaagatgtgtgtgtgagagtatgtatgtgtgtgtgtgtgtgtgtgtgtgtgcgcgcgcacgcacgccttgagcagtcagcattgcacctgctatggagaagggtattcctttattaaaatcttcctcatttggatttgctttcagttggttttcaatttgctcactggccagagacattgatggcagttcttatctgcatcactaatcagctcctggattttttttttttttttttcaaacaatggtttgaaacaactactggaatattgtccacaataagctggaagtttgttgtagtatgcctcaaatataactgactgtatactatagtggtaacttttcaaacagcccttagcacttttatactaattaacccatttgtgcattgagttttcttttaaaaatgcttgttgtgaaagacacagatacccagtatgcttaacgtgaaaagaaaatgtgttctgttttgtaaaggaactttcaagtattgttgtaaatacttggacagaggttgctgaactttaaaaaaaattaatttattattataatgacctaatttattaatctgaagattaaccatttttttgtcttagaatatcaaaaagaaaaagaaaaaggtgttctagctgtttgcatcaaaggaaaaaaagatttattatcaaggggcaatatttttatcttttccaaaataaatttgttaatgatacattacaaaaatagattgacatcagcctgattagtataaattttgttggtaattaatccattcctggcataaaaagtctttatcaaaaaaaattgtagatgcttgctttttgttttttcaatcatggccatattatgaaaatactaacaggatataggacaaggtgtaaatttttttattattattttaaagatatgatttatcctgagtgctgtatctattactcttttactttggttcctgttgtgctcttgtaaaagaaaaatataatttcctgaagaataaaatagatatatggcacttggagtgcatcatagttctacagtttgtttttgttttcttcaaaaaagctgtaagagaattatctgcaacttgattcttggcaggaaataaacattttgagttgaaatcacacagtgtaacttgcaaaaagattccatctctgagatccgaaagacttttaaggttatcagtccaatgaacccctggtgtaacaagaagtcccgtgcttttgtcattaggcgggtctttcccaaggtatccttgcaaaaagaaaagtgtctcccagagtacagagaatagcgatgctgtttttagggagacaagaaacctacctgctgtcagaaacgtgctcatagctggtatcaaaattaacttgctacgcacaggaggactgtggtgggaaatcctacagtggttcgatgctgaagaagaaagactttaccgagcacgatcaggggaagcctaactgcaaagagactttttgagctgacaggaaaaaataaataggtatattttcatacattttagaagcatcaatttaagaatgagctataaacagaccaatgttgaatctcctttaaaaaaatatatccccctaaggactttctactatatcatataccctttaaagctacatttcttcagtacaaggcgtcatttttacaaattcaaattaatttaaattattaatattactgtgtaagtacatgtatctggggaaagggccagtaagttccaaataagaacattaataaaacaaggaaaccatctcgagtttctcatacccctagagatgatggctgaaatgccaagcaggcattaaacttcaaaccgcgcccctgcctttgcagtcatggtgtctcagttgctgagtggagtgaggaatatgttagccctgtttgttttgctcacaggtgtttaagtggaattgtgaaagccgcctgtagcaggtcctatatagactcttaattcattcatgtcgtctatgggaggggtctgcatggctgtttttgaaatgtctcacagccccagaaaaagagcaggacttacttctgtcattccacagcagctcagcatactctaataaacgtcctcctcttccccttcgtagcattctgggactaatcagggtttcatctgctcacatattgtagcgatgggttctgtgtggaattaagggaaagaatctagcactgtaaggaaaaaaaaaatacaacacaaaactcccaaatataaggctctcaaacatgtatttttacttcttaaaaaatttattaaatgccattgatttgactggaaccagcaaaagtgctcatgatattgatgaatccataagtagctgtatctagaattcatacttaagaaaaacacacaggcatgaataaaataggacacagtaatgagaatgggcagttctgcagtgtacactttctcaagcacctcagaggacacacacttaaagtacaattcttcacagacacatgaagcagaacatttgaaaatcaaaacttaaatacaagattctaaaagtgaatggcaatttaatggttaaaatttgaccagtggtgaaacaagcagcaagctacaaaatccatcaccaccaacagtttcaatgttagcactaagtattaaaccaaagtaatgcatattctggttttgcttcttcaagaacagcccgtttattctgtttcaatctctctgaacaatactggtaggtaatagttacactgacaattctcacaaaaaaacagtgttggttcttgccatcactgaatttatagtataaatctcccaaggctttaatctagttcatcagtgcagtttagataagaaagccataaaaacaaatgggcatactgtcagttaatgagaaatacaactgcacatgcacaattaatattactctctcttaatctactaagagaacagtttattagtagatttaacaaaaaccatctctagaattcaaaatagtgcccatatttccgttggttctataatataaagaattagtgtagtcaagatctgttaatactgaatcaaacccaagttcaaatgtgctgagtttccaatagcaagaagggtaatggtagccagctctcagaataatgctgtcggaaatctagcttggagcccaccgcagataaagttgctgctctatgtaggacacagaactagaagaaaaagagaacttttagctacaaatgtatattttgtatacatttacacaaaacatacattttacatgtacatttaaaaaccattaagtaatgagaactaaatgttaatgcaaagatctatgtagcaattttttaaaggttttaagaggctatacccaacaaggatgagttctctcaacacacattagaatttcccagaaatgagataccaaaaggccatgtctcttagggtgccacttggagatagaagcccctaacattcgccctataccatgaggcttgtgacacaacaaacaggatccctagggaagtcaaagaagggtgcaagtttctgcacacatttttattcctaactagcagcacggcagcagccacgcgtacattacttcagaatcagccttgggtactactgtcaaggcagcagtggcggcaggctgcagtggggtcattaggcaggtgcgagaaagagaagctcaattagcccatacagtgcccaccctactcccttttctcaggccaaatgagaccacttttctaccttctttcacttctgacatttgcattaaaactaagtggtgtatttaggcagccaagccacactctcatctgtgttaaaacaacaacaaaccacccaaaaaaggggaaggagtattctccccttgagcaatatccccctcccgggcaaagtgtcgtattattggtccccaaggaagaggaacagttaaccagctgtgaactttcaggatgggtggaaaagagcattttcaatgacccgacttccccaccttcccacacacatttgggtaaatacatctccacattgcactcccgtcttcattttctctgactacagtctccactgtagtctaaatccttgctgcttttccacccagtcctcacaatacaaagccaagacaaaaccacaacacatgcacacagacagctcatcagaagcaggtacagtatagatagaactgggcttacagacaggagccagcggcccgagcatgaggagcagaattagatgggtgctgcttccctaaggcatgcggtataggtaggttcacaattatagtcctaggttttcagagtaaagaattaaaaatcatttttccccagaggactctgttctttgggtcataacatccttccatgcagttaaatgctaaaatagctagagaatgcttcagaagctcattctatctctggccctcatagaggcctaggaacccgatataattaaaatttaagacatgttctttatgttcgtctcattcctgtttcacatctagaagaacgaacctcagaacaatccagctttcataccaatgctagcacaacacaaaagcaaacaaagatgtcaggtattaaaaaaaaaatttttcagaattttatagggagaaaaagtcctccatcagaaaaaaagcttattttttaaaatgatgtcaactcagtctgttctactccagtgtcaacccactagtgtctagctcatgcttcttactctcaccgcatcaagactcttacaagtgaaatgtgtgattcactttgagcaccctgtgtatactactgaggttcccctttcatgacactatggtttaagaaaagaccatcagaggtttctaatgtctcagaaatcttcatcttccttgtgctcagctaactttaatggagaagagagagaacccagagaagaagactgttactagaagaggattgctctcaaaatttgatcaaacactacaaagatgcattttgtgatgctttcaaaatctggtccctgaatacctgctactttaaaaaaatatttcatcgtaaagtattaattttgcctctaattaagttcttccgtcccttctttatcatccaaactttgctcttttctcggcaaagcaactcaacctctcactcttcttttgcctcatcaatattcccaccctgtgtgagcctcaccttgcctctaagatttgtggctgctttttcactttcctgggtttatgcaagtccattctttttatttccccgcttccctccctgtgtcttacatcagtccctttcgtattccctttggcactgttcacagatgtgactcttccttccttcatccgtcctcactaatgatctgtcctcctttccactgtcctcaagatgacagagaccacccttctcaaatttcccagtgccaatctgccaacatctgatacccacatgattatggtgccatctcactaattttcttctactacctctcttgatgtttatagtttaactgtggagattatttccaacccagaacctccaataaaacttaaattatataaaaccatgatatataatgttggtagaagtcagtcatgtagctaatcctcaccctgcataataagattaatgaaagttgtgcattgggaagttactgtctaatgtattagcaaggcgatgtatttactgactaatccaaacctcccagccatcgcacctataccattataaaactcacctttcagaatataatcagttaacaagctcggaactttttcactgtcctccttcatggcacgaagaactgcaaaatagaagaggggaaaaaagacaacaattaaaaatttagatcaaaaatattatttaatttcaatcattgtggccttaatcttccttaattataggatataactgactcatacaaattataggtgattttcctgataaggggatacaacaataaaggcccaataatttattttagtggagtaagtccacaagaatttattttcggtaagaatatcccacttacctgagagttgcttccctgatttaaaatttccaaacagtggagacaaaaaaagaggaactgggttatttacaaggagccttcgctctgtaataataccggaaagtgggttactataatgtgcacctcaatgaactctgtgcagcaagaaaacctcagtgctgcagatcaagcaaactagagaaactcttaaagaaatggacagctataccctccaacaagcgtatcctgtcagactccttagaaactttcaaagaaaagacccaaatgaagaacagacttccatgaagcagcatcgggcatctgcctggctgttgcctcctctgcactagggagaaacctgggagaattagcctcattctaatttgacaaatttatcttctaagtaaaaactaaaatagaaaaagcagccacctagtataacgtttttcttttttaagctagaccattcaggttgagataaactcacaaatattatactttaacagagattgccaactcaaattttctccaaagcatcctggtaaaaatatttcactgtttttccccaccaatacgacaaggcaaaatagtgtaactgtcaaatcagccttcctgggtttaaaccctggctctgccacttcctaattgtgtgacgctgagcaagctacttaaccacttttgcaccaagctcttcatctgtgaaatgagaataataattagatttaatctcaaaggtttgttatgaggactaagctactaattcagatataaaggtcatatcatagtgcctggcacatagtacatgctatgtattaactgtaactaacactactcaaattatttaaatttttattaactattattattgcacccaaaatcccctaatagcttttatcaggaagactatatatccataaacactgtgactgtctccacagggctcttcttttggaaagaaaaggagctttggacccaaatatgtccagagattccaaactgtgacaacctataaagacatctcgatgctggaattatgatgactttagtttgaccaatgaagtatttgttcttgatctgagaagctgaggcctgccagtaattcctggcttatgagcaatatttatgttcccttctttagagggctaccatgttcagtatttctgcttccaatcatgtttaaaattttttctaacttctaaataaaatcacataccaaaaaccagcatcattaaaagtataattgcctgggaattaagagatccagctctgtaactaaccatccccctgtgtgatcttggacacatataacttaagttcttcctgaaaataagagggctagctacccagttaatctcttgattttttatttcagactggctggcatcacttgatttatccatatttaaatataaaatttttaaagaattattcaccaaaggaaagaataaaagctccaagttagcaggcagggagctaagaaataagacacttgcttctactcttaggttttccttgcccattcctcttaaatttccaaaataagtatcatcatccatactcatccatgtgatgctctttcatttggttcccccctaattagcaagtatcataaatatttaccataacaaagacctaacagatcagcaaattcaatccctccattttaacggctaattgatagtaacttagccaaaatcatacatatattaatctatttactgtgaggtaaatagaacaaatattttttatcctagtttttcaatgtaagcaaaagagagtaagtaacttccaaggatatccctgaaaataacagggatagaaccagaattcaaggctcatttgcggccatcataagagatactcaatatcatgtatatttaaatctaacaattttttttttaaggcaagccccttgaaggtgcattatctttgtctgctttgttcactaaagtatcccaagtgcccagatcatggcctggtacagagtaggccttcactaaatatttgctgaatgaatgcatgttcttaaaacatcactgtcaatttatgttctacctataactactacctcgggttctacattcataagaaataaaaagtgtttaccgtaaatctttcctattcagttactgacaatcatagatatataagatccataaggaattttatttatgataaatggatatgttcttaaacatgtaagagatgatggagatttaggttatgctttttcccaaaattttagctcataaatatttcacaaggtaagaaaaacagaaaaagtacaaatcaacactagagttactgttgtatttaatgtcacagccatttttggcttcattcttataaaaataaacaattatatgcatcatttacatcctaaactgacaaaacactaacaacaaaatcagccaactaaagtcaggtgtttaccaatcagcaaaaggaaatcaggtaatagtttcattttgtctttgctcactttttgttaatatttgaagatcttcaacagacggtggtccgttttttttctcataaggaatgactgtagtcaaatactgccaagtttaaaaaaaaaaagtgtcacttaaatcggaattaccttatctgcaaaaaatttgatactgttactagaaggtctatctgataatataaaacacaagctactttggtaatgaaaacacaggtattgtatgggaataaagttatcttactatctcagatggagctcatttttttttgcccacctctatttgcttcatacaaacctaatgtcaacagaaacaaccacctttttgcatttcagttattgaaaatttcataggactaagtctgaggatggtttactcaatttgttcatttttaaagtgaagtattatctcacattctacttgcatatttttcaacacattttaaaaatagcaatttaaactaaaaacatagttttaacagttctgcacaataaagtaatttccaacagcaaaataattctgtttcagaccatttttattctggttgaacaaatggtccacatatataacatgacaactttgaaaaacacacactagcttctttcagaagctaacaaagacacacttaaatcgttttagaaatgcgttactattattttaaattattgggtaagtcattgtaaacgtttttaggcattaaaggagaaaagttaaagtagagaatggcctaaggggaaatgaataatgaaaattaatgtctgaaattgagataaacttcctagttcactacttgtaaaggctcacaagtaacagcgccatgtcttttaactaatcccatgaaagcataatccataaatgattgtccagattttcctgttcttaccatcccaccaaaaaaatagttccgtgcatttcacatatgctcatatagaaagccagtggtttttacctggaatctcttagctccacccattatttaacctcgtgccctaaatctcagctctgtggctaatcctcactgagtgatcttatcctgcccataactgaaaaacaggataaagaaagctgctttacagtaactggatccctaagtccagaaaagtggtaaaactgcatagttcaatctctgagacacgttaagtgtcccaccgtctttttgacgtagacttcttgcaggtgatgcccaattgtataatcgaatgtttctttgtaaagaacaacaacaacaaaaggccagaaatgatgtcctcctggcttcactctgcatgtgtgaggcaacctggagaggcaccaggcggctgcaccctggcccgctctctggctacctggctgcatcttcaggcctccctgctccactgacagtctggaagattccttgtcttcttttgaggttgagagaggtgagaggcaacggaatgaaaacaaaaactcaaacatcccttcatggagctcattgatgagctgcaaatgtcagtgtgaggcttccctctttaataggaatgatctcccttccggagaaacacttaactacctgcagtaaaaactccaggcatttgatctaggcgttcttttgctaaacttcctacaagctacattttgtacaccttcttaatctgcacagaagagctaacttaaagacttttctttttcaaggtgaaaatttcatatagtgaaaggtaattttaagcatacccattaatgtgaatgtttcccatgttcatgatattccttcaatcattatacaaaaatgtgaataaaaagcaaattgctagatacttgattatatattcatatctgtgtataattttgtataaataatgtcgcatgcctcgtttggcagacaactgagttggctgactatgcaatacaccttaaaaacacatgaatttcatttaaaggaggtaagatacactctatacatatttttgacctagaactctagtctcaaacataccgacatctggccttcttcccccaaaaatgacatcttgcgacttagacttttagaattagagtcacatattttagtttagttattttcacaagcagattttctacacatgcaatacattatgttaaaatgttaatatttgtttaaaactaagagtgaaaaatggagctataaaacacgtcttaagaatgcatttaatctattgtcgaatttgaaaaatgctttttcctcaaagggaaattctgttcattatcaagactataaatataatgaacttttataataaacttttatttctctcgttaagcaatggtcttagtaaattagtaaactaccagctttcaggatcttcacttacacagaaaatgaaaaagcagagaatgaggaaaataaggataaccgagcacctgtttctctccacctgaatttccaaaggtgtggcggaggccattctgaatgacatttgagatcccttccatgctgaagcgctaaaggcggcaatatacggcccagaagaaaaggacagagaaagaagttcaaagtctattagtaagaatgtaccatgatgacttatgtgcccaagaggcaagcagttagaaaatgccactactcatccagataaagcacataaacccatgctctttttaaaatgctgttgcttccatttctttgcaagttaaatgcaaaagcaactgtttttatgctactatattcatgcaggcatttttctgatgtagctaattgttccaatgtaaatgttgtaagttgtacacatatttgttctatacaaaatttactgtgtaatttttaagatactttttgatattatttacctacattttatcagaagtctgaaaacttaagatgaacagtatgcgtattttcagcctaagtttgtataattctaccatcagtttggagaacattaacataacatttagcaaatgaaaatgctgttacttggagagctgattattgcttcccactcactcttcgggccacctgccactgccttggtgcagaaatgcgaactagaagatggcatacgcttcctggagagcagagtggatcccatgtgccagccaggcccccaaaaacttctccaaagacttttccactccgtttctaggaaacaattctactttctttctcccagcaacctccaagacttcctagaattctttgtactgaaaagggagtatttttttcctaaacaacttatcttgatttgtaaccagtctacaatgtcatcaagcatagtaaaaagcgtctggtggcacctctatggcggctgagtcaaaggagtgagatggattcttacagcatgactaattaaggggaaaggcttcgtgaaaaggaagtgaaggcctgactcacttgatggttccttcccctggaattttatagaggaaatttaaatcagtaaacacatttgaggagtcaacataaagaatattttaccaggccccaaggggcagaagaaaggaaaccaagggaatggtcatcaagaaatacatatagtttcatccagtggttctcaaagtgtagtccagggaccccctcccacccaactcctgccaagaggtccctgaaatcctctcaggagttcaggagttcaaaaatgttttcttaatactactaaaatataagttgtcttttttactctcattctctcatgacttacagtgaaatattccaaatgacagactgaatgcagaagcacatttgtgaatctagctgtctttaatcaagccagatgttaatgagattgacaaaaatgcacagcaatgatattttttcatgattttgtaaaatattatttttcataaatatatgctctttataatgaatttatttttggtatttttcatgaatttatatttttaattttatcacttttaatttgtaacatggctaatattaagtttcttggggtccttgataaattttaagagcgtaaagaagtcttgataacaaaaagctaagaaatgccaacaggctgaatcactgagggagctatgtgcttctgtatcagtcatgtaactctatggagttactgaaaatctgagttccaaattttactaccttacaggagactgtacaaatatgcctctcctcctaaaccatccaacttccctacctagcctttcccagatttgcaagccagttttgcttcagcaagaatcaaatgtctagtcaagatactttgctgactactcttcacttcactgaaagcataactattagctttgatggaggaggctagttataaaacaaaaacctacagaaaactatttctgtaaatttttccaatgctatcataattcattcatattttttaagcagattatcaaatgagataacatcttaggcttcagagtatataataacaattatgagaaactattctcaaataaccaagtcagtttattttttaaattattttcataaaatatattttctttcaaatgtgacaaagagaagagtacttgtttttaagtacttagtctatattacctggctccattatctatgagcatgtttaagctagatacaaattcataaaatctaaagctagataaaaagtttcagaatccacctatcaggaaactaagacacactttctatttggaaagcacattccaaaaacactagctttgttttcgcaatcagaaagccaattcctgtcactagaaaaaacaaaagttgatgcagtttcaatttcgtaacatgcagatgtaatttaggttttctttattaaatgtgtggttgcagacattactgagccatctcaactactgacagcctgactacttgtgaaagatggcttttgtgccaccagaattttaggcttaggggaaattagtgaaaatgcaaccccatgggaagccaaggtaacctggaaaaacaaaactaacagctgatgccagtgaaagaaggtaaagtaaggaatgcagcagaacctctgtccttgctgctgatggctgctgag

SEQ ID NO: 24 - DNA, 36303 bp, Homo sapiens
tcagtggctgcagctgacaagaccctactgtccccagctctttcactggcaatgaaccagggaagcaatgtgcagcatgtacagttcaatcgcctcttgatccattggaaaattacagaacaaggaaatcagaaggttctacaggaaaaaaaagaaaaaaaaaaaaaactaaaatctacgtctgagaacgtgctcctatcaaacgcttcagttgcaatcaactcctacctccccagaagtcaggcagcatcaatattctccctccactgcatttccaaactctccctctctactagctccttcctgacagcatcccgaaattctcaaaccctcaacctctttcctttcccaaggttttcctgaaacagctcttcccaacatcatttatttgatctggaagaccacggccacttttcagatctctgagacatccaggaatccctcctccctgaaccgcttccttggattcagagacaccacactctcccagatttccctcaccccaaccctcctgctctctagttgcttcttctaagaggggccactttcttcactcatcccttaaatgccagtgttccctaggatcttgtgcctctttcttgactcacctctcagatgctgctgtgcctctggattctgtccctatttaattatcatgcaccccctctgggtattaccaatcaatagcttcatcttctgcttccataaattgatagctccactttactaagatcagtaaagtgctaagaacagagactggcacatggcaaagctcagtacatgttagctatctttactgtaatttccctgagcttcactctgcatgtttaatggccaattggacatctgtacctattgtatgtttaatggcctattgacaagcctatgttcctgtctatatttaatatcaatatttctaaaactcatatccaaaactgaattcaatgtcctcttccccaccaaaacacaaacaaaaaaatttaaaaaccggttcctccttcagcattttattctcctcgttcattcatttcctctttctttcaaaaaacatcctgagccagataccattccaggtattagagattcagagtgaacacatcagccaaggcccctcccaccctcctgaccttacattccagcataagggggcacaaaaagcacaacacgtaaatgaagcgaatgctgcagataactaacacatgccctatccctagctccagcaaggcgattaaaaactggatgcctactttctactcggacgccagagaaaagctgcctgaggagatgacatctgagctgaaatatgacgtatacacccatcctagatgagccacaaaggacaatggacaagggagatcttctcagagaacaaaaccaaaagcagccaaccgggccaactcgggagttcacgccacagctagtgtgcaggaggtcttgcaattctcattcaatgggatggaacacagcatctatggaatatgtccattctctcctcctttttataagatgttattgcagcaaccctgtcttctctcccccgttgtgtgccaatgtgttgcaatggctggcttaactttcagaccataggttgacggaaaagagagacatcccctcatttgctggacttgaagctagaagcaggtactgctgacaagatttacatacctccctttggggaaggggcacatgtgctttatctatgtttgccctcgtcaacaatcaatgacctgaccctaactcctcccttctttggtgtccgaaacaatcaacctcaccaatatctctgaaattcaactatttatatttccatccctactttcacttccctggctcaattcttacatgaaatcgctgcaacagcctcctaattaacttatctccagttgcctccttccaattagttctccttgaaaccactgaagttctcttccaaaacagacaaacccaaacatgtaactacctgacctaaaatccatttttggttctctaaaccctttaaacaaaaaccaggtgcctcagaatgtcgtacgacgccatccattttccagcttccactgtgcaatttccatagcctcttcttgagtttcttgccccctcaccttctagctttcaccataatgagcagcctgcatttccccagatgtccctaactgtcccctccaagtggtggcacacaccaccccctctgctgtctgtgcctgttccctcctccttctacctgacctccgggtgcaactcagccttcccagacccctaaacctagctaggggcaatagtaatatatgtccaccttgaccacagaacagagccaggctcagaaaacactaaaaacgtggtagttgttttgaatatagcaattatcacaccctgtacctaggtttctccacgttgacactactgacatttccagtcagatcattcttgtcacgggggtcagtgtagtggggtgggatctccactagcacctccggtctctgttcaccaggatgccagcagtaccacccccatccttccccaccctgcccagttgtgacaacaacaaatgttttcagacattgactcatgtcccatggggggcaaaattggcctggttgagaatcaccgcactaaagcacattggatggttcatcattcaatctgcaacccatgggcagcacattcctggaaaactgaaattggttctacatccttaacacccatgccagggacacaactgtggatcaattaatgtttgcctaatagtagtataagaaaagttcaggagatggagaccatcctggctaacacagtgaaacaccatctctactaaaaatacaaaaaattcgccaggcacggtggcgggcacctgtagtcccagctactcgggaggctgaggcaggagaatggcgtgaatccgggaggcggagcttgcagtgagccgagatcgcgccactgcactccaccctggacgacagagcgagactccgtctcaaaaaaaaaaaaaagtaatcaagatatttttaatggggaggtagagtgcacaagatctaaagagaaacaataaagaaagaaaaatgaaaattctggtcaggttaatttttcataaattcaactcaagttctcaaaatccttaaggacagaaaaaaaaaaatgtcagatacttctttcatatgctccaaaaagcaaaaaccagaccagtatgtaggaggcatctgactgatagtcagtgacttaacctgcaaaacgctacagcgatcattaccaataccagtgggatacttccagatttctgtatgaaaccaggaatcatagctcaggcaaaagtcttagaatcaagaaatttttaaacggtcaacttggacagaaaagtgctgaattagaaatcgaagatctgggttctttcccagctttgccactaattgtgtgacaggagggcagtcactttactgccaaggcctgttacctcacctgtaaaacagatgattaaatcatattaactttaaacgtcttcttatttgtaaatcctacgagtctatggattaattcaatcaaaaagatccattattaagcaaattaaaaacacacacatacgcatagacacacacacactctctctctctctctataggactaaataaatgagtgctccagaatggcagaactgcacagccagatgatacattcagagtccagtggcttcattttcttatgaggaaatgactacattctgaagggagagacaggtcttaggcatctttccatccccgcagcttgcatagtgcctgggccatagcagtcatttttaaacgtctcataagctgaggctgtgacactcctaacaccagaacccaagagccttagagcctatgatcgtttcaccatatcatgaggtgtcttgacttcttgcaggagagaaagaaaaaaaaaaagcccaaaataagtggagtatgaacagaataaagatgacaggacagaacatcaagttatttttcagaagagtgaacaacggagttctggaaaagtccactgaagaatctagtctaggaaagtaagatgagattaatgaaataatcttgttggcgtggtttataaaacataaaacaatggagtgactaagaaatctcaaaccccggccaggcacggaggctcacgcctgtaatcccagcactttgggaggccgaggtgggtggatcacctgacgtcaggagttcgagaccagcctggccaacctggtgaaacctcatctctactaaaagtacaaaaattagccagacgtggtggcgggtgcctgtaatcccagctactcgagaggctcaggcaggagaatcacttgaacccgggaggcggaggttgcagtgagccaagatcgcaccattgcactccagcctgggcaatagagcgagactctgtctcaaaaaaaaaaaaaagaaatctcaaacccctagctaggataatcaaagagtagcaagtaggagatcagtgaactagaaggataaaggaaaagagagaccagggcagtgagaccaggaagaaacaggaaacgagctgtgtagagcatactgtgcattctgttttatagctgccctgtaaattccaaaaaataacacacccttttggaaggcaaaatgaattttaaaaagataaaacctcagaggcaggttaaatatactttttagtaaacgtcacctcatttagaaacactacatttacaaccttgagagcagatgcggtattattaggtgcttatggtgcttaactgggccagagggaagattttctctatgcttaaattttataagtaggaggacaaggaaagggagagacagaaggggtaggggaggagggagacagagtgggagggggaagggagggaagcatttctttgataacatttctcaaggttaaagtaccaaagacataccaaagcgccaacagaactaagtctcagggttatgaatgctgaatgaaatgaaggacattaaagatttgttactaaataaataagtaaataattttttaaaagaaaagaaaaaacaatttgttacagtttaatttaaataactgcaataacgcagtatatcagaaaacccaatcctccctccatttaatctaccatttccaagtttgaaataaagaatccaagtgttcaaattcaaagtgaagaactggtgaaaattctgaatctgaagttattttgtaattgattcatccatttcccactacgtctttaggaaggagttaatagtgctataaaatgccccctctcaggatggaatttttgataggagcccatttgtgagcagggaaatgattaagcattacagtatttactttattgttgccctcactactgacaaatgccaaagtaatgtggcaaggacggaggaagagggtattcaatacacagcttcaacaccagtatttacgctgagaatactcaccactgcctcgtggttgtttccttccctggcataagtatgtattgaaagtctcaaaatcagtccttatctggaaacttttctcagacaaaaccagtagcaacaatgtataaacaggatatagatttataaaaattcaccaaaatctgaaagacgaagaaatgggccaagatccccaaggcccatttacaacatccttgcaaagctcagaaaacgaaaattcaagccaaggatccttccatccacctctaaataactccacatcctcattccaatgcatgctggttctgtgagctaaggtccctgttaagcttttgtttactcatttatgaaatggaaataataacaataataccttctttatagagttgttgtgaaggttacatggaataatccatgcaagtacctagcaaagtgctcagcaaatatcaatattcaaaaaagtattggctataattcctaaaaataaaaaggatagaataatattttagacacaactcccaaagagaaataaccacacctttctacttttctccagaacagcaaaaacagcatttcaagttcttctaatataaataaaattgcagctgagaaaaaataaatccccgattatagttcctagctgataggactgaaaacaacataaagagacattactatctatgactaagcacagaatcagcaaaaaagcttaaacagaaaacaagttaacacttaaataattcggtctttctgaacaataaaaagaaaagagctagagaacactatgctacataaaagtgaacaagaatcagatacagtgctgcagtttgttagttcttctgtggtatcttaaatatatcagcttcttctacacacagcatattcaccagttcaaccagcttcacagaaagcaacatccaagacagggaaaagcaagacaacaaagggctcaggaaatttacactattcccgctatcaaacttgtttctcagcctcacctgacaccaactggtacagatttctaactggccaggaaatcattttaaaatctcaatatcaaagctcaatgacagaaaaaaggtttgaaaaagcactcaaggaatgcttttcatctatttctgactcttactctaatctactcttacttcattaatattctttatactcaggaggttcaaatgaaagcaaaatgaaaacataattattgttcctgataaatcacgacactttcaatagcttgaatatgattttcaaacattataaaacagtaggtttaatttttaataaattaaaataatcaaaatgaggaaacaagtgtaaaaaatttagagtaaaaatattcaaatagttaagatattatttaagatgtaaaagtaatatcttgttttaatacttcaaataattttaatgtgaattataaagcagttataaaagcaaattctaccaaaaatttttttaaaaaaacagatctaagtaaattagaaaatatacattataccaaagcagatacacacttgaaaatcttcaatattaaacaactgttatatgagtttatttttatggcagaaaaaatataatacaaagatcttcaaaattagcattaaataatttaatttttatctttgaagatctttttaaaattttttatgtaacttaatattaatgtggaaactaaactaattattgccaaatattttcaaaaccactaaaactagaatataataattgatcttggcaattttagaaattccttcctgaaacataaatactctaagtacagtcatgtgctgcttaaaaatgaggatacattctaagaaatgagtcaggcaactttgtcgtatagaccatgacagagtgtacttacacaaacctacataattgtatagtctactccatacttaggctacatggtatagtctactgctcctaggctacaaacctatatagcctgttactgtactgaataatgtagacaacagtaacacaatggtaagtatttgtgtatctaaatatagataaacatagaaaaagtacagtaaaaatacagcattataatcctgactccatcatctatctatacgcagtccatgactgaccgaaatatcactatgtggcacaagactgtatacaagaagaacacaatttcctggcacattacagcttttatgtgaacttgtttatattaacaatgacctctctaacacataaagaagtcaagaatggactcctgatttttactacattaactgatggatcatgaaagttcactattaccagcctaccaccaaaaaccattgcagaaaacaaagtgtagatgataaaaacatatcgaaaactgatgatgacatatatttctctaggtaatattatttttcataaaacaaatttgttccacatctaaagtaattcataattcataggtttgaattattaaaataatagccattatatagtcaagtttttctccaccctacaatgaccttctatcatgctaatagtaattcttagtaaatgctttgcactaaagaaaatgcagcaagatactatcaaatatcaccaacaaattattgttaaaatgtgtcaatatttacattcaagaatgcgtatcagagccgggcacggttgctcacgcctgtaatcccagcactttgggaggccgaggtgggcggatcgcttgaggccagtagttcaagaccatcctggccaacttggtgaaaccccatctctactaaaaaaaattagccgagtgtgctggcgggcgccgtcatcccagctactcaggaggctgaggcaggagaaccgcttgaacccaggaggtagaggctgcagtgggcccagatggcaccactgcactccagcctgggcaatagagcaagaccctgtctcaaaaaaaaagaatgtatgtcaggtagaatttagtatctcaagcaccactgcttagataaaggggaaaaaggctgggcactgtggctcactcctgtaatcccagcactttgggagtccagggcaggtggatcacctgagatcaggagttcaagacaagcctggccaacatggtgaaaccccacctctactgaaaaaaaaaaaaatacaaaagttagccgggcggggtggaccacacttgtaatcccagctactcgggaggctgaggcaggagaatcgctggaaccggggatgcggaggttgcagtgagccgagatcgcgcccctgcactccagcatgagcggctgagtgagactccgtctcaggaaaaaaaaaaaaaaaaaaaaaaaaaaagataaaggggaaaaaaactcatgccttttccacttctcctctaaccttcttttaaaaccacattttatcactgtgtataatacaacatttttattccttttgtttgtctccatatttagagaacactggtctacctgctcactctctttacctagccaccgggattcagacacacacagcacagaggtctgctgttagacaagaagctgccacacgtagcttggagattggctggccaaggtgtcactatcactcaggctgctgctgaaggggccccaagaaaaggaacaattcaacataatctcaagataaaacaaagtaaccttttaaatacaaactttagaaacattacagtactctttttatctatgcaaactcacttaactggagttcataactttttgttctactttttaattttactaagtctaaatcagagattggcaaactttttctgtcaagagccaggcattaattattttaggctttgtaagccacacagtctctacggcaaccactcaactctgtccatatagccccaaagctgctacagatgatatataaatgaatgaacatagctgtgttccagtaaaactgtattcataaaacaggtgaagagttgatttagcccttggacagtagtctgccttgacaatccctggtttaaatcatctgagcaaatgtgctgttaaaaaaattccaaattaaaaagaaggggggaaaaagccaggtccatgttgttttaaacagaagccccaaacaaaggctatcaatcatattataacatgagctgagtcacagaaacggagcaatgtagctgtgaaagaaaatatatacaagaaagagtgtatatcatctaagccctagctctcccccatccttcagcaattctctccctaacccccgaaaagcaataaattagggtaatgtgattctttttacagttttttcttttctctactaccgttagtcactgattgctaatatcccaaaagttgtccgtataaactttttcagggttgactgacgcagttatgcatttttgatggtggagtgtaacacagtagaaattctctcttcaaagttcaatgtcattatggcaaacagaaaagactatcgattcctatcttctcaacaagacttagcataaacaaccttacatgtgacattcaattcaaagatttgttactagatactcagagggtaactattccattttcatacatataagtttactaagttatgatgctaagcaggtaggccataatgcccagacaactgcagataacactagatgaacagcaactactaaattacaacataacctggtgttgttcaacctattcttgtattcatgagaactgctactgaaattcaatgacctgtgaatgtctgatgtaacagaagcctggctcccccaataaaaatccccaatagaacaataataattcaaatactacaaatctaaatcctccgggattcaatctcctgtacactgaagtttcaattcaacaaacatgcattaagtgatgtgcgaagaacaagaaattaggtgaggtgctgggaaagcagtgtgaatgagcctttaaagagcttgcactctgttagaagagaggtttaagccaatgattacaacacagtgtggtacttcaatatcacaagtatgtgtaccctcacaaataaatatgaaaacacctcataacaggcatataaacataataggcaactcataaaagaatacaaatggtcaacagatcaaacccattgtttgcctgtgcagtgtatattcaaaagctcttaatatctcctgttggcgggaggtgtgggaaaatggtcactcttacgctctactgggctacacttctacacaataatttgaaaagctggtcatttaaagccatttaaaactctatattctttgacttggaaattctatcagtttcagtgagtggataaactagttttgctaaccaaaagaaaataattcaattttaggaaaatggtaactttggtttaatagtataaatttcagactcctacaagatatccaaagggaattgtctagtaggcagttggacatatataacaattagcatttactcatggtgtttgctatgtttgctaatcttatatacattatttcatttaatctctccaaaattcaaagccgtctgtatgtaggacgaagattcaaacacagctcttaacccaaagctcttagtcaatattctattgtcttagacaatattcactgtccatgggtttgacacctggcagaaagatccaatatagaaatatatatttgcaagtttatctttcgttcactcattcattcagcagaaatttattaggtacctaaggttggatctacgaataccgagaaagtaaagttaaatttcttatcattagggacataaaacagagataggcatgaaaacaaaaaattgcagtatgatacaacagtgataatagaaatactaaagaggagtaacactaacccctctaaaaagagagagtagtcaactctgcatcggacagagtcatcaaggaaggttctaagaaaggaagacaatgcttggccatgattttaaaggatgagtgagaaaaaaaaaaaatcccccaagtggacagaagcagcaggtaaggaatttgattccagaagaagagcattgaatataaagaagtgtcacccaaaacagcatggtcagcacaaagaaatacatgtaggtcaccatagtgataaacagacagtgccctttaaccactctctaaccccccatcctctgctcctccagatgctgcaaaaaaggacagaggaaatataaagcctgccactctcttgagttgcacttcccaaagaggtccactcctttaacctcctcttcaaacggagaattggaaagggactaggcaggctcccttgtcaatgaggagaagcagtggtgtagtgacactctccaaaacagcttcccaacagcatcaccatctaccacctcacccagcctatggttttcttcatggcctttatttactactaaaattccatcatctattcattttttccttgtgtattgcctttctccgtcattagactataagctccaggaaggcatgggatgggcctcatctattctattctattcaccactgtggtttttctgttttttgtttgagacaaggtcgggctgtaagtggagtggtgtgaccatggcccactgcagcctcaacctcctgggctcaagcaatcctctcgcctcagactcccaagtagttgggactacaggtgcatgccaccacacctggctaattttataatttttattttgtaaagacaaggtctcactatattgcccagtctggcctcaaactcctgggctcaaggaatcctcccacctcagcctctcaaagtcctagaattaccatcatgagtcatcctgcccactgtatcttgaagaacacctgccacaggaaagcactccacaaatccttgctgaatgaatgaatattgtttcatcttacccaacatacagcattatggttattttaaacctaaaactccaatcaaaatcatatttcacttttataagataatactttctgtaacatttcacatattttcatggtctaatgaaattttccaatgctctaaatagcatccctatgccctctattttttttttcaccacaacaaatatttaaaacaaaatctgtctccaagtttaactctgctctatacccactccaatatttcattagagtatctttggaccaaagagaacaaggtaacaattttagattaagtggtatgccaactacaaattgatgtactgcttcagtgaaacatacatgaagaaaaatggaagctattctatacttaacgaaaaacttcaagtcctgctaataaaacaaaaatatctcaggcaggagaaaaaaaaaagtgacttatgttattggaaaaacaggtaacatatgacaattctaaggaggtcaccattttttaggaattagtatcactgataatagcaaactcatagagaccaggtcttgccaagactgcatgtgatataaaatgcttggtaaaatgtttggcaagctcctggaatctgtttatccaactgcctatttcactttgggtttcttttgagcaacctaccactattttcagaatcacccttccagcctacttgtgtcacagataggaaggtctttccctggcattttaggctttttattttttttttttgagacagagcctcgttctgttgcccgggctggagtacagtggagcaatcttggctcgctgcaacctccgcctcctgggttcaagcgattctcatgcctcagcctcccgagtagctgggattacaggcatgcgccaccacgcccaactaatttttgtattttttagtagaaacggagtttcaccatgttggccaggctggtcttgaactcctgacgtcaggtgatccgcccaccttggcctcccaaagtgctgggattacaggcgtgagccaccgcgcccggccgcattttaggctttaaaaggatacaggaaactagtactgcaagcttagccgtgccattactgaactgattaaaatttttgacacaaattgtaaatcaggcaaaatgcaacttcaaaagttcaaatattatgaaaaatatttactgttactatgaatctatttcaagaccaatgaaggctgaagtaagacctaactgttgccttgccaaatccatttctcaatataaatgccatgtaaaattacacaatagatttactctaaccctttttacatgggagttacaaaacgaagcctttaaacccctctagggataaatttacttttctcagccaagtgttaacagcaataaatggaggatttacatgtaatccctaaaagcaaataggcagaaattaccaaccatgctatgactttttaactaagcattttatttaatctcacttgcataacagggcaatttaaagggtgccagttctaaaaatatgagatgcaaatgcaaaatagtgtccctaatattttaaagcaaattttctaggattgtatattcaaacaaatgtgggttggttttgggtttttttcagagtactcactaggaataactaaataaacacctattcaatcaatactgccatttcttaaaaataatagatacctgagaacttttttcaaagtcagtgcataagcaatgtcctttttttttttttgagatggagtttcgcttttgttgcccaggctggagggcaatggcacgatcttggctcaccgcaatctccgcctcctgggttcaagcaattctcctgcctcagcctccagagtagctggaattacaggcatgcagcaccacacccggctaattttatatttttagtagagacggggtttctccatgttgaggctggtctcgaactcctgacctcaggtgatcagcccacctcggcctcccaaagtgctgggattacaggcgcgagccaccgtgcccagccacgatgtcctttcaaaatagatacaaatgaacattacactgaaaaaaatggatttattcataggctaattacagcataataaatgagctatatggtaagactctttttgtgttaactgctcattggtggcccaccatcctagaactgtatttccaggattccatcagcatctgcttaatgagatctatttctctgctaagtattctttagataagtaaatcagactttatcaaaaacagaactcatattattgcaagccataatagctagtttgtgatttgtaagaccatgttattgatatcacaaattaaaaagagatgtcccatttagaaaaagagatccctttacccaaccatcatactctaaaaataaaaccaacatttcttcacacacaaacaaaatcaaggtgggaatacacaagacttcaacacaaaaaaatcttatcaaagggtcagcgtttcagatgttctaaacttgttttcaccagtcagaaatagtctagtctgccaaatactaggccacatggctgatgtcacttaccttcttgtattccaacctcactgaaaaatcaggaggaacctgtttacaaacaatttgtgccttgagcagagttctgatacctctaaataaaccccttctaatttcttatcacacaactcgtaagagttcagcaaaaataagaaaaaaactactagccaaatatacataaccactcttcacattttgatgtggtaaaaatgttcacttgctaaagcttcttttcttcaagacattctccttttttgccccctcttccttccacttacatccttgctaaacatttcagtattattcagataaccagtacaccctagtcaagaaaacgactttctgaagaagctgagcacagcggtattctggaataaaatcccactgggctctgattagaagtgttaacactcctgcccatgtacaacctgtcgcacctgccaccagccccaaaggctgggactaccacagaacctgtttccagtgttcgacgcctcctgcaaaacatcacttactgtgccgggcatatgacttctctcattcttcccattctgagagctgccattttttagtttctttttcttttttgctgtttctttgtgctctttctgtttgttttgcacttcaataagaacagaaataaagctgtttttcacttccttttcaaactccagttcatctcgtaaagccaactgctgcaccagctcctcagagtactccttaatggcagtctcaatttcttccaggatttcatttaactcagacactgagagccttttcactcctgtgaccaaaagcaaaatacagcagatattaagacaaaaacatttcaaggaagcaaaacagagtagtcactaggaatgactaaataaacacctatgtaatcaatgccaaagagcaaaaatcatattccaagcaattaaattgactgtgggtgatcttctcagaattctacaacttctggagatcagatctgagtagaggtaagtgggcaggtgagtcacttctcagcttgctggcaagaaagtctaacaaaagtttttacttgtgcaaataaatcattgcatgaataatacttcagtcaagactgtgaaggcttgtacacggaaaagaacaaacacaaaagtcgagggaaagtacagaatccacctgctgacaaaaaactgcaagccgttcagcagggttctgggatatctctgaacaggaaaggagttgatctgaaagaattaaactaaatatacacttaattacataagtcctgatctgtgaattttgttgaaattggtgatatggtttgtatctgtgtccccacccaaatctcatacagaaatgtgatgctggaggtggggcttggtgggaggtcactggatcatgagggcaatttctaacagtttagtaccatccccctggtgctgttctcatgacagagttcttatgagacctggttgtttaaaagtgtgtggcactccccccagcccccttcctcctgctccagccatgtgaagatacctgctccagctctgcctcccaccatgagtaaaagctccctgaggcccccccagaagcagatgctaccaggcttcctgtatagcctgtggaaccattagccaattaaacctcttttcgtataaattacccagtttcaggcattttatagcagtgcaagaatggactaaaaaaactgggaagacaaaagggaacaatattccctgaaaccccatttcagtcttttctcagcacttctgccccttgtgacagataggacactgttaaagtcccaacccacagggcctcagaatgtggagagggggtctttaaagaggtaaattaaggttaaatgagggcatcaggataggccctaacccaacatgactggtattcttataaaatagggtgatcaggacacagtaggaagaccacgtgaagacatagggaaaaggcagccatctgcatgtcaaggagagaggcctctgaaaaaaaccaaacctgccaacaccttcatctcagacttctggcctccagaactatagaatataaattcctgttgtttaagccacccactctgtggtactttgttctggcagccctagcaaaccaatacagattttagtatctgtatcaggtatcaggactgtatttgggagtcaggacgttaacactgtttagcccatacctacaggaacatggccaaatcagaccatctgaaagaatttggtctgtctgaccatctgtatgttgaagccatacaacccaaaggtctttaggtaactaagctttggactgggcatgccataaagataaaactaggtgacaacacgagttgagcagatacagaccccatcagtgcctattcaaggaagggaccagtcatatggcaatgattagatacacaccttccaaccagcctcaggctgttcacaggctggttatatttattcagacatttttcatgaaaatcaactgtagagtttctagagttcattattctcacatatgtcaaacagatccatggttcccaaaattcttcatagtaacacacaagactatttgtacaaacaggaattgcatttgacaaataattactgaatatctattctacacttcaaatatgatggggtttcttatagactcaattgtgtcctccccaaaattcatgtgttgaagtcctaaccccaagtacctcagaatgttacagtatatggtctttaaagaggtaattacattaaaataaggtcattaggatagaccctaatccaatatgattgctgtccttgtaagaggaaatttggggccaggcaatggtggctcacgcctgtaactgcagcactttgggaggctaaggcgggcggatcatgaggtcaggagttcgagaccagcctggccaacatgacgaaaccccgtctctactaaaaatgtaaaaattagccaggcgtggtggcgggcacctgtaatcctagctacttgggaggctgaggcagaattgcttgaacccgggaggcacaggttgcagtgagctgaaatcatgccacttcattccagcttgggccaaaaagtgaaatcccgtctcaacaacaacaacaaaaatgtaaatttggacacaaacacaaacagaaggaggatgaaatcaaagctttctggagatgatcatactatacatttgatacagacaaaaagcacagaaatcattgatttcagcaggctcccttactctcttcataactgccggtactagacctcttgagagtttgaatttcctgggaaagcattgaaagccgatctgactgtgtaggggtttcatcatcttctgggtccggtgattcctgcatcatttcttcaatttcttcaataaccttcgaaaacacacacaccactgttaatcaaatatacaaaaggaaaatgatctatctggaacacagtgctcacctaaggcacccagagcagaaatgcactgaattctccattaaccaatggtgttcaggtgtggatgctccaaataatcaaaatttttactgtaggagtgaacatggtttatcaagaaaaaattacaatatactaaacacaaacctaaatagaattggatttactacgtccatcatgattcagaaggccctaacatgtcttgcaaagtctaggtaatcgtgctacaaacacataataatttacttcagacacaccagagctagatcatacagtaatcgttagaagaaagcaaataacagaaaatttaccatagacatacaagtgctcaatttaaaggtatctcatcaaaaattctgacataaaatttataaaatacaatagcacaaaaaaagtatatataaatgggaaattacctctatgacagcaaaatattctttcaacagcagaaaaataattttatatattaattcaacgaaaaaaagggattttttaaattactcaaaaagtagaagtccaaagagaaaaatgctgtgaaaagcagaaaaacttcagaaatacatttgactttaaacaatgatctactgtctagtgcagtgctgctcaaatcttacaacagtatcttgcccttttccagattctaaaagttgagaaggatatagaactcagcgaaaatgtaaaggaaacccaggaaaattactcaatagttagaaaaaaggacctaaagttaaaaaaaaaaattaaacacaattaaatgaaggagtaagacatcatgagcatatttccatgctttatgaattaacaagcttaacaggtaagatttccaccaaggaaaaacaaaggcaaatctgaaggagatgggattatgttaattataagaagagtctccaaaacaggaaatataataaggtacaatgagccactcaaggatcctacagaatctgttattctaactcttaaaaaaagggagagtttacggtattaggcaggtcagtgaggggacaatgaattctcaaggtcatgtctatccctagattaccctctacctcaaaacaaccttccaacctactattaccttgcttttttaagcaccacattcttcccacgcctagaacaattcaaccacaaatatgactaaggaggatccaaataacacaattttaatgtaaatttttaatgtactcctcccaagcttgccataatgtatctccttactgatctcatctttccagcctaaaaatagagtgtggtagaactcacagtccgagtagccatggaacattttatttcaggaagaagttctggcatcacatttcaacaatacaacagaggtgggaagagtattcagaaaatgagaaagctgattcgactggttttaagtgaatacagggttgaccttattagcgcattccacgggaataatgctcacttctttctggggcacacaatatgttactttaatacatgctaatgctttttacccagggttttgaagatactaaataatttctacacagatactttacagtttgtccgtgtttgggtaaatctaaagaaacaaggaaattcacaatatttttctggtttggggttaagcaacaacctttccttggtaccttgctataatcttcaacaaggcctctgttggaaaccttcccaactaacataatcccttctgcttcagatcacaaatacttatttttctctgtggaagagttaactattaagcctgtataaccagaaaaaaataggaaagataagtaaaccaacagccatcatttactgtcataacaagaagccgtttttcagctctagagtctcctttccttgcgttgctgaggatctcctctatacctggtctgccgtgaagaggggttcatcattaacacaggagacgatgattgagtgcatatccagctgttctctcagctcttcatcatctgatgtatcaaagagcaaactgtcacttacctaaaaacaaaaatacccatcattaaaagcaggacatcctcatcaaaacaaagttaacagctctgagtaaaagaggctgcagagaaaagcaactactaagagccttccctcaagagggagaaattattacaatcactctgtaccaggcaatgagtatatattcatcttgaacagaaatgagaagaatgtagaataaatatgaaatcactgagatgctgcataagactgctataaaagctgacagatatgcagaaaaaaatacggtattcattagcaagtcatagggaattacagtgaatagaagggagaatatgggcagggttagcaacacacactaagattggtagacgttaacattctaaagaaaacataaaagttagttacttgcaaactaacttctaatatctgagtaaaagatgatcaactccctgaaaggcgggatctgtccatgagcgtcacacttagcccttccaattgcacattccttcttaacaaaaaataataaccctaggttccaaaaacatctttaaacctaactaagctggtgattcaccactcacctcctcaccatcttccaccctgctctgcgctccaggaggacgacctttatggaagacatcaaaggatgcccttgctgtctggcttccaggtgggttaaaccccattggcagacactggcagactggaaggtgggatggtgaaggctgggcatgtattttgctgtcttcctccctgtcagactgtcatttgttgactatggctcttcaccaaagccatagctctgattagcagacttctcccacaaggccaatctcaacagtttatggtaatttctccctctccttacctcttcatgcccaggactgctaaagtaccagtttctgttgccagccctgggggattgcaccacatcaagtttgtttcccttaaccctgcccacaccaatataaacagccttagagtttagtaccagtactgattgtaaaaactttgcctgaaaaaactcagcagattaaaatgaacaagggaacatctgtctatcatctacctatctgtcatgacatgactattcaggtgttgaatggaactgaaactagaactctattcctcaggaaggattagcacacactgagtttccaaagaataaatgggcaacaaggattacgtaagaagccaccatgtcattctattacatagtgtagaactaacatgataaaattttacctggcaaaattatttatccatgcagaggatatatgtctaaagctgaggacacaatggaatggatgtaggttcctactgcttccctagttcttcataacatgagcatctgaagtttgcaggtgaacacagcctacagagtaagaaacctgcacgtgagaagggccaagggaagaagcactggcacactgactttccaacattaggctgggagaagggccccatcaacactgcacggtccacctacaaccctataatgcaactccaggctcctgcgcagtgacgacagcccttctcattttatttcttactgcaccatcagcctagcaaaggcctggccatgaccttcaacacagcatcatgaactaagaacactgtgtggaaaaaaggagaatctaaaagcttccagggagggaaaaaaaaatcacattaaaaggatcaggaatcaaaatgtcaagtgaattctcaatagcaacaatagatgctagaaaataatcgatgtaacttccatgtttggagaaattccatcctagaatcctatatccagctaaaacaagtaggcaggggctaggataaagacattttcagacatacaagttctgaaaaaatttacctctcatgtgccctttctcaaaaagctaccaggaggaaaatgaaaaagaaaaaagatatgtggtctaataacaagtgacaaagagaattctcgtaatgatagtaaagtgaaaatcccagaatgatgactgtaaagctacactagagagtaacattcagattagaatttttattgcagagagcatggagaagagggagaggtgggtggggagggagagcagaaggactagatagattacctagtgcggtccatttactaaaatgagttctacggctctattgaggctgaactggtcagctaattataaactaaataaaccacaaaaaggcaggtagtatcctaagaaaaaacctaagtagaaaaagagaaaatgtaaacataatatatcataatgggtcaactgtgcacatttaaatagtaaatataaatgatgaagattaattcaaccccagatgttatgtaattacattgggaagatggggaaagggaagtttttgggagggtatagtgtcagggattggcaaaggacagaagagagctaaaccttcaccctctgggtagaaaataaataaagtctaaaattgagaaaataatatcaagtaaaatggcatgagcatgttatctgctaaaagaaatagctgatatcatttaaagtggccacctctggtaaggagaaatcaagtggagcagagactgccttttcatttaagtcctatagttctaatttactttttaaactatgacagctgttcctttaataatttaattttttaaagaaggagaaagatcaagagactaggctcttgatgagaggggatgtatgatttctatactgtagaagatgaaaatgtggcacactgtgatcacaaaggcatgtcgataccatggagtagacctaccgattagtttcatcgatccttacacttcgaacacttcacacccccttcccaatcactcttcctatgttgcaacttgaggaccatgattttgttttatttttccccttctggcttataaatactaaaagttttaattttaaaattaatccggggtgggcacagtggctcatgcctgtaatcccagcactttgggaggctgaggcgggtggattgcttgagctcaggagtttgagaacagcctgggaaacatggtgaaaccccattgctacaaaaaattagccaggcatgatggcacatgcctgtagtcccagctattcaggaggctgaggtgggaggatcacttgagcccaggaggtggaggttgcagtgagccaagatggtgccactgcgctccagcctggacaacagagccagaccatgtctcaaaaaaaaaaaaaaaaattactcctaaggaagcccaggttgccatggctcacacctgtaatcccaacactttgggaggccaaggtgggaggatctcctgaggtcaggacatcaagaccaccctgagcaacacaggaagacctcgtctctacagaaatatattttcttaaaattagccaggcatggcggtactcgtgtgtagtctagctactcaggaagctggagcgggaagatcgcttaagcccaggagttggaggctgcagtaagctatgatggcgccactgtactccagcctgggcaacagagggagacactctctcaatctatcaatcaatcaaattgcttctgtggaagacattatctttttcttatttctactcttaaaggtataaagatgcatacaaaagtccctgtcctgaaagaaatgcacatggaaggagaaatacagcacagaagatgttcttaagctgaaatgaatgataagcattctatcatataagtatattcaagaatggcaccagatttagaatagggttatggccaaaacacatgagtaataacagtaaactattataataaatttccatgacaacagaaatgatcccagatcatctttccagagagagagccaagtccattccccacagagatggcatatgctaagagctggaatatctttcaggctttgcaaactagccctggctgtcatgggtatcattattcactcctcccttcagcattaaatgtgccttctctttgccaagcattcccatcactccttccaatgccagggttagatacagacctggtggtactgtttccctgtataaaactgaagatacatcaaatggaaaaaatgtgcccacgtaagaatttgaaagataccagttgccttactctaaaaaagacatatcaaatttagaagtgtgccatcttaatcttcaatcaaagaaaaatttaaaactgcaatctagcaaactagttttcctgaatgtttttctctttttaatgcaaaaaccattcttcttttgtcaaaaaaaatttctatacacttggttttataagtaaaataatctaaagaatgctttcaaagggtaaattttacatagcttagtcacggcacgaacagagcaccaggtggcaatggtaaatacagtcactttgccagattaaaaaaaaaatgcttaattatataccatgtaatagaatgacgtgatgacttcttacatagtccttattgcccatttgtttttcagaaactcagtgtgtgctaatcctccctgagaaacagagttctgatttcaattctattcatcaccagaatagtcatgtcacaacagagagatgataggtgaatgatcacgtgttcactttcatctgctgagtttttccaggtaaggctgtttttttttttattttttgagacagagtctcgctctgtcacccaggctggagtgcagtggcgtgatctcggcgcactgcaacctctgcctcccaggttcaagcaattctcctgcctcagcctcccaagtatctgggattacaggcatgcgccaccacgcctggctaattttgtatttttagtagagacggggtttcaccgtgttggtaaggctggtctcaaactcctgaccttaggtgatccatccacctcagcctcccaaagtgctgggattacaggcatgagccaccgcgcccagccaggtaaggctttttaatgtcagtactagcattaaactctacgtcattactaatcactgccttggccatatcattaccgtggcattgaaaataacattcaaaaggcagcaaaagggagcaacctcccaagatgcaatctaagaacaatacaagacatacagcagtccctcggtatccaagggaaatttgttccaggacaccccaatagcaaaatctgcagatgctcaagtcccttatataaagtggggtagtatttgcatataacctgtgcacatccccctataagctttaaattatatgtaaattacttataatacctaatacaaggtaaatggtttatagttatattgtatataactatacaatatttatattataaagttatattgtatcggttttcggttttttattttcattattttcaattgttgtgttgttattttattgtggggtttttttttcaagttttttaatccacagttggctaaagcagcagatgtgaaaccagagaatatggtgggcccgctgtacatttagtcaatgaaattggattaaactgtctcactcacgctgctaatgggcctgactttgaaactatgaatggaaactcttctatgctattttttaatatgtcaaaaaagtagtacccatggactggttttggtgcattctgcattattttaaataacatagaaaatatttttttaaaacctttctatcacgatctctggccctacagtttcactgacaaggctattcacttattcactcaataaaaaggtatcttcaaacttactatgtgccaggaacagtatagttactataaagacaaatacaaatacctgccgggaagtcacaatctagtgacagacccagaaacatatgattaacaagagaaggcagtggtaattttagagacagggacaaagtgatctggggactagcacccatgagacagcaaggctaaaccccgtggcacagataacaagtcctttcaaagttcacaaccccacaagtgccatcagttcccctagaggggagtgtgttttccttccgtgcaggaatttaggccatggctaaatgaccatctgtcagagatgctggaggtgaggattccccccaagtgagaggctggattaagtgtcctttaaagccccttccaactctaaaattgtataattccatgagatcagcaaaggctaaaacagtaataggaagcttcagagaggaagggaaacttgaactaagcccaaaatgatagccaggacctaagtaaacaagacagagcaaaagatccctcattttattccttttcattaaatatatgccaggtcagtctgcaaagcttaatgggattaatgactggcacagtcaggaagatttccatagctactgaatactgggaaatacagccagaattctacattttactagaaaataataacagtaggcttttctgatcacatatgtaagaaaatccttttaacaaaacattaacaatactatgtaaaatagtgacaggcctactgcactatattctcaataatcacataattacacttaatgaaatatagagcacgcaagaagatctggcatgagtttacattattatttgataaggaacttaaacagccaggccctgaaacttagataatttggatatgtttctcatcagatttaaaaaagcttaacactgtgattttccatttcaagtaattctcaacccttttctgcctccctacctgtcccaccactcgtgctcacacttcttagtcaagacatcttcactttacaataccaggatgtacacaggagaccccaagaaaggtaagttagccaagaccgtattttaaaagccaagaactaaaatatcttttaaaggttctccagtattcacaggaatacacagtctcctgacaacatatcatgtctagcaggtcactacactcattaaaaagttaactccgtgttgagattccaatactctcagtatattaaaccaaaggctagcatttttactgagggtttcagtgcaccaaattttactgagtgtttcagtgcatcatcactgccaggtgcagtggctcacacctgtaatcccagcactctgggaggtgaattacctgaggtcaggagttcgagaccagcctggccaacacagcgaaaccctgtctctactgaaaatacaaaaattagccgggactggtggctcatgcctgtaatcccagctactcgggagtttgaggtgggagaatcgcttgaacccaggaggcagaggttgtagtaagccactgtactccagcctgggcaacagagtgagactccgtcaaaaaaaaaaaaattgtgatggaaatttagctacacataaaaaatgagtgtgctggagaagaactggaggaatgtgccagcaaggtgatggccctacagccaagacatcaagacagagccacccaaactgctataatttcagaaatgagaggcaggtcagtgaatctgtgttaagcagaaatgctactgttttagttctattatcagcatcagttattcaaacactattggttcaattcctaacttacccctttttctgagaggttcagagtaagcaagtgcaaggtcctagtatgcgatgacttccagtctacaggcatcacattcccataattatctgtcagggcattccaaatccttaaaaagaagaaagctacaatcaatgaaaattaacattctgtatgtctttcttaaacaaatattcagtgaaagatgaacagcaaatgtcaaaggaaacagagaaaaacagacagcacattgtttctcagaaaaacatcaacaaagtagagtctcctaaacaaaaacgaggtgaaaaatcacataactgggctgacaagaagtcctaagggaaggtgaagccgtacagaaaactgccaataggcagtaggcaacacttccattatattaggtgctaggcactgttctaagcaatttacatgtattaactcatgtaatcttcaaaaacaacattagaagggggaaccattattaacctcattttacagatgaagaaactgaaacacagggcggggaagaaatgtttccagggtcacatgaataataaatacaactgtgcattacacttatttttcttctgtaacttcaaacaaacagaccatttcaaaaagtgaaaaatctctctcaaacaaaaggtttgttatcctttctggagaaagaattcgctctcaatgtgagaacaggatttaatatctaatttgtaataaagaagctaagcactaaggcagatacaacaaaaaggagaaagagagggagttcacaataaaacacacccaggctctatccaatgcagttacctgaccttattacccaacagcattacagggaggcaagagggtcaaatggtaagggcatttgactgagactagggaaacaaactctcatctccatccttcttcaaataacgagtcattggccaggccccttaacctccctaggtatcctctgcccttctataaatgagagacctggacaagaagcagatactccttccagctccattactctaagcattaattcaaatgaggtgtattgaagagagtcccaaaccacaccagataagaacaccaaactcaaatttcacaaccaaaattcaggtttcaataccaaagtaataatcagcacaataaaacatgatgttttatgaaaaactaaagttcagagatacaagtgacttgaaatctaattgggatacgtaaaatcaaacactaaacatacaagctacttactagctagaggtatacacaaagatgtcatgtagaaaaaaacaccctaaaatgtttattaggtatctaattgtgaataagcaataggctttctttccaagtgtgtaagacactaggatacaaaaacaatcctggctggggcataaacataagaatgaattttttttttaagttccggggtacatgggcaggatgtgcaggtttgttacacaggcaaacatgtgccatggtgatttgctgcacctatcaacccatcacctaggtattaagcccaagaatgaaaattttaaaatacagacataacatagttaacaaatagggattgggcgctaaaaagaagcaataggactgcaacggctaaaagcataacatcccaaatgatgtttggctttaaaatgcacgttagggatgggcctggtggctcatgcctgtaatcccagcattttgggaagccaaagtgggaggattgcttgaagccaggagttcaagaccagcctgggcaacaaaagggagccccacatctctacaaaaaaaaaaaaaaaaaaaaaattggccaggcctgtggcatgtgccgcagtcccagctacttttggagggtaaggcaggaggactgcttaagcccaggagctcaaggctgtagtgagctatgatggcaccactgtactccagcctgggagacagagcaagaccctgtctctaaaaatatataaataaaataaaaaataaaattagccttaataaaaatgtgaacccaaaaatctctagagaactaactcatctacccagcaaagctcccatatgcacagtccattcctattctcctctctgttggaatatctcctctaatgccagtgaagtagaattagaggcaatgtctgtggactactaaatttgctgggtttctgtggactactaaacttgctgggtttctggctcataccttcctggataagaaattttcaaacactgaatgactgtatagtccattttcatgcagctgataaagacatacccaagactgggacgaaaaagaggtttaattggacttacagttccacatggctgaggaggcctcagaatcatggtgggagacaaaaggcacttcttacatggtggcagcaagagaaaaggaggaagatgcaaaagcagaaacccctgataaactcatggacagcggccctcttctcacaaccccactaggcagtgccccagtagggactctgtgtgggggctccaaccccacatttcccttccacactgccctagcagaggttctccatgagggccctgcccctgcaacaaacttttgcctgggcatccaggcatttccatacatcttctgaatctagaaggaggttcccaaacctcaattcttgacttctgtgcacctgtgggctcaacaccacatggaagctgccaaggcttggggcttgcaccctctgaagccacagcctgagctgtactctcgcccctttcagccacagctggagcagctggtacacagggcaccaagtccctaggccgcatacagcaggcagaccctaggcccagctcacgaaaccacttgttcctttttgggcctccaggcctgtgatgggaggagctgccatgaaggtctctgacatggcctggagacattttccccatggtcgtggggattaacattagggtccttgctacttacgcaaatttctgcagctagcttgaatttctccccagaaaatgggttttttttttttctatcgcatagtcaggctgcaaattttccaaacttttatgctctgcttcccttataaaactgaatacgtttaatagcacccaagttacctcctgaatgctttactgcttagaaattttttctgccagataccctaactcatctccctcaagttcaaagttccacaaatctctagggcaggggcaaaatgccaccagttctctttgccaaaacataacaagggtcatctttgctccagttcccaacaacttcctcatctccatctgagaccacctcagcctggattttattgtccatattgctgtcagcattttgggaaagccattcaacaagtctctaggaagttccaaactttcccacattttcctgtcttctgagcccttcaaactattccaatctctacctgttccccagttccaaagtcatttccacattttcaggtatcttttcagcaacgccccactctactggtactaacttactatattaggttattttcatgcagctgataaagacatacccgagactgggaagaaaacgaggtttaattggacttacagttccacatggctggggaggcctcagaatcatggcaggaggcaaaaggcacttcttacatggcggcagcaagggaaaatgaggaaggagcaaaagcggaaacccctgataagcccatcagatcttgtaagacttattcactatcatgagaatagcataggaaagaccagcccccatgattcaattacctccccctggatccctcccacaacacgtgggaattctgggagatacaattcaagttgagatttgggtggggacacagccaaaccgtatcactgactatagacctaatcctggcaggaatttaagcaggatacactagcaggtgaagacagtccagaatttgaggagtttgctttgtctgctgatttgctccttcttaaacaaggctataagaaaaaggcaaatgggatggaatatttcacttagaagtgcctgtcagtgagtttgatcttccaagaaaatgaaagccaactaacagtctccaccaaggtcaaaacaatggtagttggaaggacacagaggcaactctagtgaaagatttctagaattctgcagtcaagacatatgaactgccttcatctttatcaccaagtattacaaccagtcgtaaacaacggtcaggacagctcttttgcaaggaggtcaaagtcctgccaacatcgaatcctgcagaatactcttataagtaaaacctcagtacgtaaatacaatccatatttaaccaaaacaaaagctgaggctcagagaagttaaatgaagtgccaagggtcggggagggctttggggtgattcaagcacattacatttacggtgtactttatttctattattacactgtcatatatcatgaaataattttacaactcaccataacgtagaatcaatgggagccctgagcttgttttcctgcaactagatggtcctatctgggggagaggggaaacagtgacagatcatcaagcattagattctcataaggtgtctgcaaactagaaccctcacttgtgcagttcacaatagggttcacgctcctatgagaatctaatgccgtggctgatctgacaggaggtggctcaggtggtaatgcaagcaatggggaggagctgtaaatacagatgaggctttgctcactcactcagccactcacctcctgtagtgtggcccagttccagtaccggtctgtggaccaggggttggggaccccttgacctaaagcacagcaatatctgagtaaaacaattatcaattatatctagtgaaaagggaactggaacagactatcaagcaatgatgagtatgtttttgcaatataaaagggacaatgccagtgatgaccagctcccaactcccggaaaaaaagagatttcatatgcattcccaagtcaagagacatgcattaccaccaacatatcatctactaaataccattaacaagagcgacaaaatatttttaagcaaaggctaaaataacctactctcctgacggtctaagtgaaacaaaaactcttaacgatctacgtacaatcctctaattgatttaagtcagctactcttgaaaaacccaaatacataatacatctacactaatttttaaatccagaatgtgtcctgggtttagtgattcagagtatctaaatctacatatacataaaattatcttctctttgggaacaggcttccaaaaatgtcatcttttgctactgacggcatgcatgtgtgtataaaatccaattataagtgtttcccttctctacacctgggcagaacactgtactgaggaaccaataaaaggagatggtctccaaacacaggtgggcctgcaccactgctcagccatgtttacaggccccactcccaaggctgtctttacaagcttcctaccttattcctgataaatctgtttgtttcacaaaggaaaggagacaatcaggctggaaactcctttccgtgcccgtgactcctgtcctgtctctcaaaaattcttccctaacctctaagagtctctccatcccatcaatgctctgaaaatccattccttcccatgtcctcatttttgagacaaagtctcgctctgacacccaggctggagtgcagttgtgcgatgatggctcactgcaaccttcgcctccaggactcaagcaatcctgccatctcagcctccgggcagctgggtccgtgggaccacggggcatgcgccaccggacttcactaatttttttttaatttttttgtaaagaccagatttcacgatattgcccagtctggtctcgaactgctggaagcaagccatcctcccaccacagcctctcaaagtgctgggatcacaggtgtgagccaccgcgcccaatcttattccactcattttaccttcagcctctccggaggctcctctccccctatatacacaacatgctcaagtccctttactctcaccattcctatttaccatcttttgggggtaaaaccatggtacctaattacaactcttacttaaccaaattataattgcggctcaggtggtttaaaaagtgccctcaggaactctgcgcctcacctctcccagctgtgataaaaccccgtgaacctcctatcattgctcttaggtgattctgtaattattaatattacttgtgtatgccttggtctccccaccagaccatgcgatcgttaccatcagtaagcagggcccttcgttttcgtctctgtggcggctcgcactctaccagggctcccgggtgggtcccacagattcctgtgccctaaatgctgcctcgcacgttggtacttatgcacaggcaatatactccacctagaacgttagacttttttccacctgccaggcacactcccactcatcagtcagatctcgtttgcacatttctttataaagtcgttccttactctccccaagacacacgcttttaagtctctcacatttggtacatataaccattgcagcgctaattacatccatacatgtatttgtttacaaatctatctcccatcctacgttgtgtcctaagtgggagggacagtgcccagtcgttttggtgttccctgtgcgaagcagcgcctggcactcagcaggtgcttaataaatgcgacttgagtgaatgaatggcgacacacgtgccccaggaggtgccccaggaggtgccgtctctaactatgggctctgcatgggcgattcccttctaatacaaacgagggtttggtttgcaggaaggagggcgggaggtaccgaggcggaggttttcagggctcgtggcggcggggggcaggggatttaaattgctgggcgagccgaggccgacggccggaggaaggaggcgagagggcggggactcccgcgtccgggcggaaggagggcgcagggaggagctgcggccgtagggctgccggtcgggcgaggggtaggggaggctcccgcccactcccgccggggccccgcactcactcgtccccctgcaggaggctgcgctccgtgatgggccgcacggccgtcctcgggggctcggcgcccggatccgaggggcggaagcacaggctcagcttctcctccaagctgcaggccggggccgggaaaccgtcggcgcccccacccgcctcggcccccgcctccgccccaggctcggggctcgcgttacagttctcctggtccaggaggctccgggccggctcctggaactcatagaaatcctgccagtccccgtccgccgccatcgccgcccggagcagtcgcgcgccccgcccaggccggcccagcccgcccagcccggccccgccccggcccgaagccccgcccccgcccgctaccgtgctgacagatcccaggctgtcccaaccctgggtctccggtggcctgagtcttcctggagttgaacgcggtgctactagaaagccaagaccttaactgccttgagtcacttcctatattctctatcatttaggaataacttccctgttagcttcccttattagcaacttggcctaaatgtggctaaaataaaggacttctttaaacttacgacctaatatcagattacgccccgaaagtcgttttccccaaggtaacctgctcctttcctaacttgccaaccagaataacagccagagtcaatgggaaatgtttaggtggtaccgaagttcattcctcaaatattttacctctcacgtgcaaagcactggctagatcctgcagaacatacaaagataattatgccagttaacatttgcaaagcacttagagcaatgcctaatagtagataccctatgtgtatgttataactaatacatgggtgccccatacacagggcttacaatgtcggagtgataagagtaacaagaaattgtttagacctcataaaaactaagtgtgattgggcggttcagttttgcaagatgaaaagagttatggagatggatggtggtgatggttccacgacagtgttaatgtgctgaatgccactgatctatacacttacagatggttaagatagtaattttatgtttattttactacaatacaaaaaaattggagtaggccaggcgaggtggctcacatctataatcccagcactttggcaggcccaggtggacggatcgcttgaggtcaggagttcaagaccaacctggccaacatggtaaaaccccgtctctactaaaaatacaaaatctagctgtcaggcctctgagcccaagccaagccatcgcatcccgtgacttgcacatatacacccagatggcctgaagtaactgaagatccacaaaagaagtaaaaatagccttaaggatgacattccaccattgtgatttgttcctgccccaccctaactgatcaatgtactttgtaatctcccccacccttaagaagtttctttgtaattctccccacccttgagaatgtactttgtgagatccacccctgcccgcaaaacattgctcttaacttcaccgcctgacccaaaacctgtaagaactaatgataatccatctcccttcgctgactctcttttcggactcagcccgcctgcatccaggtgaaataaacagccgtgttgctcacacaaagcctgtttggtggtctcttcacacggacgcacatgaaatttggtgccgtgactcggatcgggggacctcccttgggagatcaatccccctgtcctcctgctctttgctccatgagaaagatccacctacgacctcaggtcctcagaccgaccagcccaaaaaacatctcaccaatttcaaatc

(19) United States
(12) Patent Application Publication
(10) Pub. No.: US 2003/0082714 A1
(43) Pub. Date: May 1, 2003

(54) Novel nucleic acid and polypeptide

(75) Inventors: Melissa Little, Brisbane (AU); Toshiya Yamada, Brisbane (AU); Carol Linda Susan Yamada, Brisbane (AU); Gregory Holmes, New York, NY (US); Kylie Georgas, Brisbane (AU); Gabriel Kolle, Brisbane (AU); Lorine Wilkinson, Brisbane (AU)

Correspondence Address: GREENBLUM & BERNSTEIN, P.L.C., 1950 ROLAND CLARKE PLACE, RESTON, VA, 20191, US

(73) Assignee: THE UNIVERSITY OF QUEENSLAND, Brisbane (AU)

(21) Appl. No.: 10/152,724

(22) Filed: May 23, 2002

Related U.S. Application Data
(63) Continuation of application No. PCT/AU00/01435, filed on Nov. 24, 2000.

(30) Foreign Application Priority Data
Nov. 26, 1999 (AU) PQ4348

(51) Int. Cl. C12Q 1/68; C07H 21/04; A61K 48/00; C12P 21/02; C12N 5/06; C07K 7/06; C07K 14/435

(52) U.S. Cl. 435/69.1; 435/320.1; 435/325; 514/44; 530/350; 530/329; 536/23.5; 435/6

(57) ABSTRACT

A novel isolated nucleic acid which corresponds to a gene located on human chromosome 2p21-16.3 is provided, and a polypeptide encoded thereby, together with mouse and chicken orthologs. The encoded polypeptides share a PGECCPLP motif and include an insulin-like growth factor binding domain, cysteine-rich repeats, an RGD motif and transmembrane domain, and interact with members of the transforming growth factor superfamily. The nucleic acids of the invention, and polypeptides encoded thereby, may be useful in the diagnosis and treatment of diseases including eye defects, neurodegenerative diseases, renal and kidney disease, bone and tooth abnormalities, wounds and skin damage.

### CROSS-REFERENCE TO RELATED APPLICATION

This application is a continuation of International Application No. PCT/AU00/01435, filed Nov. 24, 2000, which was published in English under PCT Article 21(2), the disclosure of which is incorporated by reference herein in its entirety, and which claims priority of Australian Application No. PQ4348, filed Nov. 26, 1999.

### FIELD OF THE INVENTION

THIS INVENTION relates to a novel isolated nucleic acid, and more particularly to an isolated nucleic which corresponds to a gene located on human chromosome 2p21-16.3. The invention also relates to an encoded polypeptide which interacts with members of the transforming growth factor beta (TGFβ) superfamily. The nucleic acids of the invention, and polypeptides encoded thereby, may be useful in the diagnosis and/or treatment of diseases including eye defects, neurodegenerative diseases, renal and kidney disease, bone and tooth abnormalities, wounds and skin damage without limitation thereto.

### BACKGROUND OF THE INVENTION

Vertebrate development is a complex process involving a plethora of genes and gene products whose interactions direct crucial events such as cell fate, pattern formation, organogenesis and, at least to some extent, the development of intelligence and behaviour. Although an overview of this area is beyond the scope of this discussion, it has become clear that by understanding the genetic basis of development, the genetic basis of disease is also more properly understood.

Much effort has been directed at identifying the genes which control developmental processes and underlie particular diseases. Such genes are useful as tools for diagnosing disease and determining whether an individual is predisposed to a particular disease. This information is also useful in genetic counselling of carriers and affected individuals with respect to the possibility of transmitting the gene to offspring, and for the pre-natal detection of such genes carried by offspring.

Even more importantly, the genes (and their protein products) which underlie or contribute to particular diseases, may constitute new and efficacious treatments of those particular diseases.

There is still a great deal to be understood in terms of the genetic basis of vertebrate development and diseases arising from aberrant developmental processes.

### SUMMARY OF THE INVENTION

The present invention is broadly directed to an isolated nucleic acid which corresponds to a gene located on human chromosome 2p21-16.3, or a chromosome structurally and functionally equivalent thereto, and a polypeptide encoded thereby.

In a first aspect, the invention provides an isolated polypeptide comprising the amino acid sequence PGECCPLP (SEQ ID NO: 1).

In preferred embodiments, the isolated polypeptide has an amino acid sequence as set forth in FIG. 1, hereinafter referred to as a human CRIM1 (SEQ ID NO: 2), mouse CRIM1 (SEQ ID NO: 3) and chicken CRIM 1 (SEQ ID NO: 4) polypeptide respectively.

The invention also contemplates biologically-active fragments, variants and derivatives of CRIM1 polypeptides.

In a second aspect, the invention provides an isolated nucleic acid encoding the polypeptide of the first aspect.

In particular embodiments, the isolated nucleic acid has a sequence of nucleotides according to FIG. 2, hereinafter referred to as a human Crim1 nucleic acid (SEQ ID NO: 5), a mouse Crim1 nucleic acid.(SEQ ID NO: 6) and a chicken Crim1 nucleic acid (SEQ ID NO: 7).

In another embodiment, the isolated nucleic acid is pcDNA3-hCRIM1myc deposited under accession number NM00/16530 at AGAL on Nov. 9, 2000.

In a further embodiment, the isolated nucleic acid has a nucleotide sequence according to FIG. 3 (SEQ ID NOS: 20-24), hereinafter referred to as a human “Crim1 genomic sequence”.

CRIM1 and S52 are used interchangeably herein, due to recent changes in nomenclature during isolation of the polypeptides and nucleic acids of the invention.

In a third aspect, the invention provides an expression construct comprising an isolated nucleic acid according to the second aspect.

In a fourth aspect, the invention provides a host cell comprising the expression construct of the third aspect.

In a fifth aspect, the invention provides an antibody which is capable of binding a Crim1 polypeptide, biologically-active fragment, variant or derivative thereof.

In a preferred embodiment, the antibody is capable of binding a peptide having the amino acid sequence RVQVDSSQRMLRIAEPDARFSGFYSMQKQNHLQADNFYQTV (SEQ ID NO: 8) or the amino acid sequence KVCQPGYLNILVSKASGKPGEC (SEQ ID NO: 9).

In a sixth aspect, the invention provides a pharmaceutical composition comprising an isolated nucleic acid according to the second aspect or an isolated polypeptide according to the first aspect.

Preferably, the pharmaceutical composition comprises a pharmaceutically-acceptable carrier, diluent or excipient.

In one embodiment, the pharmaceutical composition is suitable for use in gene therapy.

In a seventh aspect, the invention provides a method of modulating the activity of a polypeptide of the TGFβ superfamily, said method including the step of administering to an animal a pharmaceutical composition according to the third aspect.

In an eighth aspect, the invention provides a method of detecting a predisposition to a genetically-inherited disease in an animal, said method including the step of identifying a CRIM1 nucleic acid mutation or polymorphism indicative of said animal being predisposed to, or suffering from, said genetically-heritable disease.

In a ninth aspect, the invention provides a CRIM1 mimetic.

In one embodiment, the mimetic is an antagonist of CRIM1.

In another embodiment, the mimetic is a mimic or agonist of CRIM1.

### BRIEF DESCRIPTION OF THE FIGURES

FIG. 1

Alignment of amino acid sequences of human (h) CRIM1 polypeptide (SEQ ID NO: 2), mouse (m) CRIM1 polypeptide (SEQ ID NO: 3) and chicken (c) CRIM 1 polypeptide (SEQ ID NO: 4). Conserved amino acids are indicated thus*. Conservative changes are indicated thus.. The conserved PGECCPLP sequence (SEQ ID NO: 1) corresponds to residues 200-207 of human CRIM1.

FIG. 2

Alignment of nucleotide sequences of human (h) Crim1 cDNA (SEQ ID NO: 5), mouse (m) Crim1 cDNA (SEQ ID NO: 6) and chicken (c) Crim1 cDNA. (SEQ ID NO: 7). Conserved nucleotides are indicated thus*.

FIG. 3

Nucleotide sequence of a partial human Crim1 gene. The 236303 bp sequence includes exons 2-17 of the Crim1 gene. The exons are located as follows: exon 2: 33104-33277; exon 3: 77747-77989; exon 4: 79104-79224; exon 5: 101023-101144; exon 6: 113378-113560; exon 7: 115986-116183; exon 8: 135708-135836; exon 9: 146472-146628; exon 10: 148762-148883; exon 11: 150045-150254; exon 12: 153816-154031; exon 13: 158581-158802; exon 14: 173983-174177; exon 15: 181007-181129; exon 16: 183613-183800; and exon 17: 185153-187765. The Crim1 genomic sequence corresponds to SEQ ID NOS: 20-24.

FIG. 4

Alignment of human and mouse CRIM1 polypeptide amino acid sequences together with a putative C. elegans ortholog.

FIG. 5

Structural analysis of human and mouse CRIM1 polypeptides.

A: Domain structure of the putative human and mouse CRIM1 ORFs compared with a C. elegans ortholog and Drosophila Sog and Xenopus chordin. The cleaved signal peptide (SP) is indicated. IGFBP=insulin-like growth factor binding domain; CR=cysteine-rich repeats; TM=transmembrane domain.

B: Alignment of cysteine-rich repeats of human CRIM1 (H-CR1-6), mouse CRIM1 (M-CR1-6) and a C. elegans ortholog (C-CR1-6).

FIG. 6

Northern analysis of Human Crim1 and mouse Crim1 RNA expression.

A: Mouse embryonic Northern blot probed with mouse Crim1-derived probe. The major Crim1 isoform of 6.4 kB is present at all stages shown.

B: Multiple Tissue Northern analysis of human Crim1 mRNA expression in the indicated postnatal human tissues. The major 6.0 kB is present in all samples except liver, whilst a minor 4.0 kB isoform is seen in placental RNA.

FIG. 7

Whole-mount in situ analysis with digoxigenin-labeled human Crim1 probe.

A: S52 expression in whole embryos from E9.5 to E13.5 and dissected kidneys from E15.5 to E16.5;

B: S52 expression in E16.5 kidney;

C: S52 expression in the floor plate and motor neurons of the spinal cord E12.5;

D: S52 in caudal somites E12.5.

FIG. 8

Whole embryo analysis of Crim1 RNA expression. Expression of Crim1 (A-E) compared with Sonic hedgehog (Shh; G-K)). F=E13.5 embryo expression in eye (a), ear (b) and vibrissae (c). At later stages (E11.5 onwards), lack of probe penetration prevents visualization of staining of internal structures. N=notochord; FP=floor plate; So=somites; MN=motor neurons; LB=limb buds; Ey=eye. Scale Bar: A, G: 80 μ; B, H: 100 μm; C, I: 120 μm; D, J: 145 μm; E, K: 160 μm; F: 300 μm; a,b,c: 40 μm.

FIG. 9

Analysis of Crim1 RNA expression in the embryonic mouse central nervous system. A-N=Expression in cervical sections across the developing spinal cord showing comparisons between S52 (A-E), sonic hedgehog (Shh; F-J) and Isl-1 (K-N) at E9.5 (A, F), E10.5 (B, G, K), E11.5 (C, H, L), E12.5 (D, I, M) and E13.5 (E, J, N). O-R=Expression in the developing brain showing comparison between S52 (O, Q) and Shh (P, R) at E10.5 (O, P=transverse section) and at E12.5 (Q, R=sagittal section). NC=notochord; FP=floor plate; MN=motor neurons; I1=dorsal interneuron subtype 1; I2 dorsal subtype 2; DRG=dorsal root ganglion; L=lens; D2=dorsal interneuron subtype (expresses Isl-1); DM=dorsal midline cells of hindbrain; RD=roof of diencephalon; VF=ventral forebrain. Scale bars: A, F: 20 μm; B, G, K: 45 μm; C, H, L: 70 μm; D, I, M: 80 μM; E, J, N: 90 μm; O, P: 80 μm; Q, R: 300 μm.

FIG. 10

Radiation hybrid panel mapping of CRIM1.

A: Primers 3′ PCR 3F and 3′ PCR 3R amplify human but not mouse cDNA. There is no intron in the genomic sequence as the band amplified from human genomic DNA is the same size as that amplified from human cDNA.

B: S52 is localized between the markers D2S1852 within band 2p21 and DS1409 within 2p16.3 on chromosome 2. pter=terminus of short arm p; qter=terminus of long arm q; Cent=centromere.

FIG. 11

Immunofluorescence of 5 day kidney explant culture with antibodies to Calbindin 28K (Sigma; used at 1:200) and polyclonal anti-S52 antibody (used at 1:150). Secondary antibodies were BIODIPY-labeled anti-mouse IgG (1:200; green fluorescence) and Cy3-labeled goat anti-rabbit (1:500; red fluorescence). A-C: 10×magnification of Calbindin 28K (A); S52 (B); and DAPI (C). D-F: 40×magnification of Calbindin 28K (D), S52 (E) and DAPI (F). G, H: Merged images of Calbindin 28K and S52 at 10×(G) and 40×(H) magnification. I: Merged images of DAPI and S52 at 20×magnification.

FIG. 12

Subcellular localization of expressed recombinant CRIM1. (A) Schematic description of N- or C-terminally myc-tagged CRIM1 and C-terminally myc-tagged ectodomain (amino acids 1-901 of SEQ ID NO: 2). (B) Immunoblotting analysis of subcellular localization of N-terminal myc-tagged CRIM1. (C) Immunoblotting analysis of subcellular localization of C-terminal myc-tagged CRIM1 ectodomain.(D) Treatment of ectodomain with N-glycosylase. Immunofluorescence analysis of (E) N-terminal myc-tagged CRIM1 in permeabilized cells with anti-myc antibody; (F) N-terminal myc-tagged CRIM1 in permeabilized cells with anti-S52 C-terminal antibody; (G) N-terminal myc-tagged CRIM1 in permeabilized cells merged; (H) N-terminal myc-tagged CRIM1 in non-permeabilized cells with anti-myc antibody; (I) CRIM1 ectodomain in non-permeabilized cells with anti-myc antibody; and (J) CRIM1 in permeabilized cells with anti-myc antibody.

FIG. 13

Interaction between CRIM1 and TGF-β superfamily members. (A) Co-immunoprecipitation of CRIM1 and BMP4 preprotein using anti-myc mAb (left panel) and anti-S52 N-terminal antibody (right panel). (B) BMP secretion determined by anti-myc immunoblotting. (C) Secretion of CRIM1 ectodomain determined by anti-myc immunoblotting. (D) Ligand blotting to detect interaction between CRIM1 and TGF-β in aqueuous humour. (E) BMP4 cell overlay assays.

FIG. 14

CRIM1 ectodomain bioassays. (A) Ectodomain purification detected by anti-myc immunoblotting (B) In ovo electroporation bioassay in chick spinal chord.GFP indicates gene expression resulting from in ovo electroporation. Isl-1=Islet 1; En1=Engrailed 1; DRG=dorsal root ganglia.

### DETAILED DESCRIPTION OF THE INVENTION

The present invention is predicated, at least in part, by the unexpected discovery of human, mouse and chicken Crim1 nucleic acids and CRIM1 polypeptides encoded thereby. Conservation of CRIM1/Crim1 and its expression during embryonic development suggests that CRIM1/Crim1 may be important for the normal development of vertebrates. In particular, it is proposed that CRIM1 may be involved in neuronal development and/or kidney and gonad development.

Furthermore, the present inventors provide evidence that CRIM1 polypeptides interact with TGF-β superfamily polypeptides, which polypeptides include TGF-β, a polypeptide known to be involved in eye defects such as cataract formation.

It is proposed herein that through binding to one or more members of the TGF-β superfamily of growth factors, CRIM1 will augment or antagonize their biological activities.

As used herein, unless the context requires otherwise, “comprise”, “comprises” or “comprising”, will be understood to imply the inclusion of a stated element or integer or group of elements or integers but not the exclusion of any other element or integer or group of elements or integers.

Throughout this specification, scientific terms are given their usual scientific meaning, although certain terms are defined herein to assist in their interpretation.

The term “recombinant” as used herein means artificially produced through human manipulation of genetic material, such as involving techniques generally falling within the scope of “recombinant DNA technology” as is well understood in the art.

By “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in recombinant or native form.

CRIM 1 Polypeptides

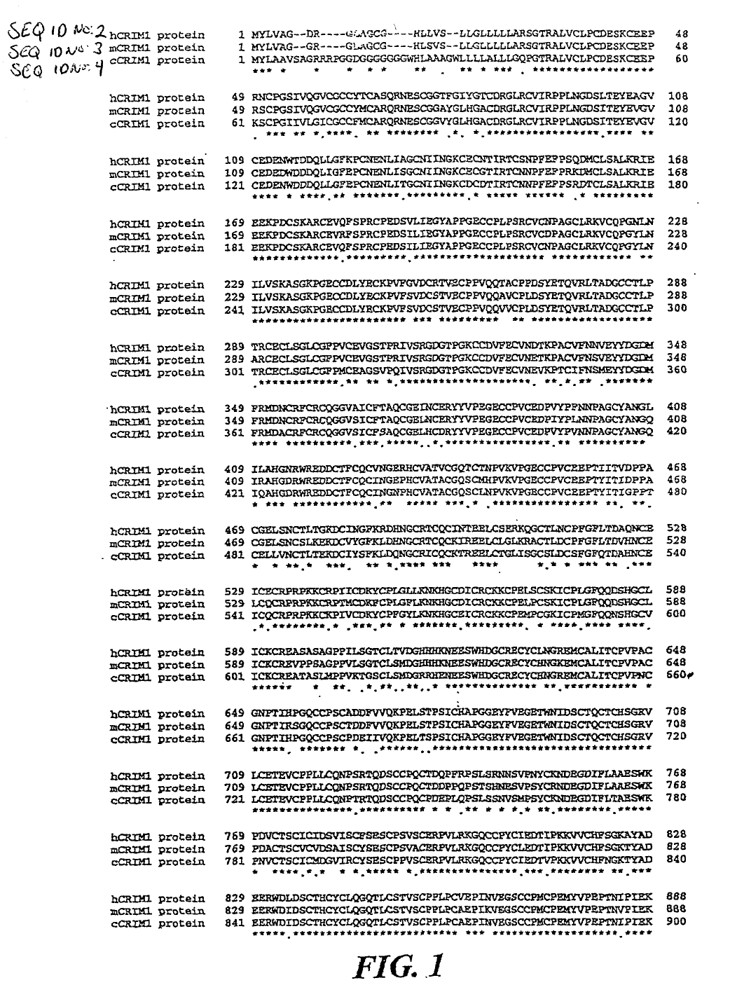

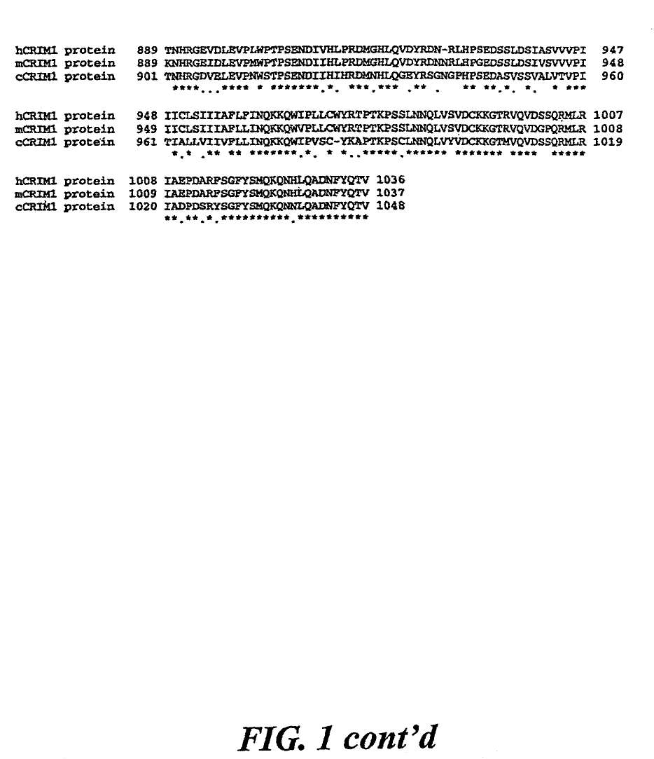

The invention provides CRIM1 polypeptides isolated from human, mouse and chicken, comprising the amino acid sequence PGECCPLP (SEQ ID NO: 1), for example as set forth in FIG. 1 (SEQ ID NOS: 2, 3 and 4 respectively).

CRIM1 polypeptides are further characterized by the presence of six cysteine-rich domains, an RGD domain, an IGFBP-like domain and a putative transmembrane domain.

By “polypeptide” is also meant “protein”, either term referring to an amino acid polymer which may include natural and/or non-natural amino acids as are well known in the art.

A “peptide” is a protein having no more than fifty (50) amino acids.

A peptide may be a “fragment” of a larger polypeptide, for example of at least 6, preferably at least 10 and more preferably at least 20 amino acids in length. Larger fragments comprising more than one peptide are also contemplated, and may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Peptide synthesis is also described in detail in Chapter 18 of CURRENT PROTOCOLS IN PROTEIN SCIENCE, Coligan et al Eds (John Wiley & Sons, 1995-2000), which is incorporated herein by reference. Alternatively, peptides can be produced by digestion of a polypeptide of the invention with proteinases such as endoLys-C, endoArg-C, endoGlu-C and staphylococcins V8-protease. The digested fragments can be purified by, for example, high performance liquid chromatographic (HPLC) techniques.

The invention also contemplates “biologically-active fragments” of CRIM1 polypeptides.

Suitably, the biologically-active fragment has at least 1%, preferably at least 10%, more preferably at least 25% and even more preferably at least 50% of the biological activity of a CRIM1 polypeptide.

An example of a biologically-active fragment is a CRIM1 ectodomain polypeptide comprising amino acids 1-901 of SEQ ID NO: 2.

As used herein,“variant” polypeptides include CRIM1 polypeptides in which one or more amino acids have been replaced by different amino acids. It is well understood in the art that some amino acids may be changed to others with broadly similar properties without changing the nature of the activity of the polypeptide (conservative substitutions).

Substantial changes in function are made by selecting substitutions that are less conservative. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Thr) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a bulky side chain (e.g., Phe or Trp) is substituted for, or by, one having a smaller side chain (e.g., Ala, Ser)or no side chain (e.g., Gly).

The term “variant” also includes CRIM1 polypeptides produced from allelic variants of the sequences exemplified in this specification.

In another embodiment, variant polypeptides share at least 60%, preferably at least 80% and more preferably at least 90% sequence identity with any one of the CRIM 1 amino acid sequences.

As used herein, “derivative” polypeptides are CRIM1 polypeptides which have been altered, for example by conjugation or complexing with other chemical moieties or by post-translational modification techniques as would be understood in the art. Such derivatives include amino acid deletions and/or additions to polypeptides of the invention.

Derivative polypeptides may include fusions of a CRIM1 polypeptide with another polypeptide or protein. Well known examples of such proteins include Protein A, glutathione S-transferase (GST), green fluorescent protein (GFP) maltose-binding protein (MBP), hexahistidine (HIS₆) and epitope tags such as FLAG, haemagglutinin and c-myc tags.

An example of a c-myc tagged CRIM1 polypeptide is provided hereinafter.

Other derivatives contemplated by the invention include, but are not limited to, modification to side chains, incorporation of unnatural amino acids and/or their derivatives during peptide, polypeptide or protein synthesis and the use of crosslinkers and other methods which impose conformational constraints on the polypeptides, fragments and variants of the invention. Examples of side chain modifications contemplated by the present invention include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulphonic acid (TNBS).

The carboxyl group may be modified by carbodiimide activation via O-acylisourea formation followed by subsequent derivitization, by way of example, to a corresponding amide.

The guanidine group of arginine residues may be modified by formation of heterocyclic condensation products with reagents such as 2,3-butanedione, phenylglyoxal and glyoxal.

Sulphydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulphides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulphonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, omithine, sarcosine, 2-thienyl alanine and/or D-isomers of amino acids.

CRIM1 polypeptides of the invention, fragments, variants and derivatives, are readily made in recombinant form, as will be described in more detail hereinafter.

Generally, recombinant proteins may be conveniently prepared by a person skilled in the art using standard protocols as for example described in Sambrook, et al., MOLECULAR CLONING. A Laboratory Manual (Cold Spring Harbor Press, 1989), incorporated herein by reference, in particular Sections 16 and 17; CURRENT PROTOCOLS IN MOLECULAR BIOLOGY Eds. Ausubel et al., (John Wiley & Sons, Inc. 1995-1999), incorporated herein by reference, in particular Chapters 10 and 16; and CURRENT PROTOCOLS IN PROTEIN SCIENCE Eds. Coligan et al., (John Wiley & Sons, Inc. 1995-1999) which is incorporated by reference herein, in particular Chapters 1, 5, 6 and 7.

With regard to polypeptide variants, these can be created by mutagenizing a polypeptide or by mutagenizing an encoding nucleic acid, such as by random mutagenesis or site-directed mutagenesis. Examples of nucleic acid mutagenesis methods are provided in in Chapter 9 of CURRENT PROTOCOLS IN MOLECULAR BIOLOGY, Ausubel et al., supra which is incorporated herein by reference.

It will be appreciated by the skilled person that site-directed mutagenesis is best performed where knowledge of the amino acid residues that contribute to biological activity is available. In many cases, this information is not available, or can only be inferred by molecular modelling approximations, for example.

In such cases, random mutagenesis is contemplated. Random mutagenesis methods include chemical modification of proteins by hydroxylamine (Ruan et al., 1997, Gene 188 35), incorporation of dNTP analogs into nucleic acids (Zaccolo et al., 1996, J. Mol. Biol. 255 589) and PCR-based random mutagenesis such as described in Stemmer, 1994, Proc. Natl. Acad. Sci. USA 91 10747 or Shafikhani et al., 1997, Biotechniques 23 304, each of which references is incorporated herein. It is also noted that PCR-based random mutagenesis kits are commercially available, such as the Diversify™ kit (Clontech).

Antibodies

The invention also provides antibodies capable of binding Crim1 polypeptides, biologically-active fragments, variants or derivatives thereof. Such antibodies may include any suitable antibodies which bind to or conjugate with a polypeptide of the invention, homolog or fragment thereof. Such antibodies may be polyclonal, obtained for example by immunizing an animal with a polypeptide, homolog or fragment thereof. It is for this purpose that peptides of the invention are particularly useful. Suitably, said animal could be a mouse, rat, rabbit or goat. Preferably, the animal is a rabbit.

Alternatively, monoclonal antibodies may be produced by a standard method such as described in CURRENT PROTOCOLS IN IMMUNOLOGY (1994, Eds. Coligan, Kruisbeek, Marguiles, Shevach and Strober; John Wiley & Sons), which is hereby incorporated by reference. Such a method would involve obtaining antibody-producing cells, such as spleen cells, from an animal immunized as described above, and immortalizing said cell, such as by fusion with an immortalized fusion partner cell.

Preferably, the antibody is a polyclonal antibody.

Advantageously, the antibody is a rabbit polyclonal antibody.

In one embodiment, the antibody is raised against the following amino acid sequence: RVQVDSSQRMLRIAEPDARFSGFYSMQKQNHLQADNFYQTV (SEQ ID NO: 8), which sequence corresponds to the C-terminal 41 amino acids of human CRIM1.

In another embodiment, the antibody is raised against the following amino acid sequence: KVCQPGYLNILVSKASGKPGEC (SEQ ID NO: 9), which sequence corresponds to the N-terminal amino acids of mouse CRIM1.

As is well understood in the art, antibodies may be conjugated with labels selected from a group including a chromogen, a catalyst, an enzyme, a fluorophore, a chemiluminescent molecule and a radioisotope.

A large number of enzymes suitable for use as labels is disclosed in United States Patent Specifications U.S. Pat. No. 4,366,241, U.S. Pat. No. 4,843,000, and U.S. Pat. No. 4,849,338, each of which is herein incorporated by reference. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme in solution.

Fluorophores may be selected from a group including fluorescein isothiocyanate (FITC), tetramethylrhodamine isothiocyanate (TRITC), allophycocyanin (APC), Texas Red (TR), Cy5 or R-Phycoerythrin (RPE). Examples of useful fluorophores may be found, for example, in U.S. Pat. No. 4,520,110 and U.S. Pat. No. 4,542,104 which are herein incorporated by reference.

CRIM1 Mimetics

The invention contemplates mimetics which antagonize or mimic one or more biological activities of CRIM1 polypeptides, or homologs of CRIM1.

It will be appreciated that CRIM1 comprises an RGD domain, cystein-rich domains, an IGFBP-like domain and transmembrane domain.

Of these, the six cysteine-rich repeats are considered to be preferred targets for the screening or design of potential CRIM1 mimetics.

The term “mimetics” is used herein to refer to molecules that resemble particular functional regions of proteins or peptides, and includes within its scope the terms “agonist”, “partial agonist”, “analogue” and “antagonist” as are well understood in the art.

The aforementioned mimetics may themselves be peptides or polypeptides, or may be other organic molecules, preferably small organic molecules, with a desired biological activity and half-life.

Mimetics may be identified by way of screening libraries of molecules such as synthetic chemical libraries, including combinatorial libraries, by methods such as described in Nestler & Liu, 1998, Comb. Chem. High Throughput Screen. 1 113 and Kirkpatrick et al., 1999, Comb. Chem. High Throughput Screen 2 211.

It is also contemplated that libraries of naturally-occurring molecules may be screened by methodology such as reviewed in Kolb, 1998, Prog. Drug. Res. 51 185.

More rational approaches to designing mimetics may employ computer assisted screening of structural databases, computer-assisted modelling, or more traditional biophysical techniques which detect molecular binding interactions, as are well known in the art.

Computer-assisted structural database searching is becoming increasingly utilized as a procedure for identifying mimetics.. Database searching methods which, in principle, may be suitable for identifying mimetics, may be found in International Publication WO 94/18232 (directed to producing HIV antigen mimetics), U.S. Pat. No. 5,752,019 and International Publication WO 97/41526 (directed to identifying EPO mimetics), each of which is incorporated herein by reference.

Generally, other methods include a variety of biophysical techniques which identify molecular interactions. Methods applicable to potentially useful techniques such as competitive radioligand binding assays, analytical ultracentrifugation, microcalorimetry, surface plasmon resonance and optical biosensor-based methods are provided in Chapter 20 of CURRENT PROTOCOLS IN PROTEIN SCIENCE Eds. Coligan et al., (John Wiley & Sons, 1997) which is incorporated herein by reference.

Crim1 Nucleic Acids

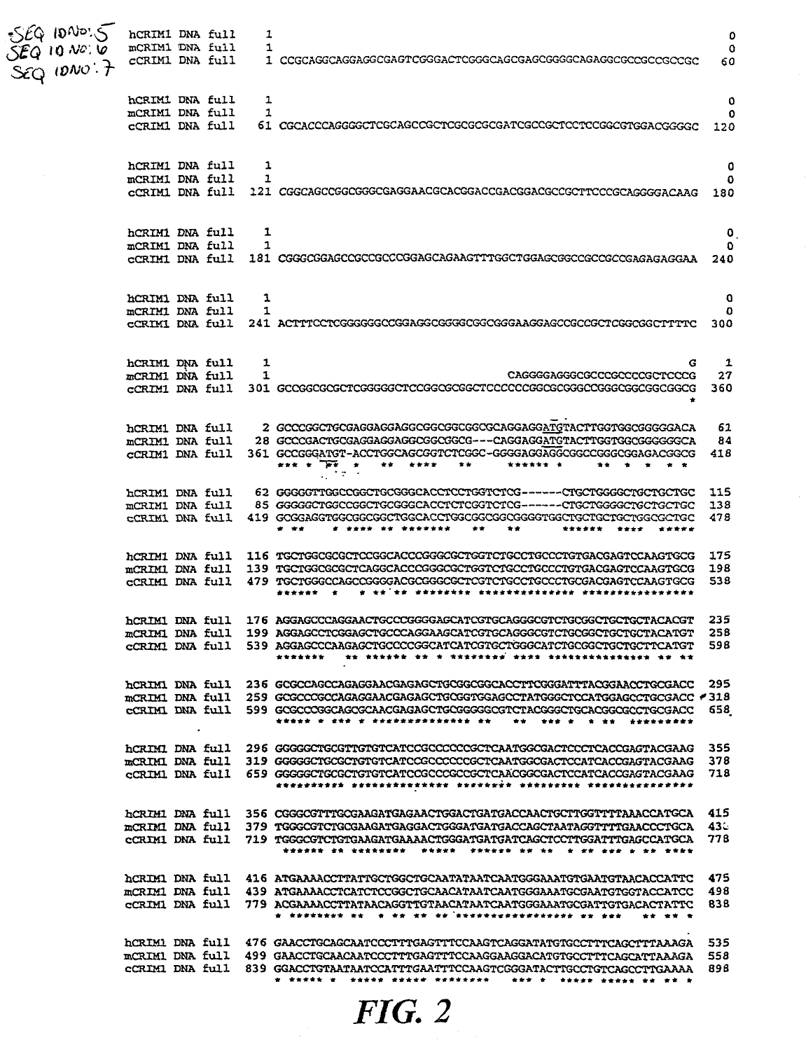

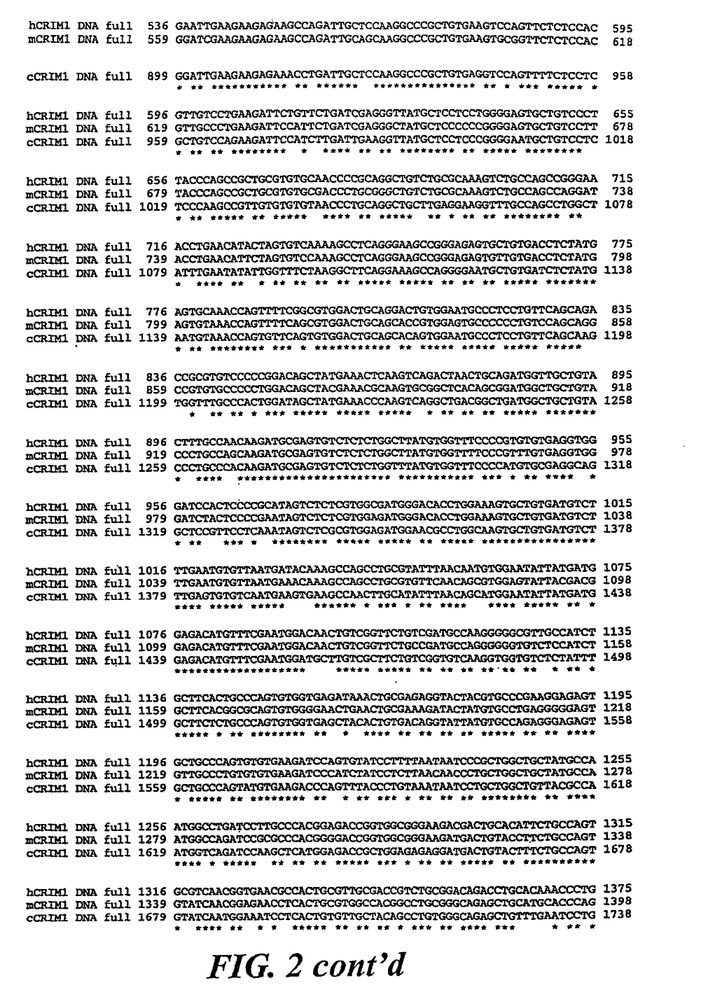

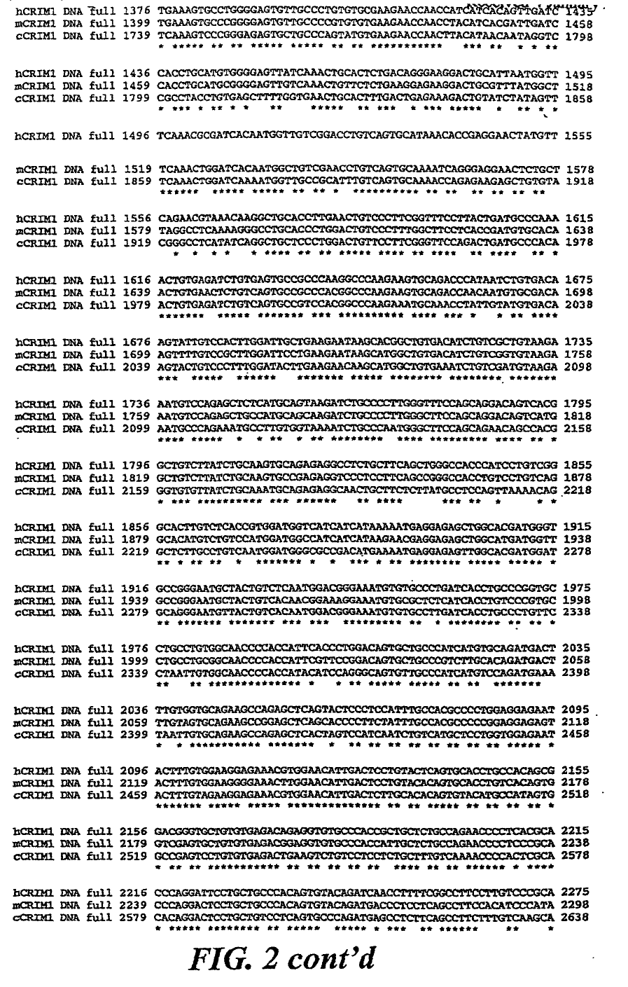

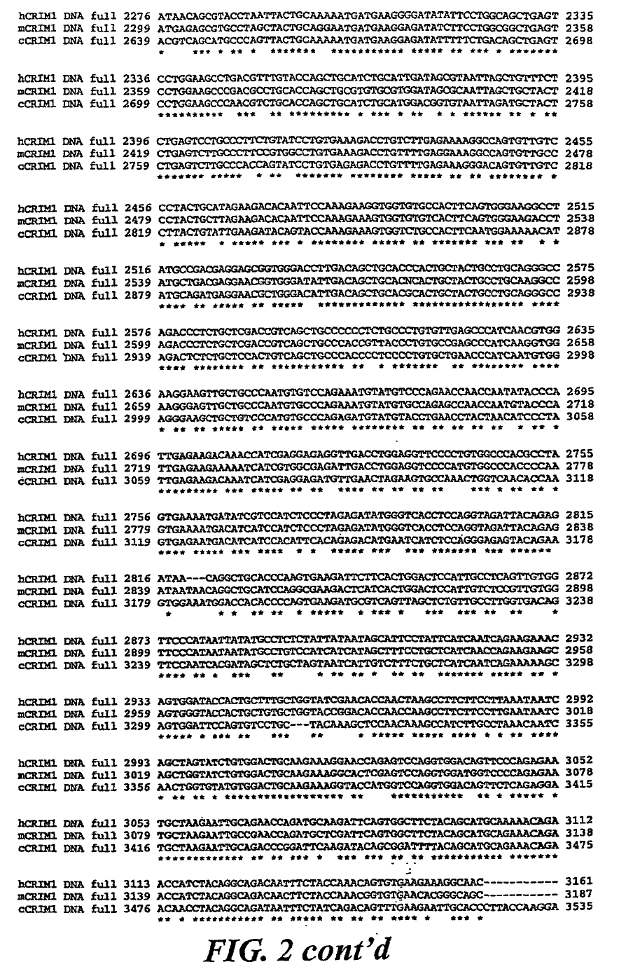

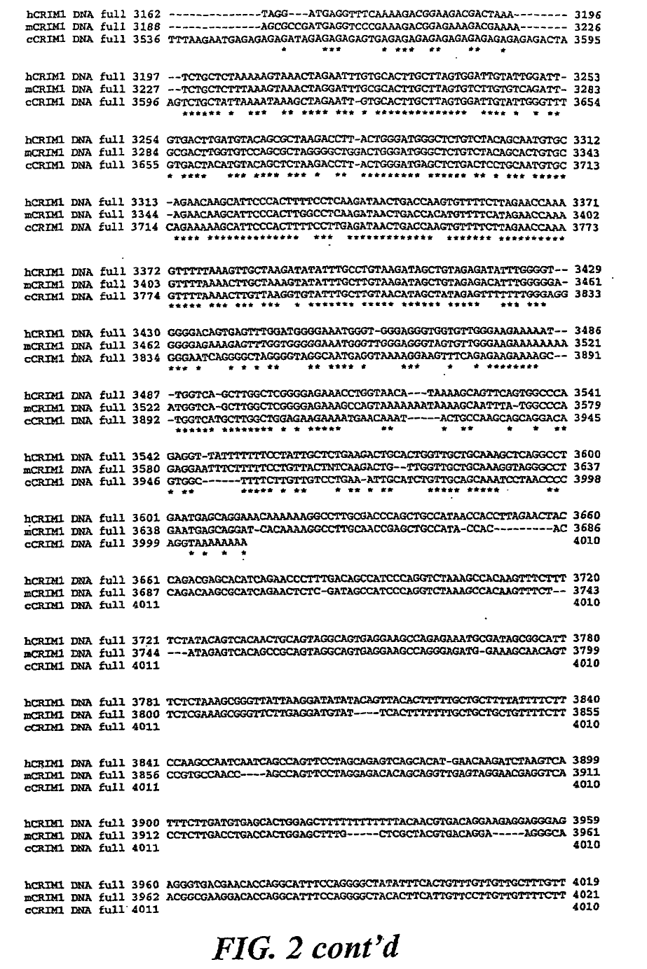

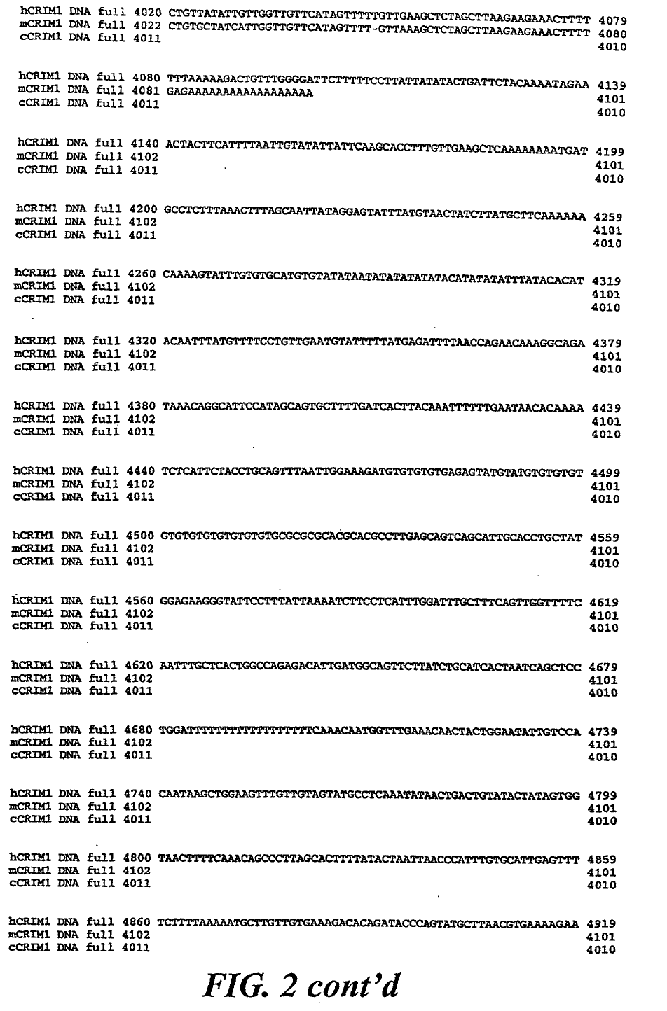

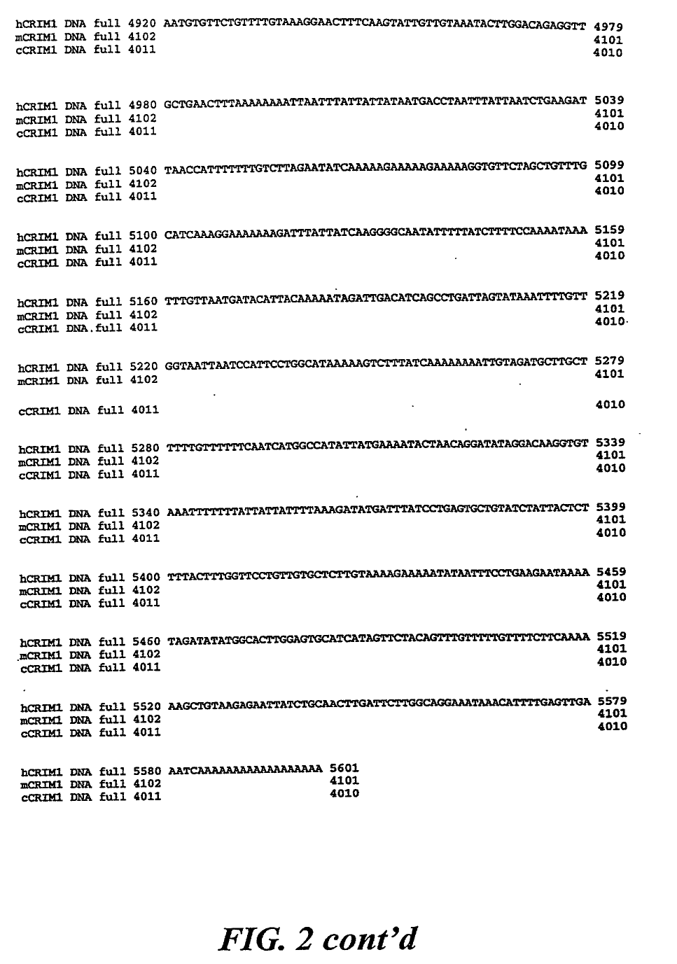

The invention provides isolated Crim1 nucleic acids, as for example set forth in FIG. 2 (SEQ ID NOS: 5-7).

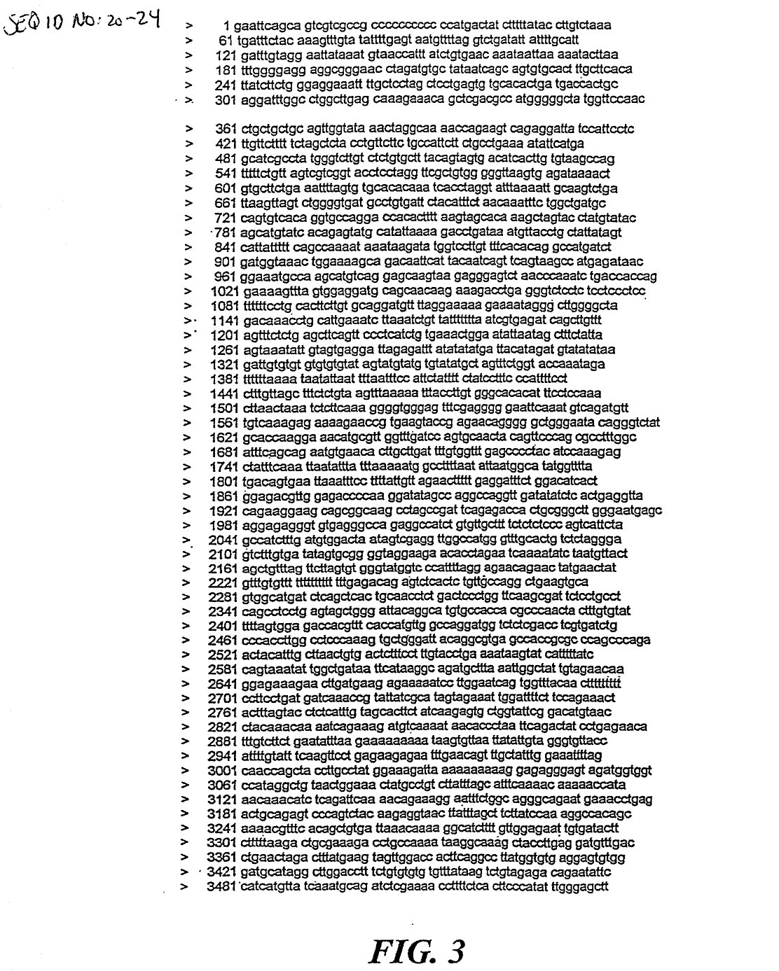

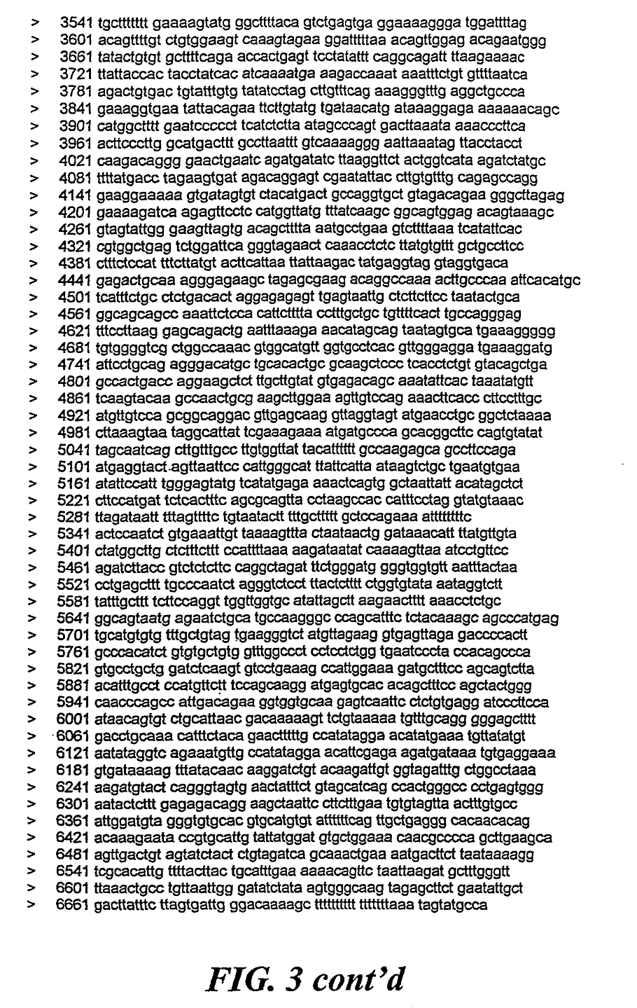

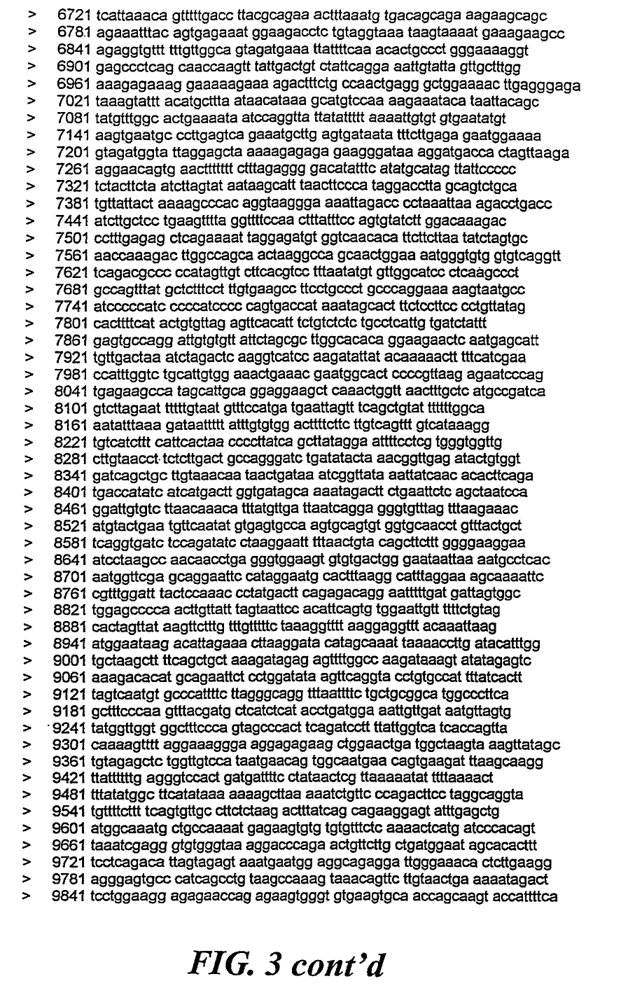

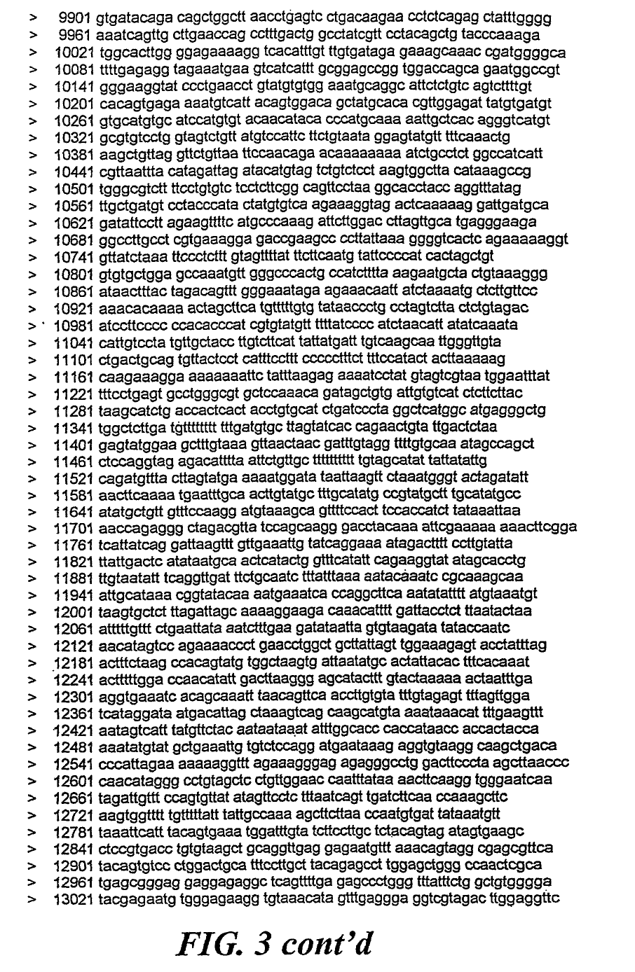

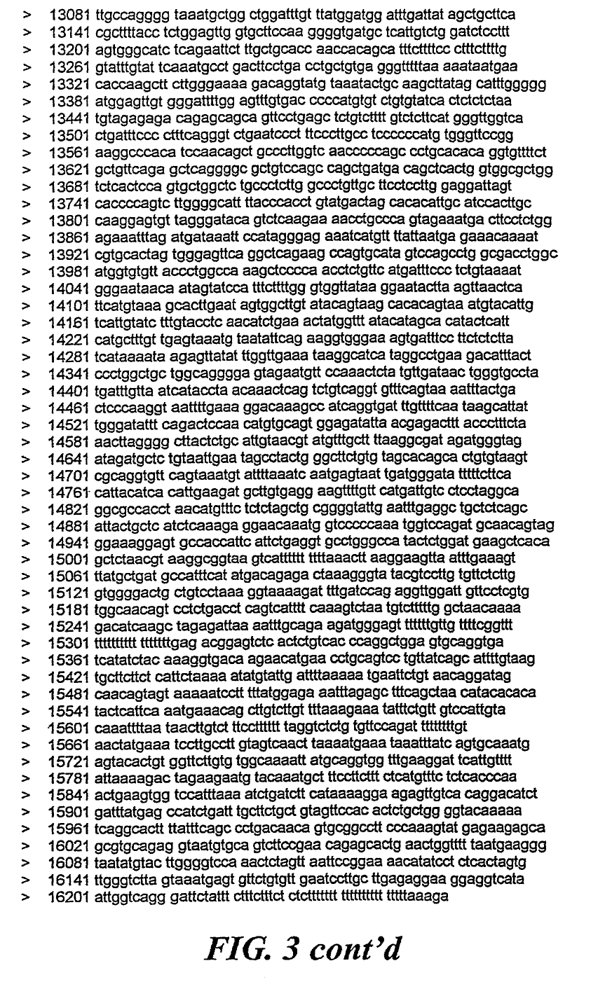

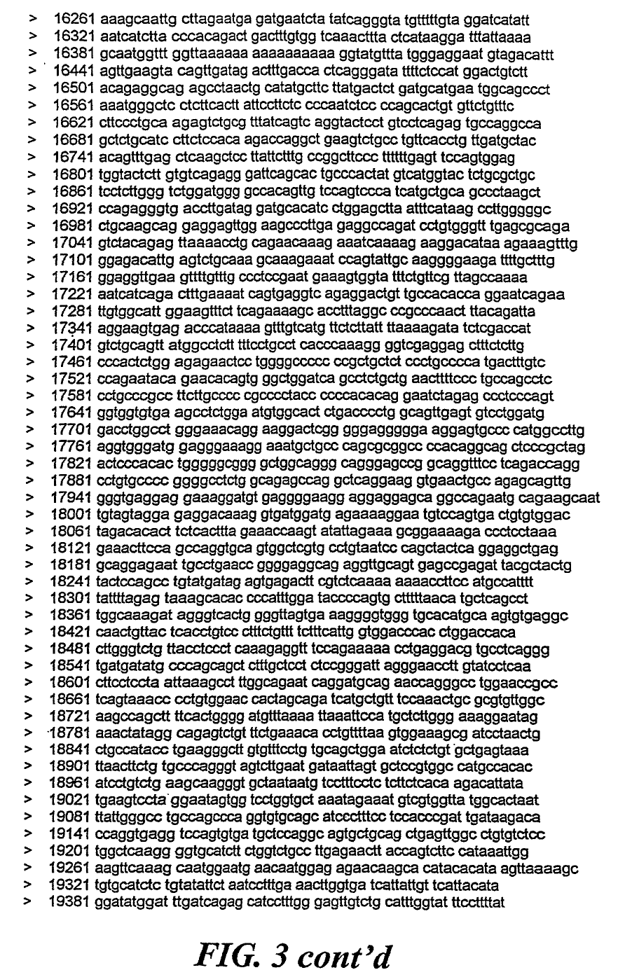

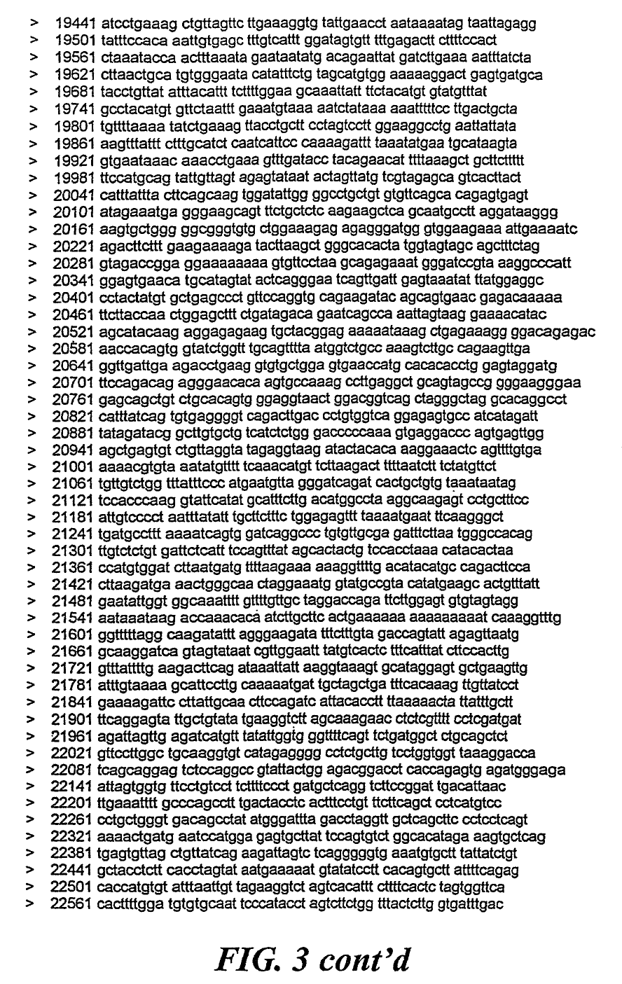

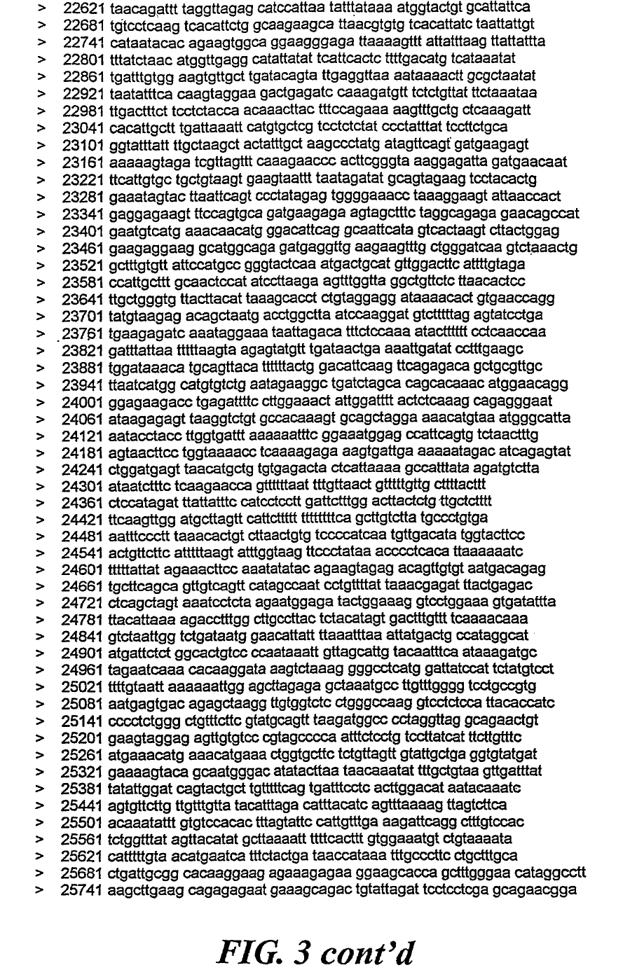

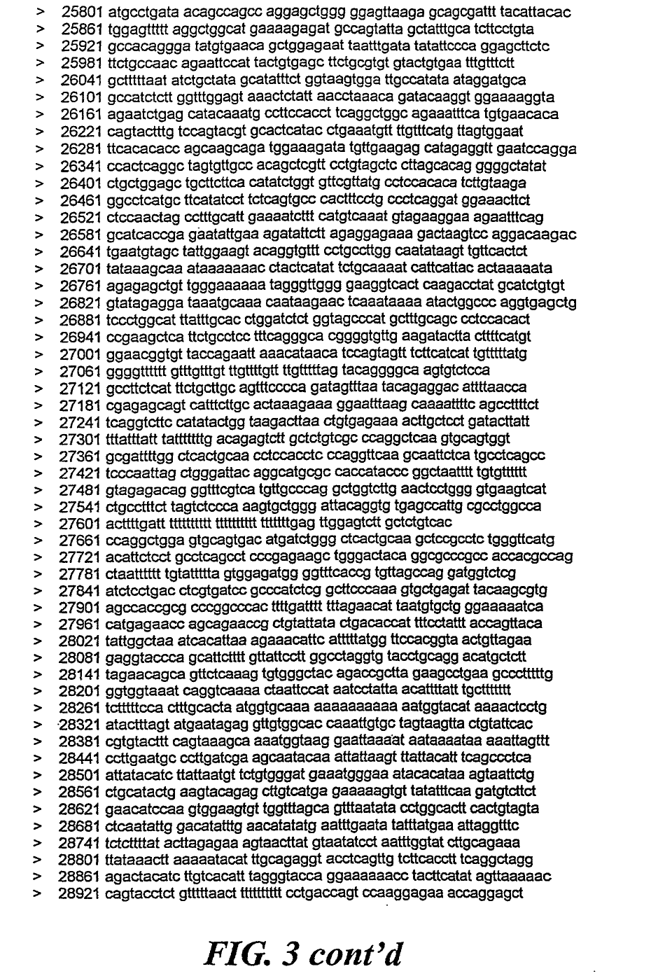

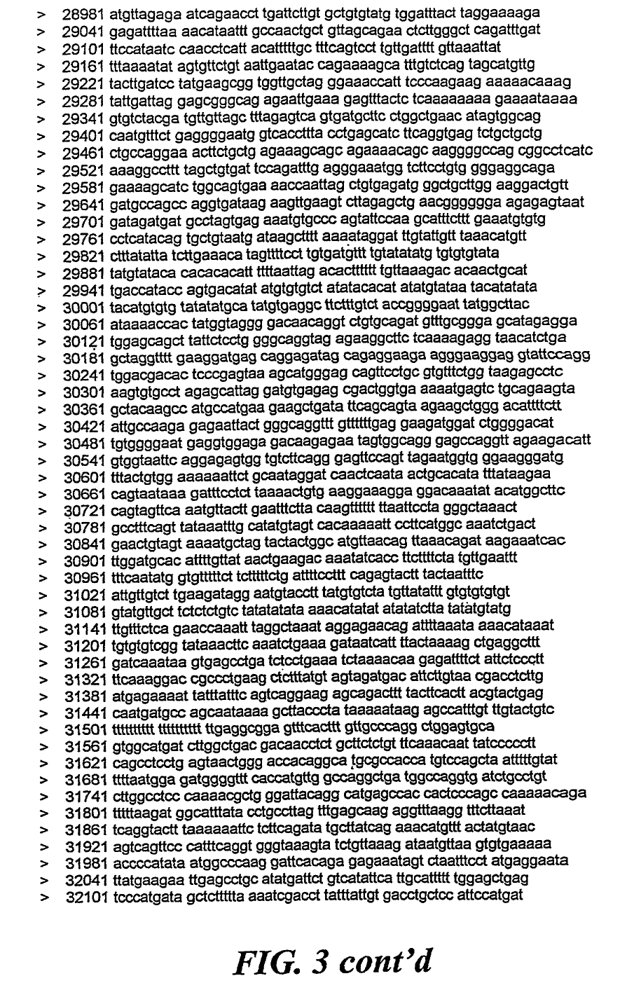

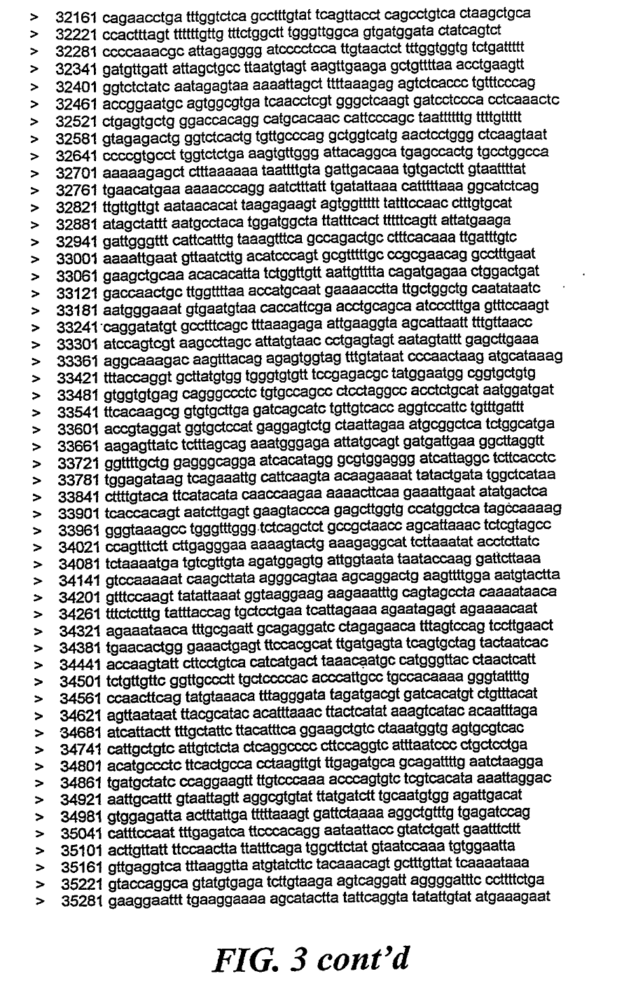

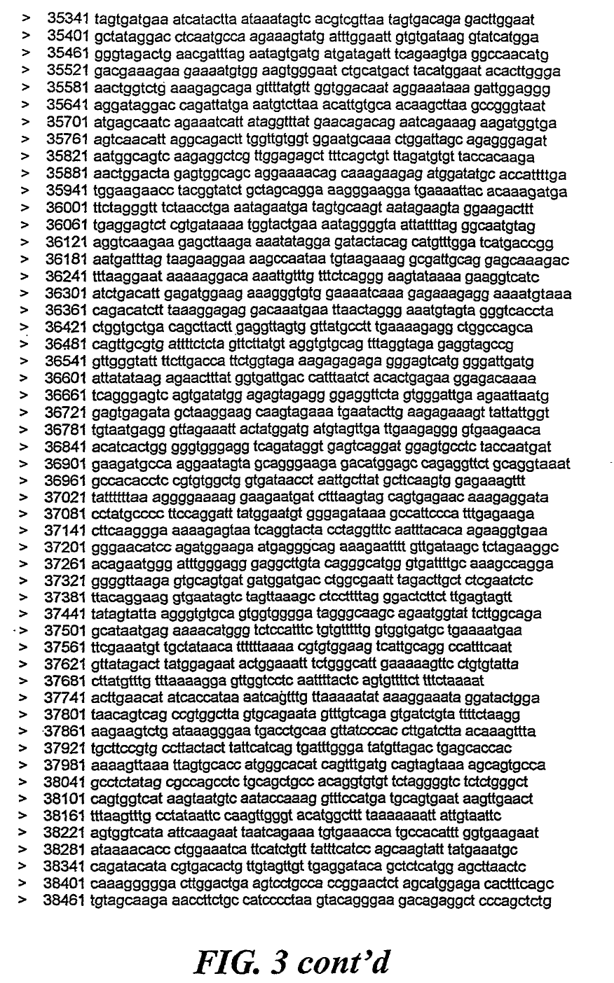

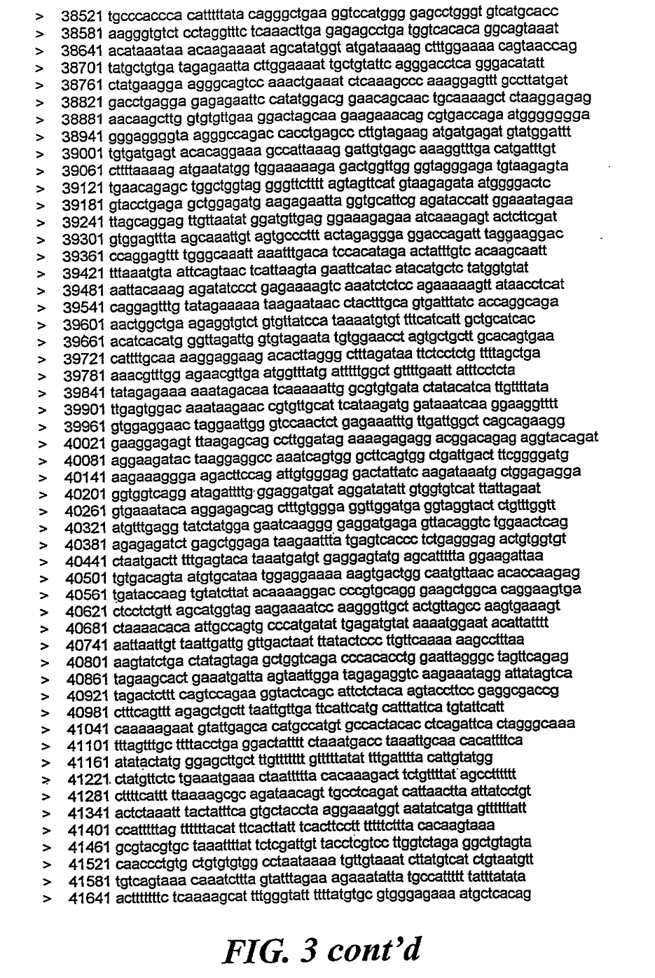

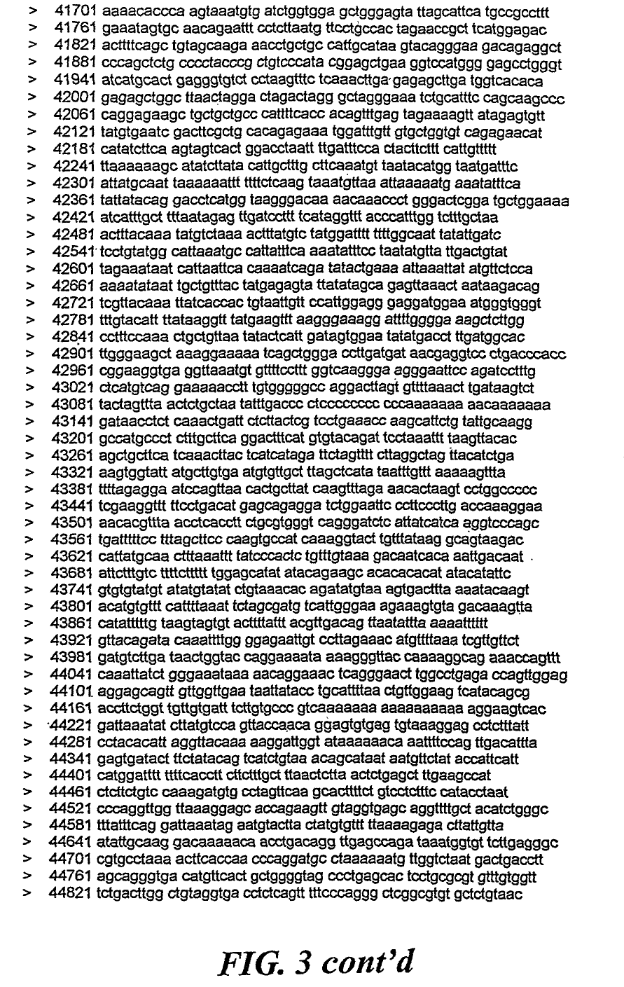

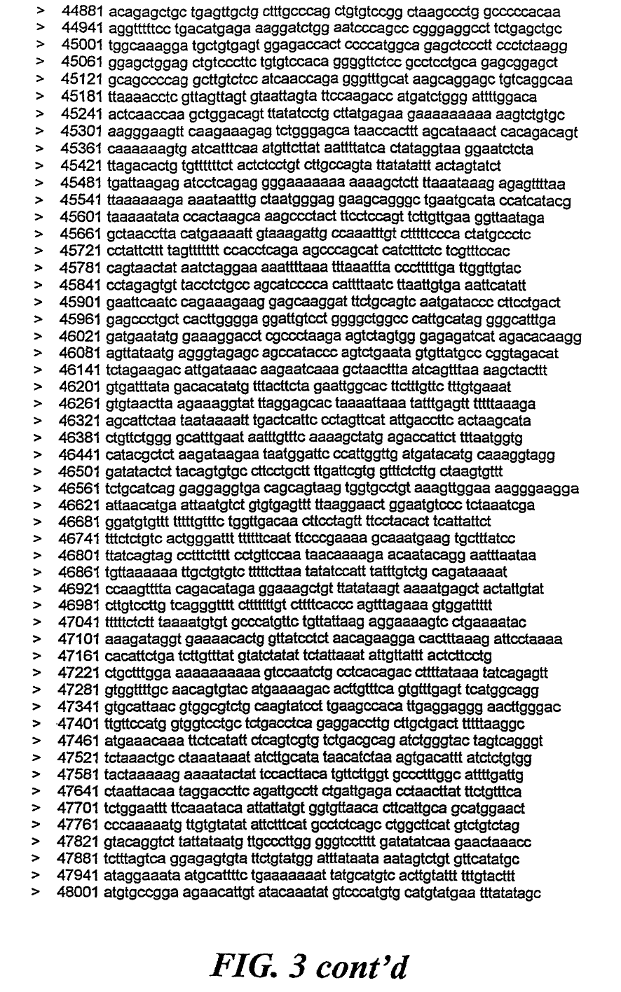

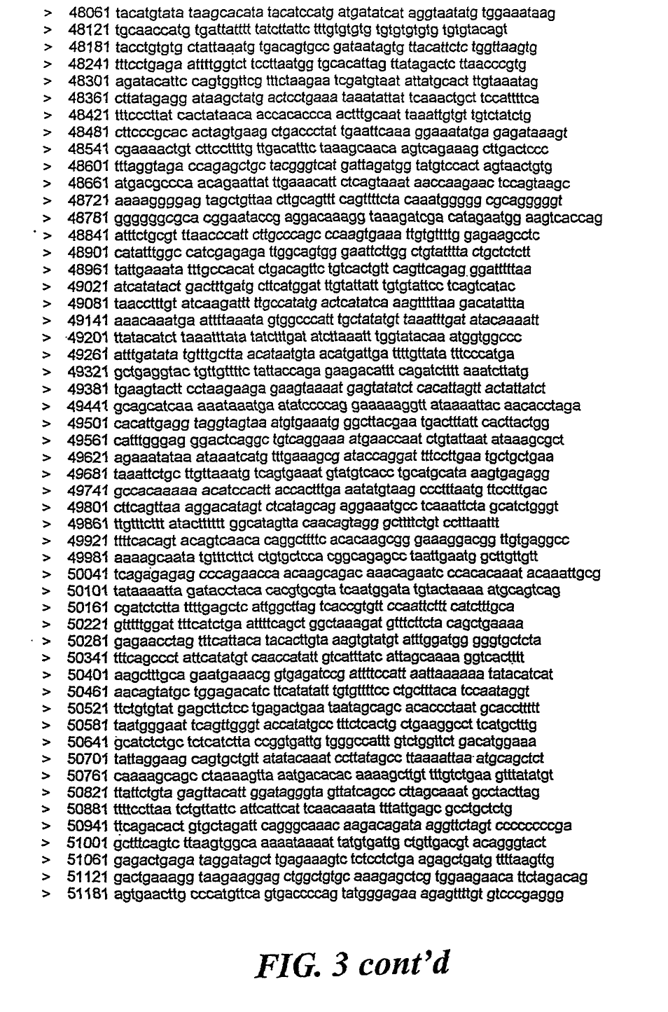

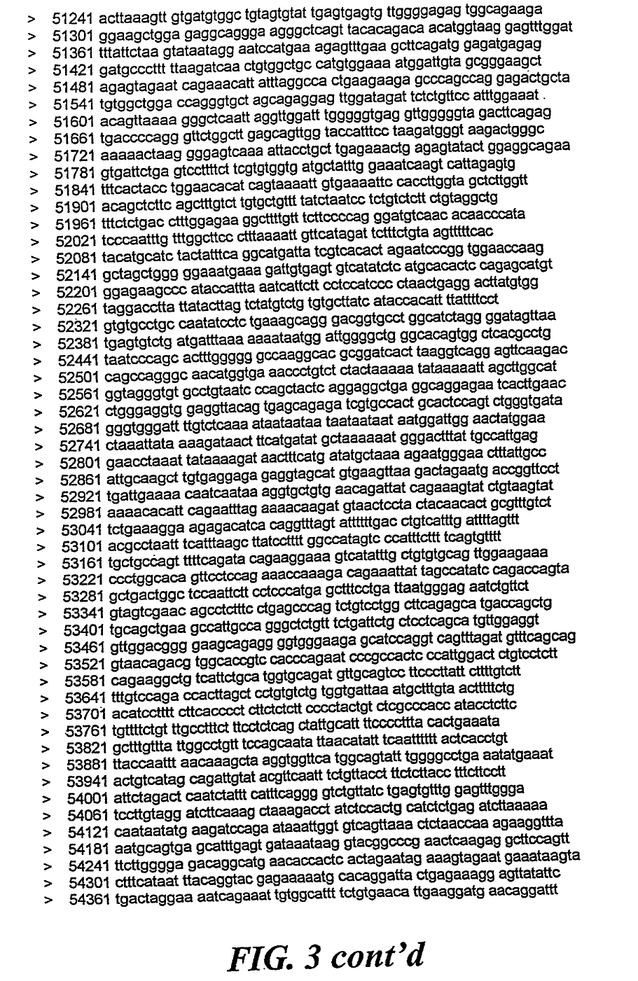

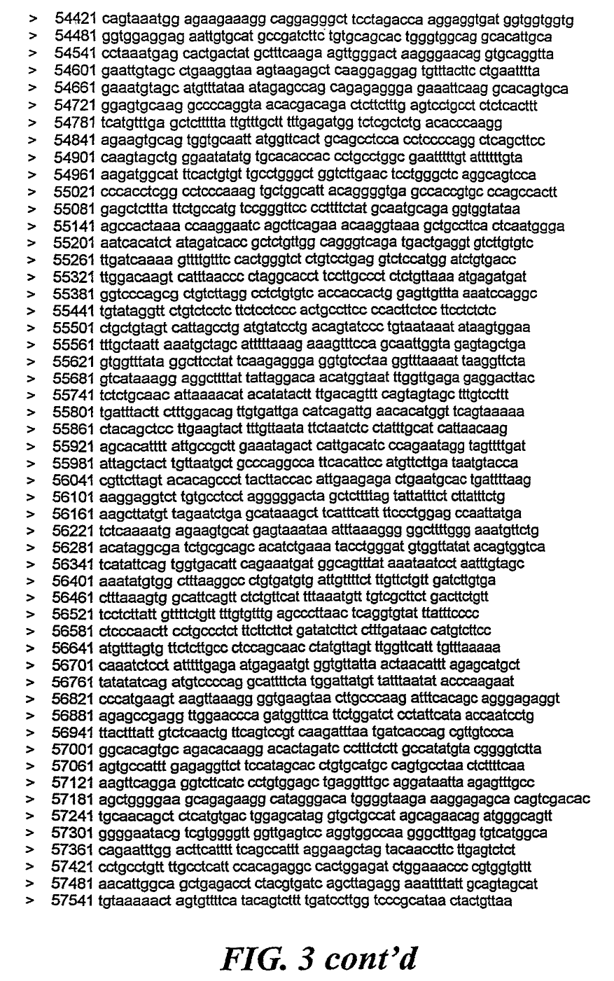

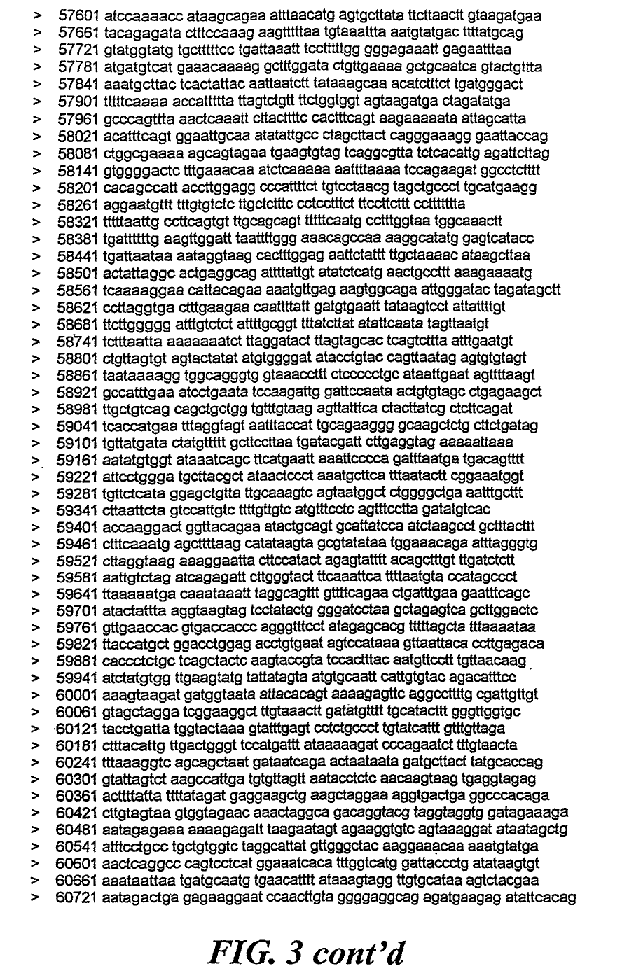

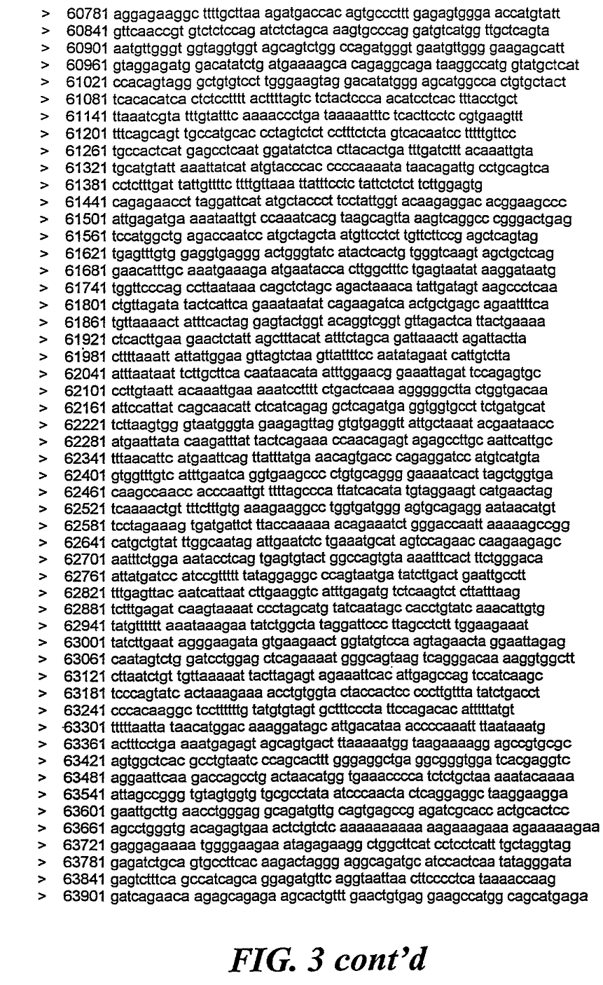

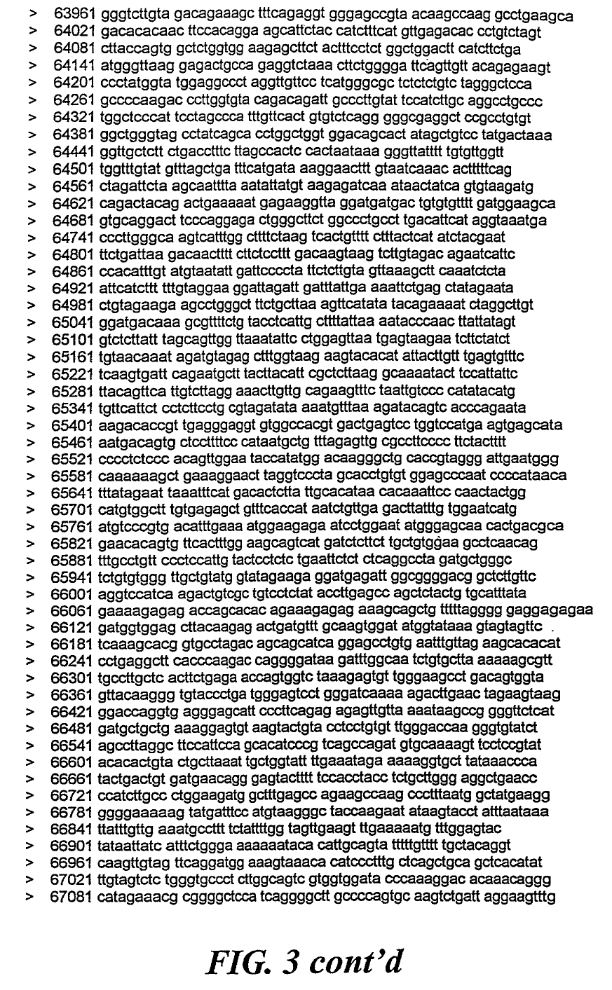

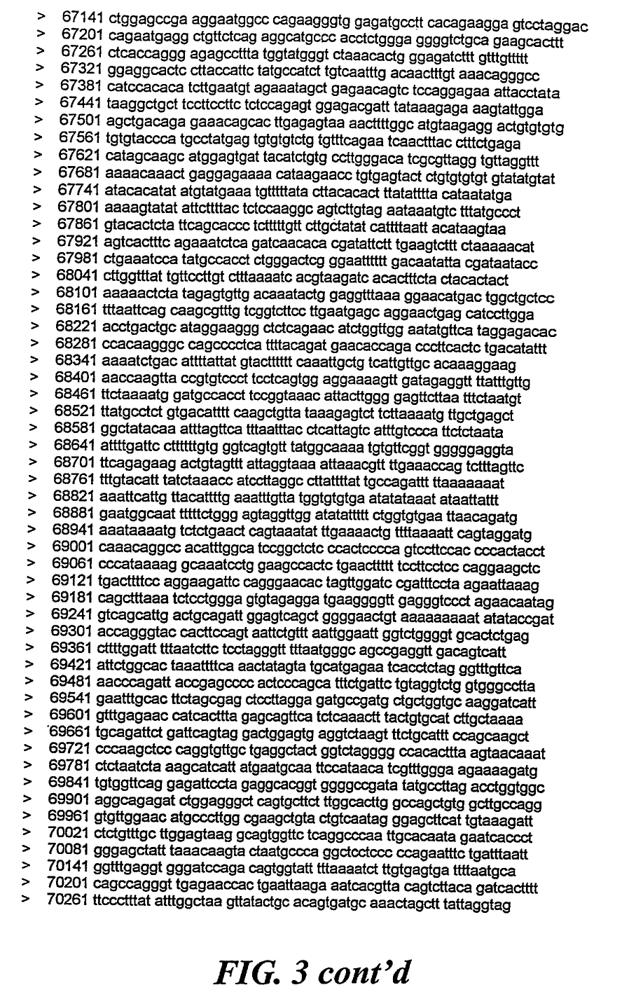

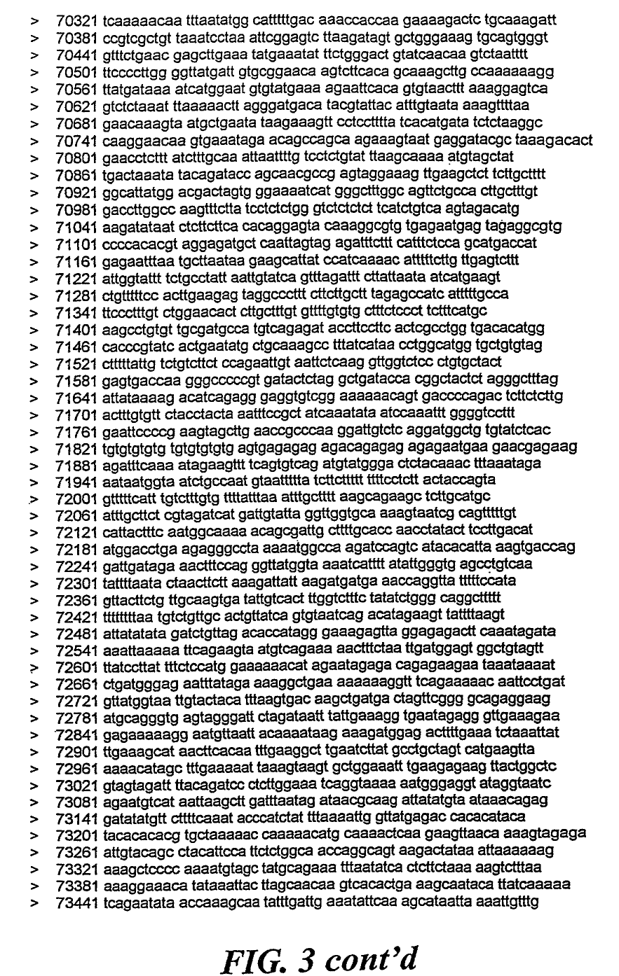

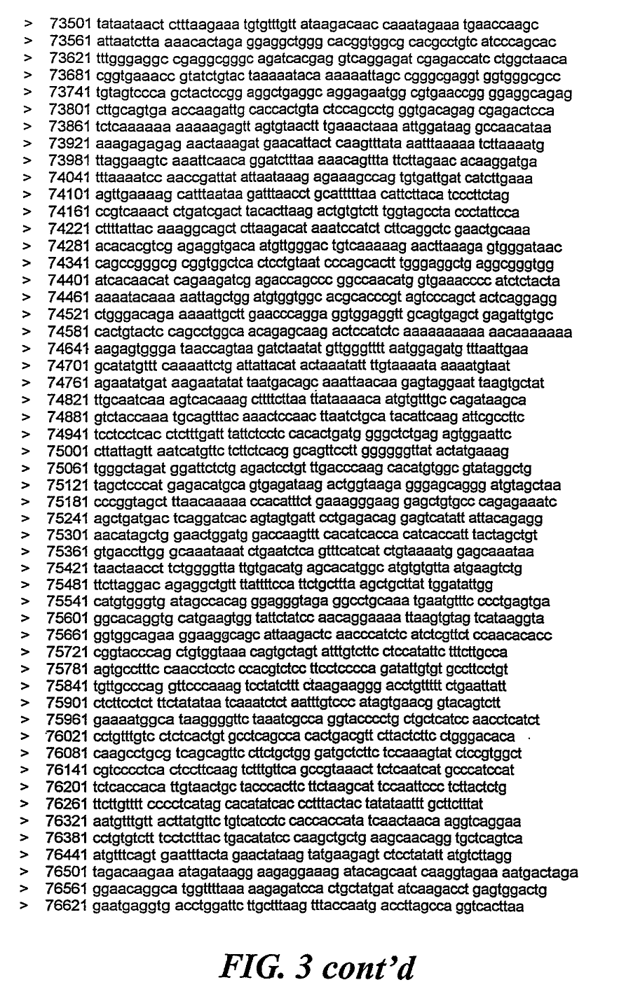

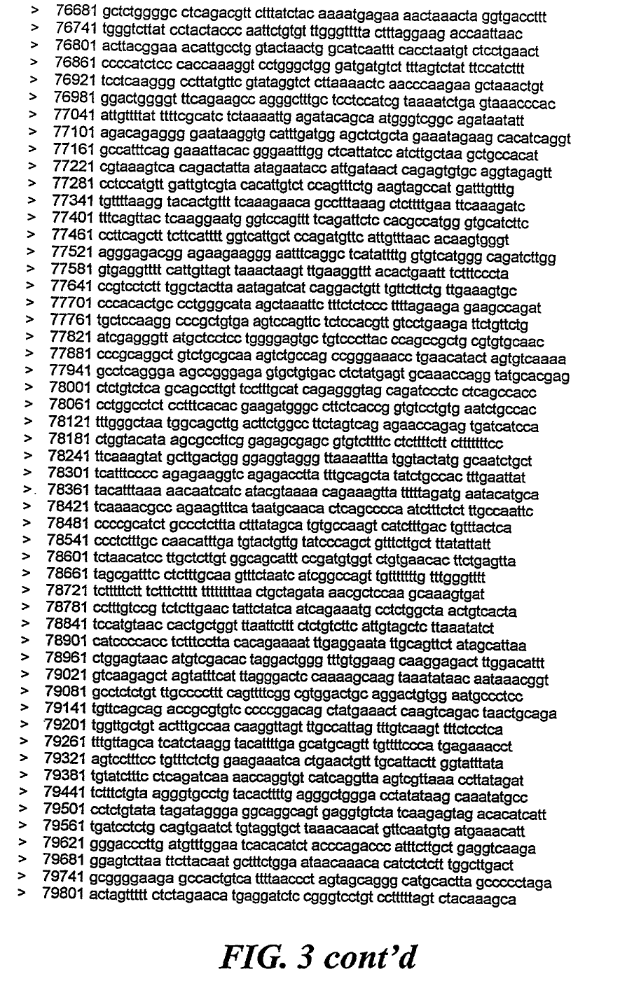

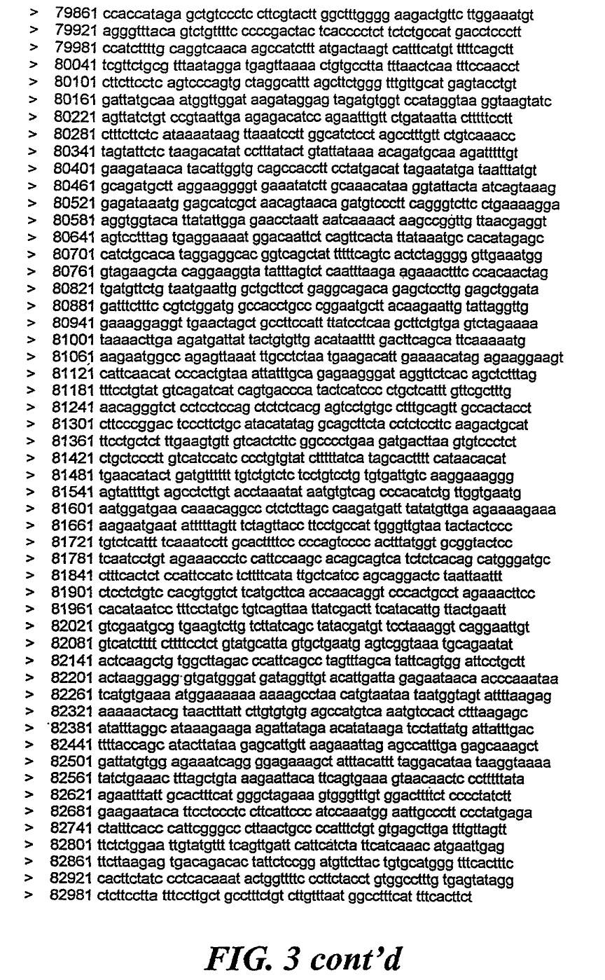

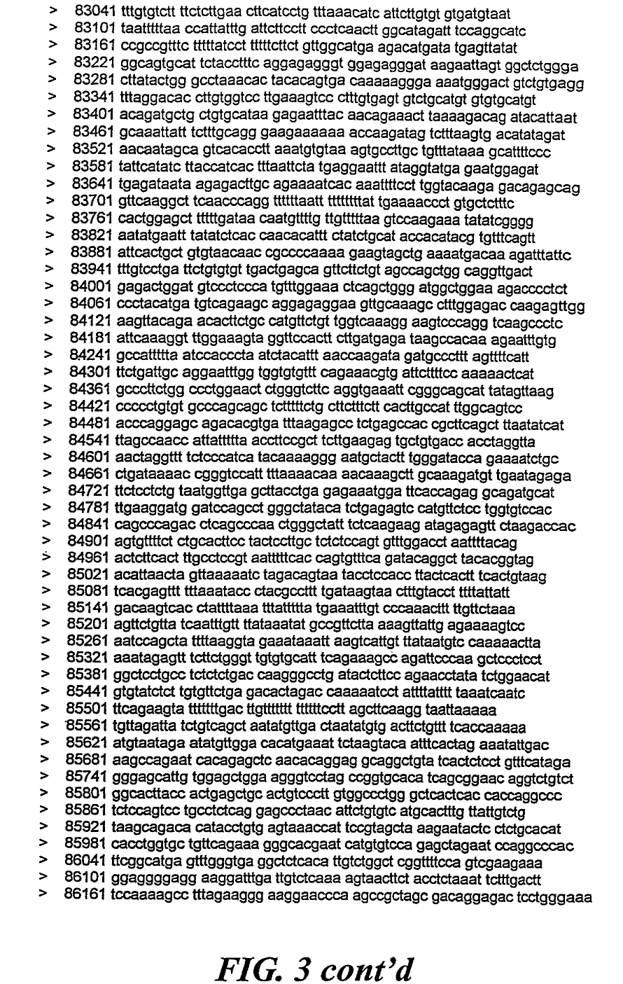

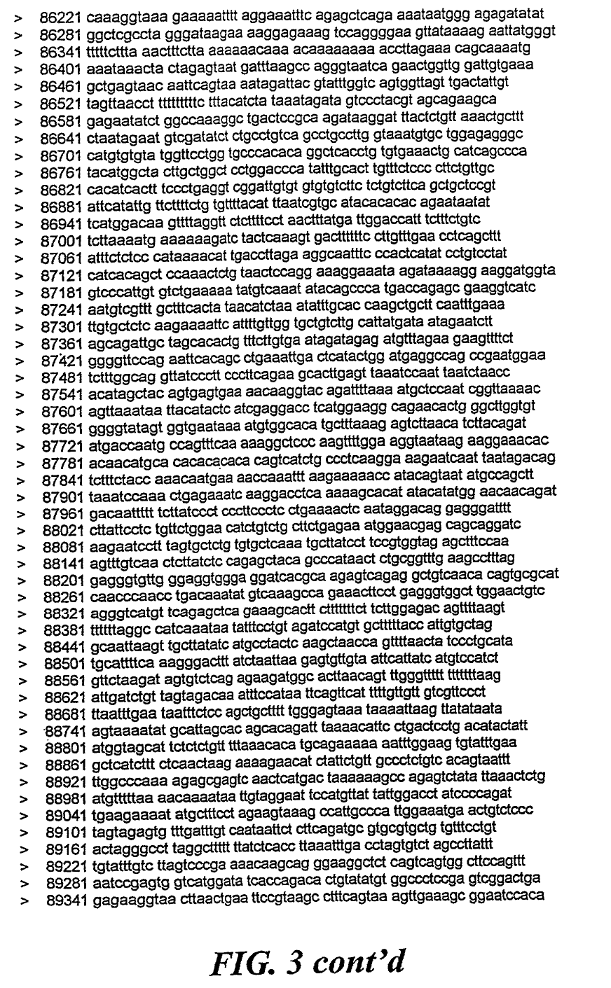

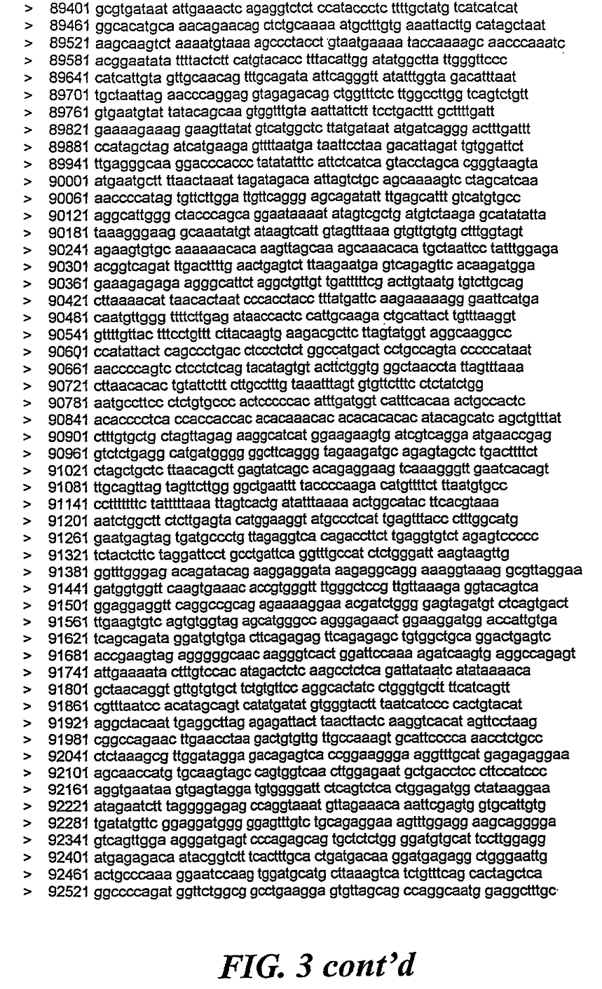

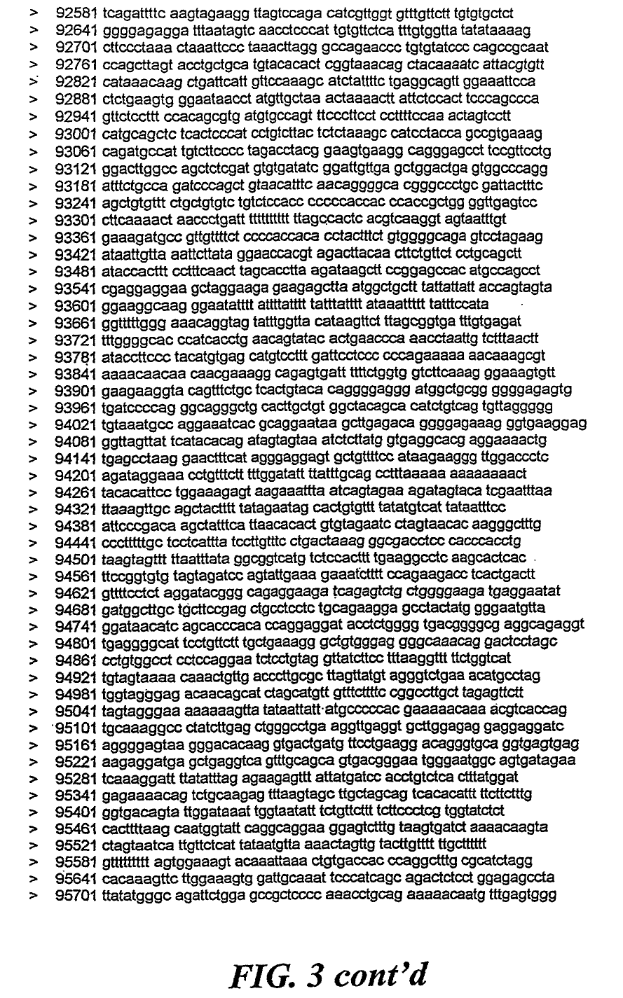

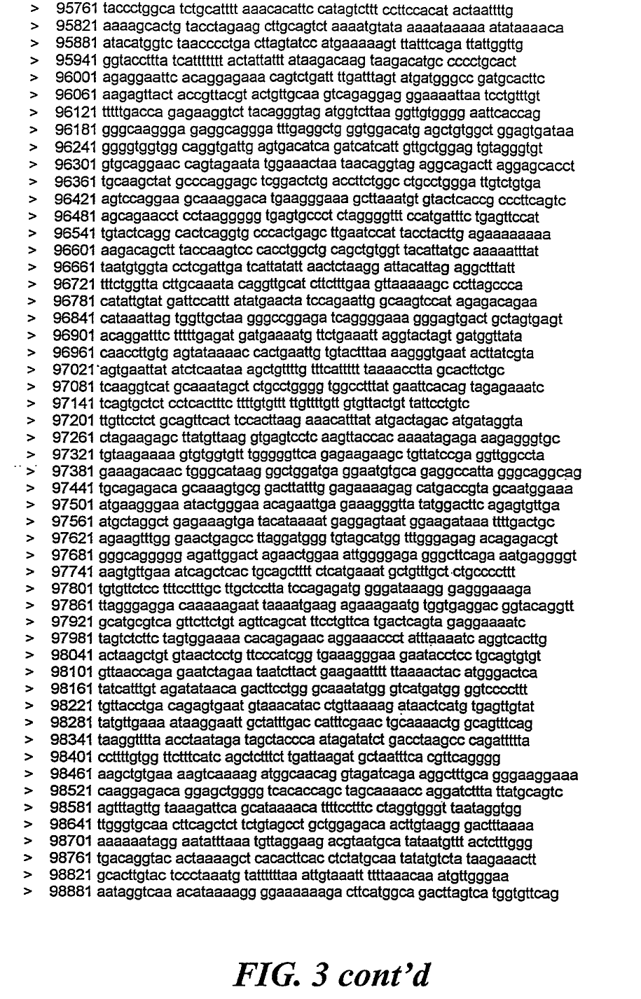

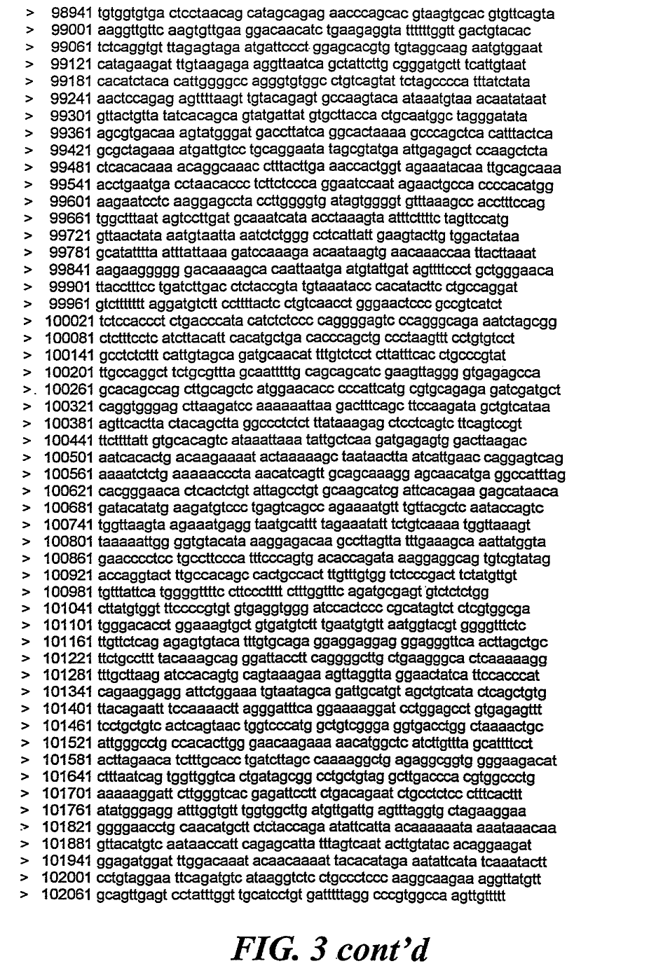

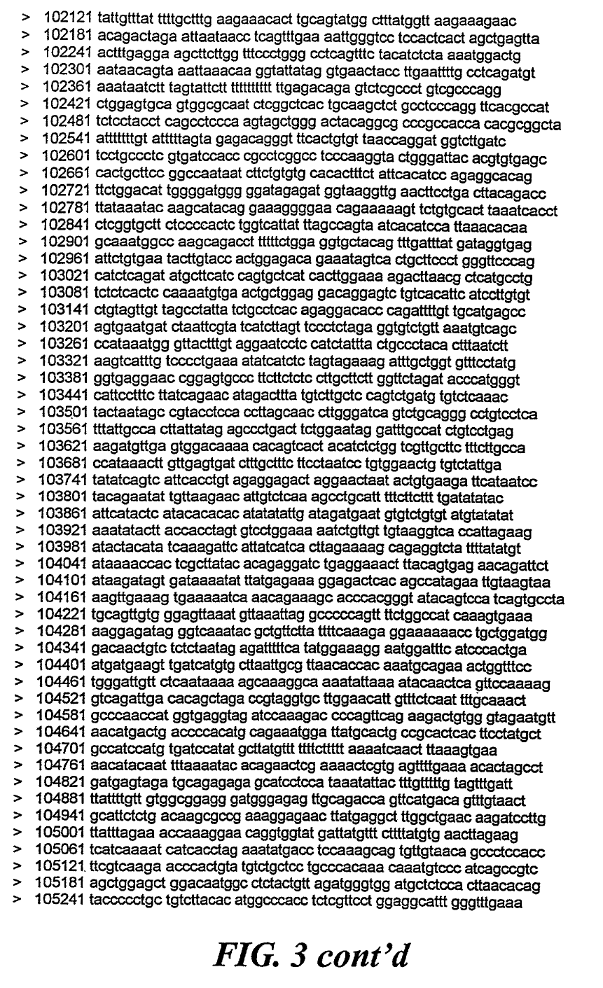

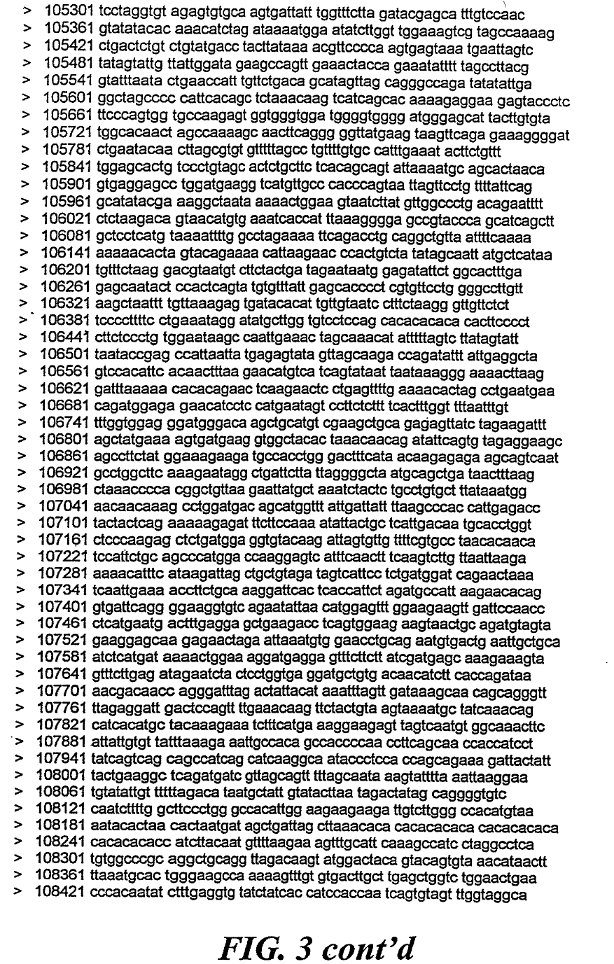

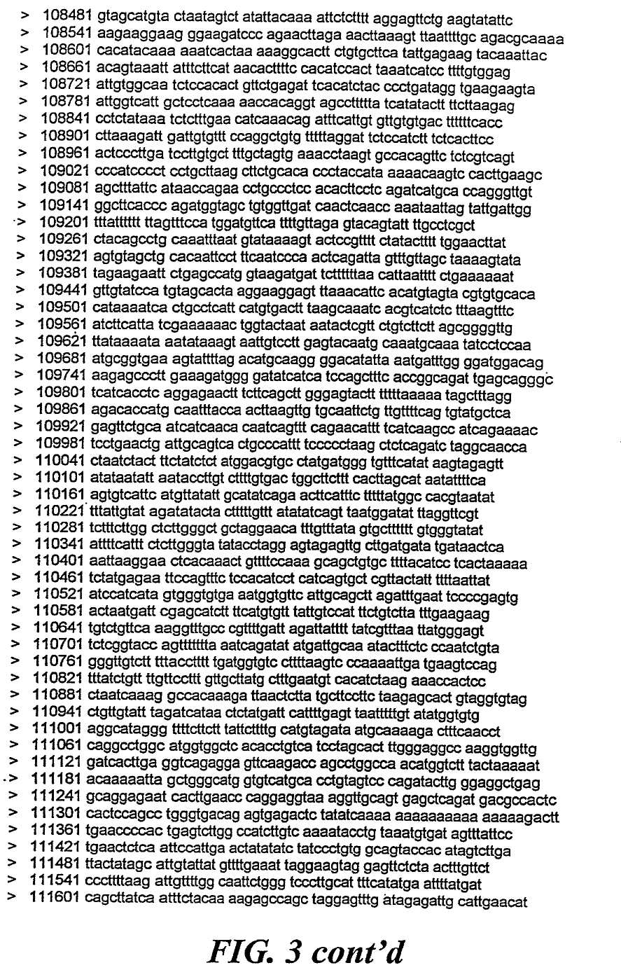

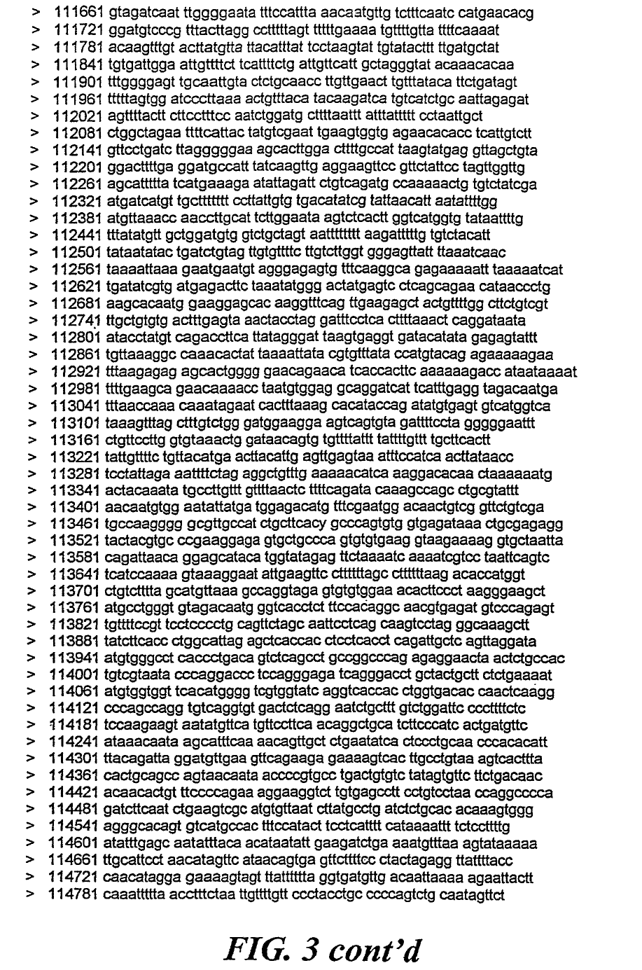

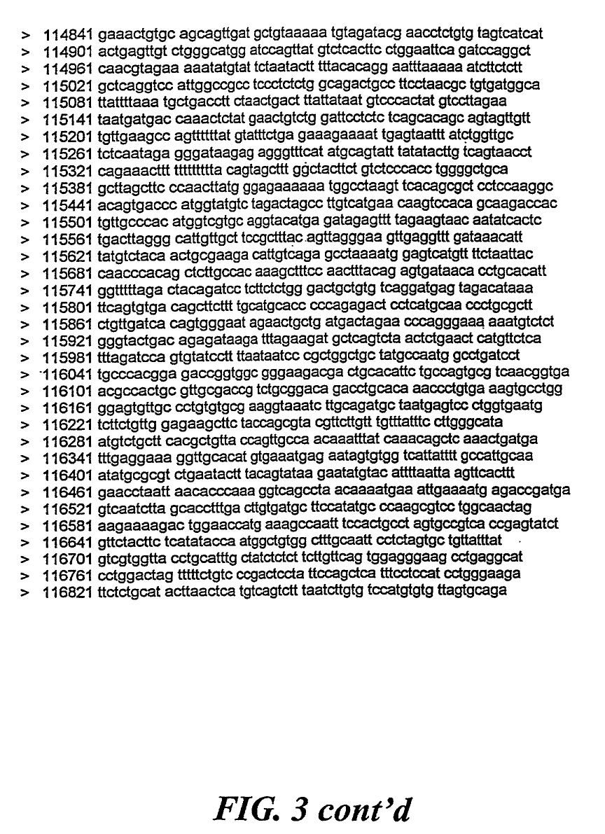

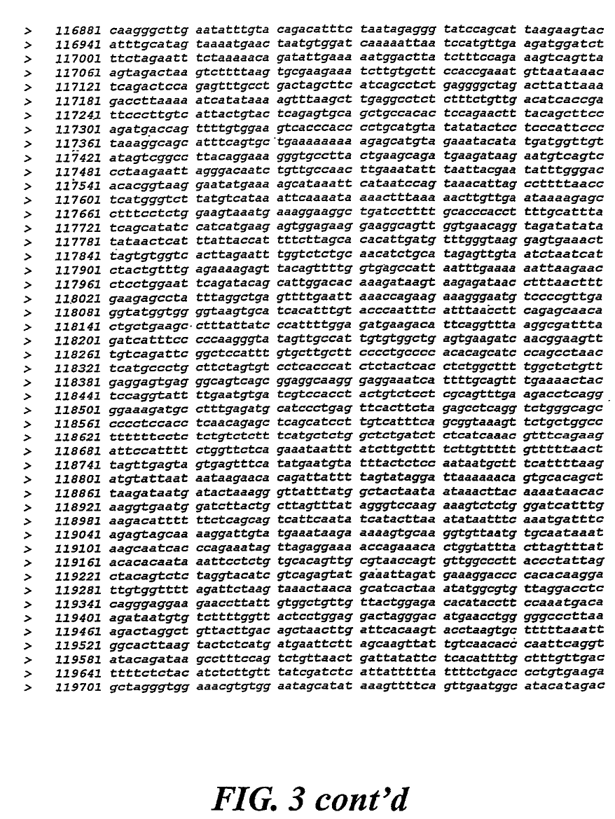

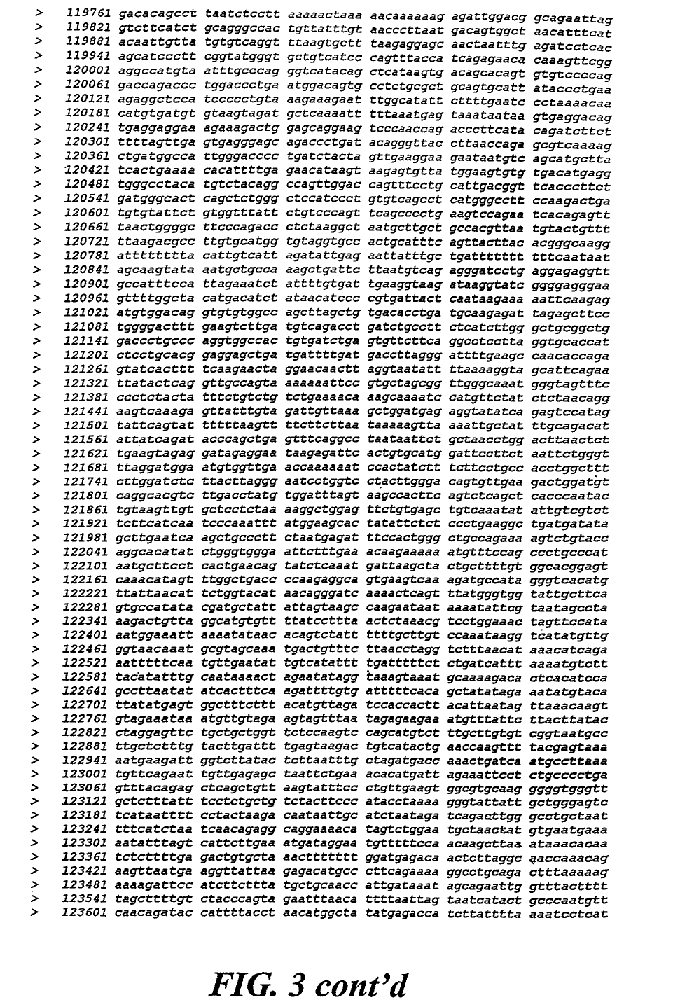

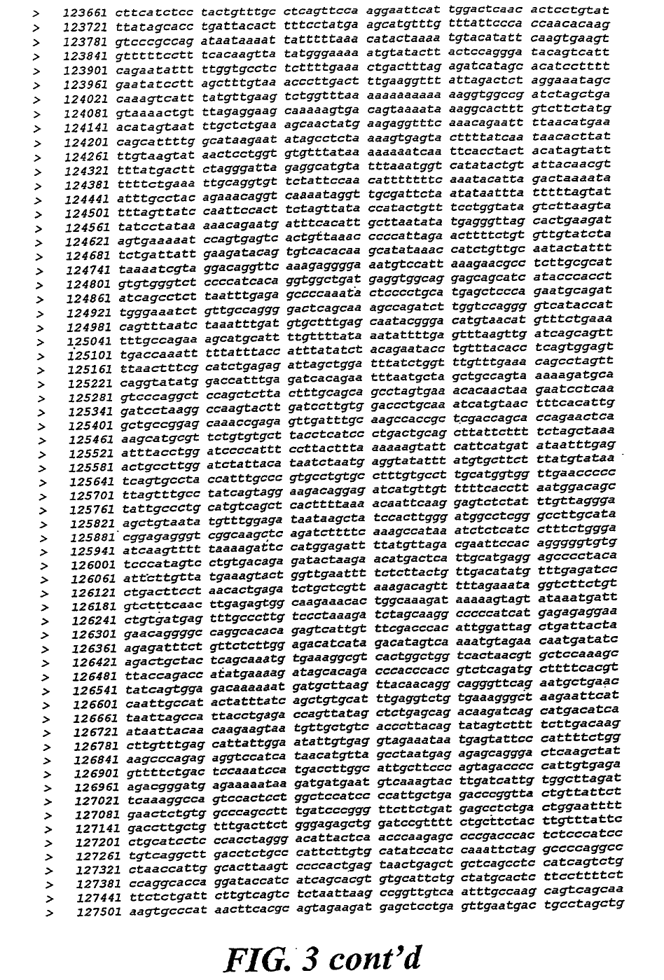

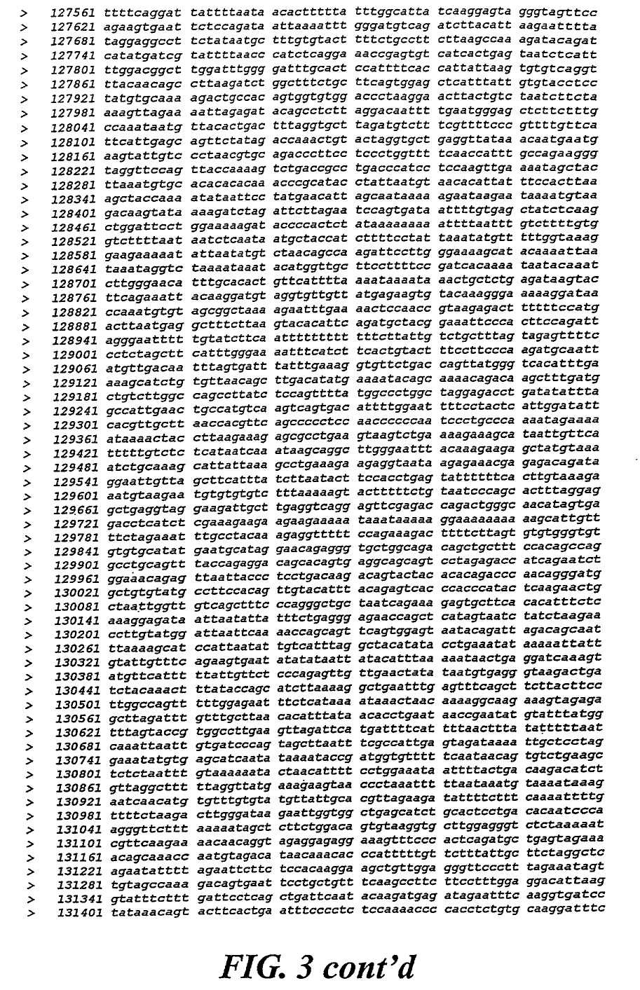

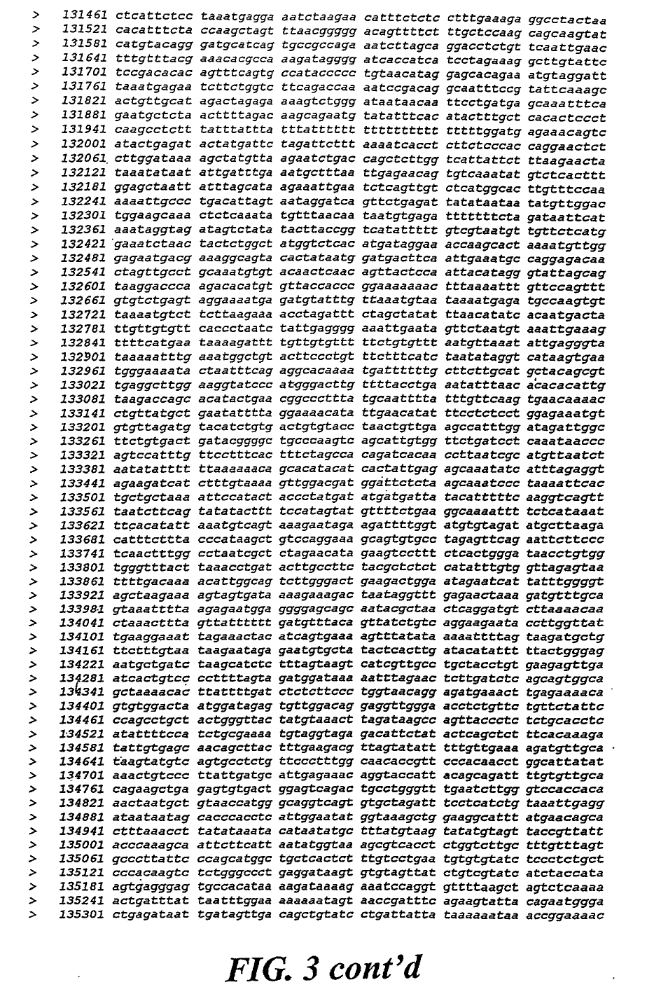

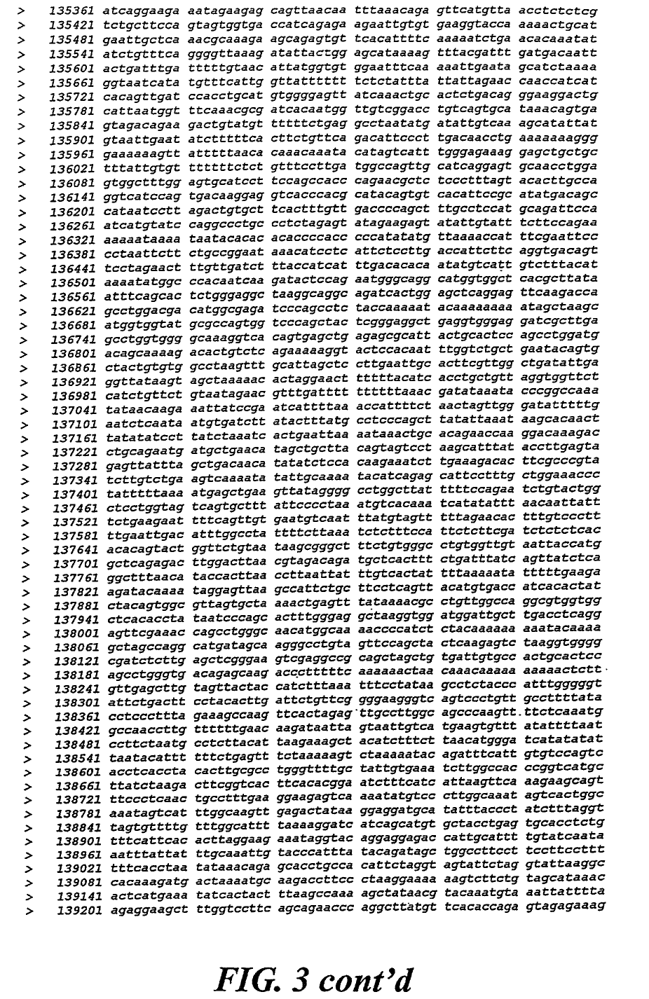

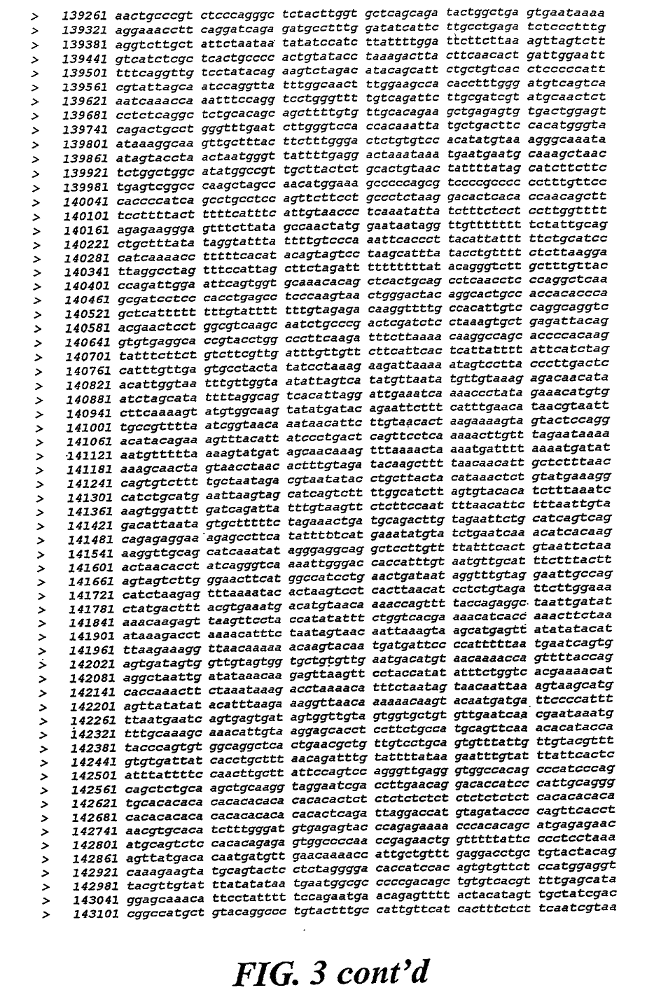

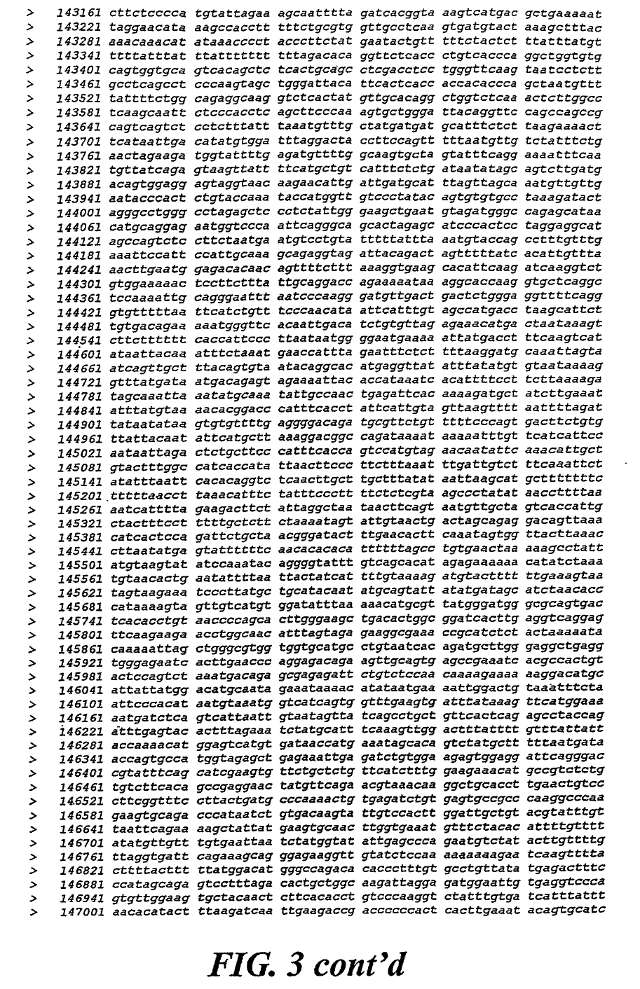

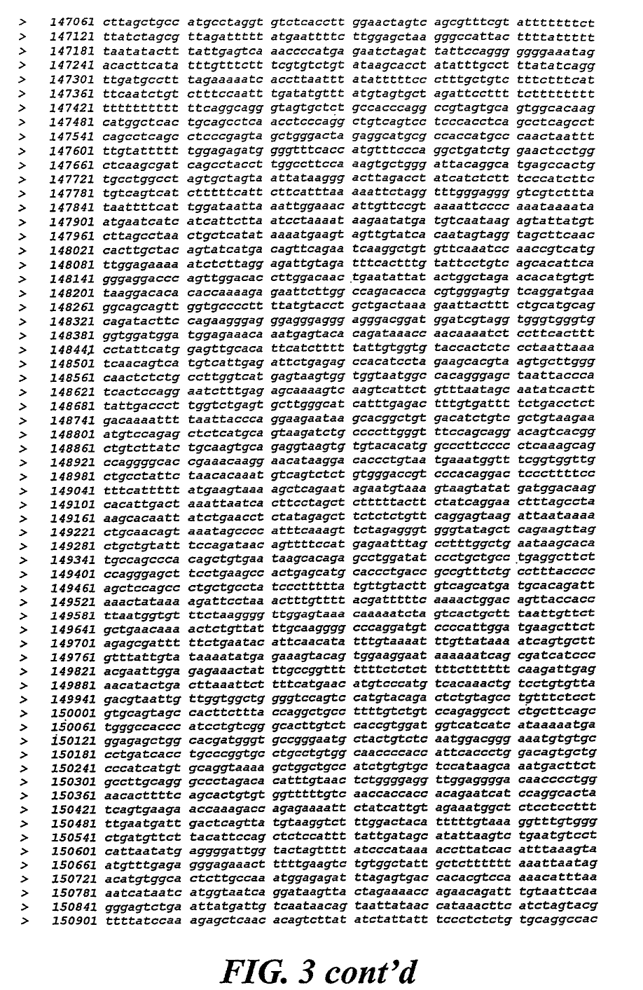

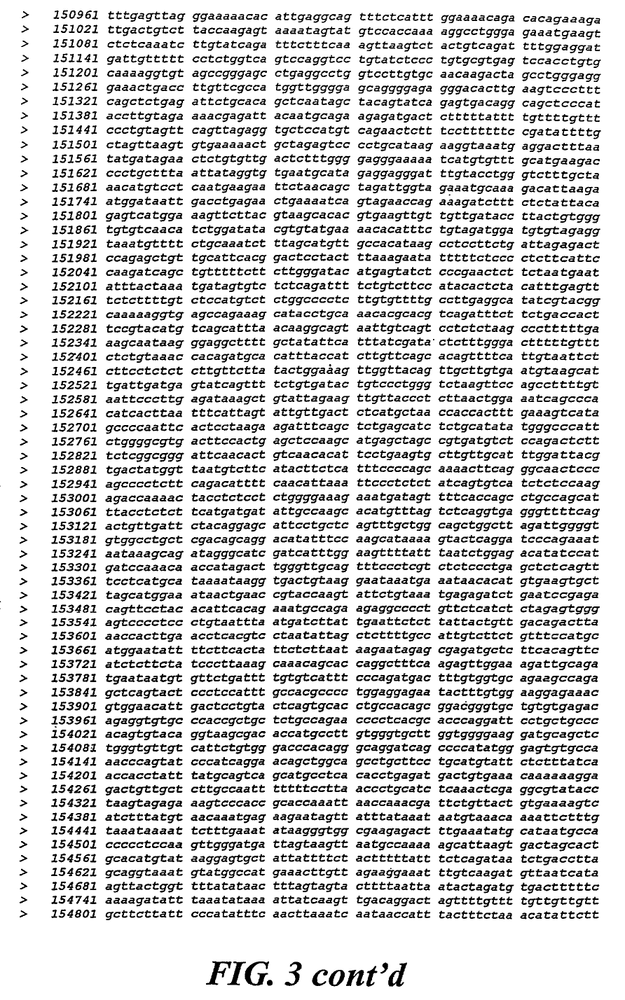

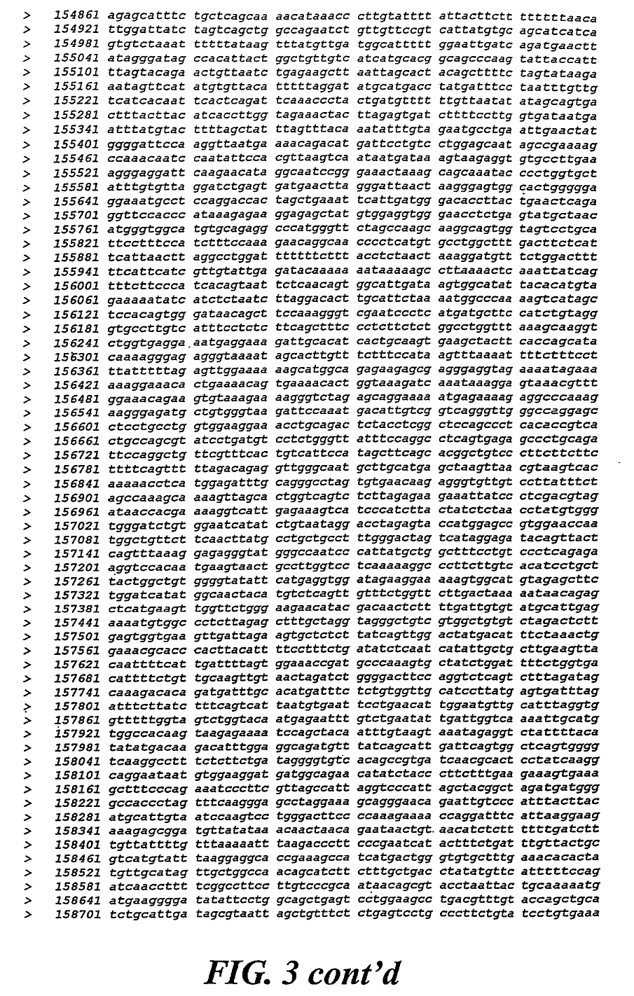

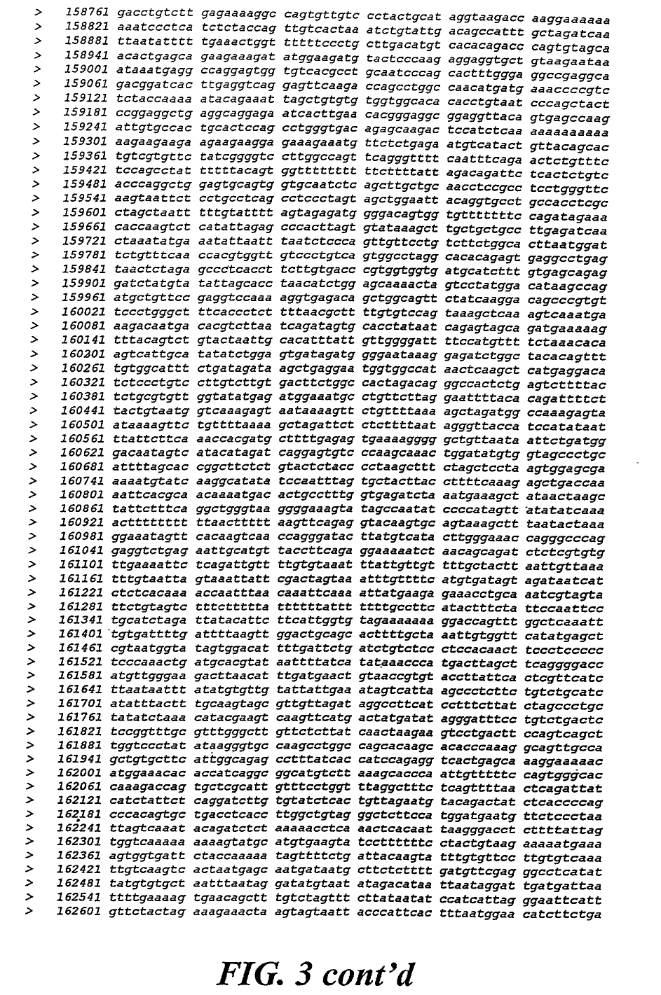

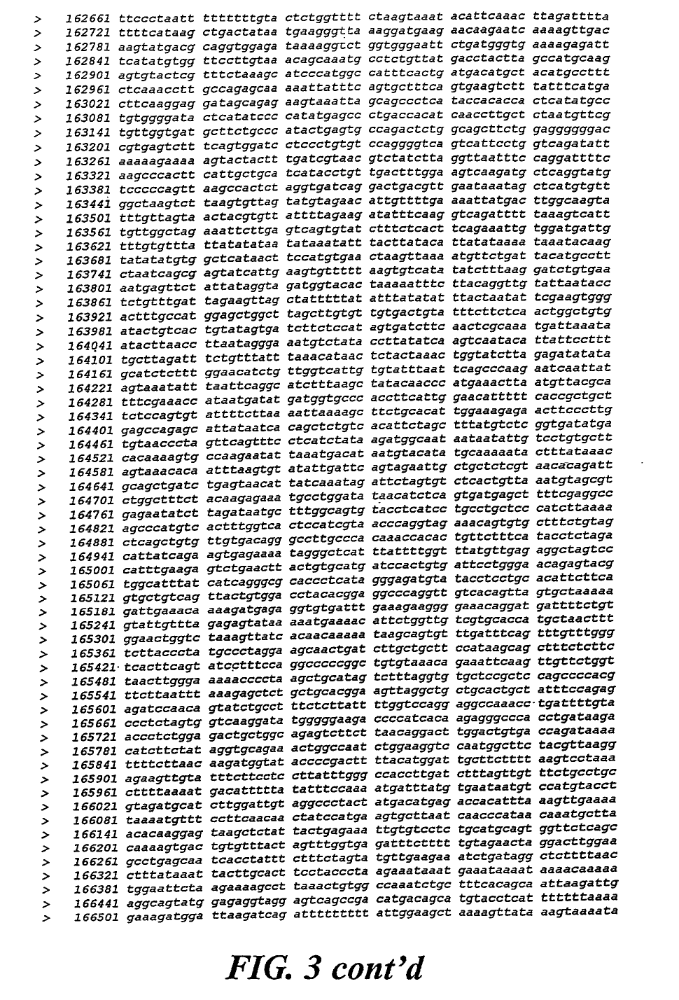

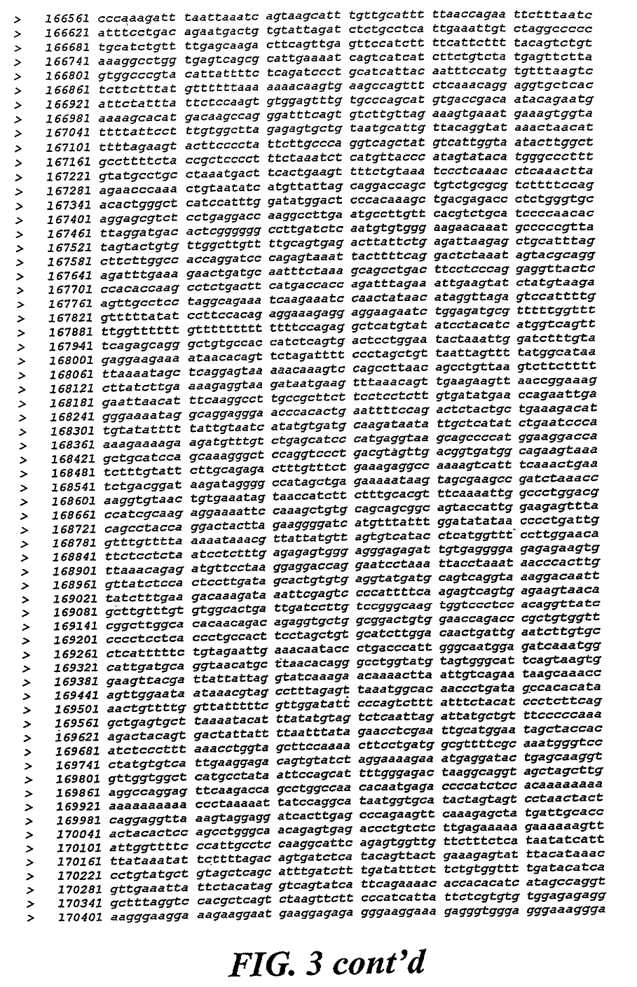

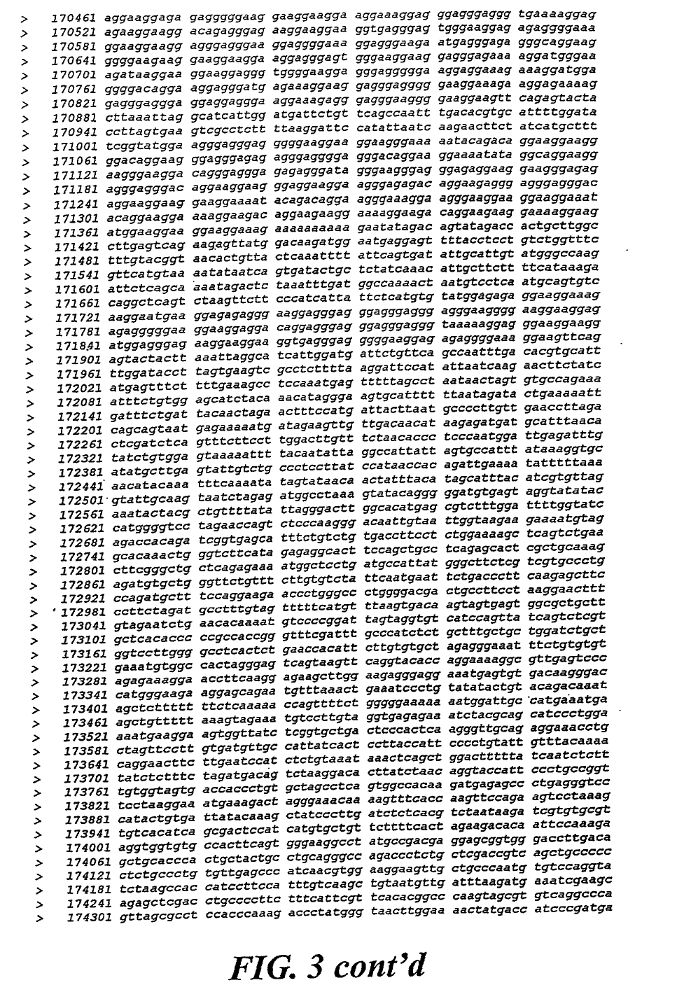

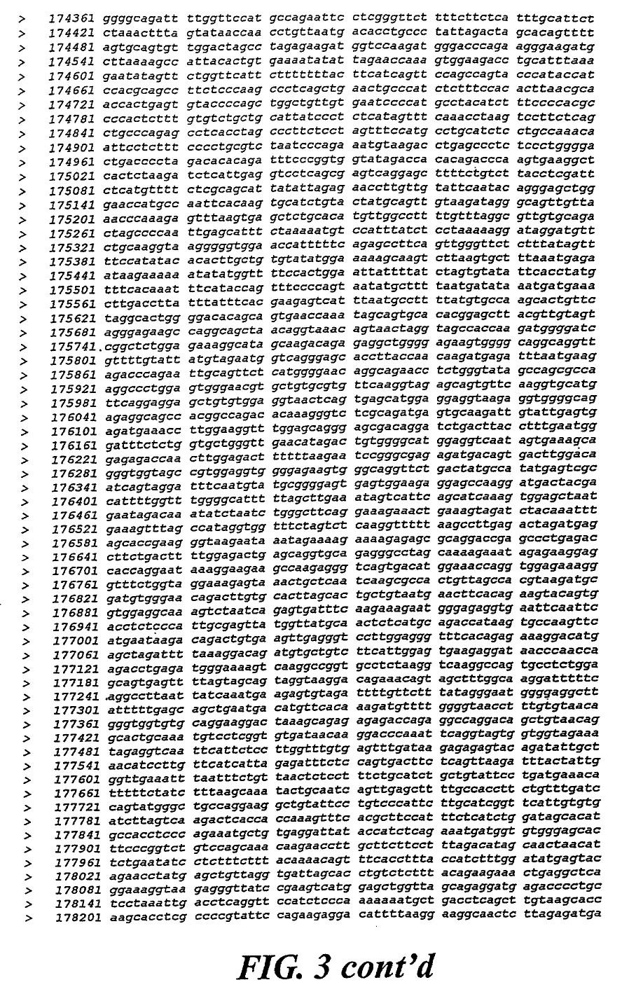

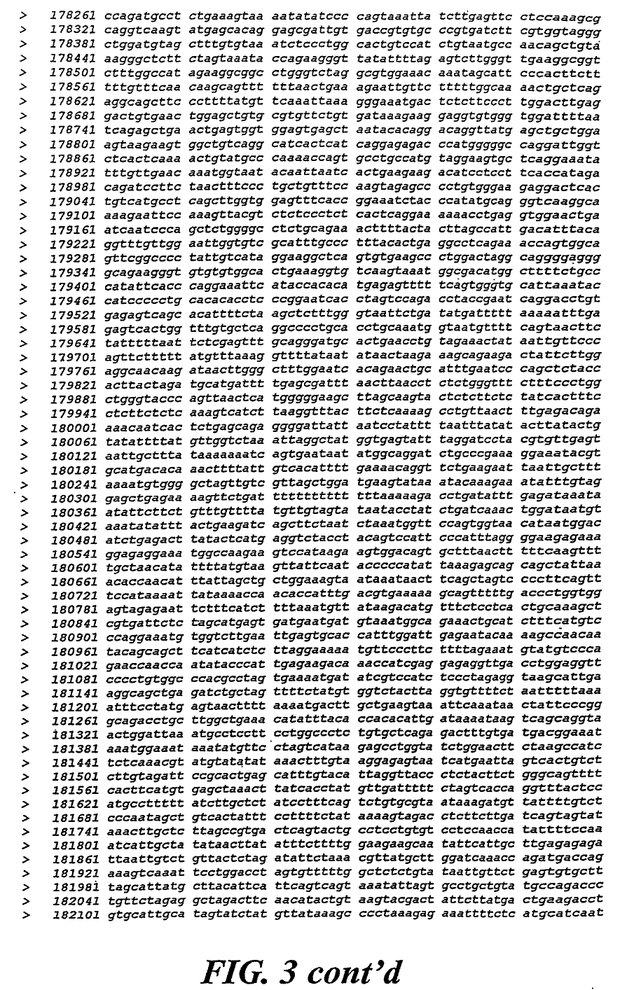

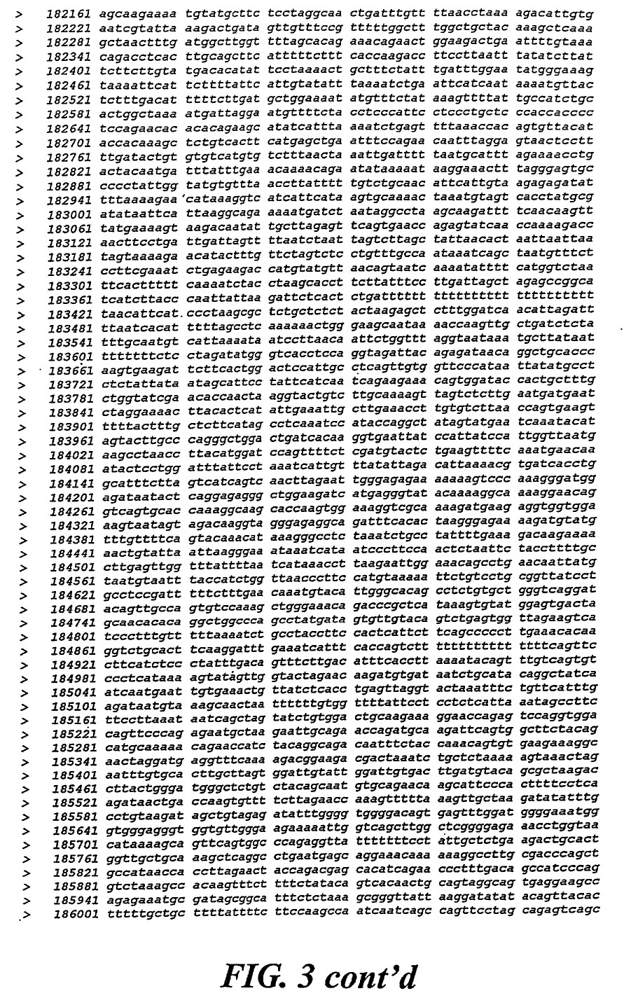

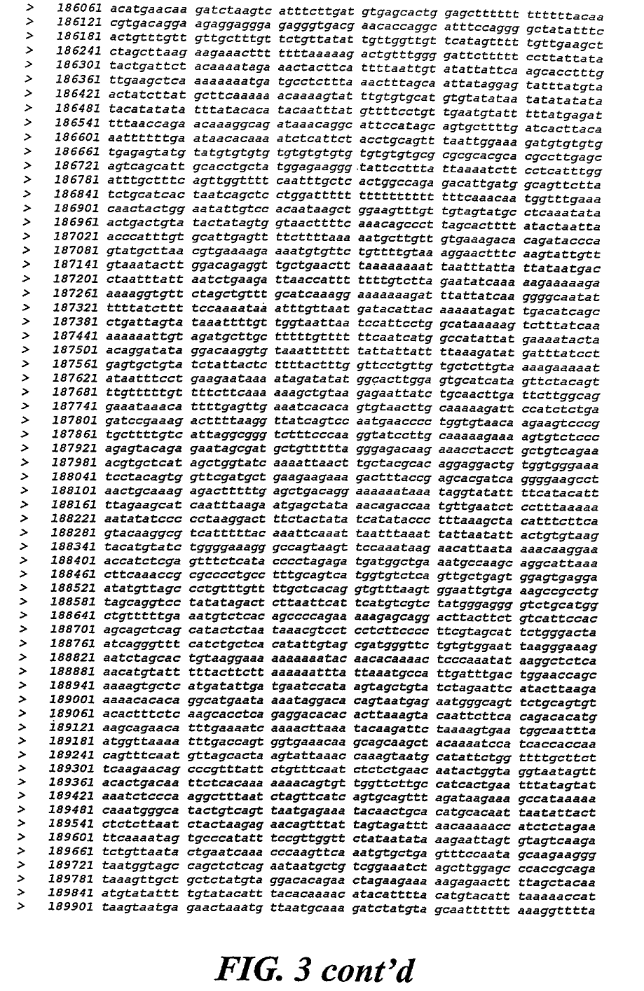

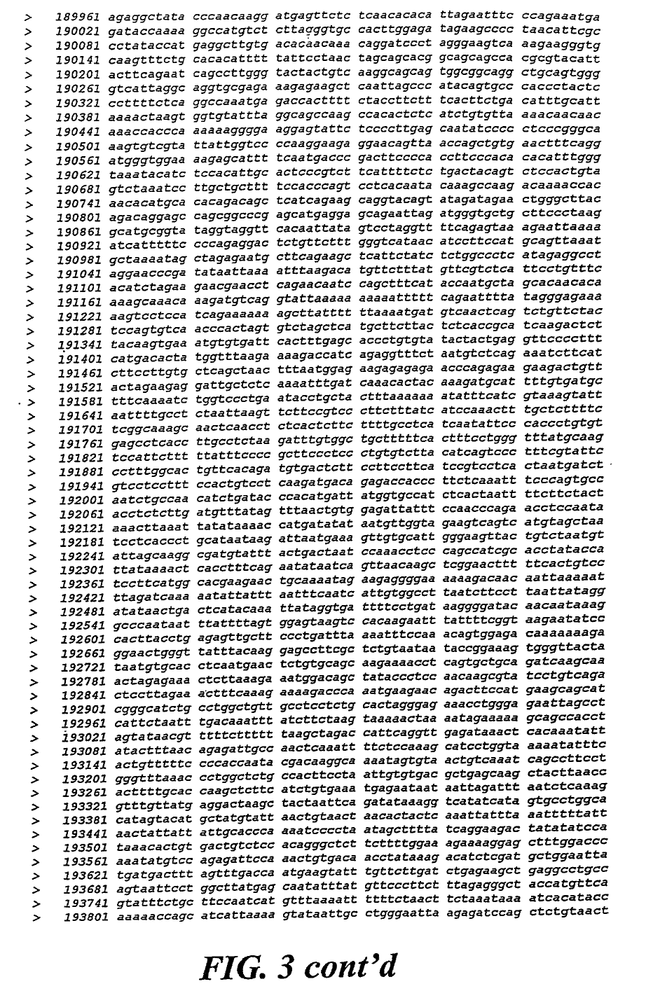

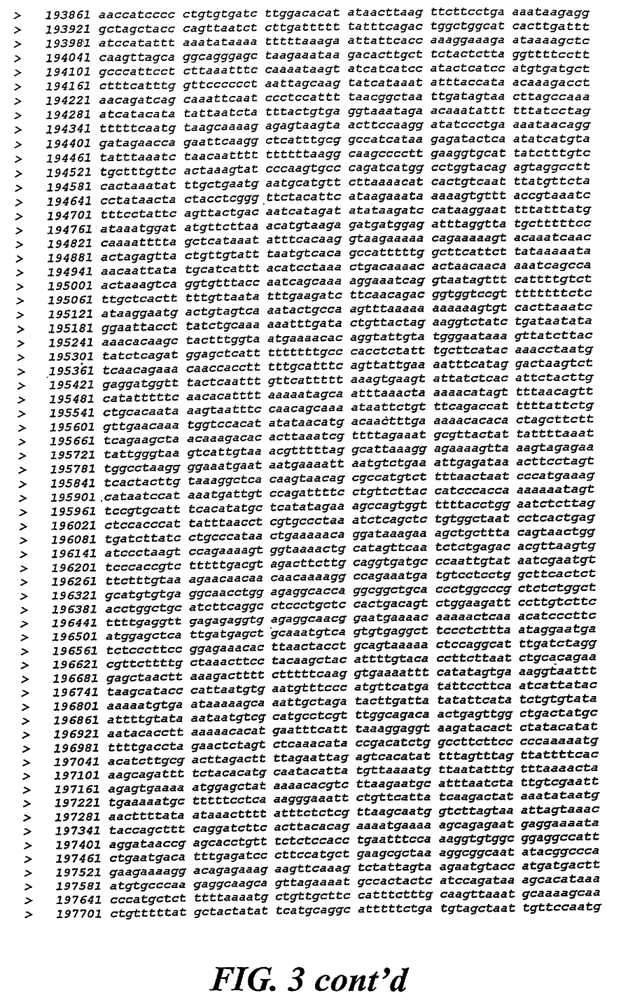

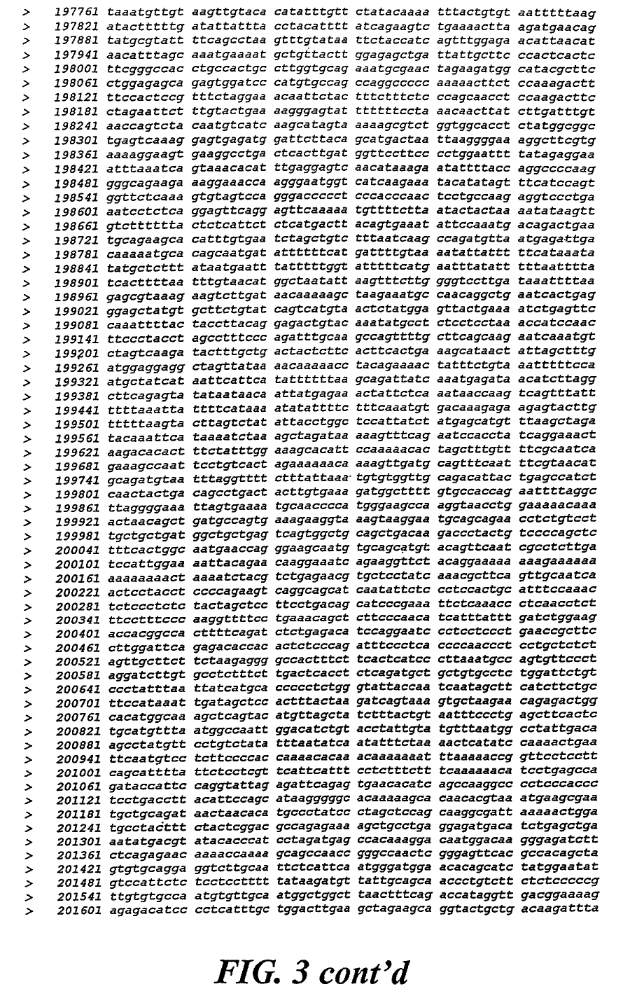

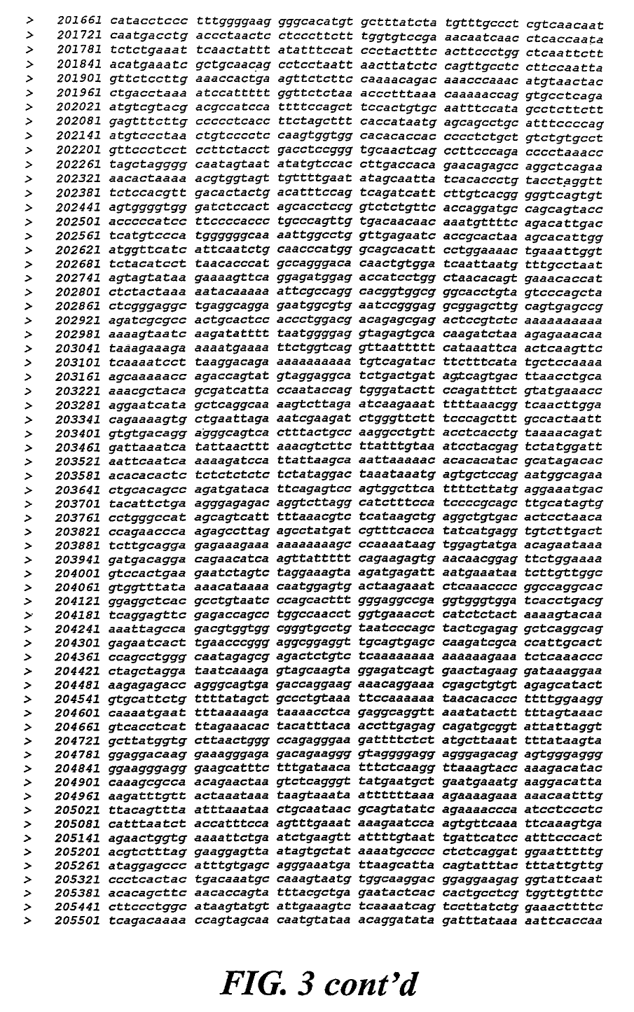

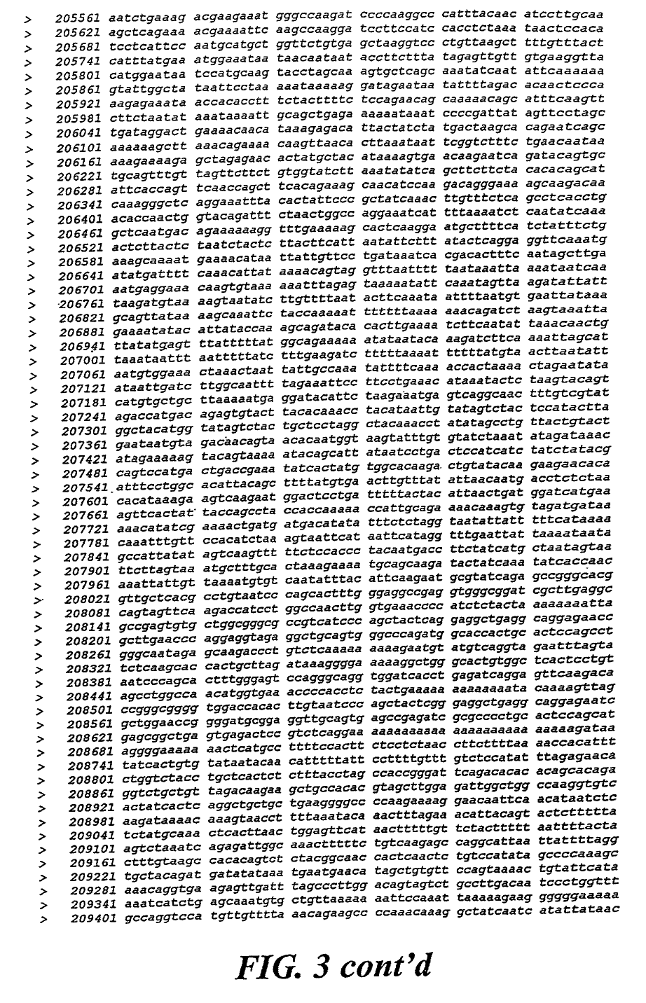

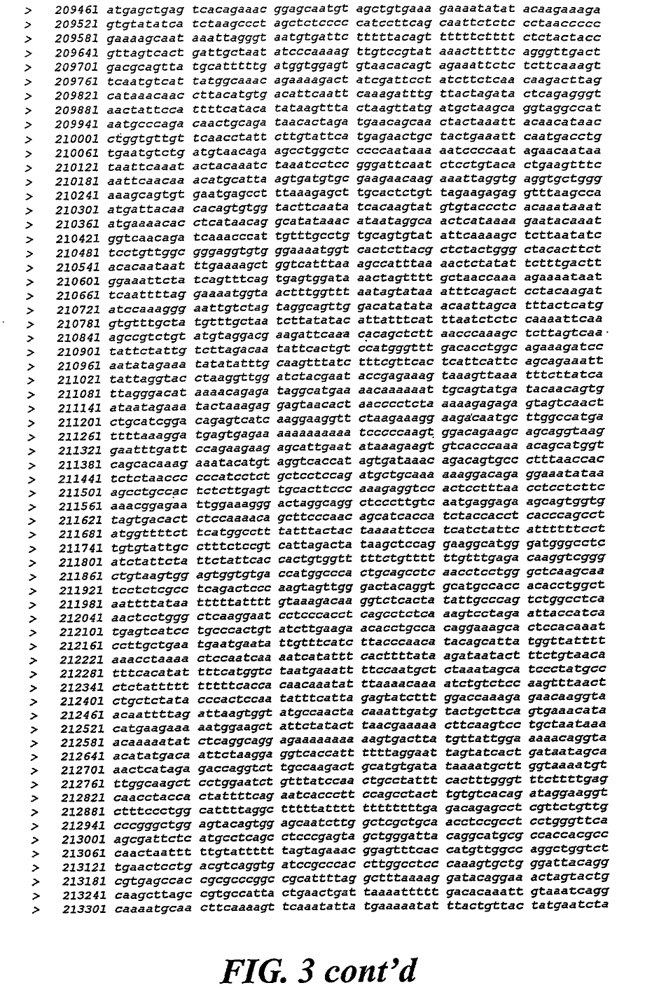

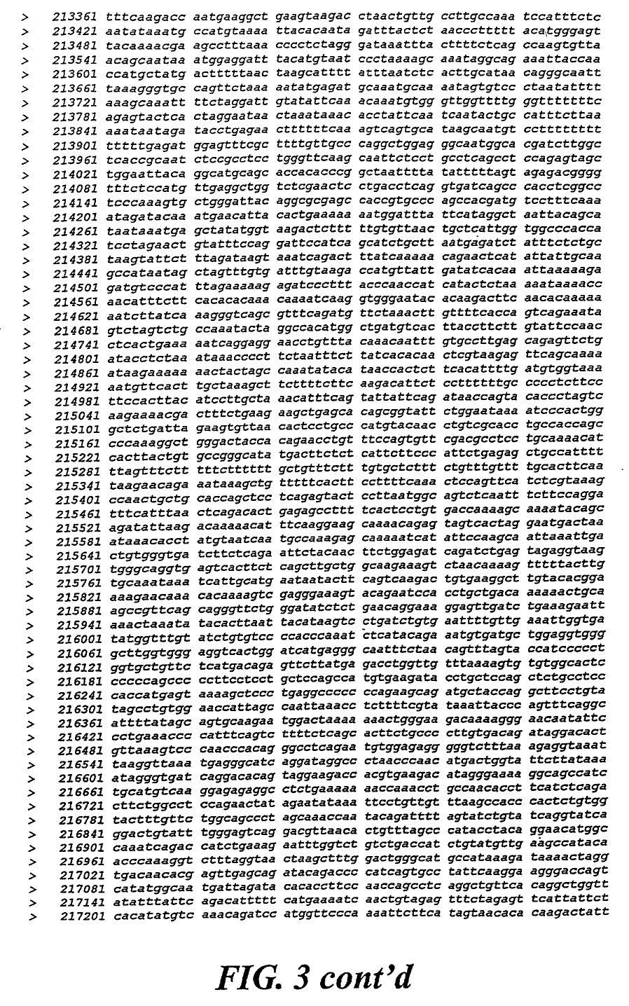

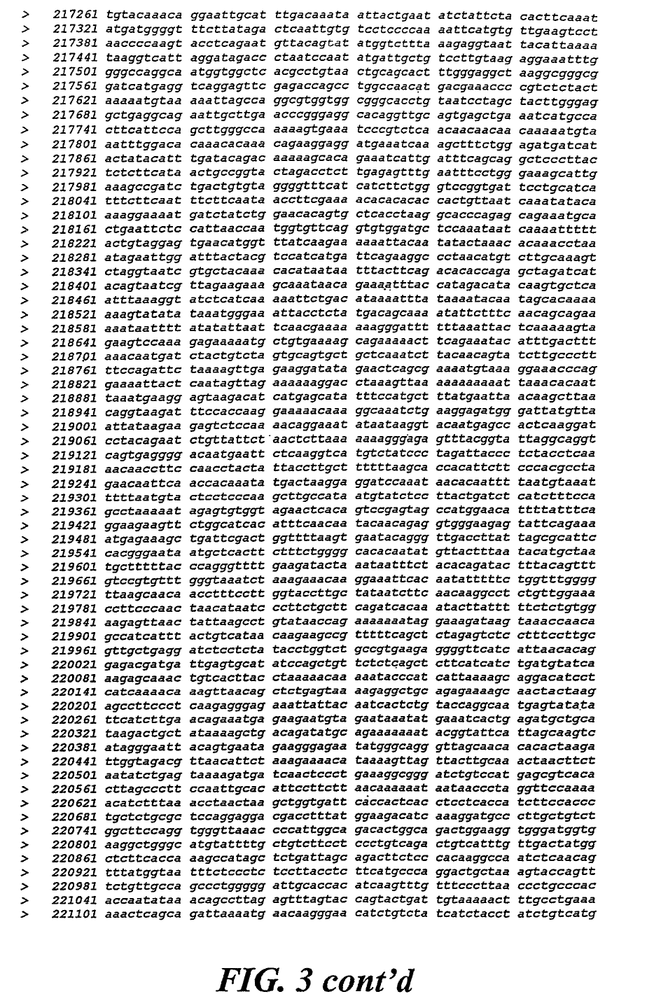

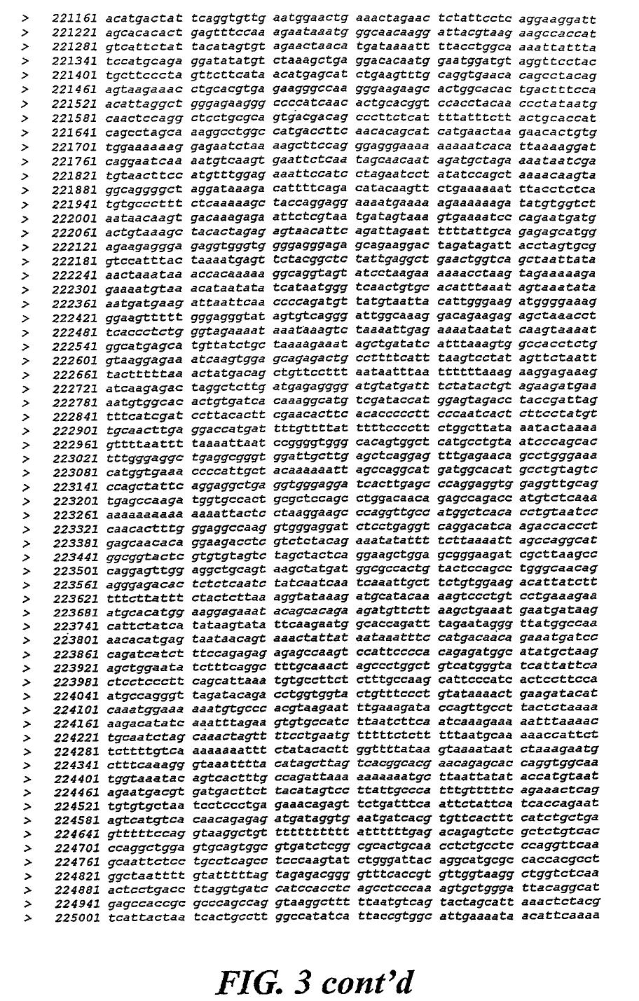

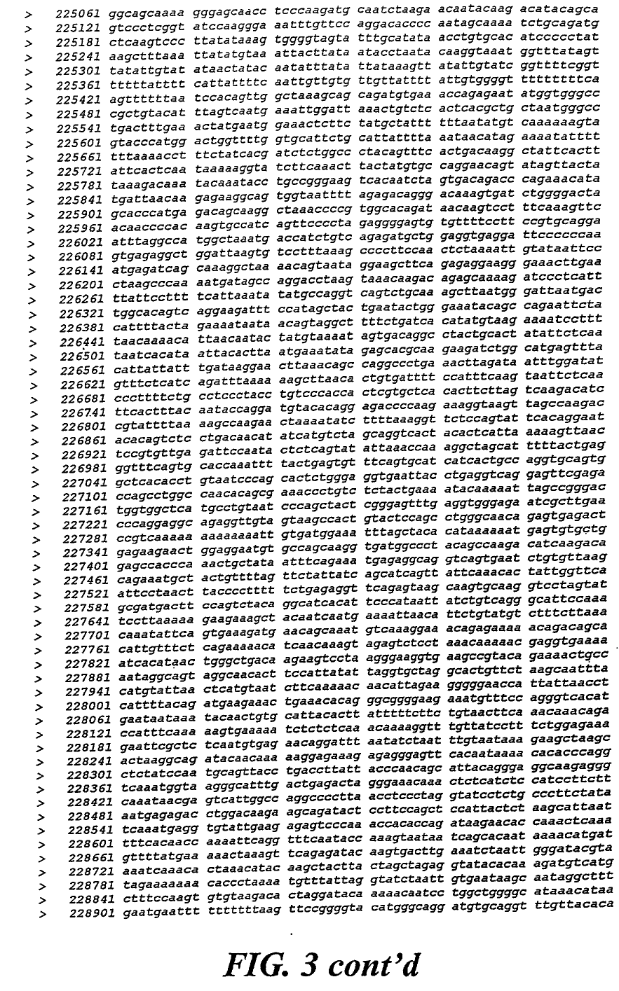

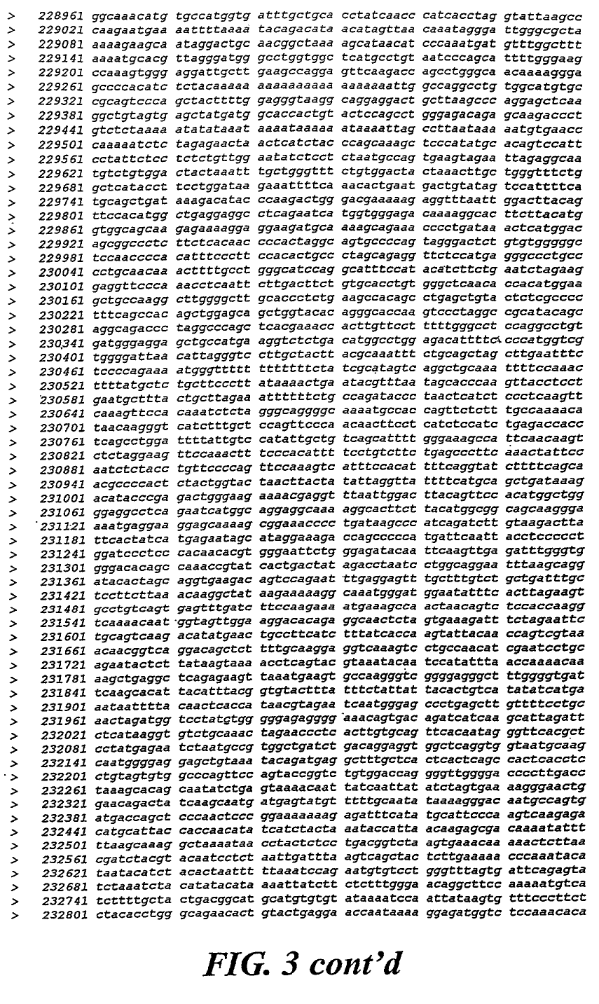

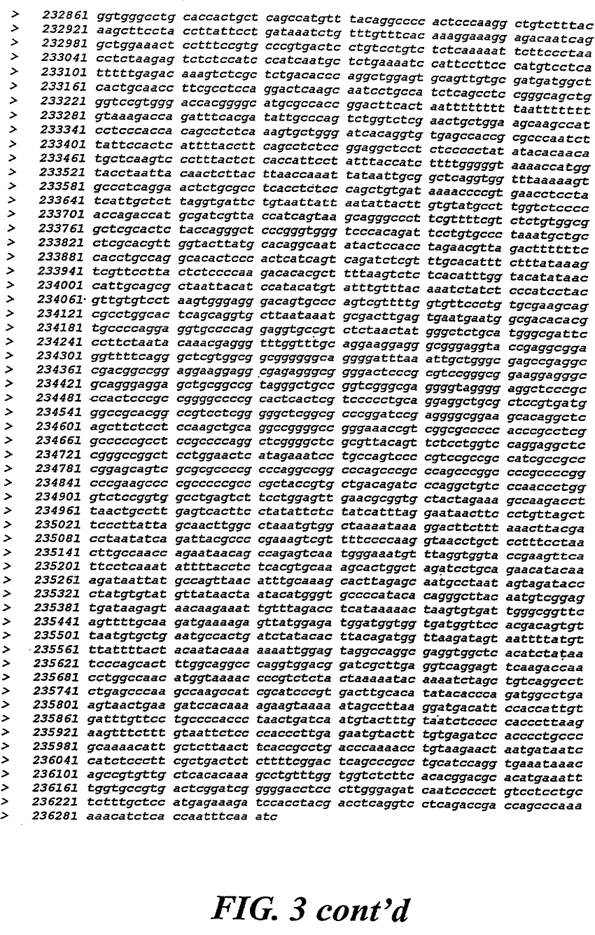

The invention also provides the genomic sequence of FIG. 3, which sequence includes exons 2-17 of the Crim1 gene located on human chromosome 2p2l-16.3 (SEQ ID NOS: 20-24).

The term “nucleic acid” as used herein designates single-or double-stranded mRNA, RNA, cRNA and DNA, said DNA inclusive of cDNA and genomic DNA.

A “polynucleotide” is a nucleic acid having eighty (80) or more contiguous nucleotides, while an “oligonucleotide” has up to eighty (80) contiguous nucleotides.

A “probe” may be a single or double-stranded oligonucleotide or polynucleotide, suitably labeled for the purpose of detecting complementary sequences in Northern or Southern blotting, for example.

A “primer” is usually a single-stranded oligonucleotide, preferably having 15-50 contiguous nucleotides, which is capable of annealing to a complementary nucleic acid “template” and being extended in a template-dependent fashion by the action of a DNA polymerase such as Taq polymerase, RNA-dependent DNA polymerase or Sequenase™.

The present invention also contemplates homologs of Crim1 nucleic acids of the invention.

In one embodiment, nucleic acid homologs encode polypeptide homologs of the invention, inclusive of variants, fragments and derivatives thereof.

In another embodiment, nucleic acid homologs share at least 60%, preferably at least 70%, more preferably at least 80%, or even more preferably at least 90% sequence identity with the nucleotide sequences of any one of SEQ ID NOS: 4-7 or SEQ ID NOS: 20-24.

As generally used herein, a “homolog” shares a definable nucleotide or amino acid sequence relationship with a nucleic acid or polypeptide of the invention as the case may be.

Included within the scope of homologs are “orthologs”, which are functionally-related polypeptides and their encoding nucleic acids, isolated from different organisms. It will be appreciated that the CRIM1 polypeptides and Crim1 nucleic acids isolated from human, mouse and chicken constitute a family of orthologs.

Terms used herein to describe sequence relationships between respective nucleic acids and polypeptides include “comparison window”, “sequence identity”, “percentage of sequence identity” and “substantial identity”. Because respective nucleic acids/polypeptides may each comprise (1) only one or more portions of a complete nucleic acid/polypeptide sequence that are shared by the nucleic acids/polypeptides, and (2) one or more portions which are divergent between the nucleic acids/polypeptides, sequence comparisons are typically performed by comparing sequences over a “comparison window” to identify and compare local regions of sequence similarity. A “comparison window” refers to a conceptual segment of typically at least 6 contiguous residues that is compared to a reference sequence. The comparison window may comprise additions or deletions (i.e., gaps) of about 20% or less as compared to the reference sequence (which does not comprise additions or deletions) for optimal alignment of the respective sequences. Optimal alignment of sequences for aligning a comparison window may be conducted by computerised implementations of algorithms (Geneworks program by Intelligenetics; GAP, BESTFIT, FASTA, and TFASTA in the Wisconsin Genetics Software Package Release 7.0, Genetics Computer Group, 575 Science Drive Madison, Wis., USA, incorporated herein by reference) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25 3389, which is incorporated herein by reference.

A detailed discussion of sequence analysis can be found in Unit 19.3 of CURRENT PROTOCOLS IN MOLECULAR BIOLOGY Eds. Ausubel et al. (John Wiley & Sons Inc NY, 1995-1999).

The term “sequence identity” is used herein in its broadest sense to include the number of exact nucleotide or amino acid matches having regard to an appropriate alignment using a standard algorithm, having regard to the extent that sequences are identical over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. For example, “sequence identity” may be understood to mean the “match percentage” calculated by the DNASIS computer program (Version 2.5 for windows; available from Hitachi Software engineering Co., Ltd., South San Francisco, Calif., USA).

Homologs therefore include nucleic acids of the invention which have nucleotide substitutions, deletions or additions which do not substantially alter functional characteristics of polypeptides encoded thereby.

In this regard, a skilled addressee would realize that advantage can be taken of codon sequence redundancy to incorporate changes in a nucleotide sequence without affecting the encoded amino acid sequence of a polypeptide encoded thereby. Furthermore, nucleic acids may be altered so as to introduce “conservative” amino acid changes which, although altering an amino acid sequence, do not affect functional characteristics of polypeptides encoded thereby.

Nucleic acid homologs of the invention may also comprise nucleic acids which hybridize with isolated Crim1 nucleic acids of the invention under at least low stringency conditions, preferably at least medium stringency conditions, or more preferably at least high stringency conditions.

“Hybridization” is used herein to denote the pairing of complementary bases of distinct nucleic acids to produce a DNA-DNA hybrid, a DNA-RNA hybrid, or an RNA-RNA hybrid according to base-pairing rules.

Typically, hybridizing nucleic acids are identified by blotting techniques that include a step whereby polynucleotides are immobilized on a matrix (preferably a synthetic membrane such as nitrocellulose), a hybridization step, a washing step and a detection step.

Southern blotting is used to identify a complementary DNA sequence; Northern blotting is used to identify a complementary RNA sequence. Dot blotting and slot blotting can be used to identify complementary DNA/DNA, DNA/RNA or RNA/RNA nucleic acids. Such techniques are well known by those skilled in the art, and have been described in CURRENT PROTOCOLS IN MOLECULAR BIOLOGY (Eds. Ausubel et al., John Wiley & Sons Inc 1995) at pages 2.9.1 through 2.9.20. According to such methods, Southern blotting involves separating DNA molecules according to size by gel electrophoresis, transferring the size-separated DNA to a synthetic membrane, and hybridizing the membrane bound DNA to a complementary nucleic acid labeled radioactively, enzymatically or fluorochromatically. In dot blotting and slot blotting, DNA samples are directly applied to a synthetic membrane prior to hybridization as above.

Similarly, a blotting step is used when identifying complementary nucleic acids in a cDNA or genomic DNA library, such as through the process of plaque or colony hybridization. A typical example of this procedure is described in Sambrook et al., MOLECULAR CLONING: A LABORATORY MANUAL 2nd Ed (Cold Spring Harbour Press 1989) Chapters 8-12, which is herein incorporated by reference.

“Stringency” as used herein, refers to the temperature and ionic strength conditions, and presence or absence of certain organic solvents, during hybridization. The higher the stringency, the higher will be the degree of complementarity between the immobilized nucleic acids and the labeled nucleic acid.

“Stringent conditions” designates those conditions under which only nucleic acids having a high frequency of complementary bases will hybridize, and remain hybridized during washing.

Reference herein to low stringency conditions includes and encompasses:—

(i) from at least about 1% v/v to at least about 15% v/v formamide and from at least about 1 M to at least about 2 M salt for hybridisation at 42° C., and at least about 1 M to at least about 2 M salt for washing at 42° C.; and

(ii) 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature.

Medium stringency conditions include and encompass:—

(i) from at least about 16% v/v to at least about 30% v/v formamide and from at least about 0.5 M to at least about 0.9 M salt for hybridisation at 42° C., and at least about 0.5 M to at least about 0.9 M salt for washing at 42° C.; and

(ii) 1% Bovine Serum Albumin (BSA), 1 mM EDTA, 0.5 M NaHPO₄ (pH 7.2), 7% SDS for hybridization at 65° C. and (a) 2×SSC, 0.1% SDS; or (b) 0.5% BSA, 1 mM EDTA, 40 mM NaHPO₄ (pH 7.2), 5% SDS for washing at 42° C.

High stringency conditions include and encompass:—

(i) from at least about 31% v/v to at least about 50% v/v formamide and from at least about 0.01 M to at least about 0.15 M salt for hybridisation at 42° C., and at least about 0.01 M to at least about 0.15 M salt for washing at 42° C.;

(ii) 1% BSA, 1 mM EDTA, 0.5 M NaHPO₄ (pH 7.2), 7% SDS for hybridization at 65° C., and (a) 0.1×SSC, 0.1% SDS; or (b) 0.5% BSA, 1 mM EDTA, 40 mM NaHPO₄ (pH 7.2), 1% SDS for washing at a temperature in excess of 65° C. for about one hour; and

(iii) 0.2×SSC, 0.1% SDS for washing at or above 68° C. for about 20 minutes.

In general, the Tₘ of a duplex DNA decreases by about 1° C. with every increase of 1% in the number of mismatched bases.

Notwithstanding the above, stringent conditions are well known in the art, such as described in Chapters 2.9 and 2.10 of. Ausubel et al, supra, which are herein incorporated be reference. A skilled addressee will also recognize that various factors can be manipulated to optimize the specificity of the hybridization. Optimization of the stringency of the final washes can serve to ensure a high degree of hybridization.

In another embodiment, nucleic acid homologs may be prepared according to the following procedure:

(i) creating primers which, optionally, are degenerate wherein each comprises a respective portion of an isolated Crim1 nucleic acid; and

(ii) using said primers to amplify, via nucleic acid amplification techniques, one or more amplification products from a nucleic acid extract.

Suitable nucleic acid amplification techniques are well known to the skilled addressee, and include polymerase chain reaction (PCR) as for example described in Chapter 15 of Ausubel et al. supra, which is incorporated herein by reference; strand displacement amplification (SDA) as for example described in U.S. Pat. No 5,422,252 which is incorporated herein by reference; rolling circle replication (RCR) as for example described in Liu et al., 1996, J. Am. Chem. Soc. 118 1587, International application WO 92/01813 and International Application WO 97/19193, which are incorporated herein by reference; nucleic acid sequence-based amplification (NASBA) as for example described by Sooknanan et al.,1994, Biotechniques 17 1077 which is incorporated herein by reference; ligase chain reaction (LCR) as for example described in International Application WO89/09385 which is incorporated by reference herein; and Q-β replicase amplification as for example described by Tyagi et al., 1996, Proc. Natl. Acad. Sci. USA 93 5395) which is incorporated herein by reference.

As used herein, an “amplification product” refers to a nucleic acid product generated by nucleic acid amplification techniques.

The nucleic acid extract may be an extract obtained from cells, tissues or biological fluids in the form of mRNA, or as cDNA reverse transcribed therefrom. The extract may be in the form of a cDNA or genomic library. In this regard, the cDNA or genomic library is preferably derived from a eukaryote, and advantageously from mammals such as humans. Such libraries may comprise genomic DNA or cDNA ligated into vectors such as will be described hereinafter.

Expression Constructs

The invention provides an expression construct which comprises an isolated Crim1 nucleic acid or homolog thereof, operably linked to one or more regulatory sequences in an expression vector.

An example of an expression construct of the invention is pcDNA3-hCRIM1myc deposited under accession number NM00/16530 at AGAL on Nov. 9, 2000.

Regulatory nucleotide sequences present in the expression vector (such as an enhancer, promoter, splice donor/acceptor signals, terminator and polyadenylation sequences) that will facilitate expression of the polypeptide of the invention. Selectable markers are also useful whether for the purposes of selection of transformed bacteria (such as bla, kanR and tetR) or transformed mammalian cells (such as hygromycin, G418 and puromycin).

Both constitutive and inducible promoters may be useful for expression of Crim 1 polypeptides according to the invention. Examples of inducible promoters are metallothionine-inducible and tetracycline-repressible systems as are well known in the art.

An expression construct may also include a fusion partner sequence as hereinbefore defined (such as myc of GFP) so that the recombinant polypeptide of the invention is expressed as a fusion polypeptide with said fusion partner.

Suitable host cells for Crim1/CRIM1 expression include bacteria (eg. E coli. DH5α), yeast, insect cells (eg. Sf9), Xenopus oocytes and mammalian cells such as CHO and COS lines.

Expression constructs also include gene therapy constructs, which employ specialized gene therapy vectors such as vaccinia, and viral vectors useful in gene therapy. The latter include adenovirus and adenovirus-associated viruses (AAV) such as described in Franceschi et al., 2000, J. Cell Biochem. 78 476, Braun-Falco et al.,1999, Gene Ther. 6 432, retroviral and lentiviral vectors such as described in Buchshacher et al, 2000, Blood 95 2499 and vectors derived from herpes simplex virus and cytomegalovirus. A general review of gene therapy vectors may be found in Robbins et al., 1998, Trends in Biotech. 16 35. An overview of viral vectors useful in endocrine gene therapy is provided in Stone et al., 2000, J. Endocrinol. 164 103.

If “anti-sense” therapy is contemplated, then one or more selected portions of said Crim1 nucleic acid may be oriented 3′→5′ in the gene therapy vector.

Pharmaceutical Compositions

The invention provides pharmaceutical compositions comprising a CRIM1 polypeptide or a Crim1 nucleic acid and a pharmaceutically-acceptable carrier, diluent or excipient. Also contemplated are pharmaceutical compositions comprising a CRIM1 mimetic or homologs of Crim1 nucleic acids and encoded polypeptides.

Pharmaceutical compositions comprising a Crim1 nucleic acid are preferably in the form of a gene therapy construct.

Such pharmaceutical compositions may be useful in prophylactic or therapeutic treatments for diseases including a neurodegenerative disease such as motor neuron disease, diseases of the eye or more particularly the eye lens, such as glaucoma, cataracts, micropthalmia, microcornea or nuclear scelorosis of the lens. Other possible diseases may relate to heart development, kidney and gonad development, tooth development and bone morphogenesis and healing of wounds and damaged skin.

“Pharmaceutically-acceptable carriers, diluents and excipients” include a solid or liquid filler, diluent or encapsulating substance which may be safely used in systemic administration. Depending upon the particular route of administration, a variety of pharmaceutically-acceptable carriers, diluents or excipients, well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline, and pyrogen-free water.

Dosage forms include tablets, dispersions, suspensions, injections, solutions, syrups, troches, capsules, suppositories, topically administered powders, aerosols and emulsions, transdermal patches, gels, pastes and the like. These dosage forms may also include controlled release devices or other forms of implants modified to act in this fashion. Controlled release of the therapeutic agent may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, the controlled release may be effected by using other polymer matrices, liposomes and/or microspheres.

Compositions of the present invention may be suitable for administration orally or by injection, and in such cases may be presented as discrete units such as capsules, sachets or tablets, as a powder or granules, or as a solution or a suspension in an aqueous liquid, a non-aqueous liquid, an oil-in-water emulsion or a water-in-oil liquid emulsion.

Administration of the gene therapy construct to an animal, preferably a human individual, may include delivery via direct oral intake, systemic injection, or delivery to selected tissue(s) or cells, or indirectly via delivery to cells isolated from the mammal or a compatible donor. An example of the latter approach would be stem-cell therapy, wherein isolated stem cells having potential for growth and differentiation are transfected with a gene therapy construct which includes a Crim1 nucleic acid or homolog. The stem-cells are cultured for a period and then transferred to the animal being treated.

Delivery of said gene therapy construct to cells or tissues of said mammal or said compatible donor may be facilitated by microprojectile bombardment, liposome mediated transfection (e.g. lipofectin or lipofectamine), electroporation, calcium phosphate or DEAE-dextran-mediated transfection, for example. A discussion of suitable delivery methods may be found in Chapter 9 of CURRENT PROTOCOLS IN MOLECULAR BIOLOGY (Eds. Ausubel et al.; John Wiley & Sons Inc., 1997 Edition), for example, which is herein incorporated by reference.

Therapeutic Uses

Conservation of CRIM1/Crim1 and its expression during embryonic development suggests that CRIM1/Crim1 may be important for normal development of vertebrates. For example, mutations of this gene may be involved in human disease. Based on data which will be provided in more detail hereinafter, CRIM1 polypeptide and Crim1 nucleic acids may be useful in treating diseases including neurodegenerative disease such as motor neuron disease, diseases of the eye or more particularly the eye lens, such as glaucoma, cataracts, micropthalmia, microcomea or nuclear scelorosis of the lens. Other possible diseases may relate to heart development, kidney and gonad development, tooth development and/or bone morphogenesis and healing of wounds and damaged skin.

Members of the TGFβ superfamily, including the bone morphogenetic proteins (BMPs), are expressed during, and are critical for, early embryo development and organogenesis of a variety of organs, including the kidney, eye (cornea and lens), heart, skeleton, tooth. limb and central nervous system. By interacting with members of the (bone morphogenic protein) BMP, GDF, activin or TGFβ family, CRIM1 may have a role in development, remodelling or repair of these various organs. More specifically, CRIM1 may be able to modulate activities such as bone remodelling, tissue regeneration and motor neuron specification. The genes may be useful in the diagnosis of diseases of these organs and the proteins or genes encoding them in some form of gene therapy construct in the treatment of such conditions.

Administration of pharmaceutical compositions comprising CRIM1 polypeptide or Crim1 nucleic acid may act to modulate the activity activity of BMP family molecules and TGFβ family members, and other molecules such as IGFs.

Chordins are another family of proteins which bind BMP family members. Referring to U.S. Pat. No. 5,846,770, U.S. Pat. No. 5,679,783 and U.S. Pat. No. 5,986,056 (each of which is incorporated herein by reference), it is clear that human chordin, for example, may inhibit or stimulate BMPs and thereby display a number of therapeutic effects.

Therefore, like chordin, it is expected that administration of CRIM1/Crim1, alone or together with other therapeutic proteins or nucleic acids encoding same, may have a variety of therapeutic effects. Examples of other therapeutic proteins contemplated by the present invention include fibroblast growth factor (FGF), activins, inhibins, insulin, insulin-like growth factor (IGF) and epidermal growth factor (EGF). In this regard, it is also noted that the presence of an IGF-binding domain in CRIM1 suggests that CRIM1 may be useful in treating IGF- and/or insulin-related conditions such as insulin-dependent diabetes, skin damage such as ulcers, burns and abrasions, in wound healing and related tissue repair.

CRIM1 is strongly expressed in developing lens. The lens expression persists postnatally but becomes restricted to the epithelial cells at the front of the lens. These cells must be actively maintained in a single layer of epithelium to prevent the obstruction of vision. Disturbances to these cells result in anterior cataract and aftercataract. CRIM1 is likely to play a role in maintaining this layer of cells as an epithelium. Hence, CRIM1 may act in an anti-cataractogenic fashion. Significant evidence exists to suggest that within the eye, TGFβ, particularly TGFβ2, can act in a cataractogenic fashion, leading to disruption in the morphology of the epithelial cells covering the front of the lens. The result of addition of recombinant TGFβ2 to lens explants cultures is the production of plaques identical in histology to those seen arising after surgery for the removal of cataracts. These anterior cataracts are referred to as ‘after cataract’. The suggestion that inhibitors of TGFβ will act in an anti-cataractogenic fashion is supported by the disclosures of International Publication WO95/13827 and International Application WO98/26784, each of which is incorporated herein by reference.

By inhibiting the activity of TGFβ on the lens epithelial cells, CRIM1 may have considerable utility as an anti-cataractogenic agent. This may be developed as a gel or infusion for insertion into the lens capsule at the time of lens replacement operations to protect from after cataract. It may also be deliverable via the aqueous humor to prevent the onset of anterior cataract. In either of these situations, CRIM1 function may be able to be mimicked by a peptide similarly antagonising TGFβ. Such a peptidomimetic may be designed from further analysis of the CRIM1-TGFβ interaction.

Several of the BMPs have the property of generating bone, as suggested by their name. These include BMP2 and 7. BMP7, also called Osteogenic Protein 1 (OP-1), is already known to have potential in bone remodelling, including the treatment of periodontal and orthopedic indications such as fractures. The current approach of mixing OP-1 and a purified collagen matrix into a paste that is applied during surgery may also be applicable to CRIM1. If CRIM1 facilitates the activity of BMP7, this would make it an important accessory for OP-1 treatments. OP-1, while well-established for inducing orthotopic and ectopic bone formation may suffer from limited clinical usefulness as a regenerative agent due to a short in vivo half-life and low specific activity (Franceschi et al, 2000, supra). If CRIM1 stabilises or facilitates an increased BMP half-life, inclusion of this protein in preparations of OP-1 may increase the duration and specificity of the effect. With regard to OP-1 and suitable formulations and delivery of same which may be applicable to the present invention, the skilled person is referred to U.S. Pat. No. 4,968,590, U.S. Pat. No. 5,597,897, U.S. Pat. No. 5,258,494 and U.S. Pat. No. 5,266,683, each of which is incorporated herein by reference.

Several of the BMPs are expressed strongly during the development of the kidney, including BMP2,4 ,5 and 7. A recent review of the filed relating to BMPs and kidney development is provided in Godin et al., 1999, Int. J. Dev. Biol. 43 405, which is incorporated herein by reference.

More particularly, a knockout mouse model of BMP7 −/− reveals significant eye and kidney defects suggesting an important role for BMP7 in the formation of these organs. The kidney defects include renal dysgenesis, cystic kidneys or agenesis. BMP7 is expressed in both the ureteric epithelium and the mesenchyme throughout embryonic development and has been shown to function as a survival factor for the nephrogenic mesenchyme. However, at high concentrations, BMP7 appears to also function as an anti-differentiation factor for the metanephric mesenchyme. CRIM1 shows overlapping expression patterns with BMP2 and BMP7, particularly during the formation of the pretubular aggregates and the comma-shaped bodies, which go on to form the proximal portion of the nephrons of the kidney. The data presented hereinafter suggesting that the presence of a BMP can lead to the liberation of secreted CRIM1 protein may suggest that CRIM1 modulates the roles of BMPs in nephron formation. Stimulation of the anti-differentiative activity of BMP7 during kidney development may make CRIM1 and important protein in the growth and expansion of metanephric mesenchymal populations. The ability to derive and expand such a population of mesenchyme fated for kidney development will be critical in the development of kidney regeneration technologies.

BMP7 expression in the kidney continues after birth, as are receptors for BMP on the podocytes within the glomeruli. Application of BMP7 has been shown to decrease the loss of kidney function associated with acute ischaemic injury (Vukicevic et al., 1998, J. Clin. Invest. 102 202). It can also inhibit tubulointerstitial fibrosis and inflammation after unilateral ureteral obstruction. CRIM1 may similarly assist in such conditions by facilitating or increasing the duration of such BMP7 activity. BMP7 (OP-1) has been found to be preventative for renal fibrosis associated with ureteral obstruction. OP-1 administration can prevent tubular atrophy and diminish the activation of tubulointerstitial inflammation and fibrosis, thereby preserving renal function (Hrusuka et al., 2000, Am. J. Renal. Physiol. 279 130).

By augmenting or potentiating OP-1 activity, CRIM1 is a candidate therapeutic adjuvant for treatment during ureteral obstruction to maintain renal function. Alternatively, if CRIM1 inhibits OP-1 activity, a CRIM1 mimetic could block the interaction between CRIM1 and OP-1 with therapeutically useful effects.

Within the kidney, TGFβ has been implicated in vascular remodelling, premature termination of normal nephrogenesis, promotion of a transition of epithelial cells to mesenchymal cells and a variety of other effects. Increases in circulating TGFβ1 occur during diabetes. This may contribute to the onset of diabetic nephropathy via the induction of collagens 3 and 1 which result in scarring and fibrosis within the kidney. By acting as an inhibitor of this process, CRIM1 may be useful as a preventative therapy for diabetic patients. This is a very large issue for indigenous populations worldwide, including the Pima Indians, Inuits, African Americans and Australian Aboriginals who have high rates of diabetes, high rates of circulating TGFβ1 and high rates of end stage renal disease.

The human condition Alport syndrome results from defects in collagen IV resulting in damage to the glomerular basement membrane and subsequent renal failure. Recent data in mice have shown that in mouse models of this disease, inhibition of TGFβ1 ameliorates the focal thickening of the basement membrane characteristic of Alport syndrome (Cosgrove et al., 2000, Am. J. Pathol. 157 1649). The present invention therefore contemplates CRIM1 acting as an inhibitor of TGFβ1 and thereby being therapeutically useful in treatment of Alport syndrome. It is also contemplated that if CRIM1 potentiates or augments TGFβ1 activity, CRIM1 mimetics may be useful in treating Alport syndrome.

Recent data using human stem cells has revealed that these cells are extremely difficult to maintain in an undifferentiated state, hampering efforts at organ regeneration. One of the genes turned on as these cells start to differentiate is BMP4. Human pluripotent teratocarcinoma stem cell lines have been investigated as a model of human stem cells and show the expression of the stem cell marker Oct4 until, treated with BMP2, upon which they differentiate into endodermal precursors (Pera & Herszfeld, 1998, Reprod. Fertil. Dev. 10 551). By inhibiting BMP2 function, CRIM1 may act to maintain human stem cells in an undifferentiated state, which would be very useful in the expansion of such cells as a potentially unlimited source of many different cell types for cell-based gene and tissue therapies. Conversely, CRIM1 may work in concert with proteins such as BMP2 for the selective reprogramming of such cells for a particular lineage.

Embryonic expression of the Crim1 gene occurs in the notochord and floor plate, which are known to be the source of the embryonic organising centre for the developing central nervous system (CNS). The nucleotide sequence, protein sequence and conservation of the CRIM1 homologues in human, mouse and chick, and C. elegans predict the essential role of CRIM1 conserved function during animal evolution. These findings suggest that CRIM1 functions as part of signalling mechanism which is required for normal CNS development. This may also be important in tissue regeneration including kidney replacement therapy.

CRIM1 protein, nucleic acids encoding said protein, and interacting proteins which selectively bind such proteins may function as a regulator for normal neuronal differentiation in the spinal cord, and migration of neural crest-derived cells, by either direct or indirect interactions with other growth factors such as BMPs, TGFβs and IGFs that are thought to be involved in the normal and/or abnormal neuronal differentiation in mammalian CNS. CRIM1 may also function as a neural cell adhesion molecule that is required for the normal development and maintenance of neurons in the CNS during normal embryonic development in adult. CRIM1 may also promote development of neuronal processes such as axons in developing CNS.

CRIM1 protein, or nucleic acids encoding CRIM1 and interacting proteins which selectively bind such proteins will also find use in screening chemical libraries for regulators of neural differentiation, cell migration, adhesion and neuronal process growth, in genetic mapping, as probes for related genes, as diagnostic reagents for genetic neurological disease and in the production of specific-cellular and animal systems for the development of neurological disease therapy, particularly for conditions such as motor neuron disease. They may also be important in the derivation and in vitro culture of neural stem cells for stem cell therapy of neurological conditions.

Diagnostic Methods

The invention provides use of Crim1 nucleic acids and CRIM1 polypeptides for diagnostic purposes.

It will be appreciated that the Crim1 nucleic acids of the present invention provide useful reagents for chromosome tagging and localization, such as in human genetic mapping studies. As will be described hereinafter, the isolated human Crim1 nucleic acid corresponds to a gene located on chromosome 2p21-16.3. It is noted that heritable diseases such as spastic paraplegia (SPG4; Hazan et al., 1994, Hum. Mol. Genet. 3 1569) and holoprosencephaly (Schell et al., 1996, Hum. Mol. Genet. 5 223) map close to this chromosomal region in humans (SIX3 and Spastin respectively). Crim1 nucleic acids may therefore be useful in mapping and isolating hitherto unknown genes in this chromosomal region underlying other diseases. Gene mapping techniques are well known in the art, and a recent review of techniques such as linkage analysis, SNP analysis and uniparental disomy is provided in Vnencak-Jones, 1999, Am. J. Clin. Pathol. 112 S19, which is incorporated herein by reference.

Furthermore, the specification hereinafter provides a variety of methods utilizing isolated Crim1 nucleic acids for analysis of cell and tissue development, which method may be useful in diagnostic, forensic and general tissue-typing applications.

For example, the invention provides a method of determining whether an animal is predisposed to a genetically-heritable disease, which method includes the steps of:

(i) obtaining a nucleic acid sample from said animal; and

(ii) determining whether said nucleic acid sample includes a Crim1 nucleic acid mutation or polymorphism indicative of said human being predisposed to, or suffering from, said genetically-heritable disease.

It will be appreciated that according to this aspect, the Crim1 nucleic acid may be used as a basis for designing PCR primers, sequencing primers or hybridization probes to assist determination of whether said Crim1 nucleic acid contains a mutation or polymorphism indicative of said individual being predisposed to said disease.

As used herein “predisposed” refers to said individual having an increased likelihood of displaying disease symptoms, or being a carrier of, a predisposing mutation or polymorphism.

Preferably, said genetically-heritable disease may be a neurodegenerative disease such as motor neuron disease, a disease of the eye or more particularly the eye lens, such as glaucoma, cataracts, micropthalmia, microcomea or nuclear scelorosis of the lens. Other possible diseases may relate to heart development, tooth development or bone morphogenesis.

Suitably, the nucleic acid sample is genomic DNA, cDNA or mRNA. Preferably, the nucleic acid sample is genomic DNA.

Preferably, step (ii) employs a nucleic acid amplification technique such as PCR.

Analysis of said amplification products may be according to relative size, in which case high resolution gel electrophoresis or capillary electrophoresis are applicable. Analysis may also be achieved by nucleotide sequencing of said amplification products.

Other approaches are relevant to full mutation analysis of Crim1, for example using single stranded conformation polymorphism (SSCP) analyses. This would involve the extraction of genomic DNA from said individual and PCR amplification of each Crim1 gene exon. Polyacrylamide gel electrophoresis under specific denaturing conditions would reveal any amplified alleles which migrate differently to wildtype DNA. These can then be directly sequenced to identify the mutation.

It will also be appreciated that detection of said mutation or polymorphism may be achieved by a variety of other techniques. These include Denaturing Gradient Gel Electrophoresis (DGGE), an example of which is provided in Folde & Loskoot, 1994, Hum. Mut. Det. 3 83 (which is herein incorporated by reference), melt curve analysis, an example of which is provided in International Publication No. WO97/46714 (which is herein incorporated by reference), or Restriction Fragment Length Polymorphism (RFLP) analysis of said amplification products.

So that the invention can be understood in more detail, the skilled person is directed to the following non-limiting examples.

### EXAMPLE 1

Isolation of Human CRIM1 (S52) cDNA

1.1 Two-Hybrid Screening

Yeast Transformation

The library used for two-hybrid analysis was a 19-23 week human fetal kidney cDNA “MATCHMAKER” library purchased from Clontech (USA). cDNA inserts were cloned into the pGAD10 plasmid so that encoded protein would be expressed as LexA-AD fusion proteins.

The library was transformed into the yeast Saccharomyces cerevisiae L40 strain (4LexAop-HIS3; 8LexAop-lacZ) which had been previously transformed with the plasmid pBTMWT1D encoding the WT1D-LexA (DBD) fusion protein “bait”.

HIS3 Reporter Activation and Growth Selection

Activation of the HIS3 reporter gene was determined qualitatively by growth on SD plates lacking uracil, lysine, tryptophan, leucine, and histidine [SD(THULL)] after incubation for 3 days at 30° C. The relative strength of interaction was determined by replica plating colonies on plates containing 3-amino-triazole (3AT; Sigma Chemical Company). The range of 3AT concentration typically used was between 0.2 and 5.0 mM in SD(THULL) plates.

lacZ Reporter Activity

β-galactosidase levels were determined qualitatively by a filter assay or in a liquid assay. For filter assays, colonies were grown as an X-shape (to increase colony mass) on replica SD(THULL) plates till thick (3-4 days). A sterile circle of 3M paper cut to the size of the 10 cm plate was placed onto one plate of the colonies and pressed firmly across the surface of the plate to encourage even adherence of the colonies to the paper. The paper was then peeled from the plate and put through two cycles of freezing and thawing in liquid N₂. The paper was then overlayed onto a pad of three similar paper circles pre-wet in Buffer Z with X-gal and incubated in a 10 cm plastic petri dish at 30° C. Incubations were allowed to continue overnight if necessary. Liquid assays were carried out by a standard assay method.

Plasmid Recovery

Cells from 1 ml of overnight culture were pelleted and resuspended in 0.5 ml of S Buffer (10 mM KPO₄ pH 7.2, 10 mM EDTA, 50 mM 2-ME, 50 μg/ml zymolase) and incubated at 37° C. for 30 minutes. 0.1 ml of Lysing Solution (0.25 M Tris-HCl pH7.5, 25 mM EDTA, 2.5% SDS) was then added and the solution was vortexed and incubated at 65° C. for 30 minutes. 166 μl of 3 M potassium acetate was added and the solution was chilled on ice for 10 minutes before centrifugation for 10 minutes. The supernatant was transferred to a fresh tube and 0.8 ml of cold ethanol was added. After 10 minutes on ice the solution was centrifuged for 10 minutes and the supernatant discarded. The pellet was then washed in 70% ethanol, air-dried, and resuspended in 40 μl sterile H₂O. This method was used to recover the library “prey” plasmids from colonies that contained both HIS3 and lacZ activity. Plasmids were recovered by transformation of the yeast miniprep into E. coli HB101 electrocompetent cells and selection on M9 minimal media plates. Bacteria not transformed with the trp+ “bait” plasmid will not grow on this media.

1.2 Library Screening and cDNA Sequencing

An insert corresponding to an S52 partial cDNA clone was isolated, and subjected to restriction endonuclease digestion with SacI and BamHI restriction enzymes to yield four sub-fragments of 900 bp, 800 bp, 600 bp and 300 bp respectively. These sub-fragments were ligated into pBluescript KSII-(Stratagene) and transformed into DH5a E. coli for sequencing. The 300 bp sub-fragment was labeled with ³²P-dCTP by random priming and used to screen a human fetal brain cDNA library in λGT10. Positive plaques identified by plaque hybridization were picked, phage isolated and inserts subcloned into pBluescript for further nucleotide sequencing. Initial sequencing was performed using standard reverse, forward, T3 or T7 primers as applicable to pBluescript. As further sequence was obtained, this was used to design additional sequencing primers.

### EXAMPLE 2

### Isolation of Mouse Crim1 (S52) cDNA

2.1 Preparation of DNA Probes

The probe used for primary library screening was derived from a PCR product of Mouse EST 551975 (Genome systems) containing the predicted 3′ end of the mouse S52 cDNA. PCR was performed as above using Mouse S52 3′ 3F (5′ GCT CAG CAC CCC TTC TAT TTG C 3′; SEQ ID NO: 10) and Mouse S52 3′ 3R (5′ GTG ATG AGT CTC GCC TGG ATG 3′; SEQ ID NO: 11) primers at an annealing temperature of 57° C. The product was cloned into pGEM-T easy (Promega). The 5′ end sequence of the human S52 gene was used as a probe for secondary library screening of primary phage positives. For this purpose, an 800 bp fragment of a human S52 cDNA clone was excised with SacI and BamHI and purified using the Agarose gel extraction kit (Boehringer-Manneheim).

Radiolabeling of DNA probes with [γ-³²P] dCTP was performed using the Redi Prime-II labelling kit (Amersham Life Science). 2.2

Library Screening

Radiolabeled probes were used to screen an E11.5 random primed whole mouse cDNA library cloned into λgt10. Briefly, Duplicate Hybond N filters were lifted from each plate, air-dried for 30 minutes and cross-linked using a GS gene linker (BioRad). Filters were pre-hybridised (3 hrs) and then hybridised with 200 ng of radioactively labeled probe (16 hrs) in Church and Gilbert buffer (0.263 M sodium phosphate buffer, pH 7.2; 1 mM EDTA; 7% SDS; 1% BSA (Boehringer-Mannheim) at 65° C. Stringency washes were performed with SSC/SDS wash solutions, down to 0.2×SSC, 0.1% SDS (Sambrook et al., 1989, supra). Hybridized filters were exposed to X-ray film (Fuji) at −70° C. with intensifying screens.

2.3 Cloning and Sequencing of Phage Inserts

Plaques identified via probe hybridization on duplicate filters were isolated and replated (Sambrook et al., 1989, supra) prior to subcloning cDNA inserts into pBluescript KS⁺. Sequencing was performed from both ends of the pBluescript KS⁺ vector using T7 and T3 primers. Additional primers were designed as sequence information was obtained.

2.4. 5′ RACE

Amplification of the 5′ end of the mouse S52 cDNA was performed using 5′ RACE with primers specific to the 5′ end of a mouse cDNA obtained from cDNA library screening. For 5′ RACE, four primers were utilized, three of which were nested primers, and the other a forward control. MS52 GSP1 (5′ GGA ATC TTC AGG GCA ACG 3′; SEQ ID NO: 12) was used for cDNA synthesis, MS52 GSP2 (5′ CAC AGC GGG CCT TGC TGC AAT C 3′; SEQ ID NO: 13), and MS52 GSP3 (5′ GCC GGA GAT GAG GTT TTC ATT G 3′; SEQ ID NO: 14) was then used for PCR amplification. MS52 RACE F (5′ CCG CCA GAG GAA CGA GAG CTG 3′; SEQ ID NO: 15), was used in conjunction with MS52 GSP3 to test for the presence of S52 cDNA at each step of the 5′ RACE protocol by PCR. Template RNA was extracted from homogenized tissue (E11.5 whole mouse embryo) using the guanidinium thiocyanate (GTC), phenol and chloroform method (Sambrook et al., 1989, supra). 5′ RACE was performed using the 5′ RACE system for rapid amplification of cDNA ends kit (GibcoBRL). 5′ RACE products were purified using PCR spinclean kit (Progen), and ligated into pGEM-T easy cloning vector as described above. The ligation was transformed into E. coli cells, grown and plasmid DNA was isolated. The resulting clones were sequenced from both ends using T7 and SP6 primers.

### EXAMPLE 3

### DNA Sequencing

Sequencing was carried out using the ABI PRISM™ BigDYE™ terminator sequencing ready reaction kit (ABI) For the sequencing reaction, 0.3-0.5 μg of double stranded plasmid DNA template and 3.2 pmole of primer was added to 8 μl of ABI terminator ready reaction mix and the volume made up to 20 μl with distilled water. The reaction was overlaid with mineral oil and incubated in a Perkin Elmer Cetus thermocycler at 96° C. for 30 seconds, 50° C. for 15 seconds and 60° C. for 4 mins for a total of 25 cycles. DNA was purified from the reactions by ethanol precipitation. Gel separation and raw sequence data analysis was performed through the DNA sequencing service at the Australian Genome Research Facility.

### EXAMPLE 4

### Amplification of DNA by the Polymerase Chain Reaction

Polymerase chain reaction (PCR) was used for probe preparation, for radioactive labeling, 5′ RACE and chromosomal localization experiments. Unless otherwise stated, PCR was performed using the following reagents: 2.0 μl of Taq DNA polymerase buffer (Boehringer-Mannheim), 2 μl of 2.5 mM dNTPs, 0.8 μl of 50 μM primer (forward and reverse), 0.2 μl of 5 U/μl Taq DNA polymerase (Boehringer-Mannheim), 1-2 μl DNA template (various concentrations dependent on template type) and water to a final volume of 20 μl. Unless elsewhere stated the reactions were incubated in a MJ Research DNA engine thermocycler, with an initial denaturing step at 94° C. for 3 mins, then 35 cycles of 94° C. for 1 min; 50-60° C. (dependent on primer) for 45 seconds and 72° C. for 1 min, with a final extension step of 72° C. for 1 minute.

### EXAMPLE 5

### Northern Hybridization

5.1 Whole Mouse mRNA Embryonic Northern

A mouse embryonic northern filter containing ES cell and whole mouse embryo RNA (E11.5-E15, and E17.5) was probed with a radioactively labeled PCR fragment generated from MS52 3F and MS52 3R primers. The filter was washed to a stringency of 2×SSC, 0.1% SDS at 65° C., and exposed to X-ray film for 5 days at −70° C. A control using a glyceraldehyde dehydrogenase (GAPDH) probe had previously been performed using the same filter, showing equal loading in all lanes.

5.2 Human Multiple Tissue Northern

A human multiple tissue northern containing mRNA from adult tissues (CLONTECH, catalog # 7760-1), was probed with human S52 (800 bp SacI-BamHI fragment) at 65° C. This filter was washed to a stringency of 1×SSC: 0.1% SDS at 65° C., and exposed to X-ray film for 2 nights at −70° C. A control with GAPDH probe was used to reveal the mRNA present in each lane.

### EXAMPLE 6

### Whole Mount in situ Hybridization

6.1 DIG-Labeled RNA Probe Preparation

Probe synthesis was carried out in a 20 μl reaction volume containing 9.5 μl of RNase-free distilled water, 4 μl of 5×transcription buffer (Promega), 4 μl of 0.1 M DTT (Promega), 2 μl of digoxygenin (DIG) nucleotide labelling mix (Boehringer-Mannheim), 1 μl of linearised template DNA, 0.5 μl of placental ribonuclease inhibitor (Promega) and 1 μl of T3 or T7 RNA polymerase (Promega). The reaction was incubated for 2 hours at 37° C. 2 μl of DNase-I (Promega) was added and reaction incubated for a further 15 mins. The probes were purified using a G-50 sephadex DNA purification column (Boehringer-Mannheim) and stored at −20° C. Mouse S52 antisense probe (RNA complementary and will hybridize to endogenous mRNA), was made by linearising MS52 clone 19 (from library screening) with SalI, and transcribing with T7 RNA polymerase. The sense probe (negative control) was made by cutting MS52 clone 19 with BamHI and transcribing with T3 RNA polymerase. Shh and Islet-1 radiolabelled probes were used as positive controls.

6.2 Mouse Embryos

Pregnant female Quakenbush mice were obtained from the Central Animal Breeding House (University of Queensland). The age of embryos were determined by designating noon of the day of the seminal plug as E0.5. Also, stereotype limb shapes characteristic of each developmental stage was used as a guide after dissection of embryos. Embryos were obtained by the dissecting the uterus and removing the uterine wall and extraembryonic membranes in ice cold PBS. The embryos were fixed in 4% paraformaldehyde (PFA) overnight and then dehydrated through a PBTX (10 mM phosphate buffered saline [PBS], 0.1% Triton X-100): methanol series (75% PBTX: 25% methanol; 50% PBTX: 50% methanol; 25% PBTX: 75% Methanol) into 100% methanol and stored at −20° C. until required.

6.3 Pre-Treatment of Embryos and Hybridization of DIG-Labeled RNA Probe

Embryos were rehydrated into PBTX by a reverse PBTX: methanol series, and treated with 10 μg/ml Proteinase K (Sigma) for 20 mins. After washing with PBTX twice for 10 mins, the embryos were refixed in 4% PFA, 0.2% glutaraldehyde (Sigma) in PBTX for 20 mins at 25° C. The embryos were incubated at 65° C. in pre-hybridization buffer (50% formamide, 5×SSC, 2% blocking powder [Boehringer-Mannheim], 0.1% Triton-X100, 0.5% CHAPS [Sigma], 1 mg/ml yeast RNA, 5 mM EDTA and 50 μg/ml heparin) for 3 hours with agitation. 1.0 μg DIG-labeled probe was then added and incubated overnight at 65° C.

6.4 Post Hybridization Washes and Immunohistochemistry

Samples were washed at 65° C. for 5 mins in S1 (50% formamide, 5×SSC, 0.1% Triton-X100, 50% CHAPS). The samples were then washed sequentially in 75% S1: 25% 2×SSC; 50% S1: 50% 2×SSC; 25% S1: 75% 2×SSC for 5 mins at 65° C., and then 2 washes each of 2×SSC, 0.1% CHAPS and 0.2×SSC, 0.1% CHAPS for 30 mins at 65° C. The embryos were washed with TBTX (50 mM Tris.HCl [pH7.5], 150 mM NaCl, 0.1% Triton-X100) twice at 25° C., and incubated in pre-block solution (10% sheep serum, 2% BSA in TBTX) at 25° C. The embryos were incubated overnight at 4° C. with pre-absorbed anti-DIG monoclonal antibody (Boehringer-Mannheim) diluted 1/2000 in pre-block solution. Preabsorption of antibody, was prepared by incubating 1 μl of antibody (0.75 units/μl) in 0.5 ml of TBTX containing 3 mg mouse embryo powder, 10% sheep serum, 2% BSA at 4 24° C. for 3 hours; removing the embryo powder by centrifugation, and diluting the supernatant to 2 ml with TBTX containing 10% sheep serum and 2% BSA.

6.5 Post Antibody Washes and Staining

The embryos were washed with 0.1% BSA in TBTX, five times for 1 hour each at room temperature, and then overnight at 4° C. The embryos were washed twice with TBTX and then three times with NTMT (100 mM NaCl, 100 mM Tris.HCl [pH9.5], 50 mM MgCl₂, 0.1% Tween-20) at 25° C. The alkaline-phosphatase colouring reaction was carried out for 2-5 hours at 25° C. in the dark, with 0.338 μg/ml NBT (Boehringer-Mannheim), and 0.175 μg/ml BCIP (Boehringer-Mannheim). The reaction was terminated once sufficient colour was observed, by washing several times in NTMT and then PBTX at 25° C. Background labeling was removed by washing in PBS containing 1% TTX100. Embryos were fixed overnight in 4% PFA and stored in 50% glycerol:PBS.

### EXAMPLE 7

### Section in situ Hybridization

7.1 Cryosectioning

Mouse embryos obtained as above were directly frozen in Tissue-tek (Sakura-Finetek) on dry ice, and stored at −20° C. Sections were cut using a Leica 3050 cryostat, with a section thickness of 14 μm. Sections were transferred immediately to pre-treated microscope slides (Superfrost) and air dried for several hours in a dust-free hood at 25° C.

7.2 Tissue Preparation

Sections were fixed in 4% PFA for 10 mins, and then washed in PBS three times for 3 mins each. The sections were then incubated in acetylation mixture (1.33% triethanolamine [Fluka], 15 mM HCl, 0.25% Acetic anhydride [Fluka]) for 10 mins, and washed three times with PBS for 5 mins each at 25° C.

7.3 Hybridization of DIG-Labeled Probe

800 μl of hybridization mix (50% formamide, 5×SSC, 5×Denhardts, 250 μg/ml bakers yeast RNA [Sigma], 500 μg/ml herring sperm DNA) was added to each slide and incubated in a 5×SSC humidified chamber for 2 hours. This was replaced with 75 μl hybridization solution containing 200 ng/ml DIG labeled RNA probe. Siliconised coverslips (dipped twice in 3% silane in chloroform and 100% ethanol, and air-dried), were placed onto slides, and incubated in humidified (5×SSC, 50% formamide) chamber overnight at 60° C.

7.4 Post Hybridization Washes and Immunohistochemistry

Coverslips were removed in 5×SSC, and then the sections were incubated in 0.2×SSC at 60° C. for one hour. After washing in 0.2×SSC at 25° C. for 5 mins, the slides were transferred into buffer B1 (0.1 M Tris pH 7.5, 0.15 M NaCl) for 5 mins at 25° C. 1 ml of B1 containing 10% sheep serum was placed on each slide, and incubated for 1 hour. 0.5 ml of anti-DIG antibody (1:5000 dilution in B1 with 0.1% sheep serum) was placed onto each slide and incubated overnight at 4° C. Slides were rinsed three times with B1, and equilibrated with Buffer B3 (0.1 M Tris pH 9.5, 0.1 M NaCl, 50 mM MgCl₂). The colouring reaction was performed by incubating the section with 0.338 μg/ml NBT and 0.175 μg/ml BCIP in buffer B3 for 2-5 hours. The reaction was terminated by rinsing several times with distilled water. Background staining was removed by washing with PBS containing 1% Triton-X100. The sections were fixed in 4% PFA and mounted using Mount Quick aqueous mounting medium (Daido Sangyo).

### EXAMPLE 8

### Photography

Photography of whole embryos was carried out on a Leica MZ8 dissecting stereo microscope with a Leica MPS48 exposure controller and camera. Photography of tissue sections was performed on an Olympus Provis AX70 microscope with an Oympus U-MCB exposure controller and camera, using Kodak Ektachrome 160T Pro film.

### EXAMPLE 9

### Chromosomal Localistion of Human 552 by Radiation Hybrid PCR Screening

9.1 Primer Design and Control PCR

Radiation hybrid screening primers 3′ PCR 1F (5′ CTA CCA AAC AGT GTG AAG AAA 3′; SEQ ID NO: 16) and 3′ PCR 1R (5′ TGG TCA GTT ATC TTG AGG AA 3′; SEQ ID NO: 17) were designed using the sequence alignment of human and mouse S52 at a region which was thought to cover the 3′ untranslated region (UTR). These primers amplified a single band of 196 bp from human S52 cDNA and human genomic template DNA (Genebridge), but neither mouse cDNA (mouse EST #551975), mouse genomic DNA (Genebridge), or hamster genomic DNA (Genebridge) at 58° C. annealing temperature.

9.2 Sample PCR Screening and Data Analysis

Screening of 93 samples comprising a human:hamster hybrid genomic DNA panel (Gyapay et al., 1996, Hum. Mol. Genet. 5 339), was performed by PCR with 3′ PCR 1F and 3′ PCR 1R primers. 10 ml of the reaction was loaded onto a 2.0% gel and each sample was scored as either negative (0), positive (1) or unclear (2). The results were analyzed at the whitehead institute database (www-genome.wi.mit.edu), which links the “vector” of 0's 1's and 2's to the closest chromosomal marker by pairwise analysis (Gyapay et al., 1996, supra). A LOD (likelihood of odds) score of greater than 15 to the closest marker was considered as statistically significant.

### EXAMPLE 10

### Immunofluorescence Analysis of Mouse Kidney

10.1 Antibody Production

Polyclonal antibodies were raised in rabbits against a pGEX fusion protein containing:

(i) a fragment of human CRIM1 corresponding to the following 41 C-terminal amino acids:

| | |
|---|---|
| RVQVDSSQRMLRIAEPDARFSGFYSMQKQNHLQADNF | (SEQ ID NO:8) |
| YQTV; and | |
| (ii) KVCQPGYLNILVSKASGKPGEC. | (SEQ ID NO:9) |

The polyclonal antibodies were subsequently affinity-purified using the immunizing fragment.

10.2 Immunofluorescence

Pregnant mice were sacrificed at 11.5 days post-coitum. Embryos were collected into ice-cold MEM medium, bisected transversely between fore and hind limbs with a scalpel, and the lower portion bisected through the spinal cord using a pair of 30 gauge needles. The metanephric mesenchyme with attached ureteric bud were removed using the same needles and collected into fresh medium. Explants were cultured in MEM/10% FCS for up to 5 days. Media was changed every 24 hrs. On the day of harvesting, explants were washed in PBS, fixed in methanol (10 min at −20° C.), washed in PBS (10 min at RT), incubated with primary antibody (1 hr at 37° C.), washed in PBS (15 min at RT) before being mounted on coverslips and examination under an Olympus fluorescence microscope.

### EXAMPLE 11

### Subcellular Localization of CRIM 1

11.1 Transfection of Cells

Transfection and co-transfection reactions were performed in 6 well tissue culture plates using 50-80% confluent COS-7 cells plated the previous day. Following manufacturer's instructions, 1 μg of highly purified DNA (Nucleobond AX 500 column, Macherey-Nagel) was used per transfection reaction with a ratio of 3 μl of FuGENE 6 transfection reagent used per 1 μg DNA per well. Following transfection, cells were incubated in OPTI-MEM I reduced serum medium (Life Technologies) at 37° C. in an atmosphere supplemented with 5% CO2 and harvested 2.5-3 days post transfection. Cell media and lysate were separately analysed for the presence of the protein(s) of interest by Western blot.

11.2 Cell Fractionation for Subcellar Localisation: Membrane and Soluble Fractions

Cells were cultured on 10 cm plates. These were put on ice, washed three times rapidly in cold buffer A (10 mM Tris pH 7.4; 1 mM EDTA), and scraped from plates in 0.5 mL buffer A containing protease inhibitors. Cells were incubated on ice for 5 minutes before being passed through a 27 gauge needle approximately 20 times. Lysate was centrifuged at 2,000 rpm for 10 min in bench top centrifuge. Supernatant was collected and spun at 45,000 rpm at 4° C. in a bench-top ultracentrifuge for 90 minutes.

The resulting supernatant (soluble fraction), was respun at 45,000 rpm for 30 min. The pellet (membrane fractions) was resuspended in buffer A

11.3 Plasma Membrane, Heavy Membranes, Cytosol and Membranes

Cells were cultured on 30 cm plates, washed in ice cold HES Buffer (20 mM Hepes, 1 mM EDTA, 250 mM sucrose, pH7.4), then scraped into 2.5 ml HES containing protease inhibitors (HES+), and then passed through a 27 gauge needle 20 times. The lysate was centrifuged at 12, 000 rpm for 20 min. The supernatant was collected as the cytosol and membrane fraction.

The pellet was resuspended in 10 ml HES+ and recentrifuged. This pellet was resuspended in 0.5 mL HES+ and layered over 10 mL 1.12 M sucrose in HES in Beckman Ultra-Clear (14×89 mm) tubes for centrifugation in a Beckman SW41 rotor for 60 min at 25 000 rpm. 1 mL of solution was collected at the interphase. 2 mL of HES+ was added for further centrifugation in a Beckman TLA 100.3 rotor in bench-top ultracentrifuge. The resulting supernatant was discarded and the pellet resuspended in HES+, and snap freeze. This represents the plasma membrane fraction.

The pellet from the sucrose phase was resuspended in HES+. This represents the heavy membrane fraction, containing mitochondria, nuclei, and other heavy membranes.

11.4 Immunofluorescence Analysis

Cells were cultured in small dished with coverslips placed in the bottom at the time of seeding with cells. For Immunofluorescence, the media was removed and the cells washed with PBS twice, before fixation in 4% PFA (in PBS pH 7.4) for 15 min, washing in PBS for 2 min three times, permeabilisation in 0.1% Triton/PBS for 5 min, washing in 0.5% BSA/PBS twice for 4 minutes and blocking in 0.5% BSA/PBS for 10 min. Blocking solution was thoroughly aspirated before the addition of the primary antibody diluted in 0.5% BSA/PBS (typical dilution of 1/100-1/200).

Coverslips were incubated with primary antibody for 1 hr at room temperature, then washed three times for 2 minutes with 0.5% BSA/PBS. Slides were aspirated thoroughly before addition of the secondary antibody (typical dilution 1/200-1/400). Coverslips were incubated for 1 hr. in secondary antibody, then washed with 0.5% BSA/PBS three times for two minutes each. Coverslips were then mounted on slides with vectorshield or other mounting medium.

### EXAMPLE 12

### Interaction Between CRIM1 and Members of the TGF-β Superfamily

12.1 Coimmunoprecipitation With BMPs

Protein A immobilised on Sepharose 4B fast flow resin (Sigma) was equilibrated in protein A buffer (0.05 M Tris, 0.15M NaCl pH 8.5) prior to antibody being bound in a ratio of 5 μl of antibody to 40 μl of resin per immunoprecipitation reaction. Resin and antibody were incubated together for 90 minutes at room temperature on a rotating wheel, and the resin washed three times in protein A buffer prior to use.

From each transfection reaction, cell media was collected, cellular debris pelleted and removed and the media incubated with the bound resin for two hours at room temperature on a rotating wheel. The cell monolayer was gently washed with PBS, resuspended in 600 μl of lysis buffer (protein A buffer pH 7.4 containing complete protease inhibitors (Roche), 0.5% Triton X-100), transferred to 1.5 ml eppendorf tubes and incubated for one hour at 4° C. on a rocking platform. Insoluble cellular debris was pelleted, removed and the cell lysate incubated with the bound resin as described above. Following incubation, the resin was washed twice in lysis buffer followed by PBS, resuspended in 40 μl of SDS-PAGE loading buffer and boiled for 3 to 5 minutes prior to loading on a SDS-polyacrylamide gel. Immunoprecipitation reactions were analysed by Western blot, and generally using a primary antibody not used in the immunoprecipitation step. Fractions of cell lysate and cell culture media were retained prior to immunoprecipitation to allow the examination of the load material for the purposes of verification of the success of the transfections.

Ligand blotting was performed as described in Scheidegger et al., 2000, J. Biol. Chem. 275 26864, which is incorporated herein by reference.

Cell overlay technique was performed as described in Brose et al, 1999, Cell 96 795, which is incorporated herein by reference.

### EXAMPLE 13

### Purification and Bioassay of Recombinant CRIM1 Ectodomain

13.1 Affinity Purification of Recombinant Ectodomain

The protocol followed for construction of an anti-myc column and purification of CRIM1 protein over the column was as described in the manufacturers instructions provided with the Affi-Gel Hz Immunoaffinity Kit (BIO-RAD, catalog number 153-6060).

The anti-myc column was allowed to reach room temperature prior to use, then conditioned using 2 to 4 bed volumes of 0.2 M glycine-Cl pH 2.5 elution buffer. Allow the column to drain then add 5 bed volumes of application buffer (PBS, pH 7.0).

To purify CRIM1 protein expressed as CrimEcto^{myc} in COS-7 cells, the cell media was collected and pelleted to remove cellular debris. 4 ml of media was concentrated using an Ultrafree-4 centrifugal filter unit (Millipore) to 1-1.5 ml then a 1/10 volume of 0.5 M phosphate buffer pH 8.0 was added to increase media pH to 8.0. Cap the bottom of the column, add the concentrated media, cap the top of the column and incubate on a rotating wheel for 1-1.5 hours at room temperature. Drain the column, retaining the column run through for protein analysis, and wash the resin three times with 10 ml of PBS. The salt concentration of PBS can be increased to 0.5 M for one wash to remove contaminants if required. Elute the protein using 5 ml of 0.2 M glycine pH 2.5, and collect 750 μl aliquot's in eppendorfs containing 250 μl of 0.5 M phosphate buffer pH 8.0 so to increase the pH hence stability of the protein. Immediately wash the column with 20 ml of PBS and store at 4° C. in PBS containing 0.02% sodium azide.

Aliquots of the starting material, column run through and eluted fractions were analysed by Western blot and eluted fractions of interest were concentrated. Protein estimation was determined by analysing the purified, concentrated CRIM1 protein on a SDS-polyacrylamide gel alongside known concentrations of bovine serum albumin and staining the gel with Silver Stain Plus (BIO-RAD, catalog number 161-0449) (see FIG. 14A).

Each round of purification produces approximately 900 ng of protein as assessed by comparative Coomassie versus BSA standards. This recombinant protein is still recognised by the N-terminal CRIM1 antibody and can be used in functional bioassays.

13.2 In Ovo Electroporation

Fertilised chick eggs were incubated in a forced-draft humidified incubator kept at 37.5° C. until the embryos reached Hamburger and Hamilton stage 10. The embryos were exposed by opening a small window on the eggshell and by removing overlaying vitelline membrane. Human Crim1 cDNAs (encoding CRIM1 ectodomain or CRIM1 full-length and with myc epitope) were inserted into a eukaryotic cell expression vector (pcDNA3). The expression construct DNA was injected into a lumen of the caudal neural tube by mouth pippetting. The concentration of DNA was 1-5 μg/μL and several nL of the DNA solution was injected into the spinal cord.

After injection of the DNA construct into the lumen of the neural tube, the injected embryo was placed between a pair of electrodes. A train of small voltage pulses (25V, 50 msec width and 5 times, BTX820 square pulse generator) was passed through the embryo to achieve the gene transfer by the electroporation.

After the electroporation, the embryo was rinsed with L15 culture medium and the window was closed by adhesive tape and returned to the incubator. The electroporated embryos were further incubated for another 48 hr.

At the end of incubation period, the electroporated embryos were removed from the eggshell and transferred into ice-cold phosphate buffered saline (PBS). The embryos were fixed with 4% paraformaldehyde in 10 mM PBS for 1 hour on ice, then immersed in 30% sucrose in 10 mM PBS for overnight. The embryos were further trimmed and froze in TissueTek compound and stored at −70° C. until analysis.

14 micron thickness frozen sections were cut and placed on glass slides. After drying the sections, they were probed with primary antibodies for cell type-specific marker proteins (anti-Engrailed1 and anti-Islet1 mouse monoclonal antibodies). The primary antibodies were detected with species-specific secondary antibodies that recognize mouse IgG and conjugated with cyc3. The sections were also counterstained with DAPI to visualize the nucleus.

The labelled sections were observed using a compound microscope and pictures taken using a digital camera.

### EXAMPLE 14

### Results

14.1. Isolation of Crim1 Nucleic Acids

Human Crim1

The human Crim1/s52 nucleic acid was initially isolated as a result of two-hybrid screening for WT1D-interacting proteins. A total of twenty-six large and 12 small colonies were identified by a filter-based β-galactosidase reporter assay.

“Prey” plasmids were isolated from 20 large and 10 small colonies, and retransformation experiments showed that none of the plasmids activated the HIS3 or the lacZ reporter genes on their own or with the negative control plasmid.

To determine which portion of the LexA-WT1D fusion protein the protein products of these plasmids interacted, yeast di-hybrid analyses were performed against a panel of LexA-WT1D deletion constructs, according to the methods of Fields & Song, 1989, supra and Vojtek et al., 1993, Cell 74 205. Interaction was assessed on the basis of growth in the absence of histidine. The results of this analysis indicated that all of the proteins interacted with the N-terminus of WT1D, and none with the zinc finger motifs within the C-terminus.

Although the inserts of the isolated plasmids could be grouped according to insert size after EcoR1 digestion, sequencing was performed to determine identity. All were sequenced with a pGAD10 5′ primer located immediately upstream of the 5′ end of each insert. From 25 plasmid sequences, 11 distinct clones were obtained, each of which were then sequenced from the 3′ end with the pGAD10 3′ primer.

S52 appeared to be a “false positive” identified by the two-hybrid assay in that it was present in the cDNA library oriented 3′→5′. The S52 partial cDNA obtained by two-hybrid screening was used to isolate a full-length cDNA, the nucleotide sequence of which is shown in FIG. 2 (SEQ ID NO: 5). The predicted amino acid sequence of the human CRIM1 polypeptide is shown in FIG. 1 (SEQ ID NO: 2).

A human genomic Crim1 sequence was obtained by BlastN searching of human genome project updates, which sequence is shown in FIG. 3. Two large regions of human genomic sequence (NH007814 & NH0501007) were identified which cover 236,303 bp. Sixteen of the seventeen Crim1 exons could be positioned within the genomic sequence. Exon 1 (defined as including the start ATG) resides on both PAC 28 and PAC 309 but not within the 236,303 bp region.

Mouse Crim1

A murine ortholog of human CRIM1/S52 was isolated, the murine Crim1/S52 nucleic acid shown in FIG. 2 (SEQ ID NO: 6) and deduced amino acid sequence shown in FIG. 1 (SEQ ID NO: 3). Initial identification was achieved by using the human S52 nucleic acid as the basis for a search of the EST database. This identified EST clone ID#551975 which was sequenced to reveal an apparent mouse ortholog of S52. Screening of an E11.5 mouse embryo DNA library was performed using the mouse EST clone, with positives secondarily screened with the human S52 clone. This resulted in the isolation of a cDNA which partially overlapped the existing murine EST. The remaining 5′ mouse S52 sequence was then obtained by 5′ RACE, to provide the mouse Crim1 nucleic acid shown in FIG. 2. Mouse Crim1 exhibits 84% nucleotide sequence identity to human Crim1.

Chicken Crim1

Chicken Crim1 cDNA clones were isolated by screening an embryonic day 5 chick brain cDNA library (Hargrave et al., 2000, Dev. Biol. 219 142) using a cDNA which corresponds to the 3′ end of mouse Crim1 coding region (fragment (756 bp in length, corresponding to amino acid positions between 680 and 932). From this cDNA library screening, six independent cDNA clones were isolated (clone # 181.1 and 41.1 cover nucleotide positions between 0 and 1085, clone #121.3 and 5.1 cover between 1800 and 3150, and clone #101.1 and 131.1 cover positions between 2800 and 4010). A cDNA clone that was missing from the contig of these cDNA clones was amplified using a set of oligonucleotide primers and polymerase chain reaction (PCR). Sequences if the forward and reverse primers for this PCR reaction were 5′-CTCGCTGTCCAGAAGATTCC-3′ (SEQ ID NO: 18) and 5′-GGTTGCCGCATTTGTCAGTG-3′ (SEQ ID NO: 19) respectively. This PCR product corresponds to the nucleotide positions between 1015 and 1920 of the contig of chick Crim1 homolog cDNA sequence. The chick Crim1 cDNA sequence is a total of 4010 nucleotides in length in which nucleotide positions between 380 and 3400 correspond to the coding region (see FIG. 2).

The predicted protein sequence of the chick CRIM1 homolog (FIG. 1; SEQ ID NO: 4) has 82.0% overall identity to that of human CRIM1 and 82.9% overall identity to that of mouse CRIM1. The predicted chick CRIM1 homolog contains putative signal peptide sequence at amino positions between 1 and 46, insulin-like growth factor binding protein-like motif at amino acid positions between 49 and 85. There are also six conserved cysteine-rich repeats at amino acid positions between 348 and 402, 415 and 468, 620 and 674, 691 and 746, 765 and 820, 831 and 885, a putative transmembrane domain at amino acid positions between 948 and 973 and a short stretch of putative cytoplasmic tail.

Wholemount in situ analysis of developing chick embryos was used to investigate the expression pattern of the chicken Crim1 gene. In chick embryos, as in mouse, Crim1 mRNA is initially expressed by the floor plate and notochord, and developing motor neuron in ventral aspect of the neural tube. This confirms chicken Crim1/CRIM1 as a true ortholog of human and mouse Crim1/CRIM1..

14.2. CRIM1 Polypeptide Structure

An alignment of the human, mouse and chicken Crim1 protein sequences shown in FIG. 1 (SEQ ID NOS: 2, 3, and 4 respectively) has identified several regions outside of the identified structural motifs which are unique to Crim1 proteins. Given that the human, mouse and chicken sequences represent orthologs from different organisms, we have defined an N-terminal motif PGECCPLP which is absolutely conserved between all three proteins. This motif, when used to search the Genbank non-redundant database to identify sequences similar to the Crim1 orthologs is a partial EST clone from Caenorhabditis elegans (emb|CAA94886.1|) which encodes hypothetical protein B0024.14 (pir∥T18649). This EST predicts an hypothetical protein containing the 6 cysteine-rich motifs and the transmembrane domain seen in all three Crim1 orthologs. In this protein, the region PGECCPLP has only two amino acid changes (PGNCCPPP). Searching with this sequence only detects hypothetical protein B0024.14, suggesting that the motif PGXCCPXP together with the presence of six cysteine rich repeats spaced in the same fashion as seen in these four proteins identifies orthologs of the CRIM1 family. Alignment of the three CRIM1/Crim1 orthologs highlighted a three amino acid conserved region consisting of RGD. This has been previously reported as a motif involved in mediation of adhesion of cells to the extracellular matrix via integrins in proteins such a latent transforming growth factor binding protein (LTBP) (Mangasser-Stephan & Gressner, 1999 297 363; Ruoslahti & Pierschbacher, 1986 44 517). The RGD motif within CRIM1 is located between the IGFBP motif and the first cysteine-rich motif.

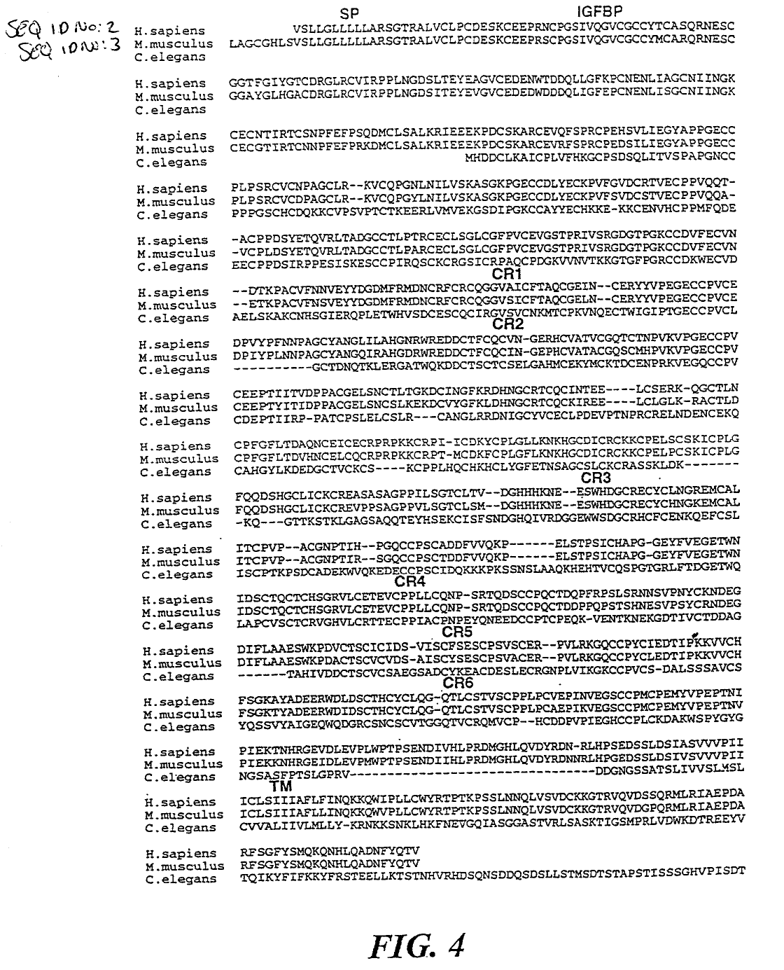

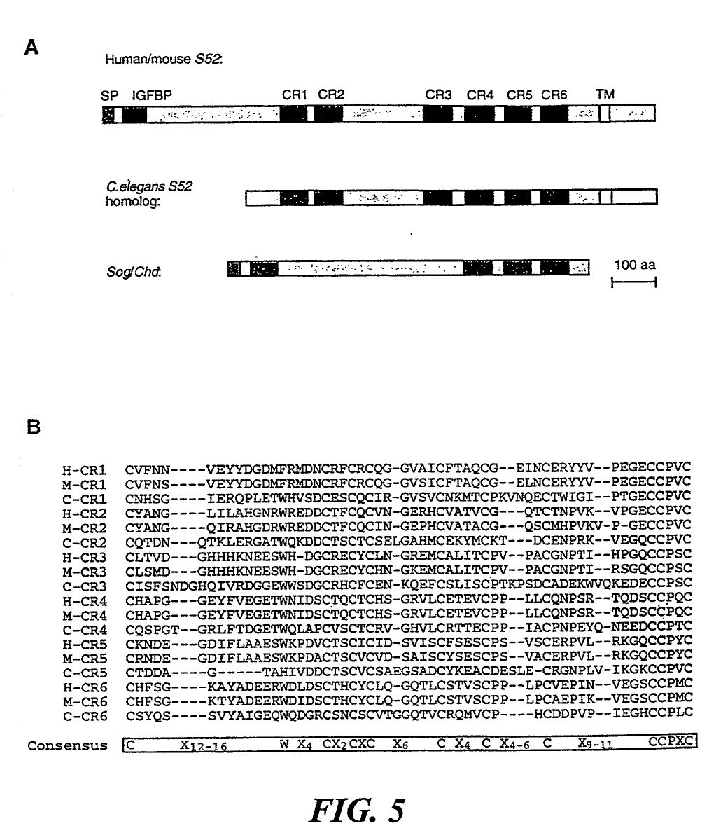

The human and mouse CRIM1 polypeptides comprise 1036 and 1029 amino acids respectively, sharing 89% sequence identity. An alignment of human and mouse CRIM1 polypeptides and a putative C. elegans ortholog is shown in FIG. 4, and a comparative overview of polypeptide domain structure is provided in FIG. 5. The major structural features of CRIM1 include a putative signal peptide (Nielsen et al., 1997, Protein Eng. 10 1), an insulin-like growth factor binding protein (IGFBP)-like domain (Drop et al., 1992, Growth Regul. 2 69), six cysteine rich repeats (CRRs; Hunt & Barker, 1987, Biochem. Biophys. Res. Comm. 144 876) and a putative transmembrane (TM) domain identified by hydropathy plot analysis (Kyte & Doolittle, 1982, J. Mol. Biol. 157 105).

The human, mouse and chicken CRIM1 amino acid sequences therefore suggested an integral membrane protein featuring cysteine-rich repeats (CRRs) with the following pattern:—

CX₁₂₋₁₆WX₄ CX₂ CXCX₆ CX₄ CX₄₋₆ CX₉₋₁₁ CCPXC

where C=cysteine; W=tryptophan; P=proline; and X=any other amino acid.

Similar CRRs are found in the Drosophila short gastrulation (Sog) gene, Xenopus chordin (Chd) gene and the human thrombospondin gene (Francois et al., 1994, Genes & Dev, 8 2602; Sasai et al. 1994, Cell, 79 779; Dixit, et al., 1986, Proc. Natl. Acad. Sci. USA 83 5449).

14.3. Tissue Distribution of CRIM1

Northern Analysis

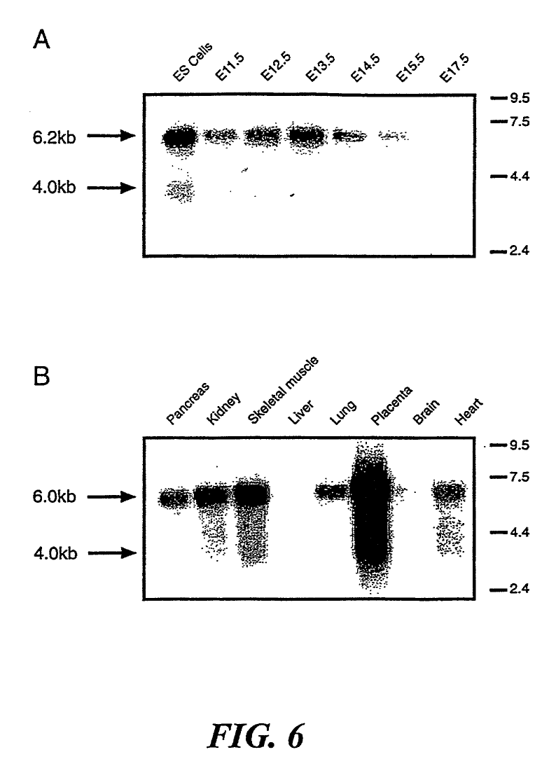

Northern hybridisation of mouse embryo mRNA at different developmental stages shows that the major murine Crim1 mRNA isoform is 6.2 kb, whilst the human ortholog in adult tissues is approximately 6.0 kb (FIG. 6). There appears to be a 4.0 kb isoform expressed at lower levels in mouse embryonic tissues as well as adult human placenta. This shorter mRNA isoform could be due to splicing of 3′ UTR, since two different ESTs which appear to be spliced isoforms have been identified on the GenBank EST database. Mouse EST 551975 has a polyA sequence which truncates the mRNA at approximately 4.0 kb of sequence. A second EST (mouse clone #13361968) is 100% identical 5′ of the polyA sequence, however, this second EST does not contain the polyA sequence at the same position as mouse EST 551975. EST 13361968 is predicted to extend much further into the 3′ direction and may represent the larger isoform seen by northern analysis.

To test for conservation of Crim1 in vertebrates, a genomic southern blot was analyzed at a high stringency with a mouse S52 radioactively labelled probe (data not shown). Positive bands were seen in human, mouse, chicken (Gallus gallus) and zebrafish (Danio rerio). This suggests homologous genomic DNA sequences are present in lower vertebrates and predicts a high level of conservation of S52 throughout evolution.

Whole Mount and Section Analysis

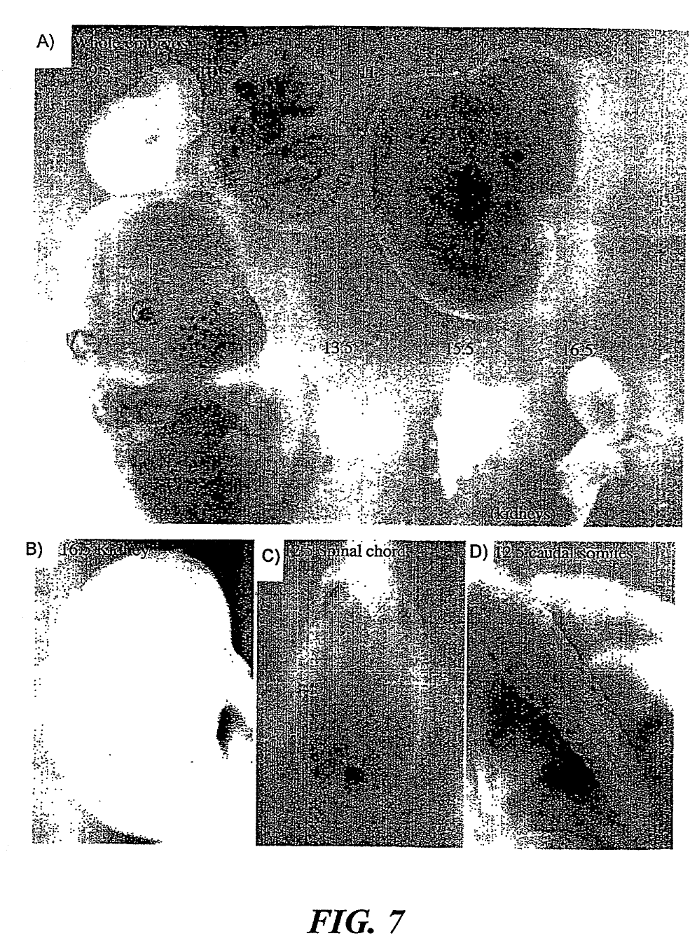

The whole mount analysis shown in FIG. 7 reveals that S52 is expressed in the ureteric compartment of the developing kidney from E10.5, and appears to be limited to the very tip of the ureteric tree. S52 is also expressed in the developing floor plate and motor neurons, the developing eye lens, the vibrissae and the pinna of the developing ear.

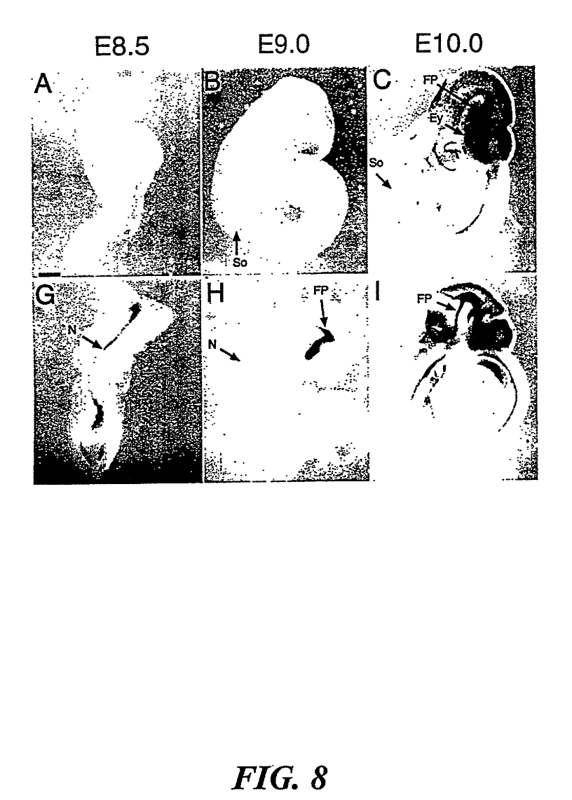

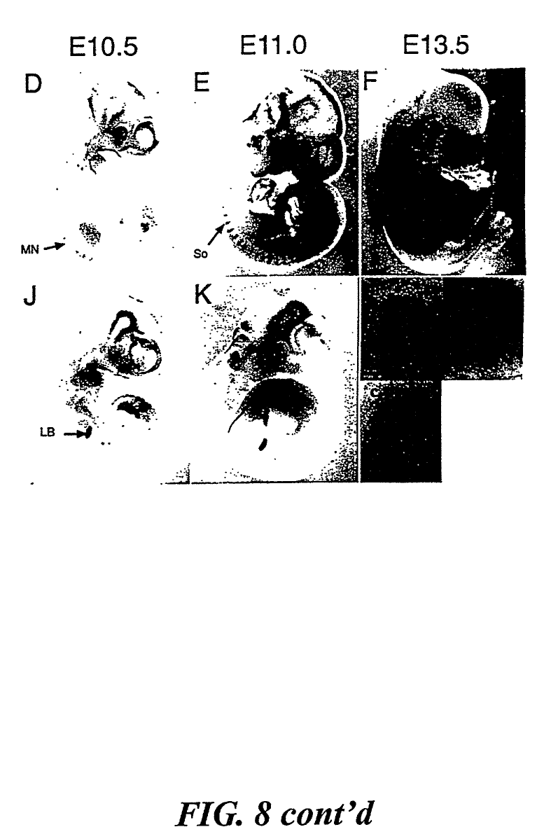

In FIG. 8, analysis of S52 expression was performed on whole embryo preparations from E8.5-E11.0, a time at which the developing neural tube forms and early patterning is thought to occur. Expression of S52 had previously been demonstrated in a small subset of cells in the neural tube including the floor plate. Therefore the expression of S52 was compared to a notochord and floor plate marker, sonic hedgehog (Shh) (Echelard et al., 1993, Cell 75 1477). Onset of S52 expression occurs between E9.0 and E10.0 (FIG. 8 B,C). There is weak expression in the somites at E9.0. Subsequently, there is expression in the somites, the anterior floor plate and developing eyes at E10.0. Expression occurs between 0.5 and 1.0 day after floor plate differentiation, as defined by Shh expression.

At E10.5 S52 expression was detected in the motor neurons, the floor plate, the somites, and the eyes. Expression in motor neurons was not detected at E10.0, a time when the first motor neurons are thought to have differentiated and migrated out of the ventricular layer. Therefore, expression of S52 appears to occur shortly after the motor neurons begin to differentiate, as revealed by expression of the early motor neuron marker, Islet-1 (Ericson et al., 1992, Science 256 1555). By E11.0, expression is detected in the somites, the eye, and very faintly in the floor plate and motor neurons.

Expression of S52 persists in older embryos from E11.5 to at least E17.5 as shown by northern hybridisation (FIG. 6). The highest level of expression is seen in E13.5 embryos. Whole mount in situ hybridisation at this stage shows expression in the lens of the eye, the ear and the vibrassae (sensory hair follicles of the snout) (FIG. 8F).

CNS Analysis

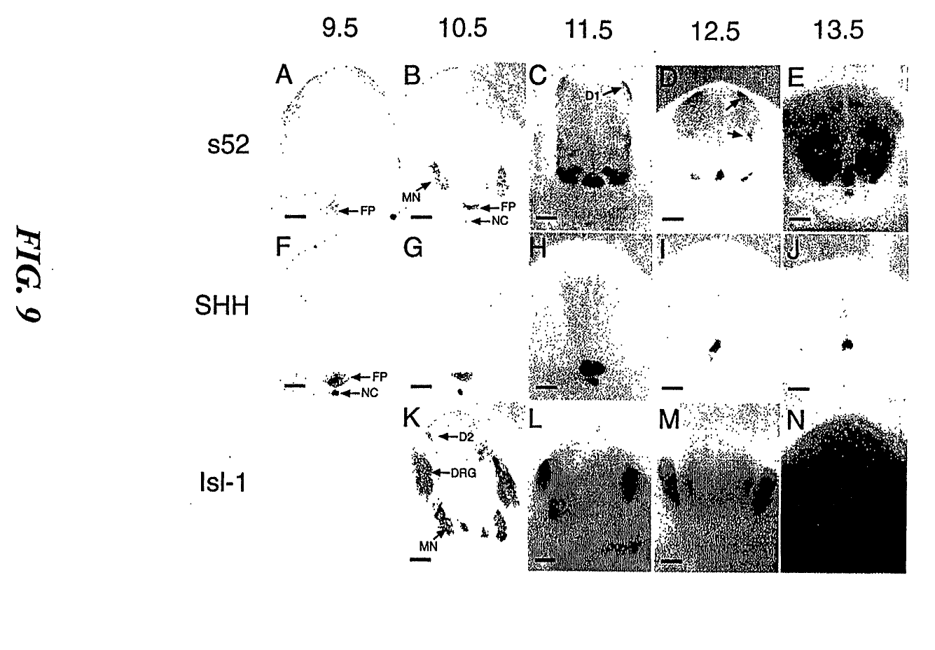

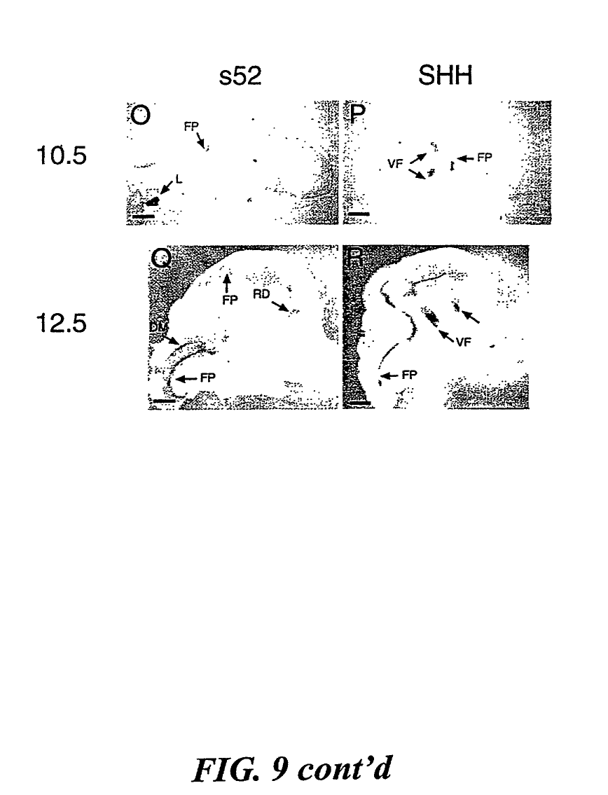

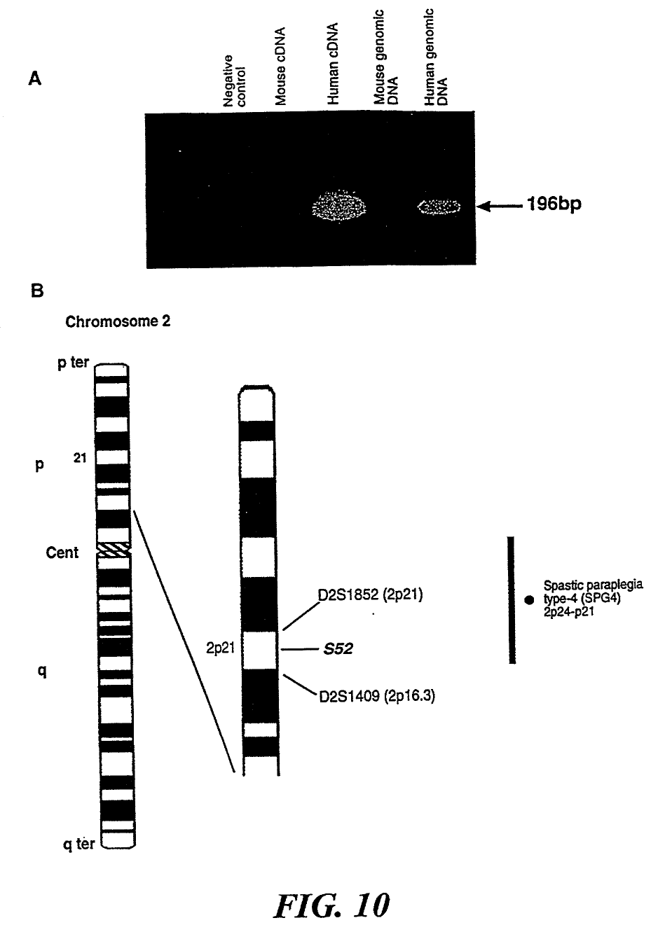

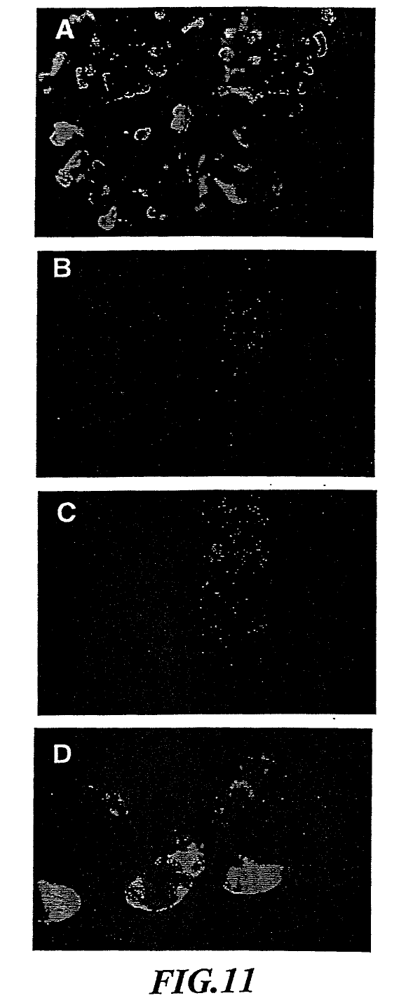

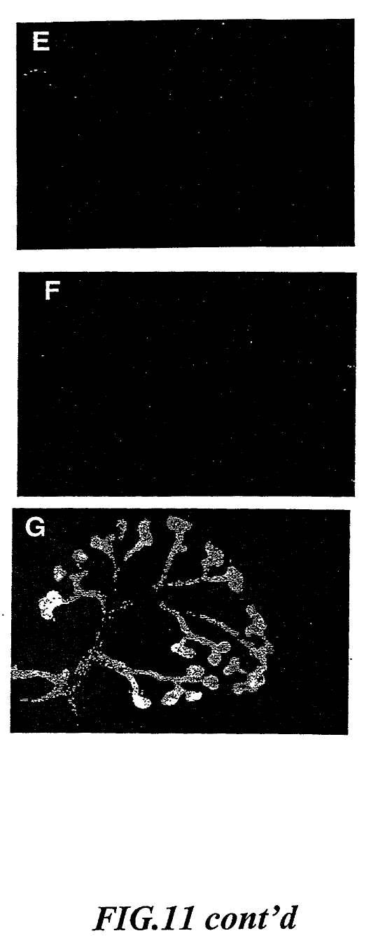

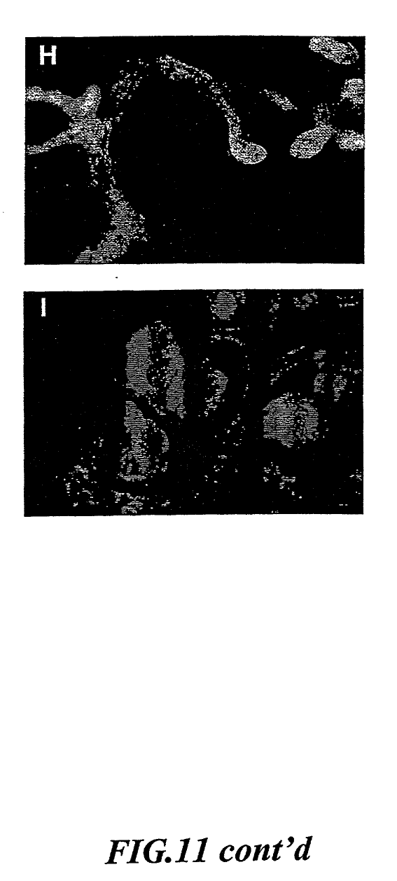

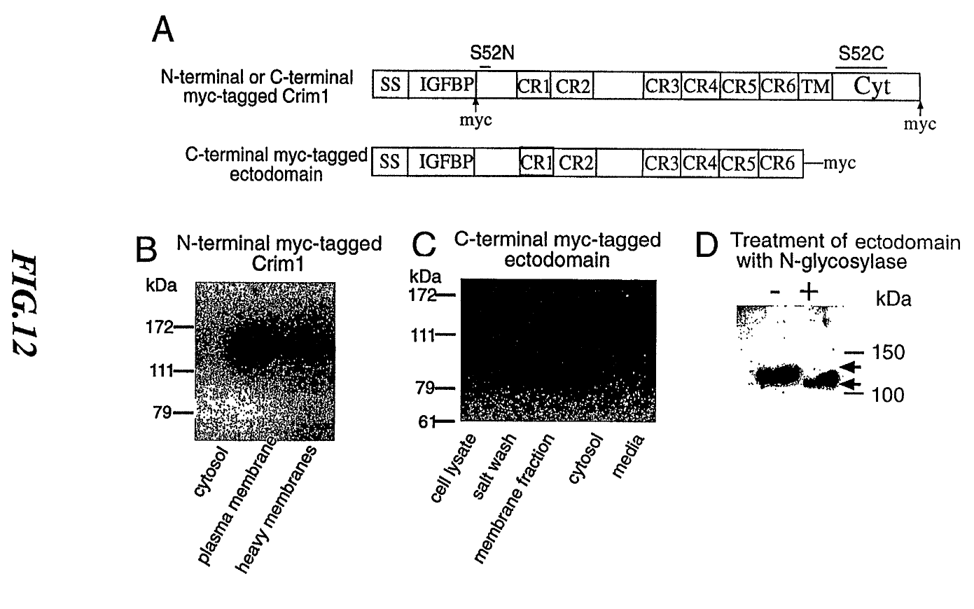

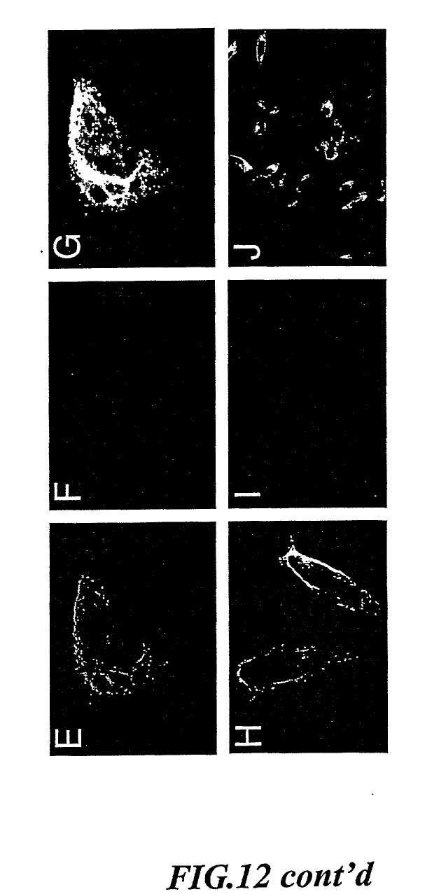

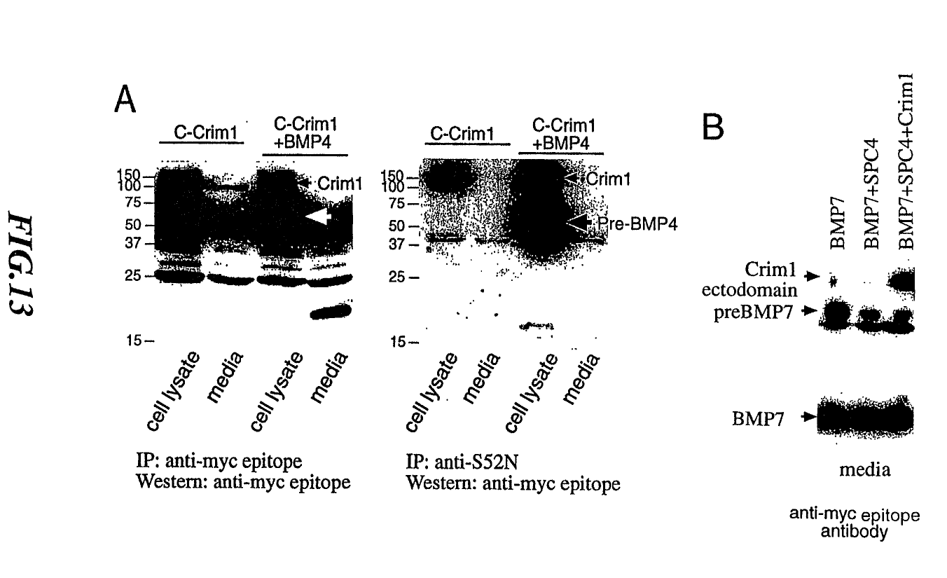

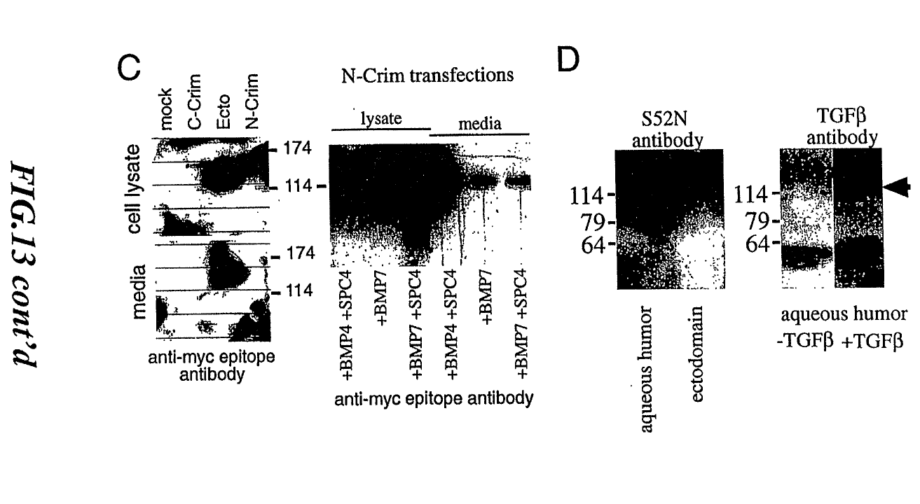

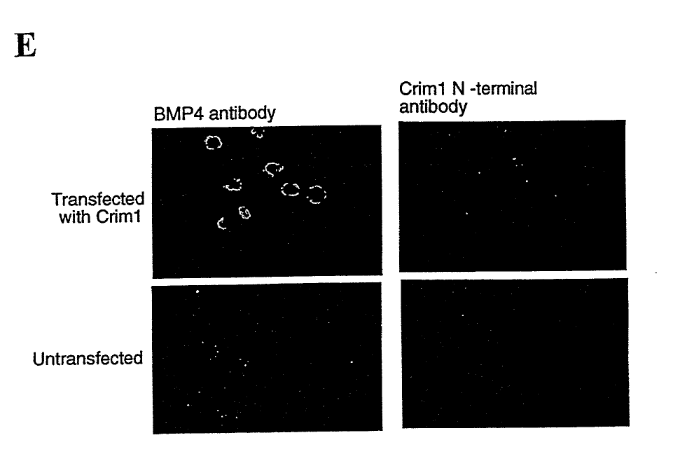

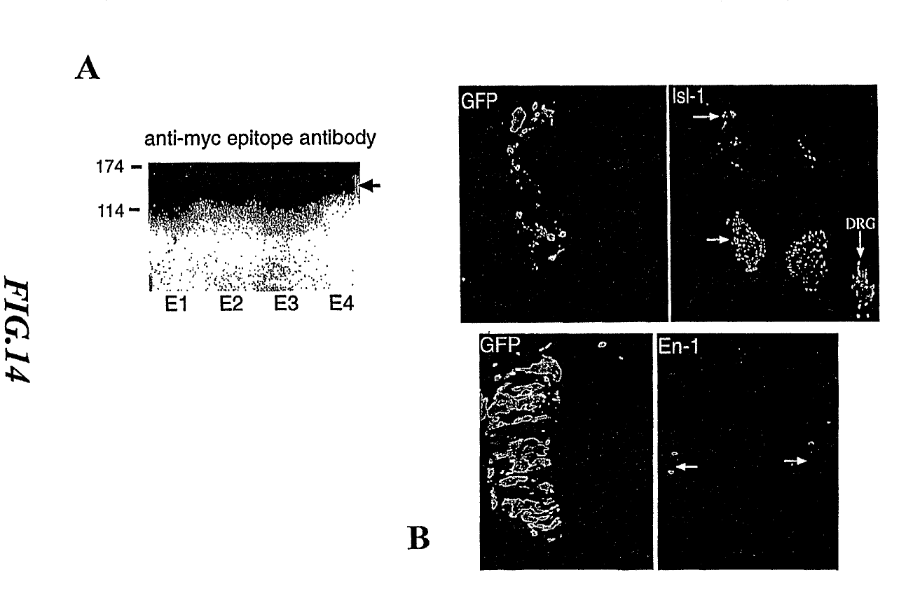

To further characterise the cell types that express mRNA in the developing CNS, embryo sections were obtained and analyzed by in situ hybridisation. Detailed morphological expression pattern analysis was performed by thin section in situ hybridisation of the cervical region of E9.5 and E10.5 embryos (FIG. 9). In addition, whole mount in situ hybridisation was performed on older embryos (E11.5-E13.5) which had been cut at the cervical level of the neural tube (FIG. 9). In all cases, analysis of expression was compared to Shh (expressed in the floor plate and notochord), and Islet-1 (Isl-1) was also used as a comparison.

Preliminary expression of S52 by whole mount and section in situ hybridisation of the brain was undertaken, and compared with that of Shh.

Whole mount in situ hybridisation suggested that onset of S52 expression in the neural tube occurs between E9.5-E10.5. Subsequently these stages were chosen for section in situ hybridisation. Expression at E9.0 is very weak in the floor plate, with possibly some staining in the notochord of the cervical section of the developing neural tube (FIG. 9A). More posterior sections did not show any expression (data not shown), suggesting that S52 expression in the anterior floor plate occurs later than E9.5. By E10.5 (FIG. 9B) stronger expression was observed in the floor plate of cervical sections of the neural tube. Other sections also revealed strong floor plate expression throughout more posterior regions of the neural tube (data not shown). There is also expression in the notochord at this stage (FIG. 9B). At E10.5, expression is seen in differentiated motor neurons as determined by Islet-1 expression (FIG. 9 B,K). Interestingly, the most newly developed motor neurons in the ventral-medial neural tube (which express Islet-1) do not express S52. This may suggest that expression of S52 occurs in more mature motor neurons.

During E11.5-E13.5 expression of S52 is maintained in the motor neurons and the floor plate. By this stage, expression is also detected in more dorsal cell types (FIG. 9 C-E). Expression is first seen in a subset of cells in the dorsal neural tube (denoted as I1, FIG. 9C) at E11.5. Staining appears to be retained in these at E12.5 and E13.5. By E12.5 there is expression within a subset of cells in the medio-lateral neural tube (denoted as 12, FIG. 9D). By E13.5 (FIG. 9E) the domain of expression is very strong in the medio-lateral subset of cells. Although the exact types of S52 expressing cells in the more dorsal neural tube were not determined in this study, based on their positions, they are likely to be subsets of dorsal interneurons including commissural or association interneurons (Wentworth, 1984, J. Comp. Neurol. 222 96).

Preliminary expression pattern analysis of Crim1 in the anterior neural tube was undertaken. Section in situ hybridisation of transverse sections of E10.5, showed strong expression in the floor plate of the midbrain (FIG. 9O). Analysis of a whole mount sagittal section of E12.5, shows expression of S52 in the floor plate of the hindbrain and the midbrain (FIG. 9R). As well, expression is found in the dorsal midline of the hindbrain and the roof of the diencephalon, which gives rise to the corpus callosum. This suggests a role for S52 in development of the midbrain and the hindbrain, and possibly the diencephalon of the forebrain.

Expression Pattern of Crim1 in the Developing Urogenital Tract

In other data not shown, during kidney development, Crim1 showed expression both in the ureteric tree, the early condensing mesenchyme and distal comma-shaped bodies. This considerably overlaps with the expression patterns of BMP2 and BMP7. The strong expression in pretubular aggregate and early comma shaped bodies is identical to that of BMP2. As the nephron elongates, Crim1 becomes expressed in the proximal end of the S-shaped bodies together with BMP7 and WT1. Crim1 also displays a striking male-specific expression pattern in the fetal gonads, its expression strongest in the Sertoli cells of the developing testis. This sexually dimorphic pattern of expression in not likely to determine sex, but may be important in normal testicular maturation, where the IGFs and members of the TGFβ superfamily are involved in testicular development.

14.5. Chromosomal Localisation of Human CRIM1

Based on the restricted pattern of expression and the highly level of conservation of CRIM1, it is likely that CRIM1 may have an important role in normal embryonic development. Therefore, chromosomal localisation was performed to identify any known human diseases which localize to the same chromosomal position as CRIM1.

Radiation Hybrid Mapping of CRIM1

Crim1 was localized using the Genebridge 4 radiation hybrid DNA panel (Gyapay et al., 1996, supra). This assay depends upon being able to distinguish between human and hamster sequences at the locus of interest. Comparison of human and mouse Crim1 cDNA sequences revealed a region with a number of differences. Primers were made from this sequence (3′ PCR 1F and 1R) which amplified human cDNA, human genomic DNA, but neither mouse cDNA nor mouse genomic DNA (FIG. 10A). This allowed assaying of the human-hamster radiation hybrid panel by only amplifying PCR products from hybrids which contained the human Crim1 locus.

Crim1 was positioned on chromosome 2 between D2S1852 (on band 2p21) and D2S1736 (on band 2p16.3) (FIG. 10B), by pairwise analysis with a high statistical significance (a maximum LOD score of greater than 16).

Confirmation of this location was determined by fluorescence in situ hybridization (FISH). Three human PAC clones containing Crim1 (PAC28; PAC309 5′ end; E2A4 3′ end) were labeled with biotin or digoxygenin and hybridized to human metaphase chromosomes, thereby revealing specific and identical fluorescent signals on both chromatids of the short arm of human chromosome 2, in the region of p21-22. This was confirmed for each of the three PAC clones in a total of 104 chromosomes. The Crim1 gene therefore localized to chromosome 2p21-16.3.

14.5 Immunofluorescence Analysis of CRIM1

A rabbit polyclonal antibody raised against the C-terminal 41 amino acids of CRIM1 was used to examine the localization of S52 in developing kidney, as shown in FIG. 11. This analysis indicated that the predominant site of CRIM1 protein expression is in the developing and branching ureteric tree of the kidney. This is in agreement with the RNA in situ hybridization analysis of mouse kidney. Some background staining was evident in the epithelializing mesenchyme, which is likely to be derived from the Cy3-labeled secondary antibody. Labeling is much stronger in the ureteric tree, especially at the growing tips.

14.6 Biochemical Characterisation and Subcellular Localisation of CRIM1

Using the antibodies previously described to both the N- and C-terminal ends of the CRIM1 protein and the use of myc epitope tagging, the present inventors have been able to examine characteristics of the CRIM1 protein by transfection of Crim1-containing expression constructs into cell lines.

Mammalian expression constructs were created from human CRIM1 using the pcDNA3 vector which employs a CMV promoter. These contained either the full length CRIM1 protein with an N-terminal myc epitope tag, full length with a C-terminal myc epitope tag or an ectodomain construct which does not include the transmembrane domain or C-terminal end of the protein. This ectodomain construct had a myc epitope tag at its C-terminal end, just past CR6 (see FIG. 12A)

The present inventors have also established via Western blot analysis of cell fractions and immunofluorescence of transfected cells that the full length CRIM1 protein is targeted for insertion into the plasma membrane and that it acts as a Type 1 transmembrane protein with the N-terminal end facing the media and the cytoplasmic tail facing the cytoplasm. In Western analyses of cellular fractions, full length CRIM1 protein is predominantly located within the light membrane fraction, which predominantly includes the plasma membrane, with some protein present in the heavy membrane fractions, which includes the endoplasmic reticulum (ER; FIG. 12B). Immunofluorescence of permeabilised cells with antibodies to either end of full length CRIM1 reveal a staining pattern consistent with the ER, suggesting that trafficking is occurring (FIG. 12 E,F&G). Immunofluorescence of non-permeabilised cells reveals uniform staining of the Crim1 protein all over the cell surface using the N-terminal antibody (FIG. 12H). Cells transfected with ectodomain was detectable within the ER of permeabilised cells using an anti-myc epitope antibody, but was not seen on non-permeabilised cells, suggesting that the secreted ectodomain protein is not attaching to the cell surface (FIGS. 12 I&J). Hence, the putative transmembrane domain does insert into the plasma membrane with the potential growth factor binding sites facing the location of secreted growth factors (Type 1 transmembrane protein).

As assessed by Western analysis after cell culture on FCS-free OPTIMEM media, full length CRIM1 protein does not appear to be secreted into the cell culture media. In contrast, an ectodomain construct of CRIM1 which does not contain the transmembrane domain or the C-terminal region, is freely secreted into the cell culture media (FIGS. 12 C,I &,J). This secreted protein does not associate with the outside surface of the cell as evidenced by a lack of visible immunofluorescence of cells transfected with the ectodomain construct as detected by an anti-myc epitope antibody (FIG. 12I).

The size of the ectodomain protein within the secretory pathway inside the cell appears smaller than once it has been secreted into the media (see arrows, FIG. 12C). To investigate further whether this resulted from glycosylation of the protein, protein harvested from cell culture media was treated with N-Glycosidase F(Boehringer Mannheim). The reduced the size of the protein (by 10-15 kDa), suggested the existence of N-linked glycosylation sites (FIG. 12D). An analysis of the sequence revealed three potential N-linked glycosylation sites, NES at the end of the IGFBP motif, NPT within CR3 and NNS between CR4 and CR5. This reveals that the protein is decorated with carbohydrates.

Anti-CRIM1 antibodies were used to investigate the size of endogenous CRIM1 protein to look for evidence of proteolytic cleavage or multiple isoforms. Aqueous and vitreous humor was used as a potential source of CRIM1 protein due to the high expression of the Crim1 gene within the epithelial cells of the lens and the abundance of TGFβ superfamily and insulin-like growth factors within the humors. A protein of approximately 150 kD was detected in both aqueous and vitreous humor using antibodies to both the N- and C-terminal ends of CRIM1. This suggest that processing of CRIM1 into a soluble form can occur in vivo (see FIG. 12D).

14.7 Characterisation of Interactions Between CRIM1 and Members of the TGFβ Superfamily

To directly investigate the ability of CRIM1 protein to interact with members of the TGFβ superfamily, myc-tagged BMP4 and Crim1 constructs were co-transfected into COS cell lines and immunoprecipitation studies were performed under non-denaturing conditions using the N-terminal CRIM1 antibody. This was suboptimal as this antibody prefers to recognise denatured protein. However, these studies showed that the BMP4 preprotein within the cell lysate was immunoprecipitated together with CRIM1. Only occasional weak evidence existed to show that the secreted processed form of BMP4 was binding CRIM1 ectodomain. This may mean that CRIM1 only interacts with the preprotein or that the levels of BMP4 and CRIM1 present in the media were too low to detect an interaction. However, this is preliminary evidence that CRIM1 does interact with BMP4 (see FIG. 13A).

Co-transfections were also examined before immunoprecipitation. The presence of CRIM1 did not increase the degree of processing and secretion of either BMP4 or BMP7 (see FIG. 13B). However, the presence of either BMP4 or BMP7 within the cell resulted in the secretion of the full length CRIM1 protein (see FIG. 13C). Hence, either directly or indirectly, BMP can elicit the secretion of a CRIM1-derived protein of a similar size to that detected in aqueous humour. It is not clear how this occurs. However, this ability of BMPs to elicit processing of CRIM1 is further evidence for a functional interaction between the BMPs and CRIM1.

Ligand blotting was used to see if the CRIM1-like protein within aqueous humour could interact with recombinant TGFβ2. Aqueous humour was electrophoresed under non-denaturing conditions and transferred to PVDF membranes. Membranes were then incubated with or without recombinant TGFβ2, before being bound with TGFβ2 antibodies. A band equivalent to the CRIM1-like protein was detected with the TGFβ2 antibody, suggesting that the TGFβ2 does interact with this protein. The same band was detected in vitreous humour (see FIG. 13D).

Cell overlay assays were also used to investigate whether recombinant BMP4 could bind to CRIM1 expressing cells. COS cells were transfected with full length N-terminal myc-tagged Crim1 constructs. Recombinant protein was added to these cells in culture before analysis via immunofluorescence using antibodies to BMP4. CRIM1-expressing cells did stain positive with the BMP4 antibody only when the recombinant protein had been added to the cultures. This pattern was not seen in untransfected cells, indicating that BMP4 was binding to cells expressing full length CRIM1 protein but not to non-expressing cells (see FIG. 13E).

14.8 Purification and Bioassay of Recombinant CRIM1 Ectodomain

For the purposes of functional assays, a subclone of Crim1 encoding only the N-terminal extracellular portion of the protein (amino acids 1-901) including an N-terminal myc epitope tag was generated as a mammalian expression construct. This ectodomain construct contains all 6 cysteine-rich motifs plus the IGFBP, but not the putative transmembrane domain or the cytoplasmic tail. The construct was transiently transfected into COS7 cells. Western blot analysis of cell lysates versus cell culture media confirmed that this fragment is freely secreted. A stable transformant producing CRIM1 ectodomain has been produced although the level of protein production is lower than post transfection.

Purification of myc-tagged CRIM1 ectodomain protein from transiently transfected cells was performed using an anti-myc epitope monoclonal antibody affigel column as described hereinbefore (se FIG. 14A).

In the developing neural tube, the dorsal cell type differentiation appears to be controlled, at least in part, by BMPs and members of the TGF-β superfamily which are secreted by the roof plate while differentiation of motor neuron in the ventral spinal cord is inhibited by these dorsally-derived factors.

A possible biological activity for the CRIM1 protein was examined by ectopically expressing the full length or ectodomain CRIM1 proteins in embryonic chick spinal cord using in ovo electroporation technique. Such spinal cord electroporations have resulted in disruption to the migration of neural crest cells from the spinal cord to form the dorsal root ganglia. Ectopic expression of CRIM1 in the dorsal neural tube in chick embryo also appears to repress the determination of dorsal interneurons, as assessed by the number of engrailed-1 (En-1) expressing cells, and ventral motor neurons, as assessed by the number of Islet-1 (I1-1) expressing cells (see FIG. 14B). These results suggest a role for CRIM1 in neural tube and neural crest development in vertebrates.

### EXAMPLE 15

### Discussion

CRIM1 Structure

The human, mouse and chicken Crim1 nucleic acids of the invention are predicted to encode a relatively large transmembrane protein of approximately 120 kD, with several highly conserved domains shared with other known proteins. Three lines of evidence suggest that this protein is a single pass integral membrane protein with a large extracellular domain. Firstly, there is a hydrophobic stretch of amino acids at the N-terminus, which is predicted to be a signal peptide (Nielsen et al., 1997a, supra).

Secondly, there is a transmembrane domain toward the C-terminus predicted by hydropathy plot analysis. Finally, the N-terminal domain contains six cysteine rich repeat (CRR) motifs, an RGD motif and an IGFBP-like motif, which are found within secreted proteins or within the extracellular domain of integral membrane proteins (Shimasaki & Ling, 1991, Prog. Growth. Factor Res. 3 243; Francois et al., 1994, Genes Dev. 8 2602). There is a high level of conservation of the N-terminal domain in the human and mouse CRIM1 sequences, and the C. elegans homolog. This suggests that CRIM1 is important in processes involving extracellular protein-protein interactions.

Cysteine rich repeats (CRRs) are motifs (58-70 amino acids) containing 10 conserved cysteines in a defined spacing pattern. These motifs have been found in a variety of proteins including the secreted precursor of type II collagen (Ryan & Sandell, 1990, J. Biol. Chem. 265 10334), the adhesion factors thrombospondin (Dixit et al., 1986, supra) and Von Willebrand Factor (Hunt & Barker, 1987, supra), NEL (Atsuhashi et al., 1995, supra), NEL-like proteins (NELL1 and NELL2; Watanabe et al., 1996, Genomics 38 273), and the cell patterning proteins, chordin (Sasai et al., 1994, supra; Streit et al., 1998, Dev. Suppl. 125 507) and short gastrulation (Francois et al., 1994, supra). The CRR in thrombospondin corresponds to a region which can bind to collagen (Dixit et al., 1986, supra), whilst in procollagen it appears important for oligomerization of mature collagen (Ryan & Sandell, 1990, supra). Furthermore, the CRRs in Sog and Chd appear important in binding to members of the TGF-β family (Piccolo et al., 1996, Cell 86 589). Therefore, these motifs in CRIM1 may be important in protein-protein interactions with other proteins found in the extracellular space.

At the predicted N-terminus of the mature CRIM1 protein is a domain which is found in a variety of proteins, which domain is characterized by the presence of an Insulin-like growth factor binding protein (IGFBP) like motif. This motif, which includes 8 highly conserved cysteines (Kim et al., 1997, Proc. Natl. Acad. Sci USA 94 12981), has been found in all six known IGFBPs (Shimasaki & Ling, 1991, supra). Furthermore, this domain has been found in the secreted proteins MAC25 (Murphy et al., 1993, Cell Growth Differ. 4 715), NOV (Martinerie et al., 1992, Oncogene 7 2529) connective tissue growth factor (CTGF) (Bradham et al., 1991, J. Cell Biol. 114 1285) CYR61 (O'Brien et al., 1990, Mol. Cell. Biol. 10 3569) and Drosophila twisted gastrulation (TSG) (Mason et al., 1994, Genes Dev. 8 1489). Mutation and structural analysis has revealed that this domain may fold as a separate tertiary structure through disulfide bonds (Drop et al., 1992, supra; Forbes et al., 1998, J. Biol. Chem. 273 4647). Furthermore, in vitro studies have shown that this domain is important for binding to growth factors such as IGF and insulin (Oh et al., 1996, J. Biol. Chem. 271 30322; Kim et al., 1997, supra). This suggests that CRIM1 may be actively involved in binding to and mediating the activity of certain growth factors in the extracellular space.

A remarkable outcome of this study was the striking conservation of CRIM1/Crim1 throughout evolution. An uncharacterized putative ortholog in C. elegans (B0024.4; Wilson et al., 1994, Nature 368 32) was identified by BlastP searching and demonstrated significant similarity to the human and mouse Crim1 polypeptide sequences. The C. elegans polypeptide has a very similar domain structure to the human and mouse CRIM1 polypeptides, but appears to be truncated at the N-terminus, containing neither a signal peptide or an IGFBP domain. However, the C. elegans ORF was predicted by a combination of genomic sequencing and overlapping EST clones, and may not encode a full length protein sequence (Wilson et al., 1994, supra). In all three species, 95% of cysteine residues were found to be conserved in the extracellular domain, suggesting that these homologs and orthologs have very similar structures.

The presence of Crim1 genes in chicken (Gallus gallus) and zebrafish (Danio rerio) predicts that highly homologous proteins are present in a variety of vertebrate species. Due to this conservation, it is likely that Crim1/CRIM1 and its homologues have important and conserved roles in both vertebrates and invertebrates.

The expression pattern of Crim1 together with sequence data suggests that this gene may be involved in dorsal-ventral patterning by regulating BMP mediated signalling patterning in the CNS. BMP proteins are expressed widely throughout the developing central nervous system. Antagonism of BMP proteins by secreted factors such as Noggin, Chordin and Follistatin has been shown to be an important conserved mechanisms of patterning throughout vertebrate development (Holley et al., 1995, Nature 376 249; Sasai et al., 1995, supra; Zimmerman et al., 1996, Cell 86 599; Fainsod et al., 1997, Mech. Dev. 63 39).

A gradient of signalling across the newly developed neural tube is thought to establish a basic dorsal-ventral polarity, determining the fates of neurons and glia by the dorsal-ventral position of their precursor cells in the neural tube (Tanabe & Jessell, 1996, supra). This patterning is thought to be controlled by Shh from the notochord and floor plate, and BMP proteins (BMP4, BMP5, BMP7 and DSL-1) from the epidermal ectoderm and roof plate (Roelink et al., 1994, Cell 76 761; Liem et al., 1995, Cell 82 969; Roelink et al., 1995, Cell 81 445; Liem et al., 1997, Cell 91 127).

A key structural feature of CRIM1 is the multiple CRRs. Of the proteins which contain CRRs, Drosophila Sog and Xenopus chordin are of most interest because their overall structural organisation is similar to that of CRIM1. Both Chd and Sog gene products are thought to bind to TGF-β superfamily members and antagonize intercellular signalling (Sasai et al., 1994, supra; Piccolo et al., 1996, supra; Yu et al., 1996, supra). In vitro binding assays have shown that Chd protein can bind BMP4 with high affinity (Piccolo et al., 1996, supra). In Xenopus, direct inhibition of BMP4 by Chd (secreted from the organiser), has been shown to act as a dorsalising factor during gastrulation, and result in formation of dorsal structures such as neural tissue (Sasai et al., 1994, supra; Piccolo et al., 1996, supra). In Drosophilia, Sog has been shown by genetic epistasis to inhibit the signalling of decapentapegic (a TGF-β superfamily member which very similar to Xenopus BMP4), resulting in ventralisation of the gastrulating fly embryo (and induction of neural tissue) (Francois et al., 1994, supra). Since overexpression of Sog in Xenopus, can rescue null mutants of Chd, it has been postulated that Sog and Chd encode for proteins which are functionally equivalent (Schmidt et al., 1995a, Development 121 4319).

However, there is significant homology between the two proteins only within the CRRs (Francois & Bier, 1995, Cell 80 19). Although the exact structural and biochemical properties of CRRs are largely known, it is likely that the CRRs in Sog and Chd are important for binding to TGF-β molecules.

CRIM1 and the Eye

CRIM1 is strongly expressed in developing lens. and may play a role in maintaining this layer of cells as an epithelium. Hence, CRIM1 may act in an anti-cataractogenic fashion. Significant evidence exists to suggest that within the eye, TGFβ, particularly TGFβ2, can act in a cataractogenic fashion, leading to disruption in the morphology of the epithelial cells covering the front of the lens. The result of addition of recombinant TGFβ2 to lens explants cultures is the production of plaques identical in histology to those seen arising after surgery for the removal of cataracts. The suggestion that inhibitors of TGFβ will act in an anti-cataractogenic fashion is supported by the disclosures of International Publication WO95/13827 and International Application WO98/26784, each of which is incorporated herein by reference.

As described herein, CRIM1 is capable of interacting with TGFβ superfamily members such as TGFβ2 and BMP4. This strongly suggests that CRIM1 is involved in regulating the activities of TGFβ superfamily members and may therefore have therapeutic value in cataractogenesis (via interaction with TGFβ2) and bone formation and remodelling (via interaction with BMPs).

CRIM1 and Bone Development

TGFβ superfamily, including the bone morphogenetic proteins (BMPs), are expressed during, and are critical for, early embryo development and organogenesis of a variety of organs, including the kidney, eye (cornea and lens), heart, skeleton, tooth. limb and central nervous system. By interacting with members of the (bone morphogenic protein) BMP, GDF, activin or TGFβ family, CRIM1 may have a role in development, remodelling or repair of these various organs. More specifically, CRIM1 may be able to modulate activities such as bone remodelling, tissue regeneration and motor neuron specification. The genes may be useful in the diagnosis of diseases of these organs and the proteins or genes encoding them in some form of gene therapy construct in the treatment of such conditions. Several of the BMPs have the property of generating bone, including BMP2 and BMP7 (OP-1), and OP-1 is already known to have potential in bone remodelling, including the treatment of periodontal and orthopedic indications such as fractures.

CRIM1 and Kidney/Gonad Development

The expression pattern of Crim1 in the kidney and gonads overlaps with the expression patterns of BMP2 and BMP7. Several of the BMPs are expressed strongly during the development of the kidney, including BMP2,4 ,5 and 7, as for example reviewed in Godin et al., 1999, supra. More particularly, an important role for BMP7 has been shown in the formation of the kidney and defects including renal dysgenesis, cystic kidneys or agenesis.

BMP7 expression in the kidney continues after birth, as are receptors for BMP on the podocytes within the glomeruli. Application of BMP7 has been shown to decrease the loss of kidney function associated with acute ischaemic injury (Vukicevic et al., 1998, J. Clin. Invest. 102 202). It can also inhibit tubulointerstitial fibrosis and inflammation after unilateral ureteral obstruction. CRIM1 may similarly assist in such conditions by facilitating or increasing the duration of such BMP7 activity. BMP7 (OP-1) has been found to be preventative for renal fibrosis associated with ureteral obstruction. OP-1 administration can prevent tubular atrophy and diminish the activation of tubulointerstitial inflammation and fibrosis, thereby preserving renal function (Hrusuka et al., 2000, Am. J. Renal. Physiol. 279 130).

Within the kidney, TGFβ has been implicated in vascular remodelling, premature termination of normal nephrogenesis, promotion of a transition of epithelial cells to mesenchymal cells and a variety of other effects. Increases in circulating TGFβ1 occur during diabetes. This may contribute to the onset of diabetic nephropathy via the induction of collagens 3 and 1 which result in scarring and fibrosis within the kidney.

Therefore, through interactions with TGFβ and or BMPs such as OP-1, CRIM1 may regulate kidney and gonad development (such as testicular maturation), and thereby constitute a therapeutic candidate for renal and gonadal diseases such as described above.

CRIM1 and Neuronal Development

The spatial and temporal control of neuron differentiation by adjacent cells along the dorsal-ventral axis are important even after initial cell patterning by Shh and BMPs. Whilst early patterning by Shh and BMP proteins was thought to be sufficient to specify the cellular subtypes along the dorsal-ventral axis, various in vivo experiments have shown that other signalling factors must also be involved at later stages of dorsal-ventral patterning. The control of differentiation of ventral interneurons which express the homeodomain containing gene, Engrailed-1 (En-1), was shown to be dependant on the concentration of Shh-N in vitro (Ericson et al., 1996, supra). However, this class of ventral interneuron was missing in the mouse in which more ventrally located motor neurons are eliminated by the targeted inactivation of Islet-1, a gene essential for generation of motor neurons (Pfaff et al., 1996, Cell 84 309). It is possible that other intercellular signalling molecules expressed by motor neurons which are involved in the process of En-1 interneuron differentiation. Such factors may effect dorsal-ventral patterning by interacting with either Shh or BMPs, and regulating the local concentration of these signalling.

CRIM1 may be actively involved in patterning neighbouring cells after initial dorsal-ventral pattern has been established by Shh and BMPs. Expression of S52 mRNA in the motor neurons appears at the right time (E10.5) to influence differentiation of interneurons expressing En-1 (Pfaff et al., 1996, supra). CRIM1 could conceivably achieve this by inhibition of BMP proteins or other TGF-β signalling molecules, which are expressed in the dorsal neural tube (Liem et al., 1995, supra; Liem et al., 1997, supra). By inhibiting BMP proteins, CRIM1 could allow differentiation of these ventral interneurons. Expression of CRIM1 in other populations of neurons such as dorsal interneurons may have similar functions in cell patterning, allowing specification of adjacent cell types.

Another function of signalling molecules expressed by the floor plate is the control of axon guidance. The secreted signalling molecule NETRIN-1, and various cell surface proteins, such as those of the immunoglobulin superfamily are expressed by the floor plate and are known to be involved in axon guidance in the central and peripheral nervous system (Tessier-Lavigne & Goodman, 1996, supra).

The results of expression pattern analysis presented in this study, suggest that CRIM1 may be involved in axon guidance. Firstly, onset of expression in the floor plate occurs around E9.5-E10.5 in mouse (FIG. 9), a time when commissural neurons are thought to form and begin sending axons toward the floor plate (Colamarino & Tessier-Lavigne, 1995, Cell 81 621). Secondly, expression occurs in distinct subsets of neurons along the lateral neural tube during early development defining a pathway by which axons of commissural, association, and sensory neurons move throughout the lateral neural tube (Wentworth, 1984, supra). Since Crim1 appears to encode a transmembrane bound protein which could be involved in extracellular interactions, this molecule may be involved in cell-cell contact dependant guidance of axons in the neural tube.

Embryonic expression of the Crim1 gene occurs in the notochord and floor plate, which are known to be the source of the embryonic organising centre for the developing central nervous system (CNS). The nucleotide sequence, protein sequence and conservation of the CRIM1 homologues in human, mouse and chick, and C. elegans predict the essential role of CRIM1 conserved function during animal evolution. These findings suggest that CRIM1 functions as part of signalling mechanism which is required for normal CNS development. This may also be important in tissue regeneration including kidney replacement therapy.

CRIM1 protein, nucleic acids encoding said protein, and interacting proteins which selectively bind such proteins may function as a regulator for normal neuronal differentiation in the spinal cord, and migration of neural crest-derived cells, by either direct or indirect interactions with other growth factors such as BMPs, TGFβs and IGFs that are thought to be involved in the normal and/or abnormal neuronal differentiation in mammalian CNS. CRIM1 may also function as a neural cell adhesion molecule that is required for the normal development and maintenance of neurons in the CNS during normal embryonic development in adult. CRIM1 may also promote development of neuronal processes such as axons in developing CNS.

CRIM1 protein, or nucleic acids encoding CRIM1 and interacting proteins which selectively bind such proteins will also find use in screening chemical libraries for regulators of neural differentiation, cell migration, adhesion and neuronal process growth, in genetic mapping, as probes for related genes, as diagnostic reagents for genetic neurological disease and in the production of specific-cellular and animal systems for the development of neurological disease therapy, particularly for conditions such as motor neuron disease. They may also be important in the derivation and in vitro culture of neural stem cells for stem cell therapy of neurological conditions.

Conclusion

In light of the foregoing, it will be appreciated that the human Crim1 nucleic acid, the Crim1 genomic sequence and the CRIM1 polypeptide isolated by the present inventors, may provide new and useful diagnostic and therapeutic tools applicable to one or more of a variety of diseases such as those described above. The Crim1 nucleic acids of the invention are also useful in chromosomal mapping studies and analysis of tissue type and tissue and organ development.

Throughout this specification the aim has been to describe the preferred embodiments of the invention without limiting the invention to any one embodiment or specific collection of features. It will therefore be appreciated by those of skill in the art that, in light of the instant disclosure, various modifications and changes can be made in the particular embodiments exemplified without departing from the spirit and scope of the present invention.

It will also be appreciated that all patent and scientific literature and computer programs described in this specification are incorporated in their entirety herein by reference.

1. An isolated nucleic acid which encodes a polypeptide comprising the amino acid sequence PGECCPLP (SEQ ID NO: 1).

2. The isolated nucleic acid of claim 1, which comprises a sequence of nucleotides selected from the group consisting of SEQ ID NO: 5, SEQ ID NO: 6 and SEQ ID NO: 7.

3. The isolated nucleic acid of claim 1, which comprises a nucleotide sequence as set forth in FIG. 3 (SEQ ID NOS: 20-24).

4. The isolated nucleic acid of claim 1 as deposited in pcDNA3-hCRIM1myc under accession number NM00/16530 at AGAL on Nov. 9, 2000.

5. A nucleic acid homolog of the isolated nucleic acid of claim 2, wherein said homolog is capable of hybridizing to said isolated nucleic acid under at least medium stringency wash conditions.

6. An expression construct comprising the isolated nucleic acid of claim 1.

7. A host cell comprising the expression construct of claim 6.

8. A pharmaceutical composition for gene therapy comprising the expression construct of claim 6 and a pharmaceutically acceptable carrier, diluent or excipient.

9. An isolated polypeptide encoded by the isolated nucleic acid of claim 1.

10. An isolated polypeptide comprising the amino acid sequence PGECCPLP (SEQ ID NO: 1).

11. An isolated polypeptide according to claim 10, further characterized by the presence of six cysteine-rich domains, an RGD domain, an IGFBP-like domain and a transmembrane domain.

12. The isolated polypeptide of claim 10, wherein the polypeptide comprises an amino acid selected from the group consisting of the sequences set forth in SEQ ID NO: 2, SEQ ID NO: 3 and SEQ ID NO: 4.

13. A biologically-active fragment, variant or derivative of the polypeptide of claim 12.

14. The biologically-active fragment of claim 13, which consists essentially of an amino acid sequence selected from the group consisting of:
(i) amino acids 1-901 of SEQ ID NO: 2;
(ii) SEQ ID NO: 8; and
(iii) SEQ ID NO: 9.

15. An antibody capable of binding the isolated polypeptide of claim 9.

16. The antibody of claim 15, which antibody is capable of binding a peptide having an amino acid sequence selected from the group consisting of:
(i) SEQ ID NO: 8; and
(ii) SEQ ID NO: 9.

17. A pharmaceutical composition comprising the isolated polypeptide, biologically active fragment, variant or derivative thereof according to claim 9 and a pharmaceutically acceptable carrier, diluent or excipient.

18. A mimetic which antagonizes or mimics a biological activity of a CRIM1 polypeptide according to claim 9.

19. A method of modulating the biological activity of a polypeptide of the BMP family, said method including the step of administering to an animal a pharmaceutical composition according to claim 8.

20. The method of claim 19, wherein the biological activity of the polypeptide of the BMP family is increased.

21. The method of claim 19, wherein the biological activity of the polypeptide of the BMP family is decreased.

22. The method of claim 21, wherein the pharmaceutical composition is administered to the animal to prophylactically or therapeutically treat an eye disease.

23. The method of claim 22, wherein the eye disease is cataract formation.

24. A method of modulating the biological activity of a polypeptide of the BMP family, said method including the step of administering to an animal a pharmaceutical composition according to claim 17.

25. The method of claim 24, wherein the biological activity of the polypeptide of the BMP family is increased.

26. The method of claim 24, wherein the biological activity of the polypeptide of the BMP family is decreased.

27. The method of claim 26, wherein the pharmaceutical composition is administered to the animal to prophylactically or therapeutically treat an eye disease.

28. The method of claim 27, wherein the eye disease is cataract formation.

29. A method of determining whether an animal is predisposed to a genetically-heritable disease, which method includes the steps of:—
(i) obtaining a nucleic acid sample from said animal; and
(ii) determining whether said nucleic acid sample includes a mutation or polymorphism in an isolated nucleic acid according to claim 1, said mutation or polymorphism being indicative of said animal being predisposed to, or suffering from, said genetically-heritable disease.

30. The method of claim 19, wherein the animal is a mammal.

31. The method of claim 30, wherein the mammal is a human.

32. The method of claim 29, wherein the animal is a mammal.

33. The method of claim 32, wherein the mammal is a human.

34. An isolated polypeptide encoded by the nucleic acid homologue of claim 5.
